(19) United States
(12) Patent Application Publication
(10) Pub. No.: US 2013/0040978 A1
(43) Pub. Date: Feb. 14, 2013

(54) SPIRO ISOXAZOLINE COMPOUNDS AS SSTR5 ANTAGONISTS

(71) Applicants: Joseph L. Duffy, Cranford, NJ (US); Jianming Bao, Princeton, NJ (US); Debra L. Ondeyka, Fanwood, NJ (US); Sriram Tyagarajan, Edison, NJ (US); Patrick Shao, Fanwood, NJ (US); Feng Ye, Scotch Plains, NJ (US); Revathi Katipally, Monmouth Junction, NJ (US); Paul E. Finke, Milltown, NJ (US); Yi Zang, Princeton, NJ (US); Michael A. Plotkin, Frenchtown, NJ (US); F. Anthony Romero, Westfield, NJ (US); Remond Moningka, Rahway, NJ (US); Zahid Hussain, Dayton, NJ (US)

(72) Inventors: Joseph L. Duffy, Cranford, NJ (US); Jianming Bao, Princeton, NJ (US); Debra L. Ondeyka, Fanwood, NJ (US); Sriram Tyagarajan, Edison, NJ (US); Patrick Shao, Fanwood, NJ (US); Feng Ye, Scotch Plains, NJ (US); Revathi Katipally, Monmouth Junction, NJ (US); Paul E. Finke, Milltown, NJ (US); Yi Zang, Princeton, NJ (US); Michael A. Plotkin, Frenchtown, NJ (US); F. Anthony Romero, Westfield, NJ (US); Remond Moningka, Rahway, NJ (US); Zahid Hussain, Dayton, NJ (US)

(21) Appl. No.: 13/641,767

(22) PCT Filed: May 13, 2011
(86) PCT No.: PCT/US11/36367

§ 371 Date: Oct. 17, 2012

Related U.S. Application Data
(60) Provisional application No. 61/345,683, filed on May 18, 2010.

(51) Int. Cl. C07D 498/10; C07D 519/00; A61P 25/22; A61P 3/04; A61P 3/00; A61P 25/24; A61K 31/438; A61P 3/10

(52) U.S. Cl. 514/278; 546/19

(57) ABSTRACT

Substituted spirocyclic amines of structural formula (I) are selective antagonists of the somatostatin subtype receptor 5 (SSTR5) and are useful for the treatment, control or prevention of disorders responsive to antagonism of SSTR5, such as Type 2 diabetes, insulin resistance, lipid disorders, obesity, atherosclerosis, Metabolic Syndrome, depression, and anxiety.

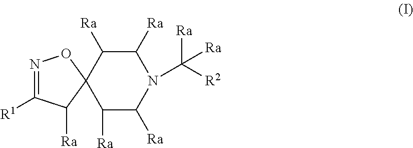

### FIELD OF THE INVENTION

The instant invention is concerned with substituted spiro isoxazoline compounds, which are selective antagonists of the somatostatin subtype receptor 5 (SSTR5) and are useful for the treatment, control or prevention of disorders responsive to antagonism of SSTR5, such as Type 2 diabetes mellitus, insulin resistance, obesity, lipid disorders, atherosclerosis, Metabolic Syndrome, depression, and anxiety.

### BACKGROUND

Diabetes is a disease derived from multiple causative factors and characterized by elevated levels of plasma glucose (hyperglycemia) in the fasting state or after administration of glucose during an oral glucose tolerance test. There are two generally recognized forms of diabetes. In Type 1 diabetes, or insulin-dependent diabetes mellitus (IDDM), patients produce little or no insulin, the hormone which regulates glucose utilization. In Type 2 diabetes, or noninsulin-dependent diabetes mellitus (NIDDM), insulin is still produced by islet cells in the pancreas. Patients having Type 2 diabetes have a resistance to the effects of insulin. These patients often have normal levels of insulin, and may have hyperinsulinemia (elevated plasma insulin levels), as they compensate for the reduced effectiveness of insulin by secreting increased amounts of insulin (Polonsky, Int. J. Obes. Relat. Metab. Disord. 24 Suppl 2:S29-31, 2000). The beta cells within the pancreatic islets initially compensate for insulin resistance by increasing insulin output. Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver. Eventually, a patient may be become diabetic due to the inability to properly compensate for insulin resistance. In humans, the onset of Type 2 diabetes due to insufficient increases (or actual declines) in beta cell mass is apparently due to increased beta cell apoptosis relative to non-diabetic insulin resistant individuals (Butler et al., Diabetes 52:102-110, 2003).

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with Type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, effective therapeutic control of glucose homeostasis, lipid metabolism, obesity, and hypertension are critically important in the clinical management and treatment of diabetes mellitus.

Patients who have insulin resistance often exhibit several symptoms that together are referred to as Syndrome X or Metabolic Syndrome. According to one widely used definition, a patient having Metabolic Syndrome is characterized as having three or more symptoms selected from the following group of five symptoms: (1) abdominal obesity, (2) hypertriglyceridemia, (3) low levels of high-density lipoprotein cholesterol (HDL), (4) high blood pressure, and (5) elevated fasting glucose, which may be in the range characteristic of Type 2 diabetes if the patient is also diabetic. Each of these symptoms is defined clinically in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with Metabolic Syndrome, whether they have or develop overt diabetes mellitus, have an increased risk of developing the macrovascular and microvascular complications that occur with Type 2 diabetes, such as atherosclerosis and coronary heart disease.

There are several available treatments for Type 2 diabetes, each of which has its own limitations and potential risks. Physical exercise and a reduction in dietary intake of calories often dramatically improves the diabetic condition and are the usual recommended first-line treatment of Type 2 diabetes and of pre-diabetic conditions associated with insulin resistance. Compliance with this treatment is generally very poor because of well-entrenched sedentary lifestyles and excess food consumption, especially of foods containing high amounts of fat and carbohydrates. Pharmacologic treatments have largely focused on three areas of pathophysiology: (1) hepatic glucose production (biguanides such as phenformin and metformin), (2) insulin resistance (PPAR agonists such as rosiglitazone and pioglitazone), (3) insulin secretagogues (sulfonylureas such as tolbutamide, glipizide, and glimepiride); (4) incretin hormone mimetics (GLP-1 derivatives and analogs, such as exenatide and luraglitide); and (5) inhibitors of incretin hormone degradation (DPP-4 inhibitors, such as sitagliptin, vildagliptin, saxagliptin, and alogliptin).

Recent research has focused on pancreatic islet-based insulin secretion that is controlled by glucose-dependent insulin secretion. This approach has the potential for stabilization and restoration of β-cell function. In this regard, research has been done on the affects of antagonizing one or more of the somatostatin receptors. Somatostatin (SST) is a cyclic tetradecapeptide hormone that is widely distributed throughout the body and exhibits multiple biological functions that are mostly inhibitory in function, such as the release of growth hormone, pancreatic insulin, glucagon, and gastrin.

SST hormone activity is mediated through SST-14 and SST-28 isoforms that differentially bind to the five different SST receptor subtypes (SSTR1-5). In humans SSTR1 and SSTR2 are found in the pituitary, small intestine, heart and spleen with SSTR2 predominately in the pancreas, pituitary and the stomach. SSTR3 and SSTR4 are found in the pituitary, heart, liver, spleen stomach, small intestine and kidney. SSTR5 is found in high concentration in the pituitary, as well as the pancreas. It has been shown that S-28 and S-14 bind with similar affinity to SSTR1, SSTR2, SSTR3, and SSTR4. The receptor SSTR5 can be characterized by its preferential affinity for S-28 (Chisholm et al., Am. J. Physiol Endocrinol Metab. 283:E311-E317 (2002)).

SSTR5 is expressed by human islet β cells that are responsible for producing insulin and amylin. Therefore, binding to the SSTR5 could affect insulin secretion. For example, by using in vitro isolated perfused pancreas preparations from 3-month-old mice, it was demonstrated that SSTR5 global knockout mice pancreata have low basal insulin production, but a near normal response to glucose stimulation. It was theorized that, since along with SSTR5, SSTR1 is also expressed in islet 0 cells up-regulated SSTR1 compensates for the loss of SSTR5 in young knockout mice. As the mice aged, however, SSTR1 expression decreased in both the knockout mice and the aged-control wild-type mice. With lower SSTR1 expression in vivo, SSTR5 knockout mice had increased basal and glucose stimulated insulin secretion due to near complete lack of SSTRs on the knockout mice islet β cells with subsequent loss of the inhibitory SST response (Wang et al., Journal of Surgical Research, 129, 64-72 (2005)).

The proximity of D cells producing S-28 and L-cells containing GLP-1 in the ileum suggest that S-28 acting through SSTR5 may additionally participate in the direct regulation of GLP-1 secretion. To determine if S-28 acting through SSTR5 participates in the direct regulation of GLP-1 secretion, fetal rat intestinal cell cultures were treated with somatostatin analogs with relatively high specificity for SSTR2-5. GLP-1 secretion was inhibited by an SSTR5-selective analog more potently that S-14 and nearly as effectively as S-28 (Chisholm et al., Am. J. Physiol Endocrinol Metab. 283:E311-E317, 2002). A selective antagonist of SSTR5 is anticipated to block the suppression of GLP-1 secretion by endogenous somatostatin peptides, thereby elevating circulating GLP-1 levels. Elevated endogenous GLP-1 levels are associated with beneficial effects in the treatment of Type 2 diabetes (Arulmozhi et al., European Journal of Pharmaceutical Sciences, 28, 96-108 (2006)).

US 2008/0293756 discloses 4,4 disubstituted piperidine derivatives as SST Receptor Subtype 5 antagonists useful to treat diabetes.

Small molecule SSTR antagonists are also disclosed in US 20080249101; WO 2008031735; WO 2008019967; WO 2006094682; WO 2006128803; WO 2007025897; WO 20070110340 and WO 2008000692.

Other small molecule and peptide SSTR antagonists known in the art are disclosed in Wilkinson et al., British Journal of Pharmacology 118, 445-447 (1996); Hocart et al., J. Med. Chem. 41, 1146-1154 (1998); Hay et al., Bioorg. Med. Chem. Lett. 11, 2731-2734 (2001), Martin et al., J. Med. Chem. 50, 6291-6295 (2007) and Guba et al., J. Med. Chem. 50, 6295-6298 (2007).

Described herein are selective, directly acting SSTR5 antagonists, which are useful as therapeutically active agents for the treatment and/or prevention of diseases that are associated with the modulation of SSTR5. Diseases that can be treated or prevented with SSTR5 antagonists include diabetes mellitus, impaired glucose tolerance and elevated fasting glucose.

### SUMMARY

The present invention is directed to compounds of structural formula I, and pharmaceutically acceptable salts thereof:

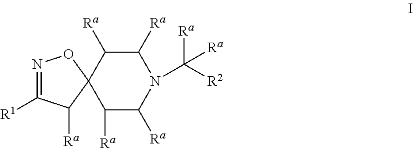

wherein each occurrence of R^{a} is independently selected from the group consisting of hydrogen, halogen, C₁-C₁₀alkyl and halogen-substitutedC-C₁₀alkyl; R¹ is selected from the group consisting of phenyl and heterocycle, wherein the phenyl and heterocycle are substituted with at least one substituent selected from α; R² is selected from the group consisting of phenyl and heterocycle, wherein the phenyl and heterocycle is substituted with 1-3 substituents independently selected from α; and α is selected from the group consisting of halogen, —C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

These substituted spiro isoxazoline are effective as antagonists of SSTR5, and are useful for the treatment, control or prevention of disorders responsive to antagonism of SSTR5, such as Type 2 diabetes, insulin resistance, lipid disorders, obesity, atherosclerosis, Metabolic Syndrome, depression, and anxiety.

The present invention also relates to pharmaceutical compositions comprising the compounds of the present invention and a pharmaceutically acceptable carrier.

The present invention also relates to methods for the treatment, control, or prevention of disorders, diseases, or conditions responsive to antagonism of SSTR5 in a subject in need thereof by administering the compounds and pharmaceutical compositions of the present invention.

The present invention also relates to methods for the treatment, control, or prevention of Type 2 diabetes, hyperglycemia, insulin resistance, obesity, lipid disorders, atherosclerosis, and Metabolic Syndrome by administering the compounds and pharmaceutical compositions of the present invention to a subject in need thereof.

The present invention also relates to methods for the treatment, control, or prevention of depression and anxiety by administering the compounds and pharmaceutical compositions of the present invention in a subject in need thereof.

The present invention also relates to methods of enhancing GLP-1 secretion by administering the compounds and pharmaceutical compositions of the present invention to a subject in need thereof.

The present invention also relates to methods for the treatment, control, or prevention of obesity by administering the compounds of the present invention in combination with a therapeutically effective amount of another agent known to be useful to treat obesity.

The present invention also relates to methods for the treatment, control, or prevention of Type 2 diabetes by administering the compounds of the present invention in combination with a therapeutically effective amount of another agent known to be useful to treat Type 2 diabetes.

The present invention also relates to methods for the treatment, control, or prevention of atherosclerosis by administering the compounds of the present invention in combination with a therapeutically effective amount of another agent known to be useful to treat atherosclerosis.

The present invention also relates to methods for the treatment, control, or prevention of lipid disorders by administering the compounds of the present invention in combination with a therapeutically effective amount of another agent known to be useful to treat lipid disorders.

The present invention also relates to methods for treating Metabolic Syndrome by administering the compounds of the present invention in combination with a therapeutically effective amount of another agent known to be useful to treat Metabolic Syndrome.

The present invention also relates to methods for the treatment, control, or prevention of depression and anxiety by administering the compounds of the present invention in combination with a therapeutically effective amount of another agent known to be useful to treat depression or anxiety.

The present invention also relates to the use of the compounds of the present invention in the manufacture of a medicament for the treatment, control or prevention of disorders, diseases, or conditions responsive to antagonism of SSTR5.

The present invention also relates to the use of the compounds of the present invention in the manufacture of a medicament for the treatment, control or prevention of Type 2 diabetes, hyperglycemia, insulin resistance, obesity, lipid disorders, atherosclerosis, and Metabolic Syndrome.

The present invention also relates to the use of the compounds of the present invention in the manufacture of a medicament for the treatment, control or prevention of depression, and anxiety.

The present invention also relates to the use of the compounds of the present invention in the manufacture of a medicament for the suppression of GLP-1 secretion in a subject in need thereof.

The present invention also relates to the use of the compounds of the present invention in the manufacture of a medicament that also includes a therapeutically effective amount of another agent for the treatment of diabetes.

### DETAILED DESCRIPTION

The present invention is concerned with substituted spiro isoxazoline useful as antagonists of SSTR5. Compounds of the present invention are described by structural formula I and pharmaceutically acceptable salts thereof:

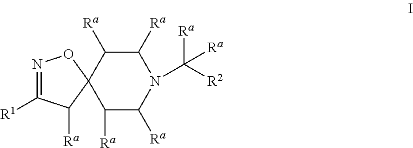

wherein each occurrence of R^{a} is independently selected from the group consisting of hydrogen, halogen, C₁-C₁₀alkyl and halogen-substitutedC₁-C₁₀alkyl; R¹ is selected from the group consisting of phenyl and heterocycle, wherein the phenyl and heterocycle are substituted with at least one substituent selected from α; R² is selected from the group consisting of phenyl and heterocycle, wherein the phenyl and heterocycle is substituted with 1-3 substituents independently selected from α; and α is selected from the group consisting of halogen, C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments, at every occurrence of R^{a}, R^{a} is hydrogen, as shown in formula Ia:

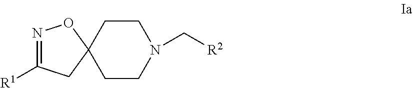

wherein R¹ is selected from the group consisting of phenyl and heterocycle, wherein the phenyl and heterocycle are substituted with at least one substituent selected from α; R² is selected from the group consisting of phenyl and heterocycle, wherein the phenyl and heterocycle is substituted with 1-3 substituents independently selected from α; and α is selected from the group consisting of halogen, C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, R¹ is a heterocycle. In some embodiments R¹ is a heterocycle group, wherein the heterocycle group is a 5 or 6 membered ring having at least one nitrogen, oxygen or sulfur. Examples include but are not limited to pyridine, pyrazine, pyrimidine, tetrazole, triazole, imidazole, pyrazole, pyrrole, isoxazole, isothiazole, pyrazole, oxazole, thiadiazole, thiazole, imidazole, furan, triazine, thiophene, indole, dihydrobenzothiene.

In certain embodiments of the compounds described herein, R¹ is a heterocycle. In some embodiments R¹ is a heterocycle group, wherein the heterocycle group is a 5 or 6 membered ring having at least one nitrogen, examples include but are not limited to pyridine, pyrazine, pyrimidine, tetrazole, triazole, imidazole, pyrazole and pyrrole. For example, in certain embodiments of the compounds described herein, R¹ is pyridine.

When R¹ is pyridine the nitrogen can be any one of the following positions:

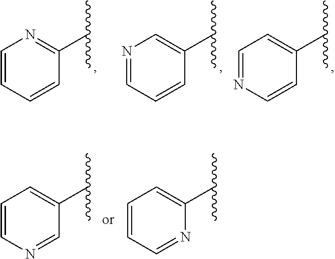

In still other embodiments of the compounds described herein, R¹ can be phenyl or pyridine. In other embodiments of the compounds described herein, R¹ is phenyl.

In some embodiments of the compounds described herein, wherein R¹ is phenyl or heterocycle, R¹ can be substituted with at least one substituent selected from α, wherein α includes halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments, R¹ is substituted with one substituent selected from α. In other embodiments, R¹ is substituted with two substituents selected from α. In still other embodiments, R¹ is substituted with three substituents selected from α.

Described herein are compounds wherein R¹ is substituted with at least one —COOH or —O—C₁-C₁₀alkyl. Also described herein are compounds wherein R¹ is substituted with at least one halogen, C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R¹ is substituted with at least one —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle and —O-halogen-substituted aryl.

In certain embodiments of the compounds described herein, R² is a heterocycle. In some embodiments R² is a heterocycle group, wherein the heterocycle group is a 5 or 6 membered ring having at least one nitrogen, oxygen or sulfur. Examples include but are not limited to pyridine, pyrazine, pyrimidine, tetrazole, triazole, imidazole, pyrazole, pyrrole, isoxazole, isothiazole, pyrazole, oxazole, thiadiazole, thiazole, imidazole, furan, triazine, thiophene, indole, dihydrobenzothiene.

In certain embodiments of the compounds described herein, R² is a heterocycle, wherein the heterocycle group is a bicyclic ring having at least one nitrogen, oxygen or sulfur. Examples include but are not limited to:

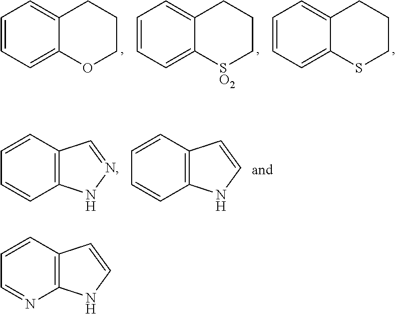

In some embodiments R² is a heterocycle group, wherein the heterocycle group is a bicyclic ring or a 5 or 6 membered ring having at least one nitrogen, examples include but are not limited to pyridine, pyrazine, pyrimidine, tetrazol, triazol, imidazol, pyrazol, pyrrole,

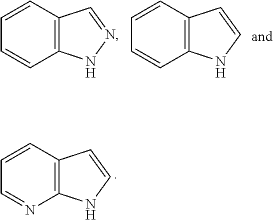

example, in certain embodiments of the compounds described herein, R² is selected from the group consisting of pyridine,

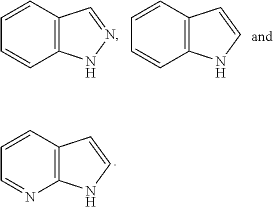

In some embodiments R² is a heterocycle group, wherein the heterocycle group is a 5 or 6 membered ring having at least one nitrogen, examples include but are not limited to pyridine, pyrazine, pyrimidine, tetrazol, triazol, imidazol, pyrazol and pyrrole. For example, in certain embodiments of the compounds described herein, R² is indole, indazole or pyrrolo[2,3-b]pyridine. For example, in certain embodiments of the compounds described herein, R² is pyridine, imidiazole or pyrazole. In certain embodiments, R² is pyridine. In other embodiments, R² is imidizole. In still other embodiments, R² is pyrazole.

In some embodiments R² is a heterocycle group, wherein the heterocycle group is a bicyclic ring or a 5 or 6 membered monocyclic ring having at least one oxygen, examples include but are not limited to furan, tetrahydropyran and benzotetrahydropyran. For example, in certain embodiments of the compounds described herein, R² is benzotetrahydropyran.

In other embodiments of the compounds described herein, R² is phenyl. In still other embodiments of the compounds described herein, R² is phenyl or pyrazole. In still other embodiments of the compounds described herein, R² can be phenyl or pyridine. In yet other embodiments of the compounds described herein, R² can be phenyl, pyrazole or pyridine.

In some embodiments of the compounds described herein, wherein R² is phenyl or heterocycle, R² can be substituted with 1-3 substituents selected from α, wherein α is includes halogen, C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₃-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments, R² is substituted with one substituent selected from α. In some embodiments, R² is substituted with five substituents selected from α. In other embodiments, R² is substituted with three substituents selected from α. In still other embodiments, R² is substituted with three substituents selected from α. In yet other embodiments, R² is substituted with two substituents selected from α. In certain embodiments, R² is substituted with one substituents selected from α.

In certain embodiments described herein R² is substituted with 1-3 substituents selected from the group consisting of halogen, C₁-C₁₀alkyl, C₃-C₆cycloalkyl, halogen-substitutedC₁-C₁₀alkyl, —O—C₁-C₁₀alkyl, —O-halogen-substitutedC₁-C₁₀alkyl, aryl, heterocycle, halogen-substituted heterocycle, C₁-C₁₀alkyl-substituted heterocycle, halogen-substituted aryl and —COOH. In certain embodiments, wherein R² is substituted with C₃-C₆cycloalkyl, the C₃-C₆cycloalkyl may include a carbon from R². In other embodiments, wherein R² is substituted with C₃-C₆cycloalkyl, the C₃-C₆cycloalkyl may not include a carbon from R².

In certain embodiments described herein R² is substituted with 1-3 substituents selected from the group consisting of halogen, C₁-C₁₀alkyl, halogen-substitutedC₁-C₁₀alkyl, —O—C₁-C₁₀alkyl, —O-halogen-substitutedC₁-C₁₀alkyl, aryl, heterocycle, halogen-substituted heterocycle, C₁-C₁₀alkyl-substituted heterocycle, halogen-substituted aryl and —COOH. In other embodiments of the compounds described herein, R² is substituted with 1-3 substituents selected from the group consisting of halogen, C₁-C₁₀alkyl, —O—C₁-C₁₀alkyl, —O-halogen-substitutedC₁-C₁₀alkyl, heterocycle, halogen-substituted heterocycle or halogen-substituted aryl. In yet other embodiment, R² is substituted with 1-3 substituents selected from the group consisting of —O—C₁-C₁₀alkyl, —O-halogen-substitutedC₁-C₁₀alkyl and halogen-substituted aryl. In yet other embodiment, R² is substituted with 1-3 substituents selected from the group consisting of C₁-C₁₀alkyl.

Also described herein are compounds wherein R² is substituted with 1-3 substituents selected from the group consisting of halogen, C₁-C₁₀alkyl, halogen-substitutedC₁-C₁₀alkyl, —C₃-C₁₀cycloalkyl and halogen-substitutedC₃-C₁₀cycloalkyl. Other examples of embodiments of the compounds described herein, include embodiments wherein R² is substituted with 1-3 substituents selected from the group consisting of —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle and —O-halogen-substituted aryl.

In certain embodiments described herein R² is substituted with 2-3 substituents selected from the group consisting of halogen, C₁-C₁₀alkyl, C₃-C₆cycloalkyl, halogen-substitutedC₁-C₁₀alkyl, —O—C₁-C₁₀alkyl, —O-halogen-substitutedC₁-C₁₀alkyl, aryl, heterocycle, halogen-substituted heterocycle, C₁-C₁₀alkyl-substituted heterocycle, halogen-substituted aryl and —COOH.

In certain embodiments described herein R² is substituted with 2-3 substituents selected from the group consisting of halogen, C₁-C₁₀alkyl, halogen-substitutedC₁-C₁₀alkyl, —O—C₁-C₁₀alkyl, —O-halogen-substitutedC₁-C₁₀alkyl, aryl, heterocycle, halogen-substituted heterocycle, halogen-substituted aryl and —COOH. In other embodiments of the compounds described herein, R² is substituted with 2-3 substituents selected from the group consisting of halogen, C₁-C₁₀alkyl, —O—C₁-C₁₀alkyl, —O-halogen-substitutedC₁-C₁₀alkyl, heterocycle, halogen-substituted heterocycle or halogen-substituted aryl. In yet other embodiment, R² is substituted with 2-3 substituents selected from the group consisting of —O—C₁-C₁₀alkyl, —O-halogen-substitutedC₁-C₁₀alkyl and halogen-substituted aryl.

Also described herein are compounds wherein R² is substituted with 2-3 substituents selected from the group consisting of halogen, C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R² is substituted with 2-3 substituents selected from the group consisting of —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle and —O-halogen-substituted aryl.

Also described herein are compounds of structural formula Ib:

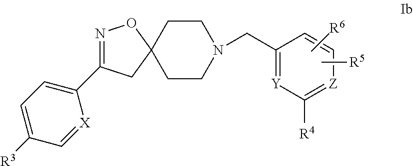

or a pharmaceutically acceptable salt thereof, wherein X, Y and Z are independently selected from —N—, —CH— and —C—, wherein —C— can be substituted with R⁵ or R⁶; R³, R⁴, R⁵ and R⁶ are independently selected from the group consisting of hydrogen, halogen, C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle; and wherein R³, R⁴, R⁵ and R⁶ are not all simultaneously hydrogen.

In certain embodiments of the compounds described herein, X is —N—. In other embodiments, of the compounds described herein, Y is —N—. In still other embodiments, Z is —N—. In certain embodiments of the compounds described herein, X is —CH—. In other embodiments, of the compounds described herein, Y is —CH—. In still other embodiments, Z is —CH. In still other embodiments, Z is —C—, wherein —C— can be substituted with R⁵ or R⁶. In other embodiments, of the compounds described herein, Y is —C—, wherein —C— can be substituted with R⁵ or R.

For example, in certain embodiments of the compounds described herein X is —N— and Y and Z are —CH—. In other embodiments, X, Y and Z are —CH—. In still other embodiments, X and Z are —CH— and Y is —N—. In yet other embodiments, X and Y are —CH— and Z is —N—. In still other embodiments, X and Z are —N— and Y is —CH—. In yet other embodiments, X and Y are —N— and Z is —CH—.

In certain embodiments of the compounds described herein X is —N— and Y and Z are —CH— or —C—, wherein —C— can be substituted with R⁵ or R⁶. In other embodiments, X, Y and Z are —CH— or —C—, wherein —C— can be substituted with R⁵ or R⁶. In still other embodiments, X and Z are —CH— or —C—, wherein —C— can be substituted with R⁵ or R⁶ and Y is —N—. In yet other embodiments, X and Y are —CH— or —C—, wherein —C— can be substituted with R⁵ or R⁶ and Z is —N—. In still other embodiments, X and Z are —N— and Y is —CH— or —C—, wherein —C— can be substituted with R⁵ or R⁶. In yet other embodiments, X and Y are —N— and Z is —CH— or —C—, wherein —C— can be substituted with R⁵ or R⁶.

In certain embodiments of the compounds described herein, R³ can be selected from the group consisting of hydrogen, halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, R³ can be selected from the group consisting of halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

Described herein are compounds wherein R³ is —COOH or —O—C₁-C₁₀alkyl. Also described herein are compounds wherein R³ is selected from the group consisting of halogen, C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R³ is —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle or —O-halogen-substituted aryl. In certain embodiments, R³ is —COOH.

In certain embodiments of the compounds described herein, R⁴ can be selected from the group consisting of hydrogen, halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, R⁴ can be selected from the group consisting of halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

Described herein are compounds wherein R⁴ is hydrogen, halogen, —O—C₁-C₁₀alkyl, C₁-C₁₀alkyl, halogen-substituted heterocycle and halogen-substituted aryl. Also described herein are compounds wherein R⁴ is halogen, —O—C₁-C₁₀alkyl, C₁-C₁₀alkyl, halogen-substituted heterocycle and halogen-substituted aryl. In certain embodiments, R⁴ is selected from the group consisting of halogen, C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R⁴ is —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle or —O-halogen-substituted aryl. In yet other embodiments, R⁴ can be selected from the group consisting of halogen, C₁-C₁₀alkyl, —O—C₁-C₁₀alkyl, —O-halogen-substitutedC₁-C₁₀alkyl, heterocycle, halogen-substituted heterocycle, halogen-substituted aryl. Examples of halogen include chlorine, bromine and fluorine. Examples of C₁-C₁₀alkyl include methyl and t-butyl. Examples of —O—C₁-C₁₀alkyl include methoxy, ethoxy and isopropoxy. Examples of —O-halogen-substitutedC₁-C₁₀alkyl include trifluoromethoxy. Examples of heterocycle include thiazole. Examples of halogen-substituted heterocycle include fluoropyridine. Examples of halogen-substituted aryl include fluorophenyl, difluorophenyl, trifluorophenyl and chlorofluorophenyl. In still other embodiments, R⁴ is selected from the group consisting of halogen and —O—C₁-C₁₀alkyl. In yet other embodiments, R⁴ is —O—C₁-C₁₀alkyl.

In certain embodiments of the compounds described herein, R⁵ can be selected from the group consisting of hydrogen, halogen, C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, R⁵ can be selected from the group consisting of halogen, —C₁-C₁₀alkyl, halogen-substitutedC-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

Described herein are compounds wherein R⁵ is hydrogen, halogen, —O—C₁-C₁₀alkyl, C₁-C₁₀alkyl, halogen-substituted heterocycle and halogen-substituted aryl. Also described herein are compounds wherein R⁵ is halogen, —O—C₁-C₁₀alkyl, —C₁-C₁₀alkyl, halogen-substituted heterocycle and halogen-substituted aryl. In certain embodiments, R⁵ is selected from the group consisting of halogen, —C₁-C₁₀alkyl, halogen-substitutedC₁-C₁₀alkyl, —C₃-C₁₀cycloalkyl and halogen-substitutedC₃-C₁₀cycloalkyl. Other examples of embodiments of the compounds described herein, include embodiments wherein R⁵ is —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle or —O-halogen-substituted aryl. In yet other embodiments, R⁵ can be selected from the group consisting of halogen, —C₁-C₁₀alkyl, —O—C₁-C₁₀alkyl, —O-halogen-substitutedC₁-C₁₀alkyl, heterocycle, halogen-substituted heterocycle, halogen-substituted aryl. Examples of halogen include chlorine, bromine and fluorine. Examples of C₁-C₁₀alkyl include methyl and t-butyl. Examples of —O—C₁-C₁₀alkyl include methoxy, ethoxy and isopropoxy. Examples of —O-halogen-substitutedC₁-C₁₀alkyl include trifluoromethoxy. Examples of heterocycle include thiazole. Examples of halogen-substituted heterocycle include fluoropyridine. Examples of halogen-substituted aryl include fluorophenyl, difluorophenyl, trifluorophenyl and chlorofluorophenyl. In still other embodiments, R⁵ is selected from the group consisting of halogen and —O—C₁-C₁₀alkyl. In yet other embodiments, R⁵ is —O—C₁-C₁₀alkyl.

In certain embodiments of the compounds described herein, R⁶ can be selected from the group consisting of hydrogen, halogen, C₁-C₁₀alkyl, halogen-substitutedC-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, R⁶ can be selected from the group consisting of halogen, —C₁-C₁₀alkyl, halogen-substitutedC₁-C₁₀alkyl, —C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, C₁-C₁₀alkyl-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

Described herein are compounds wherein R⁶ is hydrogen, halogen, —O—C₁-C₁₀alkyl, C₁-C₁₀alkyl, halogen-substituted heterocycle and halogen-substituted aryl. Also described herein are compounds wherein R⁶ is halogen, —O—C₁-C₁₀alkyl, —C₁-C₁₀alkyl, halogen-substituted heterocycle, C₁-C₁₀alkyl-substituted heterocycle, and halogen-substituted aryl. In certain embodiments, R⁶ is selected from the group consisting of halogen, —C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R⁶ is —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle or —O-halogen-substituted aryl. In yet other embodiments, R⁶ can be selected from the group consisting of halogen, —C₁-C₁₀alkyl, —O—C₁-C₁₀alkyl, —O-halogen-substitutedC₁-C₁₀alkyl, heterocycle, halogen-substituted heterocycle, halogen-substituted aryl. Examples of halogen include chlorine, bromine and fluorine. Examples of C₁-C₁₀alkyl include methyl and t-butyl. Examples of —O—C₁-C₁₀alkyl include methoxy, ethoxy and isopropoxy. Examples of —O-halogen-substitutedC₁-C₁₀alkyl include trifluoromethoxy. Examples of heterocycle include thiazole. Examples of halogen-substituted heterocycle include fluoropyridine. Examples of halogen-substituted aryl include fluorophenyl, difluorophenyl, trifluorophenyl and chlorofluorophenyl. In still other embodiments, R⁶ is selected from the group consisting of halogen and —O—C₁-C₁₀alkyl. In yet other embodiments, R⁶ is —O—C₁-C₁₀alkyl.

In certain embodiments, R⁴, R⁵ and R⁶ are independently selected from the group consisting of halogen, —C₁-C₁₀alkyl, —O—C₁-C₁₀alkyl, —O-halogen-substitutedC₁-C₁₀alkyl, heterocycle, halogen-substituted heterocycle, halogen-substituted aryl.

In certain embodiments of the compounds described herein, R⁴, R⁵ and R⁶ can be in any one of the following configurations:

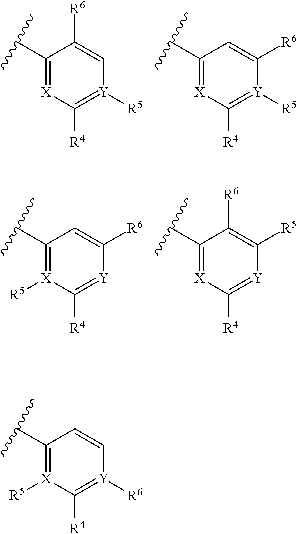

In certain embodiments, R⁴, R⁵ and R⁶ are not hydrogen. In other embodiments, R⁴ is hydrogen and R⁵ and R⁶ are not hydrogen. In still other embodiments, R⁵ is hydrogen and R⁴ and R⁶ are not hydrogen. In other embodiments, R⁶ is hydrogen and R⁵ and R⁴ are not hydrogen. In all the embodiments described herein, R⁴, R⁵ and R⁶ are not simultaneously hydrogen.

Also described herein are compounds of structural formula Ic, or pharmaceutically acceptable salts thereof:

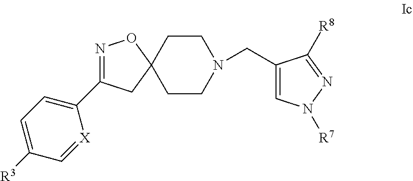

wherein X is selected from —N— and —CH—; R³, R⁷ and R⁸ are independently selected from α; and wherein α includes halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

In certain embodiments of the compounds described herein, X is —N—. In other embodiments of the compounds described herein, X is —CH—.

In certain embodiments of the compounds described herein, R³ can be selected from the group consisting of halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

Described herein are compounds wherein R³ is —COOH or —O—C₁-C₁₀alkyl. Also described herein are compounds wherein R³ is selected from the group consisting of halogen, C₁-C₁₀alkyl, halogen-substitutedC₁-C₁₀alkyl, C₃-C₁₀cycloalkyl and halogen-substitutedC₃-C₁₀cycloalkyl. Other examples of embodiments of the compounds described herein, include embodiments wherein R³ is —OH, —O—C₁-C₁₀alkyl, —O-halogen-substitutedC₁-C₁₀alkyl, —O—C₃-C₁₀cycloallyl, —O-halogen-substitutedC₃-C₁₀cycloalkyl, —O-aryl, —O-heterocycle, —O-halogen-substituted heterocycle or —O-halogen-substituted aryl. In certain embodiments, R³ is —COOH.

In certain embodiments of the compounds described herein, R⁷ can be selected from the group consisting of halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle. For example, in certain embodiments, R⁷ is —C₁-C₁₀alkyl. Examples of —C₃-C₁₀alkyl include t-butyl.

In certain embodiments of the compounds described herein, R⁸ can be selected from the group consisting of halogen, C₁-C₁₀alkyl, halogen-substitutedC₁-C₁₀alkyl, C₃-C₁₀cycloalkyl, halogen-substitutedC₃-C₁₀cycloalkyl, —OH, —O—C₁-C₁₀alkyl, —O-halogen-substitutedC₁-C₁₀alkyl, —O—C₃-C₁₀cycloalkyl, —O-halogen-substitutedC₃-C₁₀cycloalkyl, —O-aryl, —O-heterocycle, —O-halogen-substituted heterocycle, —O-halogen-substituted aryl, —NR^{b}S(O)₂R^{d}, —NR^{b}R^{c}, —CN, —NR^{b}C(O)R^{c}, aryl, heterocycle, halogen-substituted heterocycle, halogen-substituted aryl, —S(O)₂R^{d}, —S(O)₂NR^{b}R^{c}, —C(O)NR^{b}R^{c}, —NR^{b}C(O)OR^{c}, —NR^{b}C(O)NR^{c}R^{d}, —NR^{b}C(O)NH₂, —NR^{b}S(O)₂R^{d}, —NO₂, —C(O)R^{d}, —COOH, —CO₂R^{d}, —OC(O)R^{d}, wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle. For example, in certain embodiments, R⁸ is halogen-substituted aryl.

In other embodiments of the compounds described herein, R⁷ and R⁸ are independently selected from the group consisting of halogen, C₁-C₁₀alkyl, —O—C₁-C₁₀alkyl, —O-halogen-substitutedC₁-C₁₀alkyl, heterocycle, halogen-substituted heterocycle, halogen-substituted aryl. Examples of halogen include chlorine, bromine and fluorine. Examples of C₁-C₁₀alkyl include methyl and t-butyl. Examples of —O—C₁-C₁₀alkyl include methoxy, ethoxy and isopropoxy. Examples of —O-halogen-substitutedC₁-C₁₀alkyl include trifluoromethoxy. Examples of heterocycle include thiazole. Examples of halogen-substituted heterocycle include fluoropyridine. Examples of halogen-substituted aryl include fluorophenyl, difluorophenyl, trifluorophenyl and chlorofluorophenyl.

Examples of the compounds described herein include those shown in Tables 1 and 2:

TABLE 1

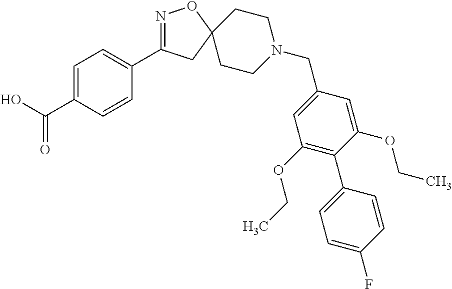 [SMILES: CCOc1cc(CN2CCC3(CC2)CC(c2ccc(C(=O)O)cc2)=NO3)cc(OCC)c1-c1ccc(F)cc1]

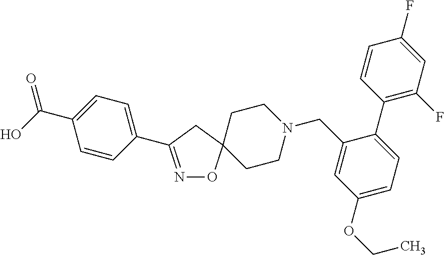 [SMILES: CCOc1ccc(-c2ccc(F)cc2F)c(CN2CCC3(CC2)CC(c2ccc(C(=O)O)cc2)=NO3)c1]

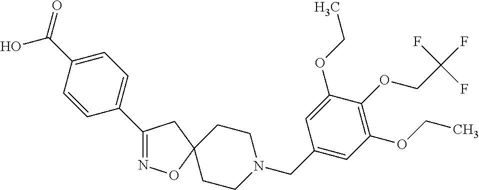 [SMILES: CCOc1cc(CN2CCC3(CC2)CC(c2ccc(C(=O)O)cc2)=NO3)cc(OCC)c1OCC(F)(F)F]

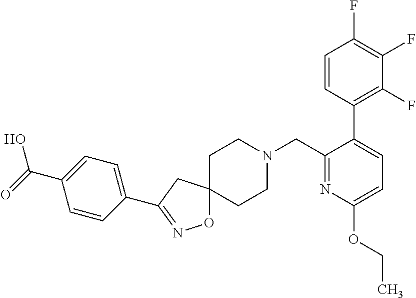 [SMILES: CCOc1ccc(-c2ccc(F)c(F)c2F)c(CN2CCC3(CC2)CC(c2ccc(C(=O)O)cc2)=NO3)n1]

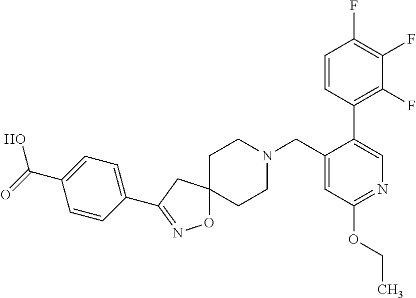 [SMILES: CCOc1cc(CN2CCC3(CC2)CC(c2ccc(C(=O)O)cc2)=NO3)c(-c2ccc(F)c(F)c2F)cn1]

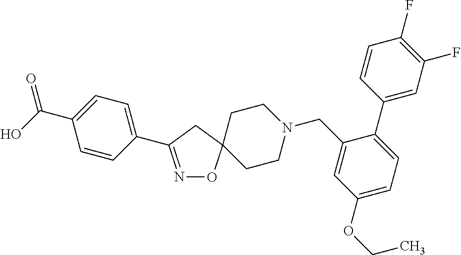 [SMILES: CCOc1ccc(-c2ccc(F)c(F)c2)c(CN2CCC3(CC2)CC(c2ccc(C(=O)O)cc2)=NO3)c1]

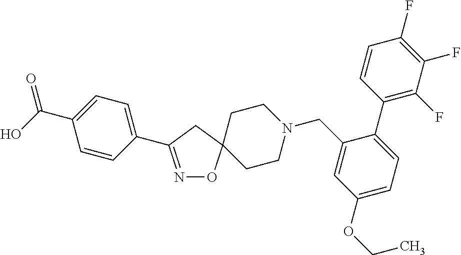 [SMILES: CCOc1ccc(-c2ccc(F)c(F)c2F)c(CN2CCC3(CC2)CC(c2ccc(C(=O)O)cc2)=NO3)c1]

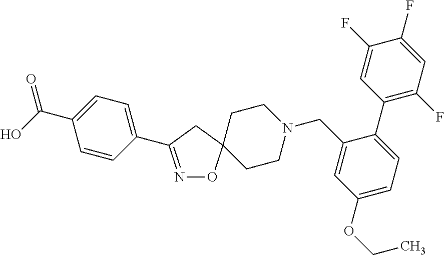 [SMILES: CCOc1ccc(-c2cc(F)c(F)cc2F)c(CN2CCC3(CC2)CC(c2ccc(C(=O)O)cc2)=NO3)c1]

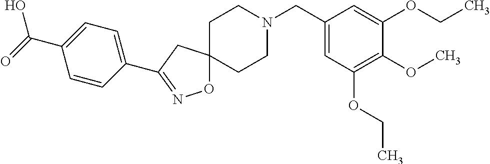 [SMILES: CCOc1cc(CN2CCC3(CC2)CC(c2ccc(C(=O)O)cc2)=NO3)cc(OCC)c1OC]

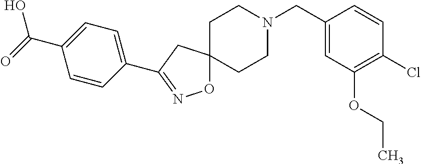 [SMILES: CCOc1cc(CN2CCC3(CC2)CC(c2ccc(C(=O)O)cc2)=NO3)ccc1Cl]

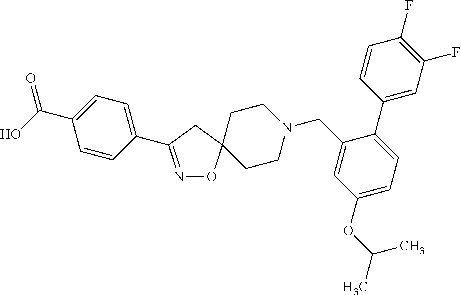 [SMILES: CC(C)Oc1ccc(-c2ccc(F)c(F)c2)c(CN2CCC3(CC2)CC(c2ccc(C(=O)O)cc2)=NO3)c1]

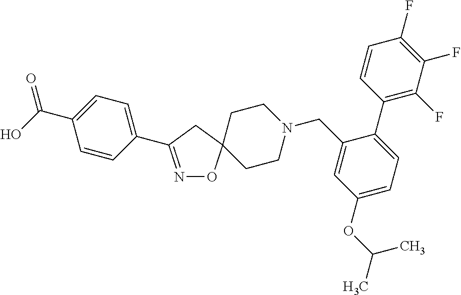 [SMILES: CC(C)Oc1ccc(-c2ccc(F)c(F)c2F)c(CN2CCC3(CC2)CC(c2ccc(C(=O)O)cc2)=NO3)c1]

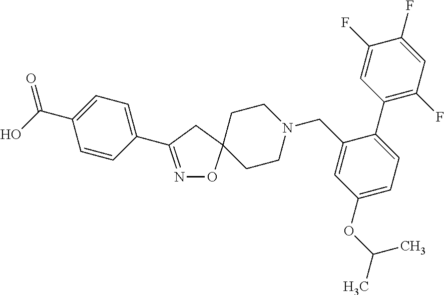 [SMILES: CC(C)Oc1ccc(-c2cc(F)c(F)cc2F)c(CN2CCC3(CC2)CC(c2ccc(C(=O)O)cc2)=NO3)c1]

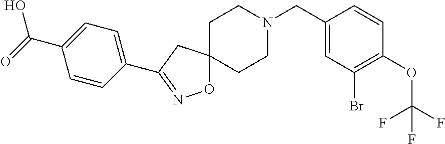 [SMILES: O=C(O)c1ccc(C2=NOC3(CCN(Cc4ccc(OC(F)(F)F)c(Br)c4)CC3)C2)cc1]

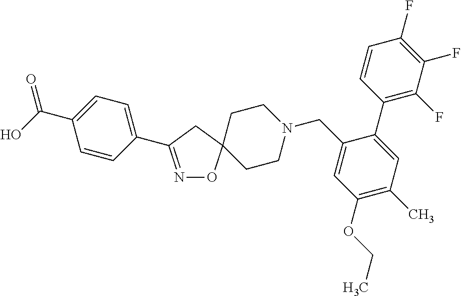 [SMILES: CCOc1cc(CN2CCC3(CC2)CC(c2ccc(C(=O)O)cc2)=NO3)c(-c2ccc(F)c(F)c2F)cc1C]

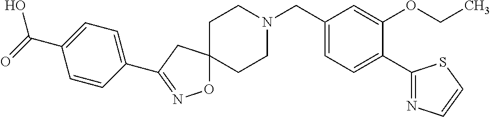 [SMILES: CCOc1cc(CN2CCC3(CC2)CC(c2ccc(C(=O)O)cc2)=NO3)ccc1-c1nccs1]

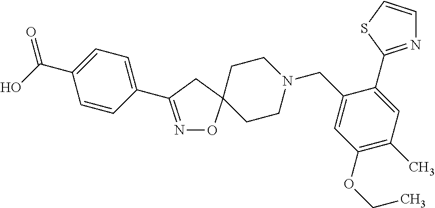 [SMILES: CCOc1cc(CN2CCC3(CC2)CC(c2ccc(C(=O)O)cc2)=NO3)c(-c2nccs2)cc1C]

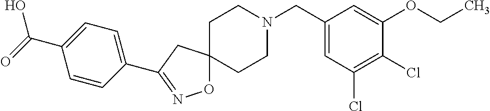 [SMILES: CCOc1cc(CN2CCC3(CC2)CC(c2ccc(C(=O)O)cc2)=NO3)cc(Cl)c1Cl]

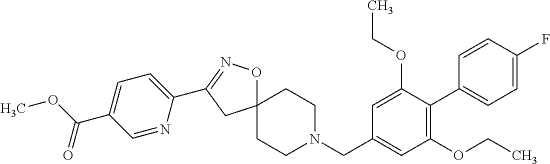 [SMILES: CCOc1cc(CN2CCC3(CC2)CC(c2ccc(C(=O)OC)cn2)=NO3)cc(OCC)c1-c1ccc(F)cc1]

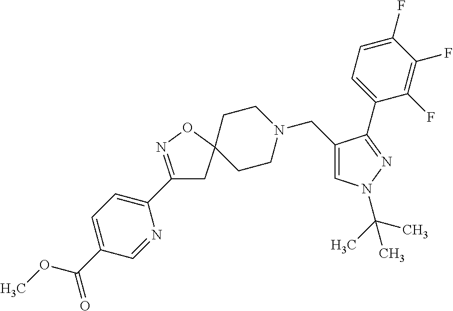 [SMILES: COC(=O)c1ccc(C2=NOC3(CCN(Cc4cn(C(C)(C)C)nc4-c4ccc(F)c(F)c4F)CC3)C2)nc1]

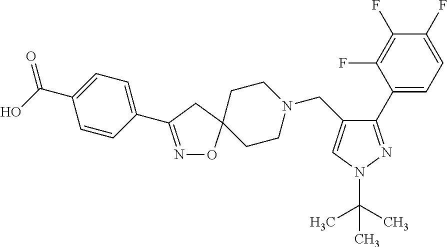 [SMILES: CC(C)(C)n1cc(CN2CCC3(CC2)CC(c2ccc(C(=O)O)cc2)=NO3)c(-c2ccc(F)c(F)c2F)n1]

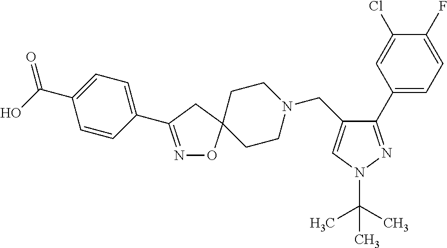 [SMILES: CC(C)(C)n1cc(CN2CCC3(CC2)CC(c2ccc(C(=O)O)cc2)=NO3)c(-c2ccc(F)c(Cl)c2)n1]

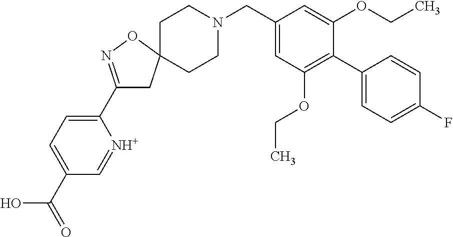 [SMILES: CCOc1cc(CN2CCC3(CC2)CC(c2ccc(C(=O)O)c[nH+]2)=NO3)cc(OCC)c1-c1ccc(F)cc1]

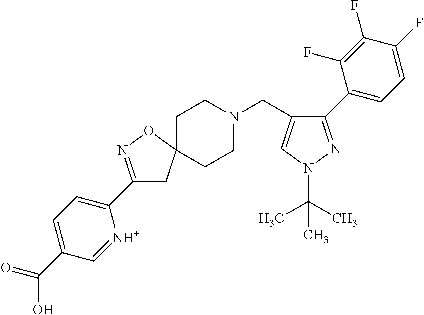 [SMILES: CC(C)(C)n1cc(CN2CCC3(CC2)CC(c2ccc(C(=O)O)c[nH+]2)=NO3)c(-c2ccc(F)c(F)c2F)n1]

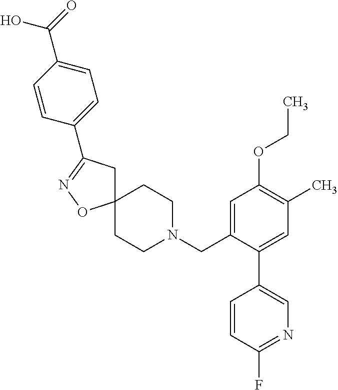 [SMILES: CCOc1cc(CN2CCC3(CC2)CC(c2ccc(C(=O)O)cc2)=NO3)c(-c2ccc(F)nc2)cc1C]

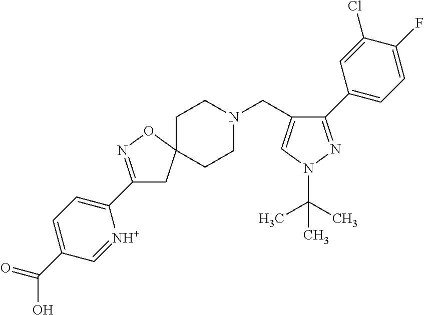 [SMILES: CC(C)(C)n1cc(CN2CCC3(CC2)CC(c2ccc(C(=O)O)c[nH+]2)=NO3)c(-c2ccc(F)c(Cl)c2)n1]

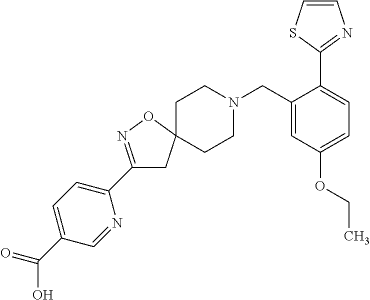 [SMILES: CCOc1ccc(-c2nccs2)c(CN2CCC3(CC2)CC(c2ccc(C(=O)O)cn2)=NO3)c1]

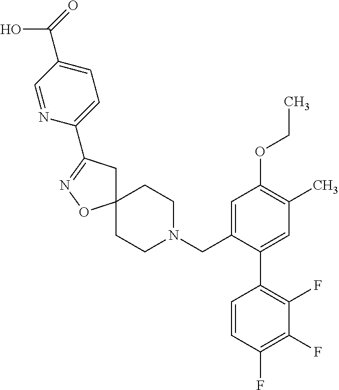 [SMILES: CCOc1cc(CN2CCC3(CC2)CC(c2ccc(C(=O)O)cn2)=NO3)c(-c2ccc(F)c(F)c2F)cc1C]

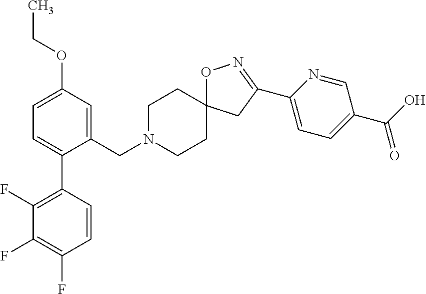 [SMILES: CCOc1ccc(-c2ccc(F)c(F)c2F)c(CN2CCC3(CC2)CC(c2ccc(C(=O)O)cn2)=NO3)c1]

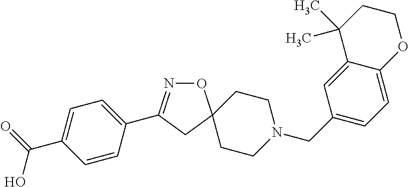 [SMILES: CC1(C)CCOc2ccc(CN3CCC4(CC3)CC(c3ccc(C(=O)O)cc3)=NO4)cc21]

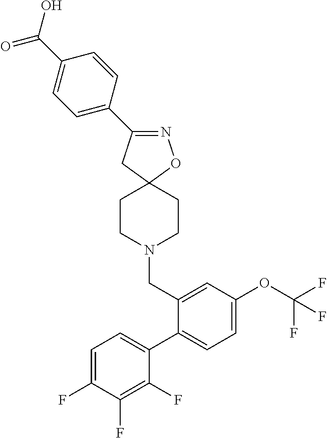 [SMILES: O=C(O)c1ccc(C2=NOC3(CCN(Cc4cc(OC(F)(F)F)ccc4-c4ccc(F)c(F)c4F)CC3)C2)cc1]

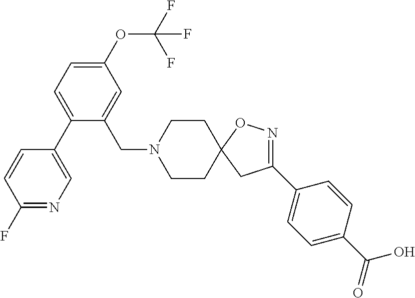 [SMILES: O=C(O)c1ccc(C2=NOC3(CCN(Cc4cc(OC(F)(F)F)ccc4-c4ccc(F)nc4)CC3)C2)cc1]

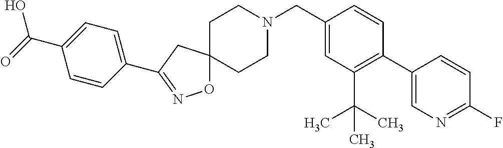 [SMILES: CC(C)(C)c1cc(CN2CCC3(CC2)CC(c2ccc(C(=O)O)cc2)=NO3)ccc1-c1ccc(F)nc1]

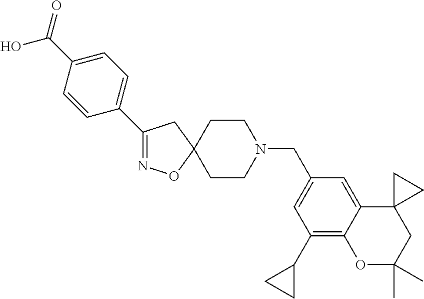 [SMILES: CC1(C)CC2(CC2)c2cc(CN3CCC4(CC3)CC(c3ccc(C(=O)O)cc3)=NO4)cc(C3CC3)c2O1]

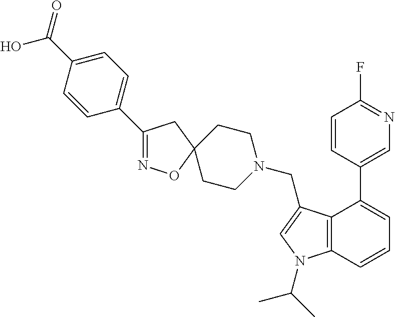 [SMILES: CC(C)n1cc(CN2CCC3(CC2)CC(c2ccc(C(=O)O)cc2)=NO3)c2c(-c3ccc(F)nc3)cccc21]

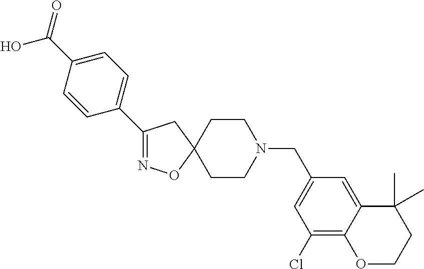 [SMILES: CC1(C)CCOc2c(Cl)cc(CN3CCC4(CC3)CC(c3ccc(C(=O)O)cc3)=NO4)cc21]

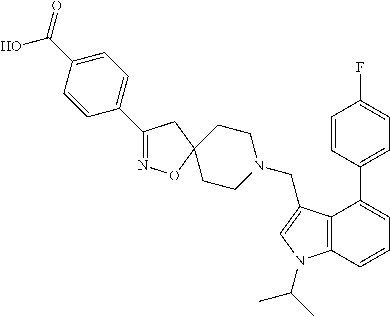 [SMILES: CC(C)n1cc(CN2CCC3(CC2)CC(c2ccc(C(=O)O)cc2)=NO3)c2c(-c3ccc(F)cc3)cccc21]

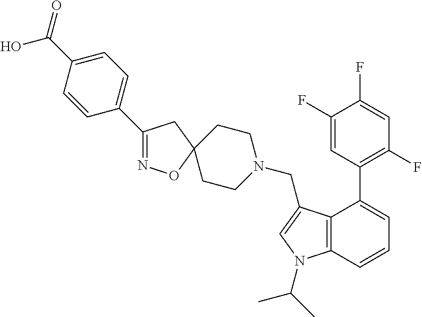 [SMILES: CC(C)n1cc(CN2CCC3(CC2)CC(c2ccc(C(=O)O)cc2)=NO3)c2c(-c3cc(F)c(F)cc3F)cccc21]

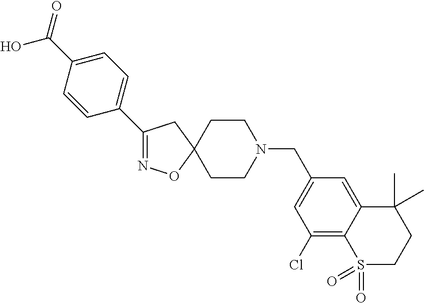 [SMILES: CC1(C)CCS(=O)(=O)c2c(Cl)cc(CN3CCC4(CC3)CC(c3ccc(C(=O)O)cc3)=NO4)cc21]

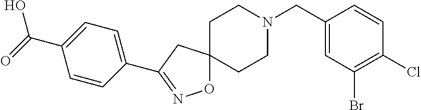 [SMILES: O=C(O)c1ccc(C2=NOC3(CCN(Cc4ccc(Cl)c(Br)c4)CC3)C2)cc1]

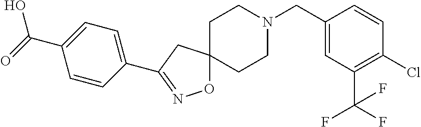 [SMILES: O=C(O)c1ccc(C2=NOC3(CCN(Cc4ccc(Cl)c(C(F)(F)F)c4)CC3)C2)cc1]

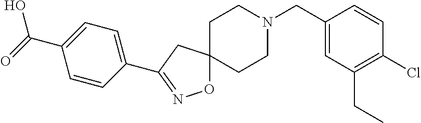 [SMILES: CCc1cc(CN2CCC3(CC2)CC(c2ccc(C(=O)O)cc2)=NO3)ccc1Cl]

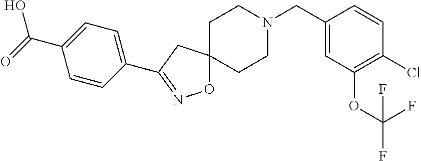 [SMILES: O=C(O)c1ccc(C2=NOC3(CCN(Cc4ccc(Cl)c(OC(F)(F)F)c4)CC3)C2)cc1]

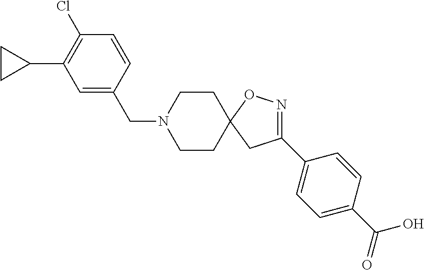 [SMILES: O=C(O)c1ccc(C2=NOC3(CCN(Cc4ccc(Cl)c(C5CC5)c4)CC3)C2)cc1]

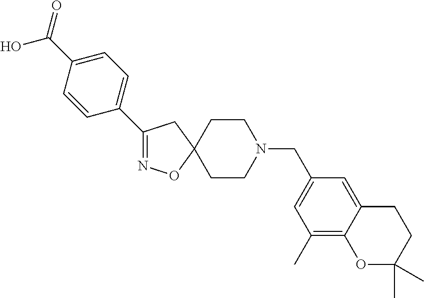 [SMILES: Cc1cc(CN2CCC3(CC2)CC(c2ccc(C(=O)O)cc2)=NO3)cc2c1OC(C)(C)CC2]

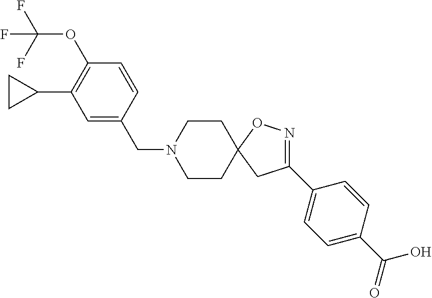 [SMILES: O=C(O)c1ccc(C2=NOC3(CCN(Cc4ccc(OC(F)(F)F)c(C5CC5)c4)CC3)C2)cc1]

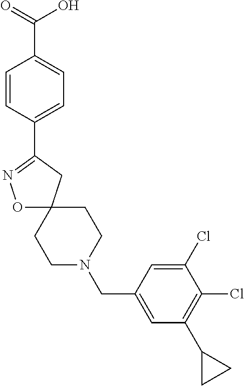 [SMILES: O=C(O)c1ccc(C2=NOC3(CCN(Cc4cc(Cl)c(Cl)c(C5CC5)c4)CC3)C2)cc1]

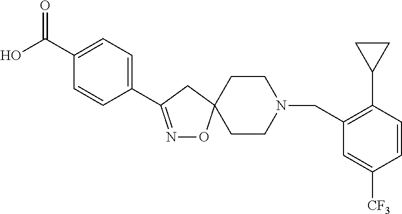 [SMILES: O=C(O)c1ccc(C2=NOC3(CCN(Cc4cc(C(F)(F)F)ccc4C4CC4)CC3)C2)cc1]

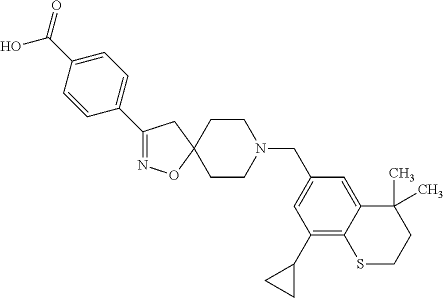 [SMILES: CC1(C)CCSc2c(C3CC3)cc(CN3CCC4(CC3)CC(c3ccc(C(=O)O)cc3)=NO4)cc21]

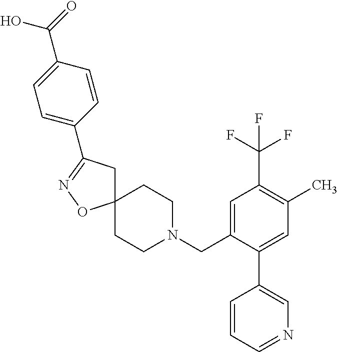 [SMILES: Cc1cc(-c2cccnc2)c(CN2CCC3(CC2)CC(c2ccc(C(=O)O)cc2)=NO3)cc1C(F)(F)F]

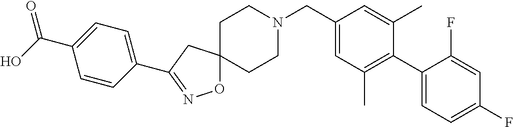 [SMILES: Cc1cc(CN2CCC3(CC2)CC(c2ccc(C(=O)O)cc2)=NO3)cc(C)c1-c1ccc(F)cc1F]

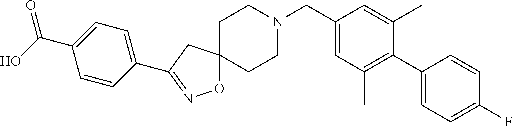 [SMILES: Cc1cc(CN2CCC3(CC2)CC(c2ccc(C(=O)O)cc2)=NO3)cc(C)c1-c1ccc(F)cc1]

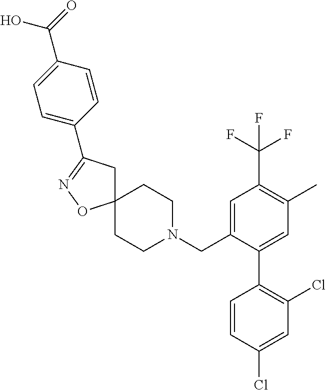 [SMILES: Cc1cc(-c2ccc(Cl)cc2Cl)c(CN2CCC3(CC2)CC(c2ccc(C(=O)O)cc2)=NO3)cc1C(F)(F)F]

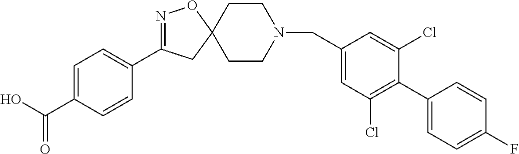 [SMILES: O=C(O)c1ccc(C2=NOC3(CCN(Cc4cc(Cl)c(-c5ccc(F)cc5)c(Cl)c4)CC3)C2)cc1]

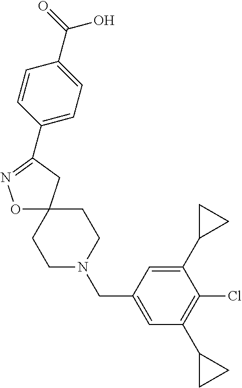 [SMILES: O=C(O)c1ccc(C2=NOC3(CCN(Cc4cc(C5CC5)c(Cl)c(C5CC5)c4)CC3)C2)cc1]

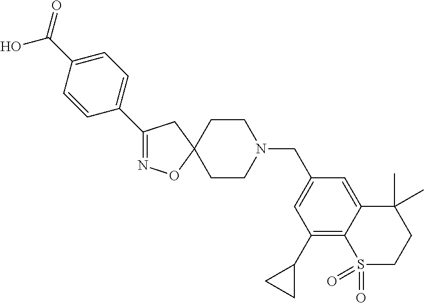 [SMILES: CC1(C)CCS(=O)(=O)c2c(C3CC3)cc(CN3CCC4(CC3)CC(c3ccc(C(=O)O)cc3)=NO4)cc21]

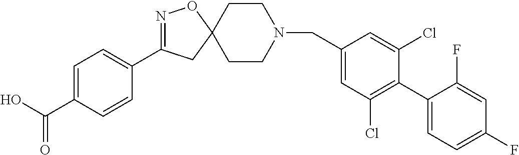 [SMILES: O=C(O)c1ccc(C2=NOC3(CCN(Cc4cc(Cl)c(-c5ccc(F)cc5F)c(Cl)c4)CC3)C2)cc1]

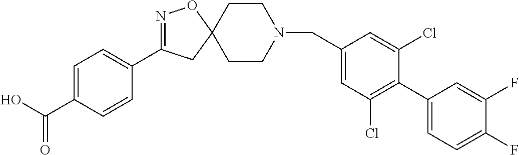 [SMILES: O=C(O)c1ccc(C2=NOC3(CCN(Cc4cc(Cl)c(-c5ccc(F)c(F)c5)c(Cl)c4)CC3)C2)cc1]

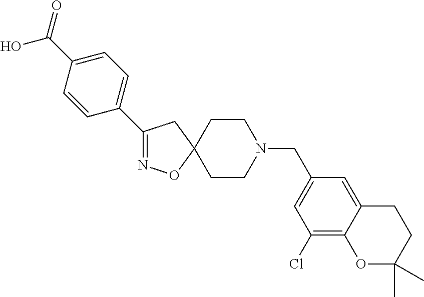 [SMILES: CC1(C)CCc2cc(CN3CCC4(CC3)CC(c3ccc(C(=O)O)cc3)=NO4)cc(Cl)c2O1]

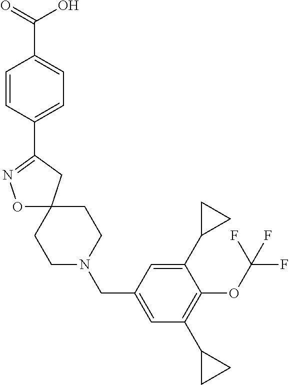 [SMILES: O=C(O)c1ccc(C2=NOC3(CCN(Cc4cc(C5CC5)c(OC(F)(F)F)c(C5CC5)c4)CC3)C2)cc1]

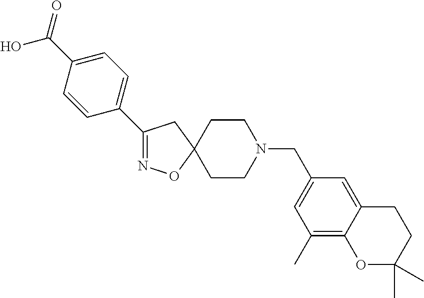 [SMILES: Cc1cc(CN2CCC3(CC2)CC(c2ccc(C(=O)O)cc2)=NO3)cc2c1OC(C)(C)CC2]

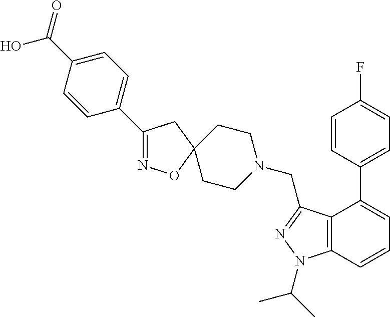 [SMILES: CC(C)n1nc(CN2CCC3(CC2)CC(c2ccc(C(=O)O)cc2)=NO3)c2c(-c3ccc(F)cc3)cccc21]

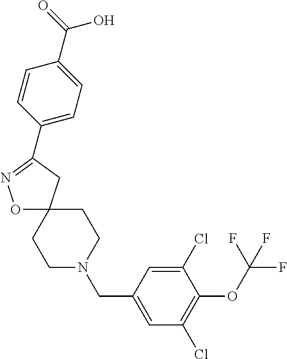 [SMILES: O=C(O)c1ccc(C2=NOC3(CCN(Cc4cc(Cl)c(OC(F)(F)F)c(Cl)c4)CC3)C2)cc1]

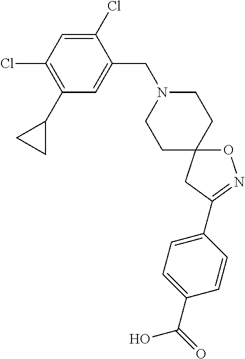 [SMILES: O=C(O)c1ccc(C2=NOC3(CCN(Cc4cc(C5CC5)c(Cl)cc4Cl)CC3)C2)cc1]

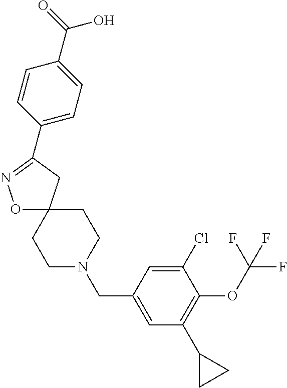 [SMILES: O=C(O)c1ccc(C2=NOC3(CCN(Cc4cc(Cl)c(OC(F)(F)F)c(C5CC5)c4)CC3)C2)cc1]

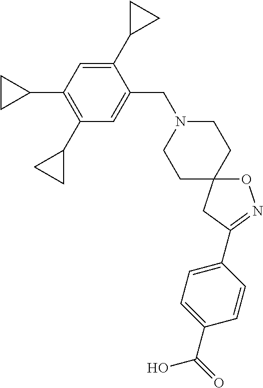 [SMILES: O=C(O)c1ccc(C2=NOC3(CCN(Cc4cc(C5CC5)c(C5CC5)cc4C4CC4)CC3)C2)cc1]

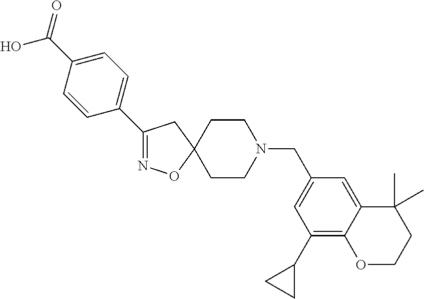 [SMILES: CC1(C)CCOc2c(C3CC3)cc(CN3CCC4(CC3)CC(c3ccc(C(=O)O)cc3)=NO4)cc21]

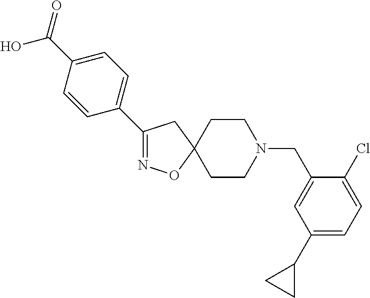 [SMILES: O=C(O)c1ccc(C2=NOC3(CCN(Cc4cc(C5CC5)ccc4Cl)CC3)C2)cc1]

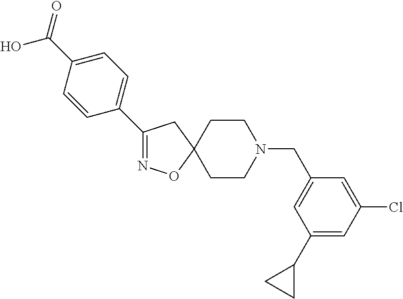 [SMILES: O=C(O)c1ccc(C2=NOC3(CCN(Cc4cc(Cl)cc(C5CC5)c4)CC3)C2)cc1]

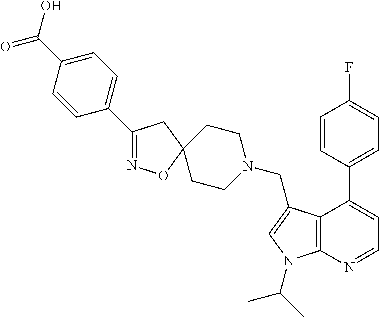 [SMILES: CC(C)n1cc(CN2CCC3(CC2)CC(c2ccc(C(=O)O)cc2)=NO3)c2c(-c3ccc(F)cc3)ccnc21]

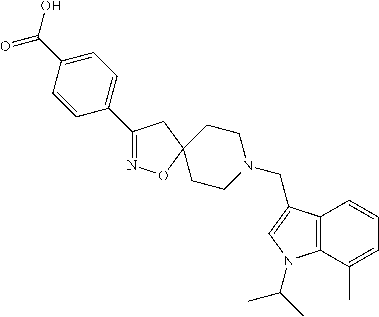 [SMILES: Cc1cccc2c(CN3CCC4(CC3)CC(c3ccc(C(=O)O)cc3)=NO4)cn(C(C)C)c12]

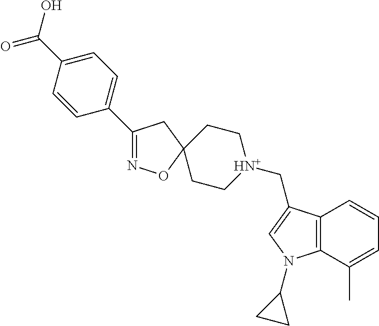 [SMILES: Cc1cccc2c(C[NH+]3CCC4(CC3)CC(c3ccc(C(=O)O)cc3)=NO4)cn(C3CC3)c12]

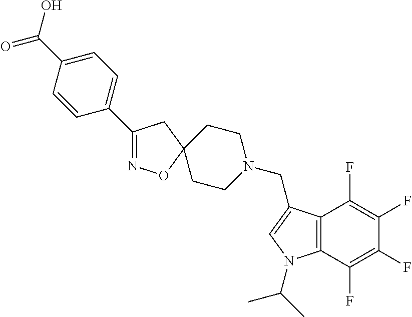 [SMILES: CC(C)n1cc(CN2CCC3(CC2)CC(c2ccc(C(=O)O)cc2)=NO3)c2c(F)c(F)c(F)c(F)c21]

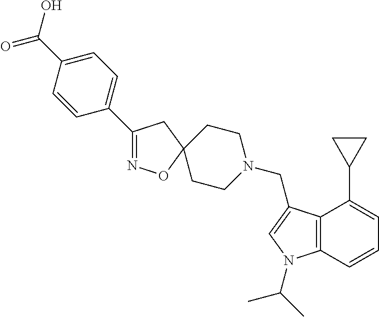 [SMILES: CC(C)n1cc(CN2CCC3(CC2)CC(c2ccc(C(=O)O)cc2)=NO3)c2c(C3CC3)cccc21]

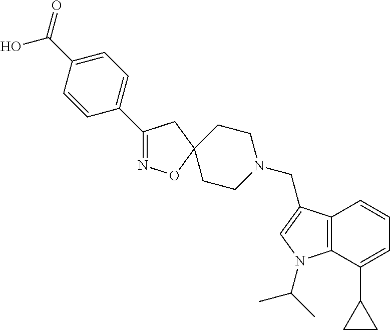 [SMILES: CC(C)n1cc(CN2CCC3(CC2)CC(c2ccc(C(=O)O)cc2)=NO3)c2cccc(C3CC3)c21]

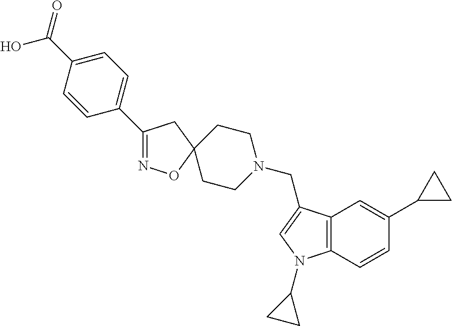 [SMILES: O=C(O)c1ccc(C2=NOC3(CCN(Cc4cn(C5CC5)c5ccc(C6CC6)cc45)CC3)C2)cc1]

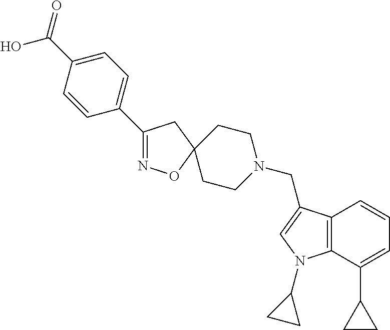 [SMILES: O=C(O)c1ccc(C2=NOC3(CCN(Cc4cn(C5CC5)c5c(C6CC6)cccc45)CC3)C2)cc1]

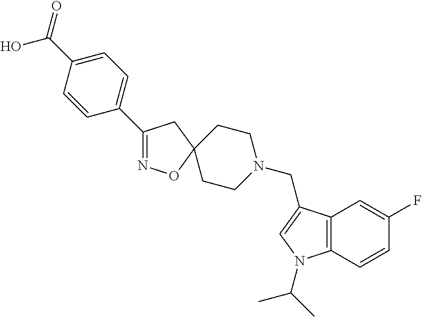 [SMILES: CC(C)n1cc(CN2CCC3(CC2)CC(c2ccc(C(=O)O)cc2)=NO3)c2cc(F)ccc21]

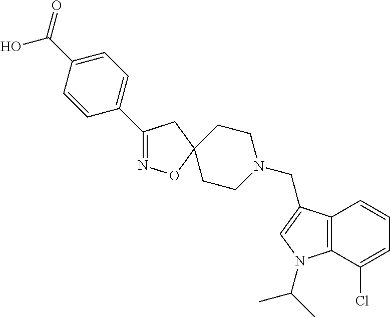 [SMILES: CC(C)n1cc(CN2CCC3(CC2)CC(c2ccc(C(=O)O)cc2)=NO3)c2cccc(Cl)c21]

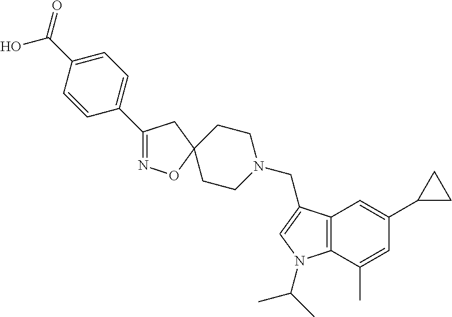 [SMILES: Cc1cc(C2CC2)cc2c(CN3CCC4(CC3)CC(c3ccc(C(=O)O)cc3)=NO4)cn(C(C)C)c12]

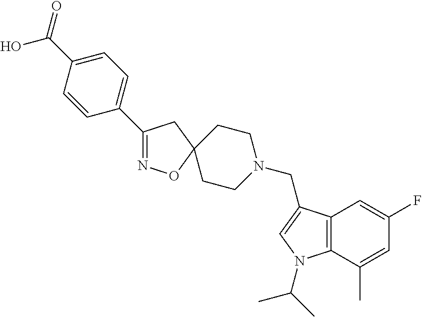 [SMILES: Cc1cc(F)cc2c(CN3CCC4(CC3)CC(c3ccc(C(=O)O)cc3)=NO4)cn(C(C)C)c12]

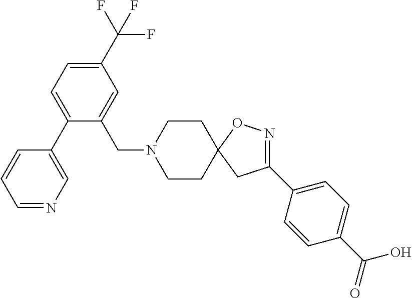 [SMILES: O=C(O)c1ccc(C2=NOC3(CCN(Cc4cc(C(F)(F)F)ccc4-c4cccnc4)CC3)C2)cc1]

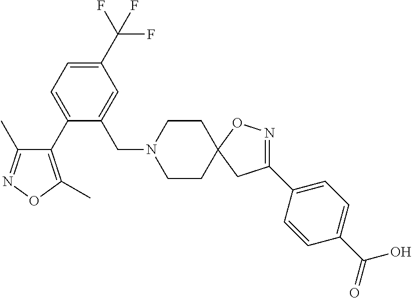 [SMILES: Cc1noc(C)c1-c1ccc(C(F)(F)F)cc1CN1CCC2(CC1)CC(c1ccc(C(=O)O)cc1)=NO2]

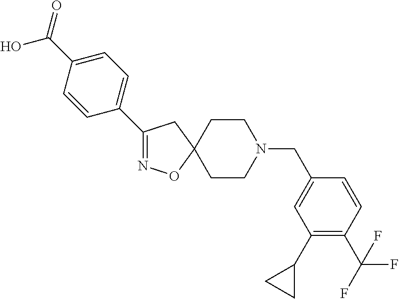 [SMILES: O=C(O)c1ccc(C2=NOC3(CCN(Cc4ccc(C(F)(F)F)c(C5CC5)c4)CC3)C2)cc1]

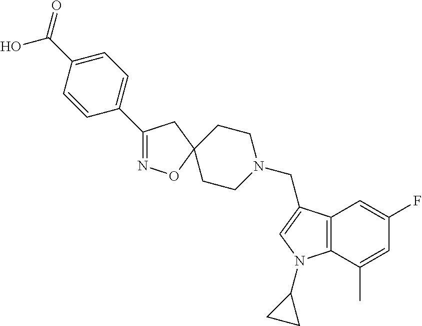 [SMILES: Cc1cc(F)cc2c(CN3CCC4(CC3)CC(c3ccc(C(=O)O)cc3)=NO4)cn(C3CC3)c12]

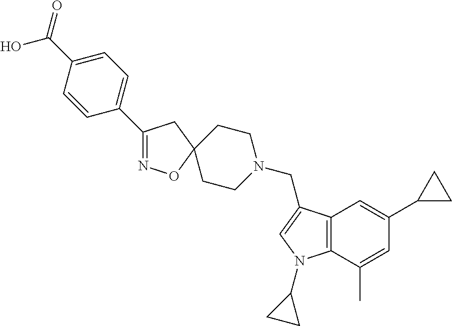 [SMILES: Cc1cc(C2CC2)cc2c(CN3CCC4(CC3)CC(c3ccc(C(=O)O)cc3)=NO4)cn(C3CC3)c12]

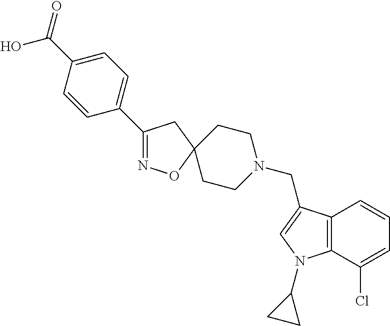 [SMILES: O=C(O)c1ccc(C2=NOC3(CCN(Cc4cn(C5CC5)c5c(Cl)cccc45)CC3)C2)cc1]

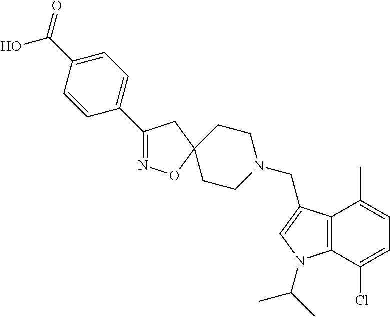 [SMILES: Cc1ccc(Cl)c2c1c(CN1CCC3(CC1)CC(c1ccc(C(=O)O)cc1)=NO3)cn2C(C)C]

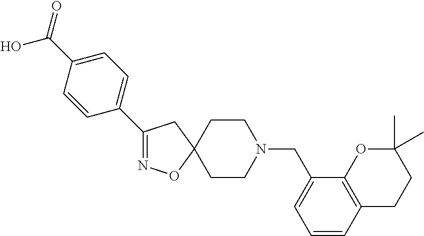 [SMILES: CC1(C)CCc2cccc(CN3CCC4(CC3)CC(c3ccc(C(=O)O)cc3)=NO4)c2O1]

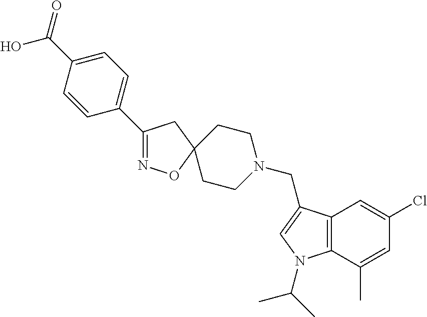 [SMILES: Cc1cc(Cl)cc2c(CN3CCC4(CC3)CC(c3ccc(C(=O)O)cc3)=NO4)cn(C(C)C)c12]

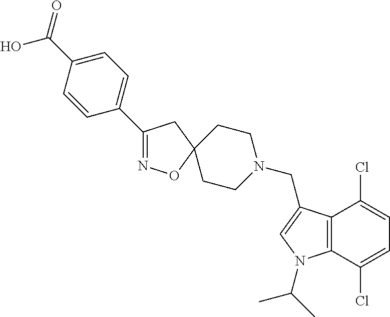 [SMILES: CC(C)n1cc(CN2CCC3(CC2)CC(c2ccc(C(=O)O)cc2)=NO3)c2c(Cl)ccc(Cl)c21]

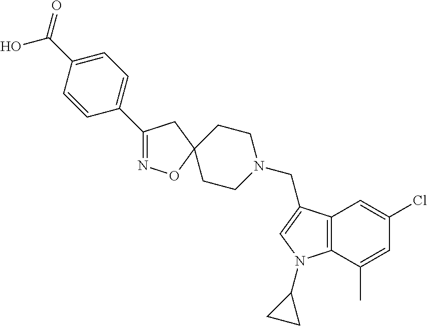 [SMILES: Cc1cc(Cl)cc2c(CN3CCC4(CC3)CC(c3ccc(C(=O)O)cc3)=NO4)cn(C3CC3)c12]

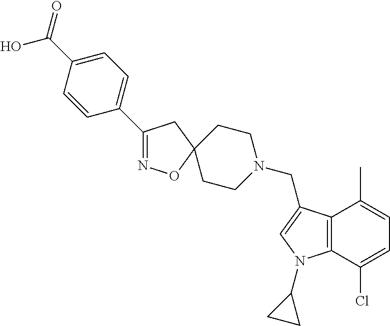 [SMILES: Cc1ccc(Cl)c2c1c(CN1CCC3(CC1)CC(c1ccc(C(=O)O)cc1)=NO3)cn2C1CC1]

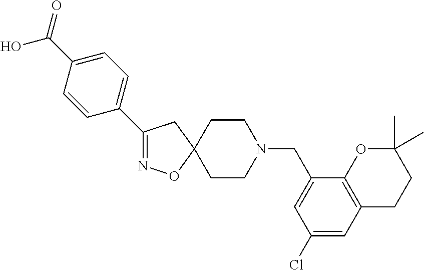 [SMILES: CC1(C)CCc2cc(Cl)cc(CN3CCC4(CC3)CC(c3ccc(C(=O)O)cc3)=NO4)c2O1]

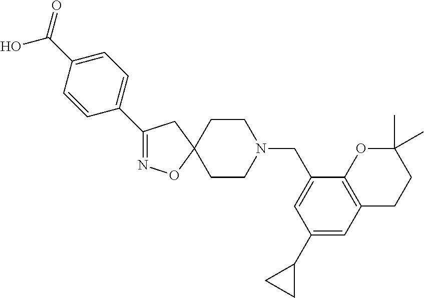 [SMILES: CC1(C)CCc2cc(C3CC3)cc(CN3CCC4(CC3)CC(c3ccc(C(=O)O)cc3)=NO4)c2O1]

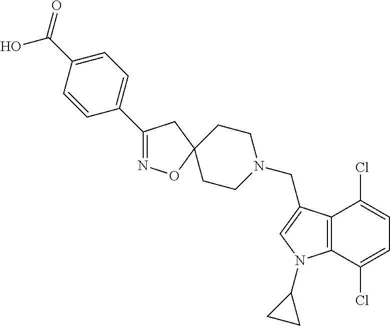 [SMILES: O=C(O)c1ccc(C2=NOC3(CCN(Cc4cn(C5CC5)c5c(Cl)ccc(Cl)c45)CC3)C2)cc1]

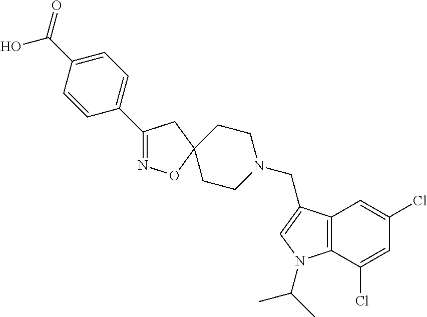 [SMILES: CC(C)n1cc(CN2CCC3(CC2)CC(c2ccc(C(=O)O)cc2)=NO3)c2cc(Cl)cc(Cl)c21]

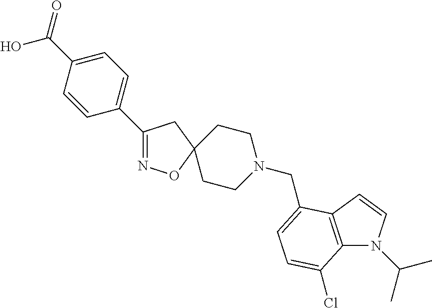 [SMILES: CC(C)n1ccc2c(CN3CCC4(CC3)CC(c3ccc(C(=O)O)cc3)=NO4)ccc(Cl)c21]

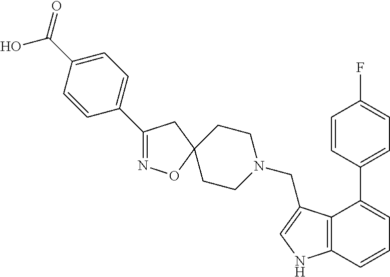 [SMILES: O=C(O)c1ccc(C2=NOC3(CCN(Cc4c[nH]c5cccc(-c6ccc(F)cc6)c45)CC3)C2)cc1]

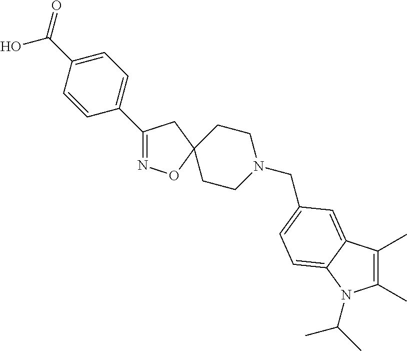 [SMILES: Cc1c(C)n(C(C)C)c2ccc(CN3CCC4(CC3)CC(c3ccc(C(=O)O)cc3)=NO4)cc12]

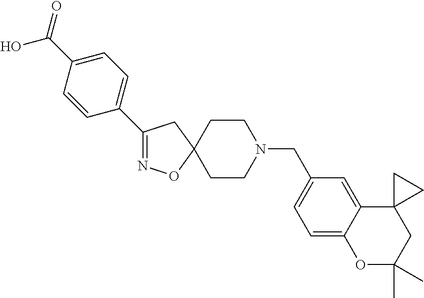 [SMILES: CC1(C)CC2(CC2)c2cc(CN3CCC4(CC3)CC(c3ccc(C(=O)O)cc3)=NO4)ccc2O1]

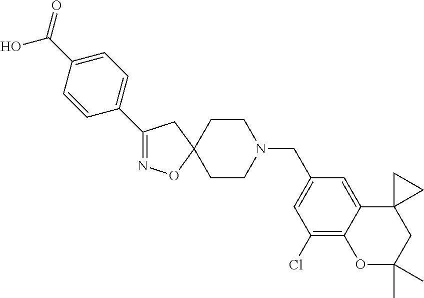 [SMILES: CC1(C)CC2(CC2)c2cc(CN3CCC4(CC3)CC(c3ccc(C(=O)O)cc3)=NO4)cc(Cl)c2O1]

For example, compounds described herein include:

TABLE 2

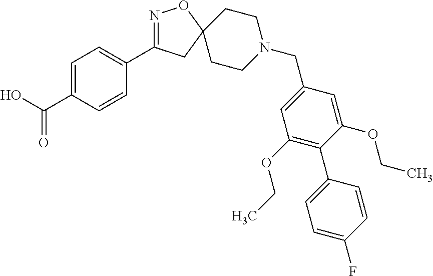 [SMILES: CCOc1cc(CN2CCC3(CC2)CC(c2ccc(C(=O)O)cc2)=NO3)cc(OCC)c1-c1ccc(F)cc1]

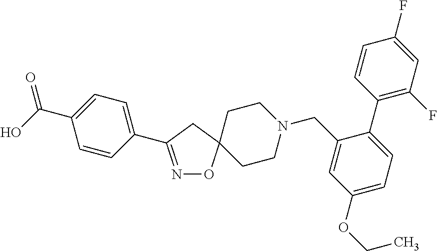 [SMILES: CCOc1ccc(-c2ccc(F)cc2F)c(CN2CCC3(CC2)CC(c2ccc(C(=O)O)cc2)=NO3)c1]

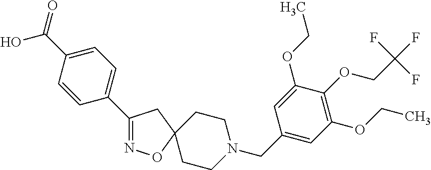 [SMILES: CCOc1cc(CN2CCC3(CC2)CC(c2ccc(C(=O)O)cc2)=NO3)cc(OCC)c1OCC(F)(F)F]

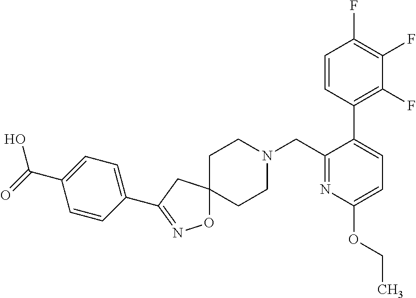 [SMILES: CCOc1ccc(-c2ccc(F)c(F)c2F)c(CN2CCC3(CC2)CC(c2ccc(C(=O)O)cc2)=NO3)n1]

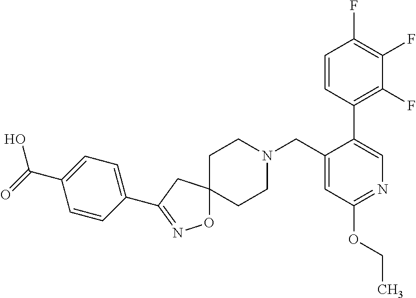 [SMILES: CCOc1cc(CN2CCC3(CC2)CC(c2ccc(C(=O)O)cc2)=NO3)c(-c2ccc(F)c(F)c2F)cn1]

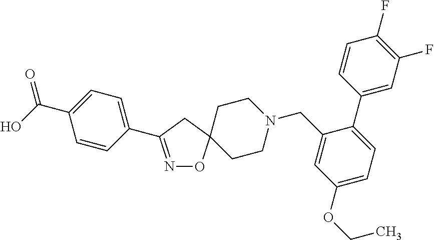 [SMILES: CCOc1ccc(-c2ccc(F)c(F)c2)c(CN2CCC3(CC2)CC(c2ccc(C(=O)O)cc2)=NO3)c1]

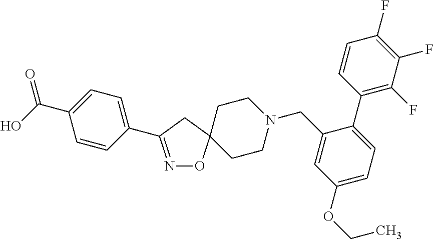 [SMILES: CCOc1ccc(-c2ccc(F)c(F)c2F)c(CN2CCC3(CC2)CC(c2ccc(C(=O)O)cc2)=NO3)c1]

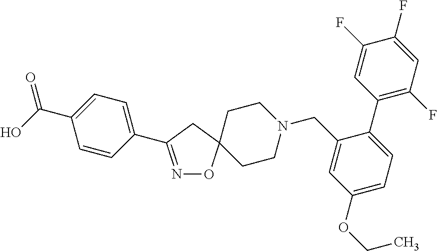 [SMILES: CCOc1ccc(-c2cc(F)c(F)cc2F)c(CN2CCC3(CC2)CC(c2ccc(C(=O)O)cc2)=NO3)c1]

### DEFINITIONS

Examples of “halogen” include a fluorine atom, a chlorine atom, a bromine atom, and an iodine atom.

“C₃-C₁₀cycloalkyl” encompasses cycloalkyl having 3 to 10 carbons, forming one or more carboxylic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“—OC₁-C₁₀alkyl” refers to an alkyl group having 1 to 10 carbons linked to oxygen, also known as an alkoxy group. Examples include methoxy, ethoxy, butoxy, isopropoxy and propoxy. “—OC₁-C₁₀halogen-substituted alkyl” refers to an alkoxy group, wherein one or more hydrogens is replaced with a halogen. Examples include trifluoromethoxy.

The term “C₁-C₁₀alkyl” encompasses straight alkyl having a carbon number of 1 to 10 and branched alkyl having a carbon number of 3 to 10. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “halogen-substitutedC₁-C₁₀alkyl” encompasses C₂-C₁₀alkyl with the hydrogen atoms thereof being partially or completely substituted with halogen, examples thereof including fluoromethyl, difluoromethyl, trifluoromethyl, 2-fluoroethyl, 1,2-difluoroethyl, 2,2-difluoroethyl and the like.

“Heterocycle” unless otherwise specified, means an aromatic, partially aromatic or non-aromatic monocyclic or polycyclic (including bicyclic) ring having at least one ring heteroatom selected from O, S and N. Examples of heterocyclic groups include pyrrolyl, isoxazolyl, isothiazolyl, pyrazolyl, pyridinyl, 2-oxo-(1H)-pyridinyl (2-hydroxy-pyridinyl), oxazolyl, 1,2,4-oxadiazolyl, 1,3,4-oxadiazolyl, thiadiazolyl, thiazolyl, imidazolyl, triazolyl, tetrazolyl, furyl, triazinyl, thienyl, pyrimidinyl, pyrazinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, imidazo[1,2-a]pyridinyl, [1,2,4-triazolo][4,3-a]pyridinyl, pyrazolo[1,5-a]pyridinyl, [1,2,4-triazolo][1,5-a]pyridinyl, 2-oxo-1,3-benzoxazolyl, 4-oxo-3H-quinazolinyl, 3-oxo-[1,2,4]-triazolo[4,3-a]-2H-pyridinyl, 5-oxo-[1,2,4]-4H-oxadiazolyl, 2-oxo-[1,3,4]-3H-oxadiazolyl, 2-oxo-1,3-dihydro-2H-imidazolyl, 3-oxo-2,4-dihydro-3H-1,2,4-triazolyl, and the like. Examples of “heterocycle” also include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. “Halogen-substituted heterocycle” means an aromatic, partially aromatic or non-aromatic monocyclic or polycyclic (including bicyclic) ring having at least one ring heteroatom selected from O, S and N, wherein one or more of the hydrogens is replaced with a halogen. Examples include fluoropryidine.

“Cycloalkyl” means mono- or bicyclic or bridged saturated carbocyclic rings, each of which having from 3 to 10 carbon atoms, unless otherwise noted, such as C₁-C₆cycloalkyl. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, and the like. “Halogen-substituted aryl” means mono- or bicyclic aromatic rings containing only carbon atoms wherein one or more of the hydrogens is replaced with halogens. Examples include fluorophenyl, difluorophenyl, trifluorophenyl and clorofluororphenyl.

“Oxo” means the functional group “═O”, such as, for example, (1) “C═(O)”, that is a carbonyl group; (2) “S═(O)”, that is, a sulfoxide group; and (3) “N═(O)”, that is, an N-oxide group, such as pyridyl-N-oxide.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that, as used herein, references to the compounds of the structural formulas described herein are meant to also include the pharmaceutically acceptable salts, and also salts that are not pharmaceutically acceptable when they are used as precursors to the free compounds or their pharmaceutically acceptable salts or in other synthetic manipulations.

It will be also understood that these alcohol compounds can be converted to the esters of phosphate, amino acid, acetic acid, etc, which can be used as pro-drugs to improve pharmacokinetic or pharmaceutical properties.

Solvates, and in particular, the hydrates of the compounds of structural formula I are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds described herein. For example, different isotopic forms of hydrogen (H) include protium (¹H) and deuteriunm (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic formula can be prepared without undue experimentation by conventional techniques well known to those skilled in the art or by processes analogous to those described in the Schemes and Examples herein using appropriate isotopically-enriched reagents and/or intermediates.

When any variable (e.g., R1, α, etc.) occurs more than one time in any constituent or in formula I, its definition on each occurrence is independent of its definition at every other occurrence. Also, combinations of substituents and/or variables are permissible only if such combinations result in stable compounds.

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

### Methods of Use

The present invention relates to methods for the treatment, control, or prevention of diseases that are responsive to antagonism of SSTR5. The compounds described herein are potent and selective antagonists of the SSTR5. The compounds are efficacious in the treatment of diseases that are modulated by SSTR5 ligands, which are generally antagonists. One or more of the following diseases may be treated by the administration of a therapeutically effective amount of a compound of the formulas described herein, or a pharmaceutically acceptable salt thereof, to a subject in need thereof: (1) Type 2 diabetes (also known as non-insulin dependent diabetes mellitus, or NIDDM), (2) hyperglycemia, (3) impaired glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) abdominal obesity, (16) retinopathy, (17) Metabolic Syndrome, (18) high blood pressure (hypertension), (19) mixed or diabetic dyslipidemia, and (20) hyperapolipoproteinemia.

The present invention also relates to methods for the treatment, control, or prevention of diseases, including but not limited to, diabetes, hyperglycemia, insulin resistance, obesity, lipid disorders, atherosclerosis, and Metabolic Syndrome by administering, to a subject, the compounds and pharmaceutical compositions described herein. Also, the compounds of the formulas described herein may be used for the manufacture of a medicament for treating one or more of these diseases.

One embodiment of the uses of the compounds is directed to the treatment of one or more of the following diseases by administering a therapeutically effective amount to a subject in need of treatment: Type 2 diabetes; insulin resistance; hyperglycemia; lipid disorders; Metabolic Syndrome; obesity; and atherosclerosis.

The compounds may be used for manufacturing a medicament for use in the treatment of one or more of these diseases.

The compounds are expected to be effective in lowering glucose and lipids in diabetic patients and in non-diabetic patients who have impaired glucose tolerance and/or are in a pre-diabetic condition. The compounds may ameliorate hyperinsulinemia, which often occurs in diabetic or pre-diabetic patients, by modulating the swings in the level of serum glucose that often occurs in these patients. The compounds may also be effective in treating or reducing insulin resistance. The compounds may be effective in treating or preventing gestational diabetes.

The compounds, compositions, and medicaments as described herein may also be effective in reducing the risks of adverse sequelae associated with Metabolic Syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

By keeping hyperglycemia under control, the compounds may also be effective in delaying or preventing vascular restenosis and diabetic retinopathy.

The compounds of this invention may also have utility in improving or restoring β-cell function, so that they may be useful in treating Type 1 diabetes or in delaying or preventing a patient with Type 2 diabetes from needing insulin therapy.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound of the formulas described herein. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD-4522. The compound may also be used advantageously in combination with other lipid lowering drugs such as cholesterol absorption inhibitors (for example stanol esters, sterol glycosides such as tiqueside, and azetidinones such as ezetimibe), ACAT inhibitors (such as avasimibe), CETP inhibitors (for example torcetrapib and those described in published applications WO2005/100298, WO2006/014413, and WO2006/014357), niacin and niacin receptor agonists, bile acid sequestrants, microsomal triglyceride transport inhibitors, and bile acid reuptake inhibitors. These combination treatments may be effective for the treatment or control of one or more related conditions selected from the group consisting of hypercholesterolemia, atherosclerosis, hyperlipidemia, hypertriglyceridemia, dyslipidemia, high LDL, and low HDL.

The term “diabetes” as used herein includes both insulin-dependent diabetes (that is, also known as IDDM, Type-1 diabetes), and insulin-independent diabetes (that is, also known as NIDDM, Type-2 diabetes).

Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

The compounds and compositions described herein are useful for treatment of both Type 1 diabetes and Type 2 diabetes. The compounds and compositions are especially useful for treatment of Type 2 diabetes. The compounds and compositions described herein are especially useful for treatment and/or prevention of pre-diabetes. Also, the compounds and compositions described herein are especially useful for treatment and/or prevention of gestational diabetes mellitus.

Treatment of diabetes mellitus refers to the administration of a compound or combination described herein to treat a diabetic subject. One outcome of the treatment of diabetes is to reduce an increased plasma glucose concentration. Another outcome of the treatment of diabetes is to reduce an increased insulin concentration. Still another outcome of the treatment of diabetes is to reduce an increased blood triglyceride concentration. Still another outcome of the treatment of diabetes is to increase insulin sensitivity. Still another outcome of the treatment of diabetes may be enhancing glucose tolerance in a subject with glucose intolerance. Still another outcome of the treatment of diabetes is to reduce insulin resistance. Another outcome of the treatment of diabetes is to lower plasma insulin levels. Still another outcome of treatment of diabetes is an improvement in glycemic control, particularly in Type 2 diabetic subjects. Yet another outcome of treatment is to increase hepatic insulin sensitivity.

Prevention of diabetes mellitus, in particular diabetes associated with obesity, refers to the administration of a compound or combination described herein to prevent or treat the onset of diabetes in a subject in need thereof. A subject in need of preventing diabetes is a prediabetic subject. In certain embodiments the compounds described herein can be useful in the treatment, control or prevention of Type 2 diabetes and in the treatment, control and prevention of the numerous conditions that often accompany Type 2 diabetes, including Metabolic Syndrome X, reactive hypoglycemia, and diabetic dyslipidemia. Obesity, discussed below, is another condition that is often found with Type 2 diabetes that may respond to treatment with the compounds described herein.

The following diseases, disorders and conditions are related to Type 2 diabetes, and therefore may be treated, controlled or in some cases prevented, by treatment with the compounds described herein: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), and other disorders where insulin resistance is a component.

Dyslipidemias or disorders of lipid metabolism, include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and HDL). Dyslipidemia includes atherogenic dyslipidemia. Hyperlipidemia is associated with abnormally high levels of lipids, LDL and VLDL cholesterol, and/or triglycerides. An outcome of the treatment of dyslipidemia, including hyperlipemia, is to reduce an increased LDL cholesterol concentration. Another outcome of the treatment is to increase a low-concentration of HDL cholesterol. Another outcome of treatment is to decrease very low density lipoproteins (VLDL) and/or small density LDL.

The term “Metabolic Syndrome”, also known as Syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having Metabolic Syndrome if the person has three or more of the following symptoms: abdominal obesity, hypertriglyceridemia, low HDL cholesterol, high blood pressure, and high fasting plasma glucose. The criteria for these are defined in ATP-III.

The term “obesity” as used herein is a condition in which there is an excess of body fat, and includes visceral obesity. The operational definition of obesity is based on the Body Mass Index (BMI), which is calculated as body weight per height in meters squared (kg/m2). “Obesity” refers to a condition whereby an otherwise healthy subject has a Body Mass Index (BMI) greater than or equal to 30 kg/m2, or a condition whereby a subject with at least one co-morbidity has a BMI greater than or equal to 27 kg/m2. An “obese subject” is an otherwise healthy subject with a Body Mass Index (BMI) greater than or equal to 30 kg/m2 or a subject with at least one co-morbidity with a BMI greater than or equal to 27 kg/m2. A “subject at risk of obesity” is an otherwise healthy subject with a BMI of 25 kg/m2 to less than 30 kg/m2 or a subject with at least one co-morbidity with a BMI of 25 kg/m2 to less than 27 kg/m2.

The increased risks associated with obesity occur at a lower Body Mass Index (BMI) in Asians than that in Europeans and Americans. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m2. In Asia-Pacific, a “subject at risk of obesity” is a subject with a BMI of greater than 23 kg/m2 to less than 25 kg/m2.

As used herein, the term “obesity” is meant to encompass all of the above definitions of obesity.

Obesity-induced or obesity-related co-morbidities include, but are not limited to, diabetes, impaired glucose tolerance, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations described herein to reduce or maintain the body weight of an obese subject. One outcome of treatment may be reducing the body weight of an obese subject relative to that subject's body weight immediately before the administration of the compounds or combinations described herein. Another outcome of treatment may be decreasing body fat, including visceral body fat. Another outcome of treatment may be preventing body weight gain. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations described herein to reduce or maintain the body weight of a subject at risk of obesity. One outcome of prevention may be reducing the body weight of a subject at risk of obesity relative to that subject's body weight immediately before the administration of the compounds or combinations described herein. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

The term “subject” is a mammal, including but not limited to a human, cat and dog.

In certain embodiments, the pharmaceutical formulations described herein are useful for the treatment, control, or prevention of obesity and the conditions associated with obesity. Obesity may be due to any cause, whether genetic or environmental. Other conditions associated with obesity include gestational diabetes mellitus and prediabetic conditions such as, elevated plasma insulin concentrations, impaired glucose tolerance, impaired fasting glucose and insulin resistance syndrome. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

Also described herein, are methods of enhancing GLP-1 secretion in a subject by administering, to a subject, the compounds and pharmaceutical compositions described herein. The incretin hormone GLP-1 is believed to have several beneficial effects for the treatment of diabetes mellitus and obesity. GLP-1 stimulates glucose-dependent biosynthesis and secretion of insulin, suppresses glucaon secretion, and slows gastric emptying. Glucagon serves as the major regulatory hormone attenuating the effect of insulin in its inhibition of liver gluconeogenesis and is normally secreted by alpha cells in pancreatic islets in response to falling blood glucose levels. The hormone binds to specific receptors in liver cells that trigger glycogenolysis and an increase in gluconeogenesis through cAMP-mediated events. These responses generate glucose (e.g. hepatic glucose production) to help maintain euglycemia by preventing blood glucose levels from falling significantly. In addition to elevated levels of circulating insulin, Type 2 diabetics have elevated levels of plasma glucagon and increased rates of hepatic glucose production. Compounds that can enhance GLP-1 secretion are useful in improving insulin responsiveness in the liver, decreasing the rate of gluconeogenesis and glycogenolysis, and lowering the rate of hepatic glucose output resulting in a decrease in the levels of plasma glucose.

### Administration and Dose Ranges

Any suitable route of administration may be employed for providing a subject, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds described herein are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds described herein are indicated, generally satisfactory results are obtained when the compounds described herein are administered at a daily dosage of from about 0.1 milligram to about 100 milligram per kilogram of animal body weight, preferably given as a single daily dose or in divided doses two to six times a day, or in sustained release form. For most large subjects, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

### Pharmaceutical Compositions

Another aspect of the present invention provides pharmaceutical compositions which comprise a compound of the formulas described herein and a pharmaceutically acceptable carrier. The pharmaceutical compositions of the present invention comprise a compound of the formulas described herein or a pharmaceutically acceptable salt as an active ingredient, as well as a pharmaceutically acceptable carrier and unsubstituted or other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the formulas described herein can be combined as the active ingredient in an intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

In some instances, depending on the solubility of the compound or salt being administered, it may be advantageous to formulate the compound or salt as a solution in an oil such as a triglyceride of one or more medium chain fatty acids, a lipophilic solvent such as triacetin, a hydrophilic solvent (e.g. propylene glycol), or a mixture of two or more of these, also unsubstituted or including one or more ionic or nonionic surfactants, such as sodium lauryl sulfate, polysorbate 80, polyethoxylated triglycerides, and mono and/or diglycerides of one or more medium chain fatty acids. Solutions containing surfactants (especially 2 or more surfactants) will form emulsions or microemulsions on contact with water. The compound may also be formulated in a water soluble polymer in which it has been dispersed as an amorphous phase by such methods as hot melt extrusion and spray drying, such polymers including hydroxylpropylmethylcellulose acetate (HPMCAS), hydroxylpropylmethyl cellulose (HPMCS), and polyvinylpyrrolidinones, including the homopolymer and copolymers.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of the formulas described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds of the formulas described herein or the other drugs may have utility, where the combination of the drugs together are safer or more effective than either drug alone. Such other drug(s) may be administered, by a route and in an amount commonly used therefore, contemporaneously or sequentially with a compound of the formulas described herein. When a compound of the formulas described herein is used contemporaneously with one or more other drugs, a pharmaceutical composition in unit dosage form containing such other drugs and the compound of the formulas described here is preferred. However, the combination therapy may also include therapies in which the compound of the formulas described herein and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the formulas described herein.

Examples of other active ingredients that may be administered in combination with a compound of the formulas described herein, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin or insulin analogs, such as insulin lispro, insulin detemir, insulin glargine, insulin glulisine, and inhalable formulations of each thereof;

(4) leptin and leptin derivatives and agonists;

(5) amylin and amylin analogs, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics; and GLP-1 receptor agonists, such as exenatide, liraglutide, taspoglutide, AVE0010, CJC-1131, and BIM-51077, including intranasal, transdermal, and once-weekly formulations thereof;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelama hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-119, and GPR-40;

(22) SSTR3 antagonists, such as those disclosed in WO 2009/011836;

(23) neuromedin U receptor agonists, such as those disclosed in WO2009/042053, including, but not limited to, neuromedin S (NMS);

(24) inhibitors of stearoyl-coenzyme A delta-9 desaturase (SCD);

(25) GPR-105 antagonists, such as those disclosed in WO 2009/000087;

(26) inhibitors of glucose uptake, such as sodium-glucose transporter (SGLT) inhibitors and its various isoforms, such as SGLT-1; SGLT-2, such as dapagliflozin and remogliflozin; and SGLT-3;

(27) inhibitors of acyl coenzyme A:diacylglycerol acyltransferase 1 and 2 (DGAT-1 and DGAT-2);

(28) inhibitors of fatty acid synthase;

(29) inhibitors of acetyl-CoA carboxylase-1 and 2 (ACC-1 and ACC-2);

(30) inhibitors of acyl coenzyme A:monoacylglycerol acyltransferase 1 and 2 (MGAT-1 and MGAT-2);

(31) agonists of the TGR5 receptor (also known as GPBAR1, BG37, GPCR19, GPR131, and M-BAR); and

(32) bromocriptine mesylate and rapid-release formulations thereof.

Dipeptidyl peptidase-IV (DPP-4) inhibitors that can be used in combination with compounds of the formulas described herein include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of the formulas described herein include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl)tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of the formulas described herein include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); and 5-hydroxytryptamine-2c (5-HT2c) agonists, such as lorcaserin. For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of the formulas described herein include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{((1S)-[3-(3,5-dichlorophenyl)-5-(6-methoxy-2-naphthyl)-1H-pyrazol-1-yl]ethyl}benzoyl)-β-alanine;
N-(4-{((1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Inhibitors of stearoyl-coenzyme A delta-9 desaturase (SCD) that can be used in combination with the compounds of the formulas described herein include, but are not limited to:
[5-(5-{4-[2-(trifluoromethyl)phenoxy]piperidin-1-yl}-1,3,4-thiadiazol-2-yl)-2H-tetrazol-2-yl]acetic acid;
(2′-{4-[2-(trifluoromethyl)phenoxy]piperidin-1-yl}-2,5′-bi-1,3-thiazol-4-yl)acetic acid;
(5-{3-[4-(2-bromo-5-fluorophenoxy)piperidin-1-yl]isoxazol-5-yl}-2H-tetrazol-2-yl)acetic acid;
(3-{3-[4-(2-bromo-5-fluorophenoxy)piperidin-1-yl]-1,2,4-oxadiazol-5-yl}-1H-pyrrol-1-yl)acetic acid;
(5-{5-[4-(2-bromo-5-fluorophenoxy)piperidin-1-yl]pyrazin-2-yl}-2H-tetrazol-2-yl)acetic acid;
   and
(5-{2-[4-(5-bromo-2-chlorophenoxy)piperidin-1-yl]pyrimidin-5-yl}-2H-tetrazol-2-yl)acetic acid;
   and pharmaceutically acceptable salts thereof.

Glucokinase activators that can be used in combination with the compounds of the formulas described herein include, but are not limited to:
3-(6-ethanesulfonylpyridin-3-yloxy)-5-(2-hydroxy-1-methyl-ethoxy)-N-(1-methyl-1H-pyrazol-3-yl)benzamide;
5-(2-hydroxy-1-methyl-ethoxy)-3-(6-methanesulfonylpyridin-3-yloxy)-N-(1-methyl-1H-pyrazol-3-yl)benzamide;
5-(1-hydroxymethyl-propoxy)-3-(6-methanesulfonylpyridin-3-yloxy)-N-(1-methyl-1H-pyrazol-3-yl)benzamide;
3-(6-methanesulfonylpyridin-3-yloxy)-5-(1-methoxymethyl-propoxy)-N-(1-methyl-1H-pyrazol-3-yl)benzamide;
5-isopropoxy-3-(6-methanesulfonylpyridin-3-yloxy)-N-(1-methyl-1H-pyrazol-3-yl)benzamide;
5-(2-fluoro-1-fluoromethyl-ethoxy)-3-(6-methanesulfonylpyridin-3-yloxy)-N-(1-methyl-UH-pyrazol-3-yl)benzamide;
3-({4-[2-(dimethylamino)ethoxy]phenyl}thio)-N-(3-methyl-1,2,4-thiadiazol-5-yl)-6-[(4-methyl-4H-1,2,4-triazol-3-yl)thio]pyridine-2-carboxamide;
3-({4-[(1-methylazetidin-3-yl)oxy]phenyl}thio)-N-(3-methyl-1,2,4-thiadiazol-5-yl)-6-[(4-methyl-4H-1,2,4-triazol-3-yl)thio]pyridine-2-carboxamide;
N-(3-methyl-1,2,4-thiadiazol-5-yl)-6-[(4-methyl-4H-1,2,4-triazol-3-yl)thio]-3-{[4-(2-pyrrolidin-1-ylethoxy)phenyl]thio}pyridine-2-carboxamide; and
3-[(4-{2-[(2R)-2-methylpyrrolidin-1-yl]ethoxy}phenyl)thio-N-(3-methyl-1,2,4-thiadiazol-5-yl)-6-[(4-methyl-4H-1,2,4-triazol-3-yl)thio]pyridine-2-carboxamide; and pharmaceutically acceptable salts thereof.

Agonists of the GPR-119 receptor that can be used in combination with the compounds of the formulas described herein include, but are not limited to:
rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and pharmaceutically acceptable salts thereof.

Selective PPARγ modulators (SPPARγM's) that can be used in combination with the compounds of the formulas described herein include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and pharmaceutically acceptable salts thereof.

Inhibitors of 11β-hydroxysteroid dehydrogenase type 1 that can be used in combination with the compounds of the formulas described herein include, but are not limited to:
3-[1-(4-chlorophenyl)-trans-3-fluorocyclobutyl]-4,5-dicyclopropyl-r-4H-1,2,4-triazole; 3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and pharmaceutically acceptable salts thereof.

Somatostatin subtype receptor 3 (SSTR3) antagonists that can be used in combination with the compounds of the formulas described herein include, but are not limited to:

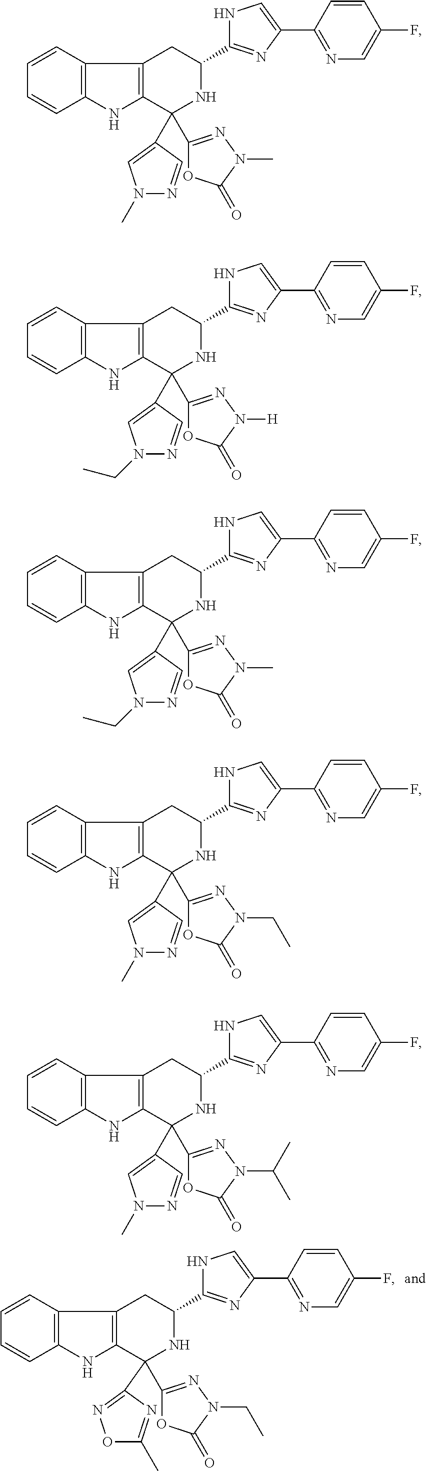

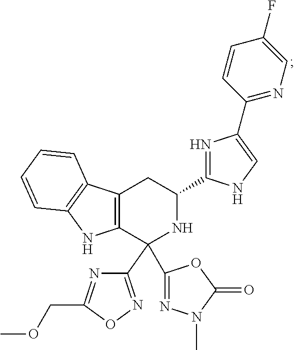 [SMILES: COCc1nc(C2(c3nn(C)c(=O)o3)N[C@@H](c3nc(-c4ccc(F)cn4)c[nH]3)Cc3c2[nH]c2ccccc32)no1]

and pharmaceutically acceptable salts thereof.

AMP-activated Protein Kinase (AMPK) activators that can be used in combination with the compounds of the formulas described herein include, but are not limited to:

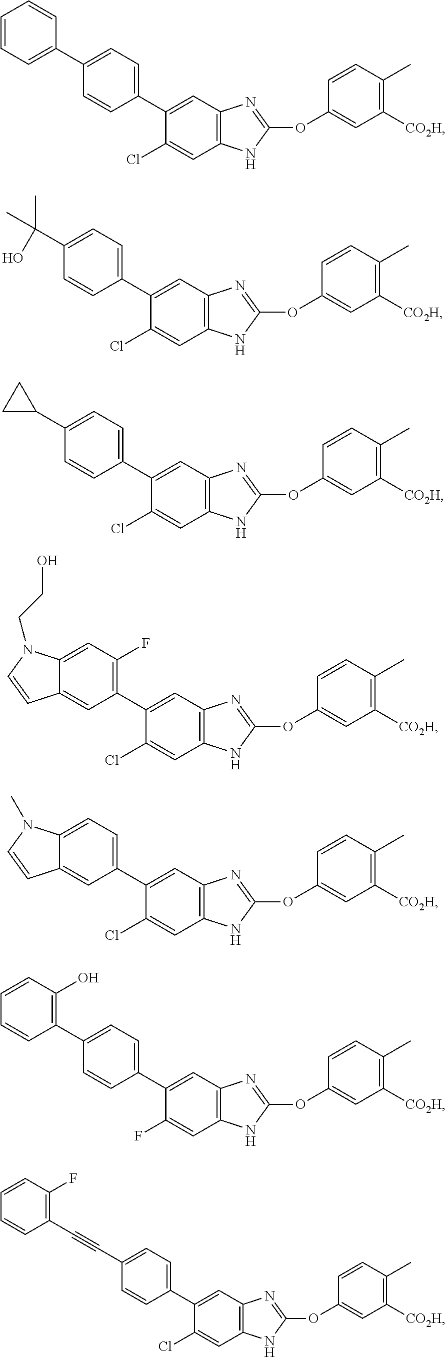

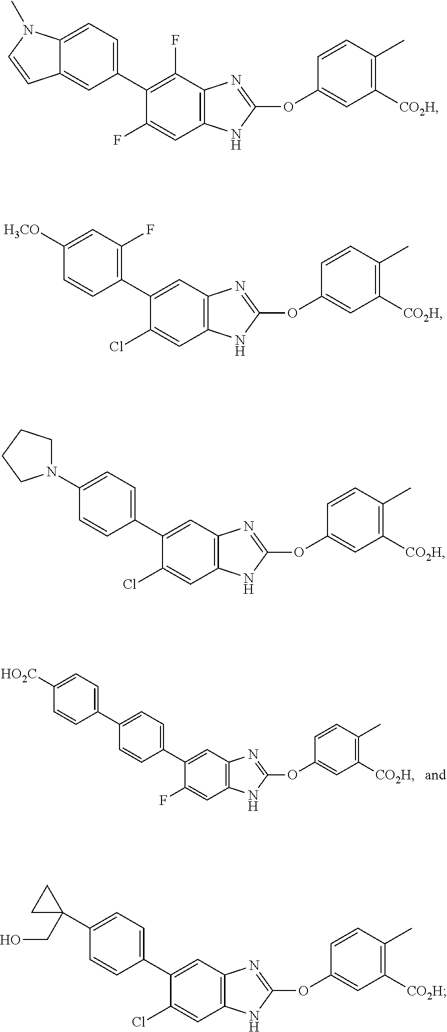 [SMILES: Cc1ccc(Oc2nc3c(F)c(-c4ccc5c(ccn5C)c4)c(F)cc3[nH]2)cc1C(=O)O ; COc1ccc(-c2cc3nc(Oc4ccc(C)c(C(=O)O)c4)[nH]c3cc2Cl)c(F)c1 ; Cc1ccc(Oc2nc3cc(-c4ccc(N5CCCC5)cc4)c(Cl)cc3[nH]2)cc1C(=O)O ; Cc1ccc(Oc2nc3cc(-c4ccc(-c5ccc(C(=O)O)cc5)cc4)c(F)cc3[nH]2)cc1C(=O)O ; Cc1ccc(Oc2nc3cc(-c4ccc(C5(CO)CC5)cc4)c(Cl)cc3[nH]2)cc1C(=O)O]

Inhibitors of acetyl-CoA carboxylase-1 and 2 (ACC-1 and ACC-2) that can be used in combination with the compounds of the formulas described herein include, but are not limited to:
3-{1′-(1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one; and
5-{1′-(1-cyclopropyl-4-methoxy-3-methyl-1H-indol-6-yl)carbonyl]-4-oxo-spiro[chroman-2,4′-piperidin]-6-yl}nicotinic acid; and
   pharmaceutically acceptable salts thereof.

In another aspect of the invention, a pharmaceutical composition is disclosed which comprises one or more of the following agents:

(a) a compound of structural formula I, formula Ia, formula Ib or formula Ic;

(b) one or more compounds selected from the group consisting of:
(1) dipeptidyl peptidase-IV (DPP-4) inhibitors;
(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;
(3) sulfonylurea and non-sulfonylurea insulin secretagogues, such as tolbutamide, glyburide, glipizide, glimepiride, mitiglinide, and meglitinides, such as nateglinide and repaglinide;
(4) α-glucosidase inhibitors (such as acarbose, voglibose and miglitol);
(5) glucagon receptor antagonists;
(6) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;
(7) HDL-raising drugs, such as niacin or a salt thereof and extended-release versions thereof; MK-524A, which is a combination of niacin extended-release and the DP-1 antagonist MK-524; and nicotinic acid receptor agonists;
(8) antiobesity compounds;
(9) agents intended for use in inflammatory conditions, such as aspirin, non-steroidal anti-inflammatory drugs (NSAIDs), glucocorticoids, and selective cyclooxygenase-2 (COX-2) inhibitors;
(10) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;
(11) glucokinase activators (GKAs), such as LY2599506;
(12) inhibitors of 11β-hydroxysteroid dehydrogenase type 1;
(13) inhibitors of cholesteryl ester transfer protein (CETP), such as torcetrapib and MK-0859;
(14) inhibitors of fructose 1,6-bisphosphatase;
(15) inhibitors of acetyl CoA carboxylase-1 or 2 (ACC1 or ACC2);
(16) AMP-activated Protein Kinase (AMPK) activators;
(17) agonists of the G-protein-coupled receptors: GPR-109, GPR-119, and GPR-40;
(18) SSTR3 antagonists;
(19) neuromedin U receptor agonists, including, but not limited to, neuromedin S (NMS);
(20) inhibitors of stearoyl-coenzyme A delta-9 desaturase (SCD);
(21) GPR-105 antagonists;
(22) inhibitors of glucose uptake, such as sodium-glucose transporter (SGLT) inhibitors and its various isoforms, such as SGLT-1; SGLT-2, such as dapagliflozin and remogliflozin; and SGLT-3;
(23) inhibitors of acyl coenzyme A:diacylglycerol acyltransferase 1 and 2 (DGAT-1 and DGAT-2);
(24) inhibitors of fatty acid synthase;
(25) inhibitors of acetyl-CoA carboxylase-1 and 2 (ACC-1 and ACC-2);
(26) inhibitors of acyl coenzyme A:monoacylglycerol acyltransferase 1 and 2 (MGAT-1 and MGAT-2);
(27) agonists of the TGR5 receptor (also known as GPBAR1, BG37, GPCR19, GPR131, and M-BAR); and
(28) bromocriptine mesylate and rapid-release formulations thereof; and

(c) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### EXAMPLES

The compounds of the present invention can be prepared according to the procedures of the following Schemes, Intermediates and Examples, using appropriate materials and are further exemplified by the following specific examples. Moreover, by utilizing the procedures described in the disclosure contained herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those previously described herein. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art.

Reactions sensitive to moisture or air were performed under nitrogen or argon using anhydrous solvents and reagents. The progress of reactions was determined by either analytical thin layer chromatography (TLC) or liquid chromatography-mass spectrum (LC-MS). Concentration of solutions was carried out on a rotary evaporator under reduced pressure. 1H NMR spectra were acquired on a 500 MHz Varian Unity INOVA NMR spectrometer in CDCl₃ solutions unless otherwise noted. Chemical shifts were reported in parts per million (ppm). Tetramethylsilane (TMS) was used as internal reference in CD₃Cl solutions, and residual CH₃OH peak or TMS was used as internal reference in CD₃OD solutions. Coupling constants (J) were reported in hertz (Hz). All temperatures are degrees Celsius unless otherwise noted. Mass spectra (MS) were measured by electron-spray ion-mass spectroscopy.

Abbreviations used in the following Schemes and Examples:

aq.: aqueous; API-ES: atmospheric pressure ionization-electrospray (mass spectrum term); Ac: acetate; AcCN: acetonitrile; Bop reagent: (benzotriazol-1-yloxy)tris(dimethylamino)phosonium hexafluorophosphate; Boc: tert-butyloxycarbonyl; B(OTMS)₃: tris(trimethylsilyl) borate; Celite™: diatomaceous earth; CDI: carbonyl diimidazole; d: day(s); d is doublet (NMR); DCM: dichloromethane; Dess-Martin reagent: 1,1,1-triacetoxy-, 1-dihydro-1,2-benziodoxol-3(1H)-one; DIBAL: diisobutylaluminum hydride; DIEA and DIPEA: N,N-diisopropyl-ethylamine (Hunig's base); DMAP: 4-dimethylaminopyridine; DMF: N,N-dimethylformamide; DMSO: dimethylsulfoxide; DTBPF is 1,1′-bis(di-tert-butylphosphino)-ferrocene; eq: equivalent(s); Et is ethyl; OEt is ethoxy; EtOAc: ethyl acetate; EtOH: ethanol; g: gram(s); h or hr: hour(s); HPLC: high pressure liquid chromatography; HPLC/MS: high pressure liquid chromatography/mass spectrum; in vacuo: rotary evaporation under diminished pressure; iPrOH or IPA: isopropyl alcohol; IPAC or IPAc: isopropyl acetate; [Ir(COD)Cl]2: chloro-1,5-cyclooctadiene iridium (I) dimmer; L: liter; LC: Liquid chromatography; LC-MS: liquid chromatography-mass spectrun; m is multiplet (NMR); M: molar; Me: methyl; MeCN: methylcyanide; MeI: methyl iodide; MeOH: methanol; Ms: methanesulfonyl; MsCl: methanesulfonyl chloride; MHz: megahertz; mg: milligram; min: minute(s); ml or mL: milliliter; mmol: millimole; MPLC: medium-pressure liquid chromatography; MS or ms: mass spectrum; N: normal; nM: nanomole(s); NMR: nuclear magnetic resonance; NMM: N-methylmorpholine; Pd2(dba)₃: tris(dibenzyldeneacetone)dipalladium(0); q is quadruplet (NMR); Rt: retention time; rt or RT: room temperature; s is singlet (NMR); satd.: saturated; SRIF is somatotropin release-inhibiting factor or somatostatin; t is triplet (NMR); TBAF is tetrabutyl ammonium fluoride; TBS is tert-butyldimethylsilyl; TBSCl is tert-butyldimethylsily chloride; TEA: triethylamine; TFA: trifluoroacetic acid; THF: tetrahydrofuran; TLC or tlc: thin layer chromatography; Tf is trifluoromethane sulfonate; and Ts is toluene sulfonyl.

The present compounds can be prepared according to the general Schemes provided below as well as the procedures provided in the Examples. The following Schemes and Examples further describe, but do not limit, the scope.

Scheme 1 illustrates the synthesis of spiro isoxazoline compounds. The condensation of an aryl aldehyde and hydroxylamine gave an oxime intermediate which can undergo 2+3 dipolar cycloaddition with appropriate olefins upon treatment of NCS and TEA. The protecting group (Boc) was removed and the piperidine nitrogen can be alkylated by treatment with an appropriate aryl methyl halide and a base or through a variety of reductive amination conditions.

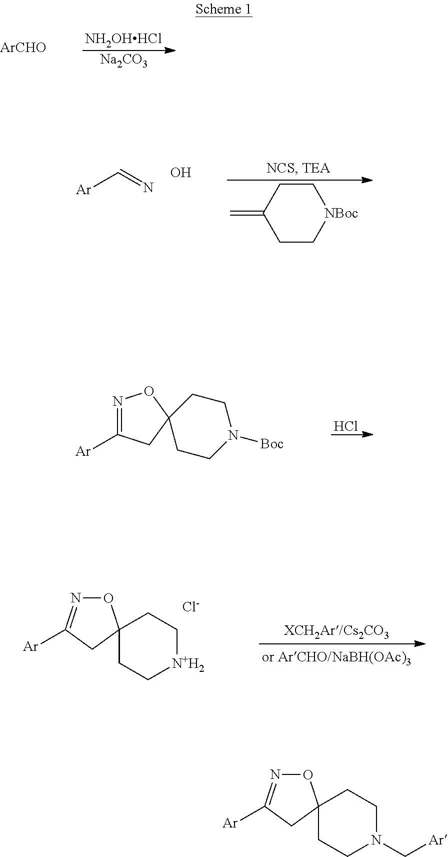

### Intermediate 1

### 3-[4-(Methoxycarbonyl)phenyl]-1-oxa-2,8-diazaspiro[4.5]dec-2-ene hydrochloride

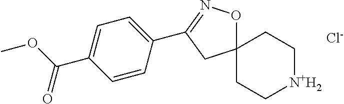

### Step A: Synthesis of methyl 4-[(E)-(hydroxyimino)methyl]benzoate

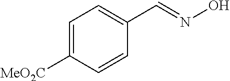 [SMILES: COC(=O)c1ccc(/C=N/O)cc1]

To a 100 mL round bottom flask, methyl 4-formylbenzoate (330 mg, 2.0 mmol), hydroxylamine hydrochloride (210 mg, 3.0 mmol), sodium carbonate (2 mL, 2M) and DCM (10 mL) were added. The resulting reaction mixture was stirred at 50° C. for 0.5 hour. The reaction mixture was diluted with ethyl acetate and water. The organic layer was separated and dried over sodium sulfate, filtered and concentrated to afford the title compound as a white solid.

¹H NMR (CDCl₃, 500 MHz): b 8.2 (s, 1H), 8.1 (d, J=8.3 Hz, 2H), 7.6 (d, J=8.4 Hz, 2H), 3.9 (s, 3H).

[M+H⁺]: m/z 180.

### Step B: Synthesis of tert-butyl 3-[4-(methoxycarbonyl)phenyl]-1-oxa-2,8-diazaspiro[4.5]dec-2-ene-8-carboxylate

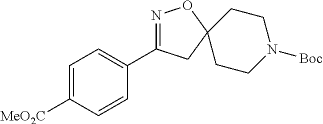 [SMILES: COC(=O)c1ccc(C2=NOC3(CCN(C(=O)OC(C)(C)C)CC3)C2)cc1]

To a 250 mL round bottom flask, methyl 4-[(E)-(hydroxyimino)methyl]benzoate (2 g, 11.2 mmol), DCM (100 mL) and DMF (10 mL) were added. NCS was added in portions to the stirred mixture at room temperature. The progress of the chlorination was monitored by NMR. tert-Butyl 4-methylidenepiperidine-1-carboxylate (3.3 g, 16.74 mmol) was then added to the mixture followed by the slow addition of TEA (4.67 mL) via a syringe pump over 3 hours. The resulting reaction mixture was stirred at room temperature overnight. Volatiles were removed and the residue was redissolved in ethyl acetate (100 mL). It was washed sequentially with 1N HCl, 10% KOH and brine and was then dried over sodium sulfate, filtered and concentrated in vacuo. The crude material was purified on a silica gel column eluting with 30% ethyl acetate in hexanes to give the title compound as a colorless oil.

¹H NMR (CD₃OD, 500 MHz): δ 8.0 (d, J=8.4 Hz, 2H), 7.8 (d, J=8.4 Hz, 2H), 3.9 (s, 3H), 3.65 (m, 2H), 3.45 (m, 2H), 3.25 (s, 2H), 1.85 (m, 2H), 1.8 (m, 2H), 1.48 (s, 9H).

[M+H⁺]: m/z 375.

### Step C: Synthesis of 3-[4-(methoxycarbonyl)phenyl]-1-oxa-2,8-diazaspiro[4.5]dec-2-ene hydrochloride

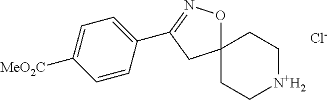

To a 250 mL round bottom flask were added tert-butyl 3-[4-(methoxycarbonyl)phenyl]-1-oxa-2,8-diazaspiro[4.5]dec-2-ene-8-carboxylate (2.0 g, 5.34 mmol) and DCM (10 mL). A solution of 4 M HCl in dioxane (6.6 mL) was added dropwise to the solution. The mixture was stirred at room temperature for 1 hour. A white precipitate was observed. The volatiles were removed under reduced pressure. The crude product was washed with ether and dried under vacuum to give the title compound (1.6 g) as a white solid.

¹H NMR (CD₃OD, 500 MHz): δ 8.0 (d, J=8.4 Hz, 2H), 7.8 (d, J=8.4 Hz, 2H), 3.95 (s, 3H), 3.65 (m, 2H), 3.45 (m, 2H), 3.25 (s, 2H), 1.85 (m, 2H), 1.8 (m, 2H).

[M+H⁺]: m/z 275.

### Intermediate 2

### 3-(4-Carboxyphenyl)-1-oxa-2,8-diazaspiro[4.5]dec-2-ene hydrochloride

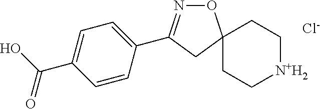

To INTERMEDIATE 1 (1600 mg, 5.15 mmol) and LiOH (432 mg, 18.0 mmol) was added water (8.6 mL) and MeOH (17.2 mL). The mixture was stirred at 5° C. for 3 hours. The MeOH was evaporated in vacuo and the solution was made acidic with 3N HCl. The precipitate was collected, washed with ether and dried under reduced pressure to provide the title compound as a white solid (1457 mg) which required no further purification.

¹H NMR (DMSO-d₆, 400 MHz) δ 9.20 (br m, 2H), 7.95 (d, J=8.4 Hz, 2H), 7.71 (d, J=8.4 Hz, 2H), 3.34 (s, 2H), 3.11 (br s, 4H), (1.98 (m, 4H).

[M+H⁺]: m/z 261.

### Intermediate 3

### 4-Ethoxy-2′,3′,4′-trifluorobiphenyl-2-carboxaldehyde

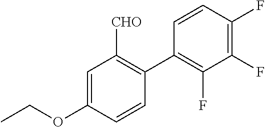 [SMILES: CCOc1ccc(-c2ccc(F)c(F)c2F)c(C=O)c1]

### Step A: Synthesis of 2-bromo-5-ethoxybenzaldehyde

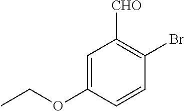

To a solution of 2-bromo-5-hydroxybenzaldehyde (5 g, 24.87 mmol) in DMF (20 mL), K₂CO₃ (6.88 g, 49.7 mmol) was added portion-wise. Iodoethane (5.82 g, 37.3 mmol) was added slowly and the resulting reaction mixture was stirred at 50° C. overnight. After cooling the reaction to ambient temperature, it was diluted with 100 mL of ether/hexanes (1:1) and 100 mL of water.

The organic layer was washed with 100 mL of brine, dried over sodium sulfate, filtered and concentrated to give the title compound 2-bromo-5-ethoxybenzaldehyde as a white solid.

¹H NMR (CDCl₃, 500 MHz): δ 10.3 (s, 1H), 7.51 (d, J=9.0 Hz, 1H), 7.39 (d, J=3.0 Hz, 1H) 7.02 (dd, J=3.5 Hz, J=8.5 Hz, 1H), 4.06 (q, 2H), 1.42 (t, J=6.5 Hz, 3H).

### Step B: Synthesis of 4-ethoxy-2′,3′,4′-trifluorobiphenyl-2-carboxaldehyde

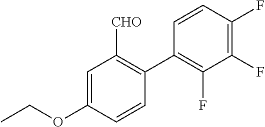 [SMILES: CCOc1ccc(-c2ccc(F)c(F)c2F)c(C=O)c1]

To a degassed solution of 2-bromo-5-ethoxybenzaldehye (300 mg, 1.31 mmol), 2-dicyclohexylphosphino-2′,6′-dimethoxy-1,1′-biphenyl (53.8 mg, 0.131 mmol; S-Phos ligand) and palladium(II) acetate (14.7 mg, 0.065 mmol) in THF (8 mL) were added K₃ PO₄ (834 mg, 3.93 mmol) and 2,3,4-trifluorophenyl boronic acid (276 mg, 1.57 mmol). The reaction mixture was stirred at 70° C. under a nitrogen atmosphere for 16 hours. After cooling to room temperature, the reaction mixture was filtered and the filtrate was concentrated by evaporation under reduced pressure. The crude material was purified on a CombiFlash silica gel column eluting with 5-10% ethyl acetate in hexanes to provide the title compound 4-ethoxy-2′,3′,4′-trifluorobiphenyl-2-carboxaldehyde as a white solid.

¹H NMR (CDCl₃, 500 MHz): δ 9.88 (s, 1H), 7.53 (s, 1H), 7.30 (m, 1H), 7.24 (dd, J=3.0 Hz, J=8.5 Hz, 1H), 7.08 (m, 2H), 4.17 (q, 2H), 1.49 (t, J=7.0 Hz, 3H).

### Intermediate 4

### 4-Ethoxy-2′,3′,4′-trifluoro-5-methylbiphenyl-2-carboxaldehyde

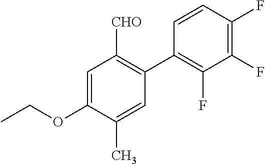 [SMILES: CCOc1cc(C=O)c(-c2ccc(F)c(F)c2F)cc1C]

### Step A: Synthesis of ethyl 2-bromo-5-ethoxy-4-methylbenzoate

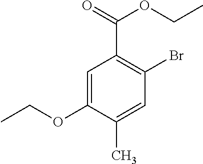 [SMILES: CCOC(=O)c1cc(OCC)c(C)cc1Br]

To a solution of ethyl 5-ethoxy-4-methylbenzoate (500 mg, 2.40 mmol) in acetic acid (10 mL) and water (10 mL) in a 50 mL flask, bromine was added at room temperature. The resulting reaction mixture was stirred at 60° C. for 1 hour. After cooling to room temperature, the reaction mixture was diluted with 100 mL of hexanes:ether (80:20). The layers were separated and the organic layer was washed with saturated Na₂CO₃, brine, dried over Na₂SO₄, filtered and concentrated. The residue was purified on a silica gel column eluting with 5-10% ethyl acetate in hexanes to yield the title compound 2-bromo-5-ethoxy-4-methylbenzoate as a light yellow solid.

¹H NMR (CDCl₃, 500 MHz): δ 7.54 (d, J=7.0 Hz, 1H), 7.47 (s, 1H), 7.17 (d, J=7.51 Hz, 1H), 4.36 (q, 2H), 4.09 (q, 2H), 2.26 (s, 3H), 1.44 (t, J=7.0 Hz, 3H), 1.38 (t, J=7.50 Hz, 3H).

### Step B: Synthesis of (2-bromo-5-ethoxy-4-methylphenyl)methanol

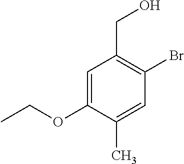

To a 100 mL round bottom flask, 2-bromo-5-ethoxy-4-methylbenzoate (660 mg, 2.23 mmol) and mL of ether were added. DIBAL in toluene (5.75 mL, 1 M) was added to the reaction mixture at 0° C. The resulting reaction mixture was stirred at 0° C. for 10 minutes. Ethyl acetate (20 mL) and wet silica gel (˜50 g silica gel and 3 mL of water) were added at 0° C. portion-wise. The resulting slurry was stirred for ˜15 minutes and was then filtered and washed with 20 mL of ethyl acetate. The organic phase was dried over sodium sulfate, filtered and concentrated. The residue was purified on a silica gel column eluting with ethyl acetate:hexanes (20:80) to give the title compound (2-bromo-5-ethoxy-4-methylphenyl)methanol as a colorless liquid.

¹H NMR (CDCl₃, 500 MHz): δ 7.28 (s, 1H), 6.93 (s, 1H), 4.68 (d, J=6.5 Hz, 2H), 4.021 (q, 2H), 2.18 (s, 3H), 1.99 (t, J=6.5 Hz, 3H).

### Step C: Synthesis of 2-bromo-5-ethoxy-4-methylbenzaldehyde

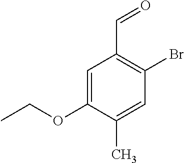

To a solution of (2-bromo-5-ethoxy-4-methylphenyl)methanol (2.30 g, 9.38 mmol) in DCM (40 mL), Dess-Martin periodinane (5.97 g, 14.08 mmol) was added. The resulting mixture was stirred at room temperature for 1 hour. The reaction mixture was diluted with ether and was concentrated to a small volume in vacuo. The residue was taken up in 30 mL of ether and then was washed with 15 mL of 1:1 10% Na₂S₂O₃: saturated aqueous NaHCO₃, followed by 10 mL of water and 10 mL of brine. The aqueous washings were back-extracted with 20 mL of ether and the organic layer was washed with water and brine. The combined organic layers were dried over Na₂SO₄ and concentrated. The residue was purified on a silica gel column eluting with 5-10% ethyl acetate in hexanes to give the title compound 2-bromo-5-ethoxy-4-methylbenzaldehyde as a white solid.

¹H NMR (CDCl₃, 500 MHz): δ 10.2 (s, 1H), 7.39 (s, 1H), 7.32 (s, 1H), 4.08 (q, 2H), 2.26 (s, 3H), 1.43 (t, J=7.0 Hz, 3H).

### Step D: Synthesis of 4-ethoxy-2′,3′,4′-trifluoro-5-methylbiphenyl-2-carboxaldehyde

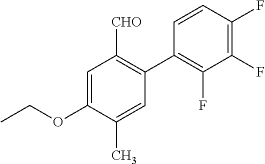 [SMILES: CCOc1cc(C=O)c(-c2ccc(F)c(F)c2F)cc1C]

To a degassed solution of 2-bromo-5-ethoxy-4-methylbenzaldehyde (300 mg, 1.31 mmol), 2-dicyclohexylphosphino-2′,6′-dimethoxy-1,1′-biphenyl (53.8 mg, 0.131 mmol; S-Phos ligand [CAS #657408-07-6] which is commercially available from Aldrich Chemicals, USA) and palladium(II) acetate (14.7 mg, 0.065 mmol) in THF (8 mL) were added K₃ PO₄ (834 mg, 3.93 mmol) and (2,3,4-trifluorophenyl) boronic acid (276, 1.57 mmol). The reaction mixture was stirred at 70° C. under a nitrogen atmosphere for 16 hours, cooled to room temperature and filtered and the filtrate was concentrated by evaporation under reduced pressure. The residue was purified on a silica gel column eluting with 5-10% ethyl acetate in hexanes to provide the title compound 4-ethoxy-2′,3′,4′-trifluoro-5-methylbiphenyl-2-carboxaldehyde as a white solid.

¹H NMR (CDCl₃, 500 MHz): δ 9.79 (s, 1H), 7.43 (s, 1H), 7.13 (s, 1H), 7.04 (m, 2H), 4.16 (q, 2H), 2.32 (s, 3H), 1.46 (t, J=7.0 Hz, 3H).

### Intermediate 5

### 5-Ethoxy-4-methyl-2-(1,3-thiazol-2-yl)benzaldehyde

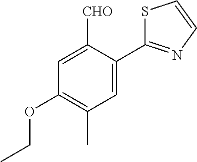 [SMILES: CCOc1cc(C=O)c(-c2nccs2)cc1C]

To a degassed solution of 2-bromo-5-ethoxy-4-methylbenzaldehyde (220 mg, 0.91 mmol) and triphenylphosphine (71.2 mg, 0.27 mmol) in THF (5 mL) were added 2-(tributylstananyl)-1,3-thiazole (372 mg, 1.0 mmol) and palladium(II) acetate (20.3 mg, 0.090 mmol). The reaction mixture was stirred at 70° C. under a nitrogen atmosphere for 16 hours and was then cooled to room temperature. The reaction mixture was diluted with ether and washed with water and brine. The ether layer was dried over Na₂SO₄ and concentrated. The residue was purified on a silica gel column eluting with 5-10% ethyl acetate in hexanes to obtain the title compound 5-ethoxy-4-methyl-2-(1,3-thiazol-2-yl)benzaldehyde as a light yellow solid.

¹H NMR (CDCl₃, 500 MHz): δ 10.49 (s, 1H), 7.91 (d, J=5.0 Hz, 1H), 7.51 (S, 1H), 7.45 (s, 1H), 7.42 (d, J=3.0 Hz, 1H), 4.16 (q, 2H), 2.33 (s, 3H), 1.43 (t, J=7.0 Hz, 3H).

### Intermediate 6

### 4,4-Dimethyl-3,4-dihydro-2H-chromene-6-carboxaldehyde

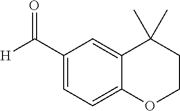

### Step A: Synthesis of 4-bromophenyl 3-methylbut-3-en-1-yl ether

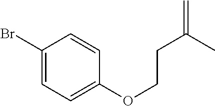

To a 250 mL round bottom flask were added 4-bromo phenol (4.08 g, 23.6 mmol), 3-methylbut-3-en-1-yl diphenyl phosphate (5.0 g, 15.7 mmol, synthesized according to a procedure in U.S., 5006550, 9 Apr. 1991), Cs₂CO₃ (15.4 g, 47.1 mmol) and DMF (25 mL). The resulting reaction mixture was heated at 130° C. for 15 minutes. It was cooled to room temperature and was diluted with 100 mL of water and 100 mL of ethyl acetate/hexanes (1:2). The layers were separated and the organic layer was washed with 30 mL of brine, dried over sodium sulfate, filtered and concentrated. The residue was purified on a silica gel column eluting with a solvent gradient from 100% hexanes to 1:9 ethyl acetate:hexanes to give the title compound (2.1 g) as light yellow oil.

¹H NMR (CDCl₃, 500 MHz): δ 7.39 (d, J=8.8 Hz, 2H), 6.80 (d, J=8.8 Hz, 2H), 4.87 (s, 1H), 4.81 (s, 1H), 4.06 (t, J=6.9 Hz, 2H), 2.51 (t; J=6.9 Hz, 2H), 1.82 (s, 3H).

### Step B: Synthesis of 6-bromo-4,4-dimethyl-3,4-dihydro-2H-chromene

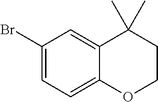

To a 100 mL round bottom flask were added AlCl₃ and CH₂Cl₂ (10 mL). A solution of 4-bromophenyl 3-methylbut-3-en-1-yl ether in CH₂Cl₂ (10 mL) was added at 0° C. The resulting reaction mixture was stirred at 0° C. for 30 minutes and was then poured into an Erlenmeyer flask containing 100 mL of 10% KOH and crushed ice. The resulting mixture was extracted with 75 mL of hexanes and the organic layers were dried over sodium sulfate, filtered and concentrated. The residue was purified on a silica gel column eluting with a solvent gradient from 100% hexanes to 1:9 ethyl acetate:hexanes to give the title compound as a colorless oil.

¹H NMR (CDCl₃, 500 MHz): δ 7.37 (s, 1H), 7.18 (d, J=8.7 Hz, 1H), 6.70 (d, J=8.7 Hz, 1H), 4.21 (m, 2H), 1.85 (m, 2H), 1.36 (s, 6H).

### Step C: Synthesis of 4,4-dimethyl-3,4-dihydro-2H-chromene-6-carboxaldehyde

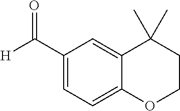

To a nitrogen flushed 100 mL round bottom flask were added 6-bromo-4,4-dimethyl-3,4-dihydro-2H-chromene (1 g, 4.15 mmol) and THF (10 mL). A solution of n-BuLi (1.76 mL, 2.6 M hexanes solution) was added via a syringe at −78° C. The resulting reaction mixture was stirred at −78° C. for 10 minutes when DMF (0.48 mL, 6.22 mmol) was added. The reaction mixture was allowed to warm to room temperature. Ethyl acetate (25 mL) and wet silica gel (10 g silica gel/0.5 mL water) were added. The resulting mixture was stirred at room temperature for minutes and then filtered. The resulting solid was rinsed with ethyl acetate and the filtrate was concentrated. The residue was purified on a silica gel column eluting with a solvent gradient from 100% hexanes to 1:4 ethyl acetate:hexanes to give the title compound as a colorless oil.

¹H NMR (CDCl₃, 500 MHz): δ 9.87 (s, 1H), 7.85 (s, 1H), 7.62 (d, J=8.4 Hz, 1H), 6.91 (d, J=8.5 Hz, 1H), 4.30 (t, J=5.5 Hz, 2H), 1.89 (t, J=5.5 Hz, 2H), 1.40 (s, 6H).

### Intermediate 7

### 1-tert-Butyl-3-(2,3,4-trifluorophenyl)-1H-pyrazole-4-carboxaldehyde

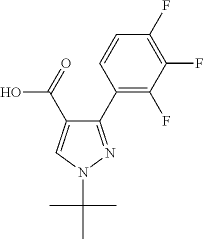 [SMILES: CC(C)(C)n1cc(C(=O)O)c(-c2ccc(F)c(F)c2F)n1]

### Step A: Synthesis of 1-tert-butyl-2-[1-(2,3,4-trifluorophenyl)ethylidene]hydrazine

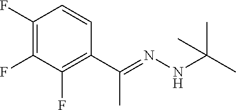 [SMILES: C/C(=N\NC(C)(C)C)c1ccc(F)c(F)c1F]

Sodium acetate (165 mg, 2.00 mmol) was added to a stirred mixture of t-butyl hydrazine hydrochloride (250 mg, 2.00 mmol) and 2,3,4-trifluorophenylacetophenone (349 mg, 2.00 mmol) in ethanol (6 mL) and the mixture was heated at 70° C. for 2 hours. The solution was cooled and concentrated to give the title compound as a mixture of E and Z 1-tert-butyl-2-[1-(2,3,4-trifluorophenyl)ethylidene]hydrazine.

### Step B: Synthesis of 1-tert-butyl-3-(2,3,4-trifluorophenyl)-1H-pyrazole-4-carboxaldehyde

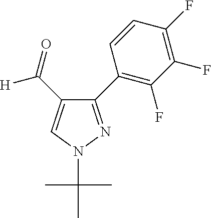 [SMILES: CC(C)(C)n1cc(C=O)c(-c2ccc(F)c(F)c2F)n1]

DMF (0.466 mL, 6.02 mmol) was added to a stirred mixture of POCl₃ (0.561 mL, 6.02 mmol) at 0° C. and the reaction mixture was stirred at room temperature for 10 minutes, tert-Butyl-2-[1-(2,3,4-trifluorophenyl)ethylidene]hydrazine (490 mg, 2.006 mmol) in DMF was added slowly to the reaction mixture at 0° C. and the reaction mixture was heated at 75° C. for 16 hours. The reaction was poured into a cooled, saturated solution of potassium carbonate (15 mL) and the mixture was extracted with ethyl acetate (2×25 mL). The organic layers were washed with water (10 mL), brine (10 mL), dried over sodium sulfate, filtered and concentrated. The residue was purified by silica gel column chromatography eluting with ethyl acetate/hexanes to give the title compound as a yellow solid.

¹H NMR (CD₃OD, 500 MHz): δ 9.77 (s, 1H), 8.49 (s, 1H), 7.37-7.34 (m, 1H), 7.24-7.20 (m, 1H), 1.66 (s, 9H).

[M+H⁺]: m/z 283.

### Intermediate 8

### 6-(1-Oxa-2,8-diazaspiro[4.5]dec-2-en-3-yl)pyridine-3-carboxylic acid dihydrochloride

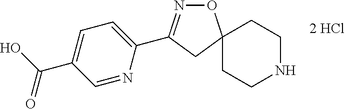 [SMILES: O=C(O)c1ccc(C2=NOC3(CCNCC3)C2)nc1]

### Step A: Synthesis of methyl 6-(1-oxa-2,8-diazaspiro[4.5]dec-2-en-3-yl)pyridine-3-carboxylate dihydrochloride

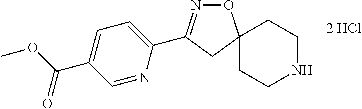 [SMILES: COC(=O)c1ccc(C2=NOC3(CCNCC3)C2)nc1]

Using essentially the same procedure as for INTERMEDIATE 1, but using methyl 6-formylpyridine-3-carboxylate in Step A, the title compound was prepared.

### Step B: Synthesis of 6-(1-oxa-2,8-diazaspiro[4.5]dec-2-en-3-yl)pyridine-3-carboxylic acid dihydrochloride

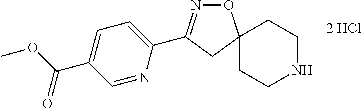 [SMILES: COC(=O)c1ccc(C2=NOC3(CCNCC3)C2)nc1]

Using essentially the same procedure as for INTERMEDIATE 2, but using methyl 6-(1-oxa-2,8-diazaspiro[4.5]dec-2-en-3-yl)pyridine-3-carboxylate dihydrochloride from Step A, the title compound was prepared.

### Intermediates 9 A-M

Using essentially the same procedures as for INTERMEDIATE 3, but using the appropriate hydroxybenzaldehyde in Step A and the appropriate boronic acid in Step B, the following aldehydes were prepared:
A 4-Ethoxy-2′,4′-difluorobiphenyl-2-carboxaldehyde
B. 4-Ethoxy-3′,4′-difluorobiphenyl-2-carboxaldehyde
C. 4-Ethoxy-2′,4′,5′-trifluorobiphenyl-2-carboxaldehyde
D. 5-Ethoxy-2-(thiazole-2-yl)benzaldehyde
E. 6-Ethoxy-3-(2,3,4-trifluorophenyl)pyridine-2-carboxaldehyde
F. 2-Ethoxy-5-(2,3,4-trifluorophenyl)pyridine-4-carboxaldehyde
G. 3′,4′-Difluoro-4-(propan-2-yloxy)biphenyl-2-carboxaldehyde
H. 4-(Propan-2-yloxy)-2′,3′,4′-trifluorobiphenyl-2-carboxaldehyde
I. 4-(Propan-2-yloxy)-2′,4′,5-trifluorobiphenyl-2-carboxaldehyde
J. 3-Ethoxy-4-(thiazole-2-yl)benzaldehyde
K. 2′,3′,4′-Trifluoro-5-(trifluoromethoxy)biphenyl-2-carboxaldehyde
L. 2-(2-Fluoropryidin-5-yl)-5-trifluoromethoxybenzaldehyde
M. 3-tert-Butyl-4-(2-fluoropryidin-5-yl)benzaldehyde

### Intermediate 10

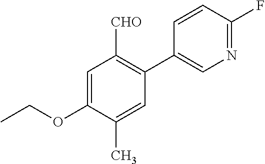 [SMILES: CCOc1cc(C=O)c(-c2ccc(F)nc2)cc1C]

Using essentially the same procedure as for INTERMEDIATE 4, Step D, but using 2-fluoropyridin-5-ylboronic acid, the title compound was prepared.

### Intermediate 11

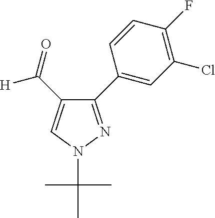 [SMILES: CC(C)(C)n1cc(C=O)c(-c2ccc(F)c(Cl)c2)n1]

Using essentially the same procedure as for INTERMEDIATE 7, but using 3-chloro-4-fluorophenylacetophenone in Step A, the title compound was prepared.

### Intermediate 12

### 2,6-Dichloro-4′-fluorobiphenyl-4-carboxaldehyde

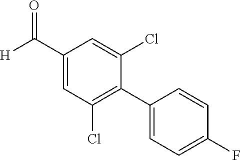 [SMILES: O=Cc1cc(Cl)c(-c2ccc(F)cc2)c(Cl)c1]

### Step A: Synthesis of (2,6-dichloro-4′-fluorobiphenyl-4-carboxylic acid

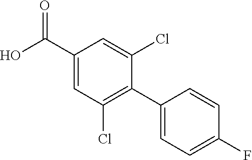 [SMILES: O=C(O)c1cc(Cl)c(-c2ccc(F)cc2)c(Cl)c1]

3,5-Dichloro-4-iodobenzoic acid (400 mg, 1.26 mmol) and 4-fluorophenylboronic acid (220 mg, 1.58 mmol) in a 20 mL microwave reaction vial were taken up in dioxane (10 mL) and 2M K₂CO₃ solution (3.2 mL, 6.4 mmol) and the vial was flushed with nitrogen. Pd(Ph₃P)₄ (84 mg, 0.076 mmol) was added and the vial was again flushed with nitrogen and sealed. The reaction was heated at 115° C. for 30 minutes. The reaction was diluted with water and 2M HCl and extracted twice with ethyl acetate. The organic layers were washed with brine, dried over sodium sulfate and concentrated. The residue was purified on silica gel (CF 12 gm column, 100% DCM, then 5% ethyl acetate/DCM, then a gradient of 5-20% ethyl acetate containing 1% HOAc/DCM) to give a single band by TLC (280 mg) containing a mixture of title compound and 3,5-dichlorobenzoic acid bi-product by LC-MS. The mixture was used directly in Step B.

### Step B: Synthesis of (2,6-dichloro-4′-fluorobiphenyl-4-yl)methanol

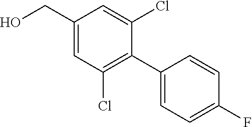 [SMILES: OCc1cc(Cl)c(-c2ccc(F)cc2)c(Cl)c1]

To a solution of 2,6-dichloro-4′-fluorobiphenyl-4-carboxylic acid (350 mg, 1.155 mmol) in THF (5 mL) was added 2M borane-methyl sulfide complex in THF (0.35 mL, 0.70 mmol) at room temperature. After 20 hours, the reaction was quenched with 2M HCl and water and was extracted twice with ethyl acetate. The organic layers were washed with brine containing sodium bicarbonate, dried over sodium sulfate and concentrated. The residue was purified on silica gel (CF 12 gm column, 100% DCM, then a gradient of 2-10% ethyl acetate/DCM) to give a single band by TLC (65 mg) containing a 1:1 mixture of title compound and 3,5-dichlorobenzyl alcohol bi-product from the Step A impurity. The mixture was used directly in Step C.

¹H NMR of clean title compound (CDCl₃, 400 MHz): δ 7.395 (s, 2H), 7.21 (m, 2H), 7.12 (m, 2H), 4.692 (s, 2H), 2.0 (br s, 1H).

### Step C: Synthesis of 2,6-dichloro-4′-fluorobiphenyl-4-carboxaldehyde

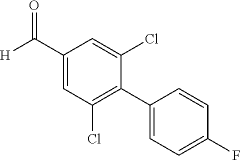 [SMILES: O=Cc1cc(Cl)c(-c2ccc(F)cc2)c(Cl)c1]

A solution of oxalyl chloride (41.3 uL, 0.47 mmol) in DCM (3 mL) under nitrogen was cooled in a dry ice/acetone bath and DMSO (84 uL, 1.18 mmol) was added slowly. After 15 minutes, the mixture of alcohols from Step B (65 mg) in DCM (2 mL) was added. After an additional hour at −78° C., DIPEA (0.412 mL, 2.4 mmol) in DCM (0.5 mL) was added and the reaction was warmed to room temperature for 1 hour. The reaction was diluted with water and 18% citric acid and was extracted twice with DCM. The organic layers were washed with brine containing sodium bicarbonate, dried over sodium sulfate and concentrated. The residue was purified on silica gel (CF 12 gm column, 100% hexanes, then a gradient of 10-20% DCM/hexanes to remove the 2,6-dichlorobenzaldehyde impurity, then 20-40% DCM/hexanes) to give the title compound (30 mg).

[M+H⁺]: m/z 269.

### Intermediate 13

### 2,6-Dichloro-2′,4′-difluorobiphenyl-4-carboxaldehyde

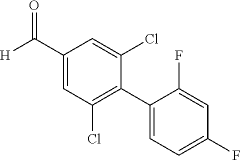 [SMILES: O=Cc1cc(Cl)c(-c2ccc(F)cc2F)c(Cl)c1]

### Step A: Synthesis of 3,5-dichloro-4-iodobenzyl alcohol

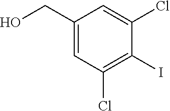 [SMILES: OCc1cc(Cl)c(I)c(Cl)c1]

Using essentially the same procedure as for INTERMEDIATE 12, Step B, 3,5-dichloro-4-iodobenzoic acid (1900 mg, 6.0 mmol) was reduced to the title compound (1050 mg) using 2M borane-methyl sulfide complex in THF (4.0 mL, 8.0 mmol).

¹H NMR (CDCl₃, 400 MHz): δ 7.342 (s, 2H), 4.62 (d, J=5.9 Hz, 2H), 1.78 (t, J=5.9 Hz, 1H).

### Step B: Synthesis of (2,6-dichloro-2′,4′-difluorobiphenyl-4-yl)methanol

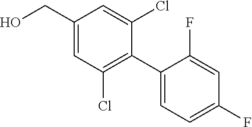 [SMILES: OCc1cc(Cl)c(-c2ccc(F)cc2F)c(Cl)c1]

Using essentially the same coupling procedure as for INTERMEDIATE 12, Step A, but heating 2,4-difluorophenylboronic acid (456 mg, 2.89 mmol) and 3,5-dichloro-4-iodobenzyl alcohol (350 mg, 1.155 mmol) in a microwave at 115° C. for 120 minutes, afforded the title compound (242 mg).

¹H NMR (CDCl₃, 400 MHz): δ 7.37 (s, 2H), 7.22 (m, 1H), 6.9-7.05 (m, 2H), 4.70 (s, 2H), 1.9 (br s, 1H).

### Step C: Synthesis of 2,6-dichloro-2′,4′-difluorobiphenyl-4-carboxaldehyde

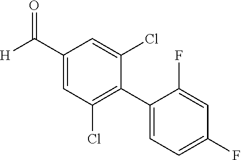 [SMILES: O=Cc1cc(Cl)c(-c2ccc(F)cc2F)c(Cl)c1]

Using essentially the same Swern procedure as for INTERMEDIATE 12, Step C, (2,6-dichloro-2′,4′-difluorobiphenyl-4-yl)methanol (450 mg, 1.557 mmol) was oxidized to the title compound (440 mg).

¹H NMR (CDCl₃, 400 MHz): δ. 9.963 (s, 1H), 7.899 (s, 2H), 7.22 (m, 1H), 6.9-7.05 (m, 2H).

### Intermediate 14

### 2,6-Dichloro-3′,4′-difluorobiphenyl-4-carboxaldehyde

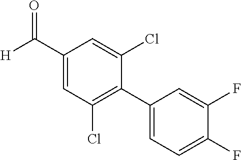 [SMILES: O=Cc1cc(Cl)c(-c2ccc(F)c(F)c2)c(Cl)c1]

Using essentially the same procedures as for INTERMEDIATE 13, Steps B-C, but using 3,4-difluorophenylboronic acid, 3,5-dichloro-4-iodobenzyl alcohol (INTERMEDIATE 9, Step A) was converted to the title compound after Swern oxidation.

¹H NMR (CDCl₃, 400 MHz): δ 9.953 (s, 1H), 7.886 (s, 2H), 7.27 (m, 1H), 7.09 (m, 1H), 6.97 (m, 1H).

### Intermediate 15

### 2,6-Dimethyl-4′-fluorobiphenyl-4-carboxaldehyde

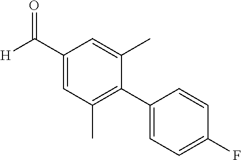 [SMILES: Cc1cc(C=O)cc(C)c1-c1ccc(F)cc1]

### Step A: Synthesis of 3,5-dimethyl-4-trifluoromethanesulfonyloxybenzaldehyde

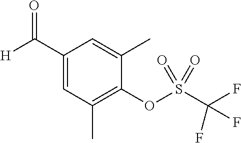 [SMILES: Cc1cc(C=O)cc(C)c1OS(=O)(=O)C(F)(F)F]

To a solution of 3,5-dimethyl-4-hydroxybenzyl alcohol (900 mg, 6.0 mmol) and N-phenyl-bis(trifluoromethanesulfonimide) (2.57 g, 7.19 mmol) under nitrogen at room temperature in anhydrous DCM (10 mL) was slowly added TEA (1.67 mL, 12 mmol). The reaction was stirred at room temperature for 20 hours and was then quenched with water and extracted three times with DCM. The organic layers were washed with brine, dried over magnesium sulfate and concentrated. The residue was purified on silica gel (CF 24 gm column, 100% hexanes, then a gradient of 0-35% ethyl acetate/hexanes) to give the title compound (1.63 g).

¹H NMR (CDCl₃, 500 MHz): δ 10.02 (s, 1H), 7.71 (s, 2H), 2.51 (s, 6H).

[M+H⁺]: m/z 282.

### Step B: Synthesis of 2,6-dimethyl-4′-fluorobiphenyl-4-carboxaldehyde

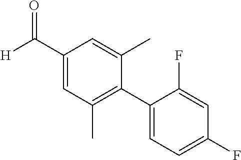 [SMILES: Cc1cc(C=O)cc(C)c1-c1ccc(F)cc1F]

3,5-Dimethyl-4-trifluoromethanesulfonyloxybenzaldehyde (100 mg, 0.35 mmol) and 4-fluoroboronic acid (74 mg, 0.53 mmol) in a 5 mL microwave reaction vial were taken up in dioxane (3 mL) and 1M K₂CO₃ solution (1.1 mL, 1.1 mmol) and the vial was flushed with nitrogen. Pd(Ph₃P)₄ (25 mg, 0.021 mmol) was added and the vial was again flushed with nitrogen and sealed. The reaction was heated in a microwave at 110° C. for 20 minutes. The reaction was diluted with water and the layers separated. The organic layer was filtered and concentrated. The residue could be used directly or be purified on silica gel (CF 12 gm column, 100% hexanes, then a gradient of 0-10% ethyl acetate/hexanes) to give the title compound (40 mg).

¹H NMR (CDCl₃, 500 MHz): δ 10.02 (s, 1H), 7.74 (s, 2H), 7.12 and 7.20 (2 m, 4H), 2.12 (s, 6H).

### Intermediate 16

### 2,6-Dimethyl-2′,4′-difluorobiphenyl-4-carboxaldehyde

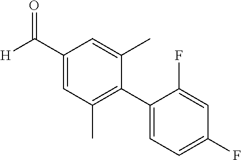 [SMILES: Cc1cc(C=O)cc(C)c1-c1ccc(F)cc1F]

Using essentially the same procedure as for INTERMEDIATE 15, Steps B, but using 2,4-difluoroboronic acid (92 mg, 0.58 mmol), 3,5-dimethyl-4-trifluoromethanesulfonyloxybenzaldehyde (110 mg, 0.39 mmol) (INTERMEDIATE 11, Step A) was converted to the title compound (94 mg crude).

[M+H⁺]: m/z 247.

### Intermediate 17

### 4-(4-Fluorophenyl)-1-(propan-2-yl)-1H-indole-3-carboxaldehyde

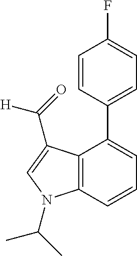 [SMILES: CC(C)n1cc(C=O)c2c(-c3ccc(F)cc3)cccc21]

### Step A: Synthesis of 4-bromo-1-(propan-2-yl)-1H-indole-3-carboxaldehyde

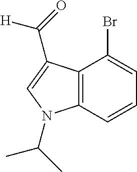

Cesium carbonate (2.18 g, 6.69 mmol) was added to a stirred, ice bath cooled solution of 4-bromo-1H-indole-3-carboxaldehyde (500 mg, 2.23 mmol) in DMF (5 mL) and the mixture was stirred at room temperature for 10 minutes. Then 2-iodopropane (0.446 mL, 4.46 mmol) was added and the solution was heated at 80° C. for 2 hours. The solution was cooled to room temperature and partitioned between ethyl acetate and water. The organic layers were washed with water (2× times), brine, dried over sodium sulfate and concentrated to give the crude title compound (480 mg) which was used directly in Step B.

¹H NMR (CDCl₃, 500 MHz): δ 10.925 (s, 1H), 8.179 (s, 1H), 7.518 (d, J=7.5 Hz, 1H), 7.440 (d, J=8.5 Hz, 1H), 7.176 (br t, J=8 Hz, 1H), 4.179 (hep, J=6.5 Hz, 1H), 1.603 (d, J=6.5 Hz, 6H).

[M+H⁺]: m/z 266, 268.

### Step B: Synthesis of 4-(4-fluorophenyl)-1-(propan-2-yl)-1H-indole-3-carboxaldehyde

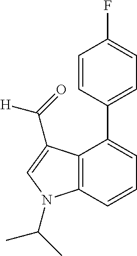 [SMILES: CC(C)n1cc(C=O)c2c(-c3ccc(F)cc3)cccc21]

A mixture of 4-bromo-1-(propan-2-yl)-1H-indole-3-carboxaldehyde (580 mg, 2.2 mmol), 4-fluoroboronic acid (396 mg, 2.83 mmol), potassium carbonate (904 mg, 6.54 mmol) and dichloro 1,1′-bis(diphenylphosphino) ferrocene palladium (II) DCM adduct (142 mg, 0.194 mmol) in dioxane (5 mL) and water (1 mL) was placed under nitrogen in a 20 mL microwave reactor vial and heated at 140° C. for 25 minutes. The mixture was cooled and the solvent was evaporated under reduced pressure. The mixture was diluted with ethyl acetate (100 mL), washed with saturated sodium bicarbonate (50 mL) and brine (50 mL), dried over sodium sulfate and concentrated. The residue was purified by column chromatography on silica gel (CF 80 g column, 0-100% ethyl acetate/hexanes) to give the title compound (515 mg).

¹H NMR (CDCl₃, 500 MHz): δ 9.475 (s, 1H), 8.125 (s, 1H), 7.45 (m, 3H), 7.391 (br t, J=8 Hz, 1H), 7.162 (m, 3H), 4.179 (hep, J=6.5 Hz, 1H), 1.603 (d, J=6.5 Hz, 6H).

[M+H⁺]: m/z 282.

### Intermediate 18

### 4-(6-Fluoropyridin-3-yl)-1-(propan-2-yl)-1H-indole-3-carboxaldehyde

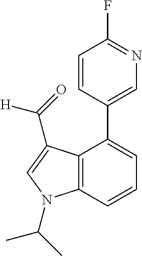 [SMILES: CC(C)n1cc(C=O)c2c(-c3ccc(F)nc3)cccc21]

Using essentially the same procedure as for INTERMEDIATE 17, Step B, but using (6-fluoropyridin-3-yl)boronic acid (344 mg, 2.44 mmol), 4-bromo-1-(propan-2-yl)-1H-indole-3-carboxaldehyde (500 mg, 1.88 mmol) (INTERMEDIATE 13, Step A) was converted to the title compound (520 mg).

[M+H⁺]: m/z 283.

### Intermediate 19

### 4-(2,4,5-Trifluorophenyl)-1-(propan-2-yl)-1H-indole-3-carboxaldehyde

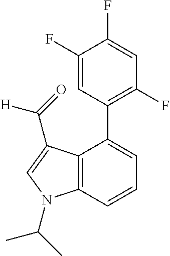 [SMILES: CC(C)n1cc(C=O)c2c(-c3cc(F)c(F)cc3F)cccc21]

Using essentially the same procedure as for INTERMEDIATE 17, Step B, but using (2,4,5-trifluorophenyl)boronic acid (79 mg, 0.45 mmol), 4-bromo-1-(propan-2-yl)-1H-indole-3-carboxaldehyde (100 mg, 0.376 mmol) (INTERMEDIATE 13, Step A) was converted to the title compound (80 mg).

[M+H⁺]: m/z 318.

### Intermediate 20

### 4-(4-Fluorophenyl)-1-(propan-2-yl)-1H-indazole-3-carboxaldehyde

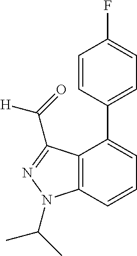 [SMILES: CC(C)n1nc(C=O)c2c(-c3ccc(F)cc3)cccc21]

### Step A: Synthesis of methyl 4-bromo-1-(propan-2-yl)-1H-indazole-3-carboxylate

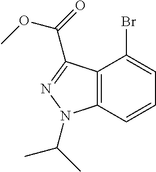 [SMILES: COC(=O)c1nn(C(C)C)c2cccc(Br)c12]

Cesium carbonate (1.92 g, 5.88 mmol) was added to a stirred, ice bath cooled solution of methyl 4-bromo-1H-indazole-3-carboxylate (500 mg, 1.96 mmol) in DMF (5 mL) and the mixture was stirred at room temperature for 10 minutes. Then 2-iodopropane (0.392 mL, 3.92 mmol) was added and the solution was heated at 80° C. for 2 hours. The solution was cooled to room temperature and partitioned between ethyl acetate and water. The organic layers were washed with water (2× times), brine, dried over sodium sulfate and concentrated to give the crude title compound (500 mg) which was used directly in Step B.

[M+H⁺]: m/z 297/299, 223/225 (100%).

### Step B: Synthesis of methyl 4-(4-fluorophenyl)-1-(propan-2-yl)-1H-indazole-3-carboxylate

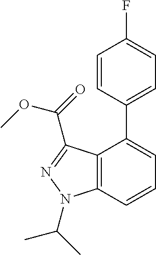 [SMILES: COC(=O)c1nn(C(C)C)c2cccc(-c3ccc(F)cc3)c12]

A mixture of methyl 4-bromo-1-(propan-2-yl)-1H-indazole-3-carboxylate (478 mg, 1.61 mmol), 4-fluoroboronic acid (293 mg, 2.09 mmol), potassium carbonate (667 mg, 4.83 mmol) and dichloro 1,1′-bis(diphenylphosphino) ferrocene palladium (II) DCM adduct (105 mg, 0.144 mmol) in dioxane (5 mL) and water (1 mL) was placed under nitrogen in a 20 mL microwave reactor vial and heated at 140° C. for 25 minutes. The mixture was cooled and the solvent was evaporated under reduced pressure. The mixture was diluted with ethyl acetate (100 mL), washed with saturated sodium bicarbonate (50 mL) and brine (50 mL), dried over sodium sulfate and concentrated. The residue was purified by column chromatography on silica gel (CF 40 g column, 0-100% ethyl acetate/hexanes) to give the title compound (200 mg).

[M+H⁺]: m/z 313, 239 (100%).

### Step C: Synthesis of (4-(4-fluorophenyl)-1-(propan-2-yl)-1H-indazol-3-yl)methanol

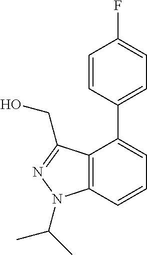 [SMILES: CC(C)n1nc(CO)c2c(-c3ccc(F)cc3)cccc21]

DIBAL-H (1.63 mL, 1 M solution in DCM) was added dropwise to a solution of methyl 4-(4-fluorophenyl)-1-(propan-2-yl)-1H-indazole-3-carboxylate (170 mg, 0.544 mmol) in dry DCM (5 mL) cooled to −78° C. under nitrogen. After stirring for 3 hours at −78° C. the reaction was quenched by adding a saturated aqueous solution of Na₂SO₄ (10 mL) and the mixture was allowed to warm to room temperature. The mixture was acidified with dilute HCl, extracted with ethyl acetate, washed with brine, dried and concentrated. The crude title compound (150 mg) was used directly in the next step.

[M+H⁺]: m/z 285, 225 (100%).

### Step D: Synthesis of 4-(4-fluorophenyl)-1-(propan-2-yl)-1H-indazole-3-carboxaldehyde

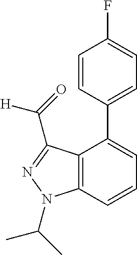 [SMILES: CC(C)n1nc(C=O)c2c(-c3ccc(F)cc3)cccc21]

To a solution of 4-(4-fluorophenyl)-1-(propan-2-yl)-1H-indazol-3-yl)methanol (100 mg, 0.352 mmol) in anhydrous DCM (5 mL) was added Dess-Martin periodinane (224 mg, 0.528 mmol). The suspension was stirred at room temperature for 16 hours. The volatiles were removed in vacuo and the residue was dissolved in ethyl acetate. The ethyl acetate solution was washed with brine, dried over sodium sulfate and concentrated. The residue was purified on a silica gel column (CF 24 g column; 0-70% ethyl acetate/hexanes) to give the title compound (70 mg).

[M+H⁺]: m/z 283, 241 (100%).

### Intermediate 21

### 4-(4-Fluorophenyl)-1-(propan-2-yl)-1H-pyrrolo[2,3-b]pyridine-3-carboxaldehyde

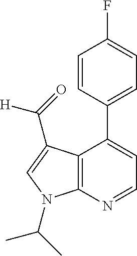 [SMILES: CC(C)n1cc(C=O)c2c(-c3ccc(F)cc3)ccnc21]

### Step A: Synthesis of 4-bromo-1-(propan-2-yl)-1H-pyrrolo[2,3-b]pyridine-3-carboxaldehyde

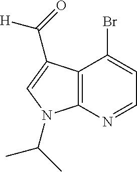

Cesium carbonate (2.17 g, 6.67 mmol) was added to a stirred, ice bath cooled solution of 4-bromo-1H-pyrrolo[2,3-b]pyridine-3-carboxaldehyde (500 mg, 2.22 mmol) in DMF (5 mL) and the mixture was stirred at room temperature for 10 minutes. Then 2-iodopropane (0.444 mL, 4.44 mmol) was added and the solution was heated at 80° C. for 2 hours. The solution was cooled to room temperature and partitioned between ethyl acetate and water. The organic layers were washed with water (2× times), brine, dried over sodium sulfate and concentrated to give the crude title compound (700 mg) which was used directly in Step B.

[M+H⁺]: m/z 267/269.

### Step B: Synthesis of 4-(4-fluorophenyl)-1-(propan-2-yl)-1H-pyrrolo[2,3-b]pyridine-3-carboxaldehyde

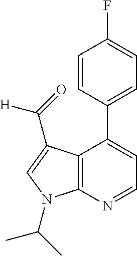 [SMILES: CC(C)n1cc(C=O)c2c(-c3ccc(F)cc3)ccnc21]

A mixture of 4-bromo-1-(propan-2-yl)-1H-pyrrolo[2,3-b]pyridine-3-carboxaldehyde (300 mg, 0.90 mmol), 4-fluoroboronic acid (251 mg, 1.80 mmol), potassium carbonate (373 mg, 2.70 mmol) and dichloro 1,1′-bis(diphenylphosphino) ferrocene palladium (II) DCM adduct (59 mg, 0.090 mmol) in dioxane (2 mL) and water (0.5 mL) was placed under a nitrogen atmosphere in a mL microwave reactor vial and heated at 140° C. for 25 minutes. The mixture was cooled and the solvent was evaporated under reduced pressure. The mixture was diluted with ethyl acetate (10 mL), washed with saturated sodium bicarbonate (10 mL) and brine (10 mL), dried over sodium sulfate and concentrated. The residue was purified by reverse phase (C-18) HPLC chromatography (5-95% acetonitrile in water with 0.1% TFA) to give the title compound (110 mg).

[M+H⁺]: m/z 283.

### INTERMEDIATE 22

### 7-Methyl-1-(propan-2-yl)-1H-indole-3-carboxaldehyde

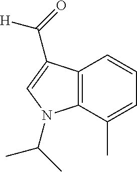

Using essentially the same procedure as for INTERMEDIATE 17, Step A, but using 7-methyl-1H-indole-3-carboxaldehyde (380 mg, 2.387 mmol), the title compound (400 mg) was obtained.

[M+H⁺]: m/z 202.

### Intermediate 23

### 7-Methyl-1-(cyclopropyl)-1H-indole-3-carboxaldehyde

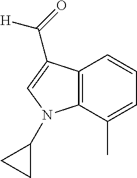

To a suspension of cyclopropylboronic acid (540 mg, 6.28 mmol), 7-methyl-1H-indole-3-carboxaldehyde (500 mg, 3.14 mmol), 2,2′-bipyridine (294 mg, 1.885 mmol) and sodium carbonate (666 mg, 6.28 mmol) in dichloroethane (15 mL) was added a suspension of copper (II) acetate (571 mg, 3.14 mmol) in hot DCE (5 mL). The mixture was warmed to 70° C. and stirred for 2 hours. The reaction mixture was cooled to room temperature and saturated aqueous NH₄Cl solution was added followed by additional water. The organic layer was separated and the aqueous layer was extracted three times with DCM. The combined organic layers were washed with brine, dried and concentrated. The residue was purified on silica gel (CF 40 gm column, 0-100% ethyl acetate/hexanes) to afford the title compound (300 mg).

¹H NMR (CDCl₃, 500 MHz): δ 9.952 (s, 1H), 8.177 (d, J=8.0 Hz, 1H), 7.694 (s, 1H), 7.207 (br t, J=7.5 Hz, 1H), 7.100 (d, J=7.5 Hz, 1H), 3.738 (m, 1H), 1.22 (m, 4H).

[M+H⁺]: m/z 200.

### Intermediate 24

### 1-(Propan-2-yl)-4,5,6,7-tetrafluoro-1H-indole-3-carboxaldehyde

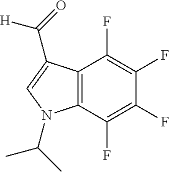 [SMILES: CC(C)n1cc(C=O)c2c(F)c(F)c(F)c(F)c21]

Using essentially the same procedure as for INTERMEDIATE 17, Step A, but using 4,5,6,7-tetrafluoro-1H-indole-3-carboxaldehyde 200 mg, 1.256 mmol), the title compound was obtained (530 mg).

[M+H⁺]: m/z 260, 218 (100%).

### Intermediate 25

### 4-(Cyclopropyl)-1-(propan-2-yl)-1H-indole-3-carboxaldehyde

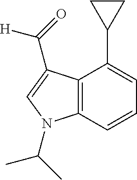 [SMILES: CC(C)n1cc(C=O)c2c(C3CC3)cccc21]

To a solution of 4-bromo-1-(propan-2-yl)-1H-indole-3-carboxaldehyde (100 mg, 0.376 mmol) (INTERMEDIATE 13, Step A), (4,4,5,5-tetramethyl-1,3,2-dioxaborolan-2-yl)cyclopropane (95 mg, 0.564 mmol), lithium hydroxide mono-hydrate (63 mg, 1.50 mmol) and dichloro 1,1′-bis(diphenylphosphino) ferrocene palladium (II) DCM adduct (25 mg, 0.038 mmol) in dioxane (2 mL) and water (0.5 mL) was placed under nitrogen in a 5 mL microwave reactor vial and heated at 120° C. for 15 minutes. The reaction was cooled and concentrated under reduced pressure. The mixture was diluted with ethyl acetate (10 mL) and the ethyl acetate was washed with saturated sodium bicarbonate (10 mL) and brine (10 ml), dried over sodium sulfate and concentrated under reduced pressure. The residue was purified by column chromatography on silica gel (CF 24 g column; eluting with 0-100% ethyl acetate in hexanes) to give the title compound (60 mg).

¹H NMR (CDCl₃, 500 MHz): δ 10.520 (s, 1H), 8.097 (s, 1H), 7.316 (d, J=8.0 Hz, 1H), 7.254 (br t, J=8.0 Hz, 1H), 7.043 (d, J=7.5 Hz, 1H), 4.727 (hep, J=6.5 Hz, 1H), 2.70 (m, 1H), 1.616 (d, J=6.5 Hz, 6H), 1.08 (m, 2H), 0.87 (m, 2H).

[M+H⁺]: m/z 228.

### Intermediate 26

### 7-(Cyclopropyl)-1-(propan-2-yl)-1H-indole-3-carboxaldehyde

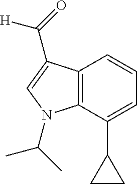 [SMILES: CC(C)n1cc(C=O)c2cccc(C3CC3)c21]

Using essentially the same procedure as for INTERMEDIATE 25, but using 7-bromo-1-(propan-2-yl)-1H-indole-3-carboxaldehyde (100 mg, 0.376 mmol) (prepared the same as Intermediate 13, Step A), the title compound was obtained (60 mg).

[M+H⁺]: m/z 228.

### Intermediate 27

### 1,5-(Dicyclopropyl)-1H-indole-3-carboxaldehyde

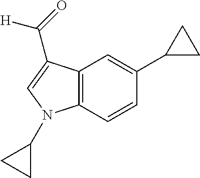 [SMILES: O=Cc1cn(C2CC2)c2ccc(C3CC3)cc12]

Using essentially the same procedure as for INTERMEDIATE 25, but using 5-bromo-1-(cyclopropyl)-1H-indole-3-carboxaldehyde (100 mg, 0.379 mmol) (prepared the same as Intermediate 19), the title compound was obtained (60 mg).

[M+H⁺]: m/z 226.

### Intermediate 28

### 1,7-(Dicyclopropyl)-1H-indole-3-carboxaldehyde

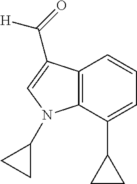 [SMILES: O=Cc1cn(C2CC2)c2c(C3CC3)cccc12]

Using essentially the same procedure as for INTERMEDIATE 25, but using 7-bromo-1-(cyclopropyl)-1H-indole-3-carboxaldehyde (100 mg, 0.379 mmol) (prepared the same as Intermediate 19), the title compound was obtained (75 mg).

[M+H⁺]: m/z 226, 198 (−28=CO, 100%).

### Intermediate 29

### 1,6-(Dicyclopropyl)-1H-indole-3-carboxaldehyde

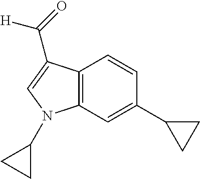 [SMILES: O=Cc1cn(C2CC2)c2cc(C3CC3)ccc12]

Using essentially the same procedure as for INTERMEDIATE 25, but using 6-bromo-1-(cyclopropyl)-1H-indole-3-carboxaldehyde (120 mg, 0.454 mmol) (prepared the same as Intermediate 19), the title compound was obtained (74 mg).

[M+H⁺]: m/z 226, 198 (−28=CO, 100%).

### Intermediate 30

### 1,3-Dibromo-5-(bromomethyl)-2-chlorobenzene

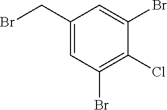 [SMILES: Clc1c(Br)cc(CBr)cc1Br]

To 1,3-dibromo-2-chloro-5-methylbenzene (1000 mg, 3.52 mmol) was added NBS (688 mg, 3.87 mmol) and AIBN (57.7 mg, 0.352 mmol). The mixture was stirred at room temperature for 30 min and then heated to 80° C. overnight. The solution was evaporated and the residue was purified by silica gel chromatography (0-5% EtOAc/hexanes) to provide the title compound (740 mg) as a clear oil.

### Intermediate 31

### 3,5-Dicyclopropyl-4-(trifluoromethoxy)benzaldehyde

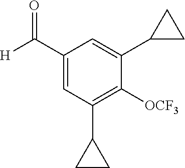 [SMILES: O=Cc1cc(C2CC2)c(OC(F)(F)F)c(C2CC2)c1]

### Step A: Synthesis of methyl 3,5-dibromo-4-{[(phenylsulfanyl)carbonothioyl]oxy}benzoate

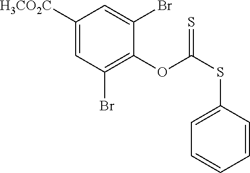 [SMILES: COC(=O)c1cc(Br)c(OC(=S)Sc2ccccc2)c(Br)c1]

Methyl 3,5-dibromo-4-hydroxybenzoate (1000 mg, 3.23 mmol) was dissolved in THF (16 mL) and N-methylmorpholine (0.709 mL, 6.45 mmol) was added. The mixture was cooled to 0° C. and phenyl chlorodithioformate (0.549 mL, 3.87 mmol) was added. The mixture was warmed to room temperature and stirred overnight. The mixture was diluted with EtOAc and washed with water. The organic layer was dried over Na₂SO₄, filtered and concentrated. The residue was purified by silica gel chromatography (0-15% EtOAc/hexanes) to yield the title compound as a clear oil (1491 mg).

[M+H⁺]: m/z 461.

### Step B: Synthesis of methyl 3,5-dibromo-4-(trifluoromethoxy)benzoate

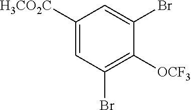 [SMILES: COC(=O)c1cc(Br)c(OC(F)(F)F)c(Br)c1]

Methyl 3,5-dibromo-4-{[(phenylsulfanyl)carbonothioyl]oxy}benzoate (1491 mg, 3.23 mmol) was placed in a polypropylene round bottom and DCM (16 mL) was added. The mixture was cooled to −78° C. and then HF-Pyridine (1303 μl, 10.49 mmol) was added slowly. 1,3-Dibromo-5,5-dimethylhydantoin (750 mg, 2.62 mmol) was added portion-wise. The mixture was slowly warmed to room temperature over 2 hours and then the mixture was stirred for another 1 hour at room temperature. The mixture was cooled to 0° C. and carefully quenched with 10% NaOH (150 mL). The mixture was extracted with DCM and the combined organic extracts were washed with brine, dried over Na₂SO₄, filtered and concentrated. The residue was purified by silica gel chromatography (0-20% EtOAc/hexanes) to yield the title compound as a clear oil (1007 mg).

[M+H⁺]: m/z 377.

### Step C: Synthesis of methyl 3,5-dicyclopropyl-4-(trifluoromethoxy)benzoate

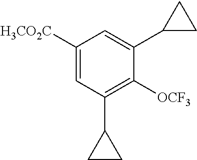 [SMILES: COC(=O)c1cc(C2CC2)c(OC(F)(F)F)c(C2CC2)c1]

To methyl 3,5-dibromo-4-(trifluoromethoxy)benzoate (500 mg, 1.32 mmol), Cs₂CO₃ (2586 mg, 7.94 mmol), Pd(OAc)₂ (11.9 mg, 0.053 mmol), potassium cyclopropyltrifluoroborate (470 mg, 3.18 mmol) and di(1-adamantyl)-n-butylphosphine (28.5 mg, 0.079 mmol) was added toluene (12 mL) and water (1.2 mL). The reaction mixture was heated to 100° C. overnight. The reaction mixture was cooled to room temperature, diluted with water, extracted with DCM and evaporated in vacuo. The residue was purified by silica gel chromatography (0-20% EtOAc/hexanes) to yield the title compound (259 mg).

[M+H⁺]: m/z 301.

### Step D: Synthesis of [3,5-d]cyclopropyl-4-(trifluoromethoxy)phenyl]methanol

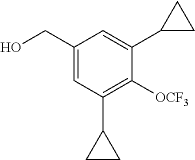 [SMILES: OCc1cc(C2CC2)c(OC(F)(F)F)c(C2CC2)c1]

Methyl 3,5-dicyclopropyl-4-(trifluoromethoxy)benzoate (259 mg, 0.863 mmol) was dissolved in DCM (8.6 mL) and the mixture was cooled to 0° C. DIBAL (1.73 mL, 1.73 mmol) was added and the reaction mixture was allowed to warm to room temperature and stirred for 2 hours. The reaction mixture was quenched with the addition of 3N HCl and water. The mixture was stirred vigorously for 30 min and extracted with DCM. The combined organic extracts were washed with brine, dried over Na₂SO₄, filtered and concentrated. The residue was purified by silica gel chromatography (0-30% EtOAc/hexanes) to yield the title compound as a clear oil (190 mg).

### Step E: Synthesis of 3,5-dicyclopropyl-4-(trifluoromethoxy)benzaldehyde

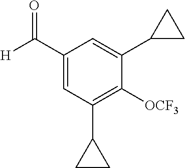 [SMILES: O=Cc1cc(C2CC2)c(OC(F)(F)F)c(C2CC2)c1]

[3,5-Dicyclopropyl-4-(trifluoromethoxy)phenyl]methanol (190 mg, 0.698 mmol) was dissolved in DCM (6 mL) and Dess-Martin periodinane (444 mg, 1.05 mmol) was added. The mixture was stirred for 1 hour at room temperature. The mixture was evaporated in vacuo and the residue was purified by silica gel chromatography (0-10% EtOAc/hexanes) to yield the title compound as a clear oil (174 mg).

[M+H⁺]: m/z 271.

### Intermediate 32

### 1,3-Dichloro-5-(dibromomethyl)-2-(trifluoromethoxy)benzene

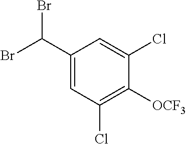 [SMILES: FC(F)(F)Oc1c(Cl)cc(C(Br)Br)cc1Cl]

### Step A: Synthesis of O-(2,6-dichloro-4-methylphenyl) S-phenyl carbonodithioate

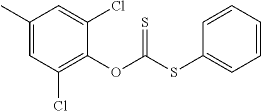 [SMILES: Cc1cc(Cl)c(OC(=S)Sc2ccccc2)c(Cl)c1]

The title compound was prepared from 2,6-dichloro-4-methylphenol (1000 mg, 5.64 mmol) following essentially the same procedure described in Step A of INTERMEDIATE 31. The title compound was obtained as a clear oil (1860 mg).

[M+H⁺]: m/z 329.

### Step B: Synthesis of 1,3-dichloro-5-methyl-2-(trifluoromethoxy)benzene

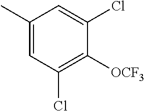

The title compound was prepared from O-(2,6-dichloro-4-methylphenyl) S-phenyl carbonodithioate (1860 mg, 5.65 mmol) following essentially the same procedure described in Step B of INTERMEDIATE 31. The residue was purified by silica gel chromatography (0-5% EtOAc/hexanes) to yield the title compound as a clear oil (1240 mg).

### Step C: Synthesis of 1,3-dichloro-5-(dibromomethyl)-2-(trifluoromethoxy)benzene

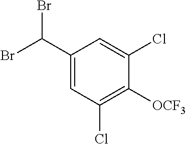 [SMILES: FC(F)(F)Oc1c(Cl)cc(C(Br)Br)cc1Cl]

To 1,3-dichloro-5-methyl-2-(trifluoromethoxy)benzene (697 mg, 2.84 mmol) was added NBS (304 mg, 1.707 mmol) and AIBN (23.36 mg, 0.142 mmol). The mixture was stirred at room temperature for 30 min and then heated to 80° C. overnight. The solution was evaporated and the residue was purified by silica gel chromatography (0-5% EtOAc/hexanes) to provide the title compound as a clear oil (646 mg).

### Intermediate 33

### 1-Bromo-5-(bromomethyl)-2,4-dichlorobenzene

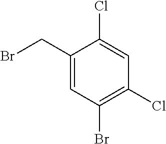 [SMILES: Clc1cc(Cl)c(CBr)cc1Br]

The title compound was prepared from 1-bromo-2,4-dichloro-5-methylbenzene (1200 mg, 5 mmol) following essentially the same procedure described for INTERMEDIATE 30. The title compound was obtained as a clear oil (1232 mg).

### Intermediate 34

### 2-Bromo-4-(bromomethyl)-1-(trifluoromethyl)benzene

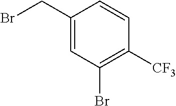 [SMILES: FC(F)(F)c1ccc(CBr)cc1Br]

The title compound was prepared from 2-bromo-4-methyl-1-(trifluoromethyl)benzene (963 mg, 3.83 mmol) following essentially the same procedure described for INTERMEDIATE 30. The title compound was obtained as a clear oil (1043 mg).

### Intermediate 35

### 5-Fluoro-1-(propan-2-yl)-1H-indole-3-carboxaldehyde

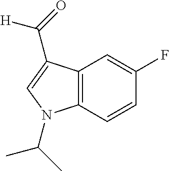

Using essentially the same procedure as for INTERMEDIATE 17, but using 5-fluoro-1H-indole-3-carboxaldehyde (500 mg, 3.06 mmol), the title compound was prepared (518 mg).

[M+H⁺]: m/z 206.

### Intermediate 36

### 7-Chloro-1-(propan-2-yl)-1H-indole-3-carboxaldehyde

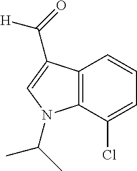

Using essentially the same procedure as for INTERMEDIATE 17, but using 7-chloro-1H-indole-3-carboxaldehyde (500 mg, 2.78 mmol), the title compound was prepared (538 mg).

[M+H⁺]: m/z 238/240.

### Intermediate 37-Cyclopropyl-7-methyl-1-(propan-2-yl)-1H-indole-3-carboxaldehyde

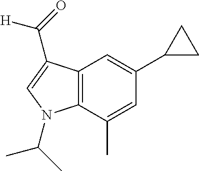 [SMILES: Cc1cc(C2CC2)cc2c(C=O)cn(C(C)C)c12]

Using essentially the same procedure as for INTERMEDIATE 25, but using 5-bromo-7-methyl-1-(propan-2-yl)-1H-indole-3-carboxaldehyde (200 mg, 0.714 mmol) (prepared as for INTERMEDIATE 13, Step A), the title compound was prepared (97 mg).

[M+H⁺]: m/z 242.

### Intermediate 38

### 5-Fluoro-7-methyl-1-(propan-2-yl)-1H-indole-3-carboxaldehyde

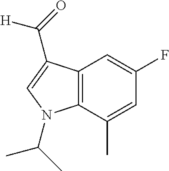 [SMILES: Cc1cc(F)cc2c(C=O)cn(C(C)C)c12]

Using essentially the same procedure as for INTERMEDIATE 25, but using 5-fluoro-7-methyl-1H-indole-3-carboxaldehyde (200 mg, 2.258 mmol), the title compound was prepared (437 mg). [M+H⁺]: m/z 220.

### Intermediate 39

### 1-(Cyclopropyl)-5-fluoro-7-methyl-1H-indole-3-carboxaldehyde

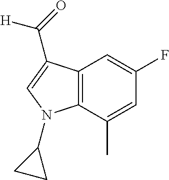 [SMILES: Cc1cc(F)cc2c(C=O)cn(C3CC3)c12]

Using essentially the same procedure as for INTERMEDIATE 23, but using 5-fluoro-7-methyl-1H-indole-3-carboxaldehyde (415 mg, 2.342 mmol), the title compound was prepared (365 mg).

[M+H⁺]: m/z 218.

### Intermediate 40

### 1,5-(Dicyclopropyl)-7-methyl-1H-indole-3-carboxaldehyde

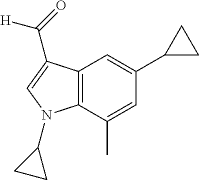 [SMILES: Cc1cc(C2CC2)cc2c(C=O)cn(C3CC3)c12]

Using essentially the same procedure as for INTERMEDIATE 25, but using 5-bromo-1-(cyclopropyl)-7-methyl-1H-indole-3-carboxaldehyde (250 mg, 0.899 mmol) (prepared as for Intermediate 19, Step A), the title compound was prepared (90 mg).

[M+H⁺]: m/z 240.

### Intermediate 41

### 7-Chloro-1-(cyclopropyl)-1H-indole-3-carboxaldehyde

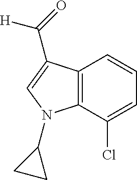 [SMILES: O=Cc1cn(C2CC2)c2c(Cl)cccc12]

Using essentially the same procedure as for INTERMEDIATE 23, but using 5-chloro-1H-indole-3-carboxaldehyde (470 mg, 2.62 mmol), the title compound was prepared (350 mg).

[M+H⁺]: m/z 220/222.

### Intermediate 42

### 7-Chloro-4-methyl-1-(propan-2-yl)-1H-indole-3-carboxaldehyde

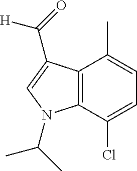 [SMILES: Cc1ccc(Cl)c2c1c(C=O)cn2C(C)C]

Using essentially the same procedure as for INTERMEDIATE 17, Step A, but using 7-chloro-4-methyl-1H-indole-3-carboxaldehyde (170 mg, 0.878 mmol), the title compound was prepared (110 mg).

[M+H⁺]: m/z 236/238.

### Intermediate 43

### 5-Chloro-7-methyl-1-(propan-2-yl)-1H-indole-3-carboxaldehyde

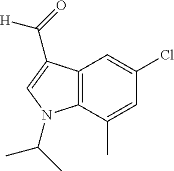 [SMILES: Cc1cc(Cl)cc2c(C=O)cn(C(C)C)c12]

### Step A: Synthesis of 5-chloro-7-methyl-1H-indole-3-carboxaldehyde

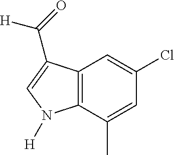

Phosphorus oxychloride (422 uL, 4.53 mmol) was added dropwise to dry DMF (2 mL) at −20° C. and stirred at −5° C. for 30 minutes. A solution of 5-chloro-7-methylindole (500 mg, 3.02 mmol) in dry DMF (3 mL) was added to the above mixture at −20° C. The cooling bath was removed and the mixture was allowed to warm to room temperature. After one hour, the reaction was poured onto ice, basified with solid NaOH and stirred for an additional 15 minutes. The resulting precipitate was filtered to give the title compound as a pale-yellow solid (450 mg).

[M+H⁺]: m/z 194/196.

### Step B: Synthesis of 5-chloro-7-methyl-1-(propan-2-yl)-1H-indole-3-carboxaldehyde

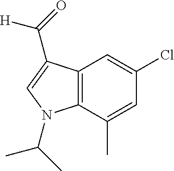 [SMILES: Cc1cc(Cl)cc2c(C=O)cn(C(C)C)c12]

Using essentially the same procedure as for INTERMEDIATE 13, Step A, but using 5-chloro-7-methyl-1H-indole-3-carboxaldehyde from Step A (300 mg, 1.55 mmol), the title compound was prepared (300 mg).

[M+H⁺]: m/z 236/238.

### Intermediate 44

### 4,7-Dichloro-1-(propan-2-yl)-1H-indole-3-carboxaldehyde

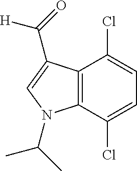 [SMILES: CC(C)n1cc(C=O)c2c(Cl)ccc(Cl)c21]

### Step A: Synthesis of 4,7-dichloro-1H-indole-3-carboxaldehyde

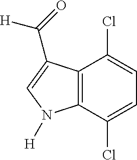 [SMILES: O=Cc1c[nH]c2c(Cl)ccc(Cl)c12]

Using essentially the same procedure as for INTERMEDIATE 43, Step A, but starting with 4,7-dichloro-1H-indole (600 mg, 2.32 mmol), the title compound (450 mg) was prepared.

¹H NMR (CDCl₃, 500 MHz): δ 10.459 (s, 1H), 8.311 (br s, 1H), 7.315 (ABq, J=8.0 Hz, 2H), 3.40 (br s, 1H).

[M+H⁺]: m/z 214/216.

### Step B: Synthesis of 4,7-dichloro-1-(propan-2-yl)-1H-indole-3-carboxaldehyde

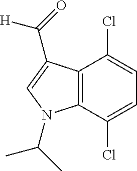 [SMILES: CC(C)n1cc(C=O)c2c(Cl)ccc(Cl)c21]

Using essentially the same procedure as for INTERMEDIATE 43, Step B, but starting with 4,7-dichloro-1H-indole-3-carboxaldehyde (100 mg, 0.47 mmol), the title compound was prepared (100 mg).

¹H NMR (CDCl₃, 500 MHz): δ 10.767 (s, 1H), 8.219 (br s, 1H), 7.204 (ABq, J=8.0 Hz, 2H), 5.737 (hep, J=6.5 Hz, 1H), 1.606 (d, J=6.5 Hz, 6H).

[M+H⁺]: m/z 256/258.

### Intermediate 45

### 5-Chloro-7-methyl-1-(cyclopropanyl)-1H-indole-3-carboxaldehyde

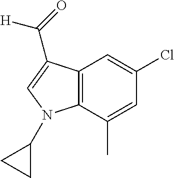 [SMILES: Cc1cc(Cl)cc2c(C=O)cn(C3CC3)c12]

Using essentially the same procedure as for INTERMEDIATE 23, but using 5-chloro-7-methyl-1H-indole-3-carboxaldehyde (300 mg, 1.55 mmol), the title compound was prepared (330 mg).

[M+H⁺]: m/z 234/236.

### Intermediate 46

### 7-Chloro-4-methyl-1-(cyclopropanyl)-1H-indole-3-carboxaldehyde

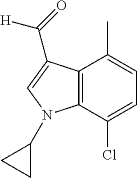 [SMILES: Cc1ccc(Cl)c2c1c(C=O)cn2C1CC1]

Using essentially the same procedure as for INTERMEDIATE 23, but using 7-chloro-4-methyl-1H-indole-3-carboxaldehyde (200 mg, 1.033 mmol), the title compound was prepared (170 mg).

[M+H⁺]: m/z 234/236.

### Intermediate 47

### 4,7-Dichloro-1-(cycpropanyl)-1H-indole-3-carboxaldehyde

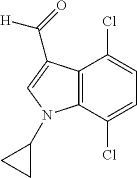 [SMILES: O=Cc1cn(C2CC2)c2c(Cl)ccc(Cl)c12]

Using essentially the same procedure as for INTERMEDIATE 23, but using 4,7-dichloro-1H-indole-3-carboxaldehyde (200 mg, 0.934 mmol) from INTERMEDIATE 40, Step A, the title compound was prepared (54 mg).

[M+H⁺]: m/z 254/256.

### Intermediate 48

### 5,7-Dichloro-1-(propan-2-yl)-1H-indole-3-carboxaldehyde

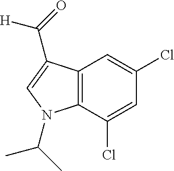 [SMILES: CC(C)n1cc(C=O)c2cc(Cl)cc(Cl)c21]

Using essentially the same procedure as for INTERMEDIATE 17, Step A, but using 5,7-dichloro-1H-indole-3-carboxaldehyde (150 mg, 0.561 mmol), the title compound was prepared (105 mg).

[M+H⁺]: m/z 256/258.

### Intermediate 49

### 5,7-Dichloro-1-(propan-2-yl)-1H-indole-3-carboxaldehyde

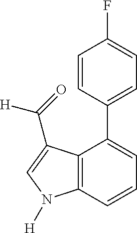 [SMILES: O=Cc1c[nH]c2cccc(-c3ccc(F)cc3)c12]

Using essentially the same procedure as for INTERMEDIATE 17, Step B, but using 4-bromo-1H-indole-3-carboxaldehyde, the title compound was prepared.

[M+H⁺]: m/z 240.

### Intermediate 50

### 7-Chloro-1-(propan-2-yl)-1H-indole-3-carboxaldehyde

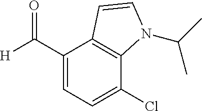

Using essentially the same procedure as for INTERMEDIATE 17, Step A, but using 7-chloro-1H-indole-4-carboxaldehyde, the title compound was prepared.

¹H NMR (CDCl₃, 500 MHz): δ 10.222 (s, 1H), 7.543 (d, J=3 Hz, 1H), 7.530 (d, J=8.0 Hz, 1H), 7.428 (d, J=3 Hz, 1H), 7.304 (d, J=8.0 Hz, 1H), 5.756 (hep, J=6.5 Hz, 1H), 1.577 (d, J=6.5 Hz, 6H).

[M+H⁺]: m/z 222/224.

### Intermediate 51

### 2,3-Dimethyl-1-(propan-2-yl)-1H-indole-3-carboxaldehyde

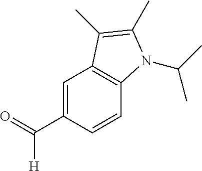 [SMILES: Cc1c(C)n(C(C)C)c2ccc(C=O)cc12]

Using essentially the same procedure as for INTERMEDIATE 17, Step A, but using 2,3-dimethyl-1H-indole-5-carboxaldehyde (500 mg, 2.89 mmol), the title compound was prepared (300 mg).

¹H NMR (CDCl₃, 500 MHz): δ 10.040 (s, 1H), 8.053 (s, 1H), 7.680 (d, J=8.5 Hz, 1H), 7.525 (d, J=8.5 Hz, 1H), 4.715 (hep, J=6.5 Hz, 1H), 2.419 (s, 3H), 2.311 (s, 3H), 1.640 (d, J=6.5 Hz, 6H).

[M+H⁺]: m/z 216, 188 (−28=CO, 100%).

### Intermediate 52

### 2-Bromo-4-methyl-5-(trifluoromethyl)benzaldehyde

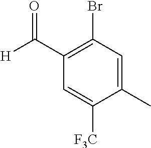

A 100 mL 3-necked round-bottom flask fitted with a thermometer was placed under nitrogen and anhydrous THF (25 mL) followed by 1.6 M n-BuLi (28.8 mL, 46 mmol) were added. The solution was cooled in a dry ice/acetone bath to −10 to −5° C. when neat tetramethylpiperidine (7.8 mL, 46 mmol) was added while maintaining the temperature of the solution around −10 to −5° C. The resulting mixture was stirred at this temperature for another 30 minutes as a light brown precipitate formed. The reaction was then cooled further to −78° C. before 5-bromo-2-trifluoromethyltoluene (5 g, 20.92 mmol) in THF (2 mL) was added dropwise. The reaction mixture was stirred at −78° C. for 45 minutes and then DMF (1.55 mL, 21 mmol) was added while maintaining the temperature at −78° C. The ice bath cooling was maintained for 10 minutes and was then removed to allow the reaction to warm to room temperature. The reaction was quenched with water (20 mL) and extracted 3 times with ether. The combined organic layers were washed with 4 M HCl and brine, dried over sodium sulfate and concentrated in vacuo. The residue was purified on a silica gel column (24 g) eluting 5-10% ethyl acetate in hexanes to give the title compound as a yellow solid (1.7 g).

¹H NMR (CDCl₃, 500 MHz): δ 10.3 (s, 1H), 8.2 (s, 1H), 7.6 (s, 1H), 2.5 (s, 3H).

### Intermediate 53

### 8-Chloro-4,4-dimethyl-3,4-dihydro-2H-chromene-6-carbaldehyde

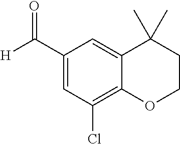

### Step A: Synthesis of 4-bromo-2-chloro-1-[(3-methylbut-3-en-1-yl)oxy] benzene

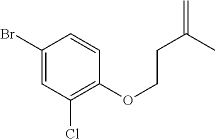

To a mixture of 4-bromo-2-chlorophenol (0.98 g, 4.71 mmol), and CsCO₃ (2.05 g, 6.28 mmol) in DMF (4.49 mL) at room temperature was added 3-methylbut-3-en-1-yl diphenyl phosphate (1.00 g, 3.14 mmol, synthesized according to a procedure in U.S., 5006550, 9 Apr. 1991) dropwise via a syringe. The reaction mixture was heated to 85° C. for 1 h. It was then allowed to cool to room temperature, and diluted with water (20.0 mL). The resulting mixture was extracted with hexane (75.0 mL, 2×), and the combined organic layers were dried over sodium sulfate, filtered, and concentrated under vacuum. The crude mixture was purified by silica gel chromatography eluting with 0-5% EtOAc/hexanes to give the title compound as a colorless oil.

¹H NMR (CDCl₃, 500 MHz): δ 7.52 (d, J=3.0 Hz, 1H), 7.33 (dd, J=8.6 Hz, 2.4 Hz, 1H), 6.82 (d, J=8.7 Hz, 1H), 4.89 (s, 1H), 4.84 (s, 1H), 4.13 (t, J=6.8 Hz, 2H), 2.58 (t, J=6.9 Hz, 2H), 1.85 (s, 3H).

### Step B: Synthesis of 6-bromo-8-chloro-4,4-dimethyl-3,4-dihydro-2H-chromene

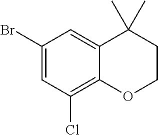

To a mixture of aluminum chloride (403 mg, 3.03 mmol) and CH₂Cl₂ (10.0 mL) cooled to −78° C. was added 4-bromo-2-chloro-1-[(3-methylbut-3-en-1-yl)oxy] benzene (758 mg, 2.75 mmol) in CH₂Cl₂ (6.00 mL) via a cannula to give a light yellow solution. The reaction mixture was allowed to warm to room temperature, stirred for 5 min, and then poured into an Erlenmeyer flask containing a cold 10% NaOH solution (75.0 mL). The mixture was extracted with hexane (40.0 mL, 3×), and the combined organic layers were dried over sodium sulfate, filtered, and concentrated under vacuum. The crude was purified by silica gel chromatography eluting with 0-5% EtOAc/hexanes to afford the desired product as a colorless oil.

¹H NMR (CDCl₃, 500 MHz) δ 7.33 (d, J=2.3 Hz, 1H), 7.29 (d, J=2.3 Hz, 1H), 4.32 (t, J=5.5 Hz, 2H), 1.87 (t, J=5.4 Hz, 2H), 1.36 (s, 6H).

### Step C: Synthesis of 8-chloro-4,4-dimethyl-3,4-dihydro-2H-chromene-6-carbaldehyde

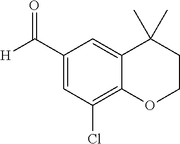

6-Bromo-8-chloro-4,4-dimethyl-3,4-dihydro-2H-chromene (483 mg, 1.75 mmol) in THF (12.0 mL) under N₂ atmosphere was cooled to −78° C., and n-BuLi (841 μL, 2.10 mmol) was added dropwise via a syringe. The reaction mixture was stirred at −78° C. for 10 min, and then DMF (543 μL, 7.01 mmol) was added dropwise via a syringe. The resulting mixture was allowed to warm to room temperature, and wet silica gel (5.0 g/0.5 mL of water) was added. The mixture was allowed to stir at room temperature for 10 min before it was filtered. The silica gel was rinsed with EtOAc, and the filtrate was concentrated under vacuum. The crude residue was purified by silica gel chromatography eluting with 0-20% EtOAc/hexanes to obtain the title compound as a white solid.

¹H NMR (CDC₃, 500 MHz): δ 9.84 (s, 1H), 7.77 (d, J=1.9 Hz, 1H), 7.74 (d, J=2.0 Hz, 1H), 4.43 (t, J=5.5 Hz, 2H), 1.93 (t, J=5.5 Hz, 2H), 1.42 (s, 6H).

### Intermediate 54

### 8-Cyclopropyl-4,4-dimethyl-3,4-dihydro-2H-chromene-6-carbaldehyde

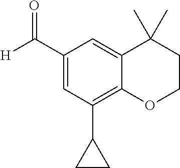 [SMILES: CC1(C)CCOc2c(C3CC3)cc(C=O)cc21]

A vial was charged with Pd(OAc)₂ (2.40 mg, 10.7 μmol), XPhos (10.2 mg, 0.02 mmol), potassium carbonate (148 mg, 1.07 mmol), potassium cyclopropyltrifluoroborate (58.0 mg, 0.39 mmol), and 8-chloro-4,4-dimethyl-3,4-dihydro-2H-chromene-6-carbaldehyde (80.0 mg, 0.36 mmol, INTERMEDIATE 53). The mixture was dissolved in cyclopropylmethyl ether (2.00 mL):water (0.20 mL), and purged with Ar. The reaction mixture was then stirred at 100° C. overnight, cooled to room temperature, and filtered through a pad of celite. The filtrate was concentrated under vacuum, and the crude residue was purified by silica gel chromatography eluting with 0-15% EtOAc/hexanes to afford the desired product as a yellow oil.

¹H NMR (CDCl₃, 500 MHz): δ 9.87 (s, 1H), 7.66 (d, J=1.9 Hz, 1H), 7.21 (d, J=1.9 Hz, 1H), 4.38 (t, J=5.3 Hz, 2H), 2.13-2.19 (m, 1H), 1.91 (t, J=5.5 Hz, 2H), 1.41 (s, 6H), 0.98 (ddd, J=10.8 Hz, 6.5 Hz, 4.6 Hz, 2H), 0.71 (ddd, J=9.7 Hz, 6.1 Hz, 4.5 Hz, 2H).

### Intermediate 55

### 8-chloro-2,2-dimethyl-3,4-dihydro-2H-chromene-6-carbaldehyde

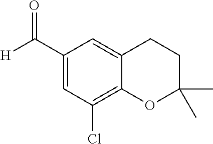

### Step A: Synthesis of 4-bromo-2-chloro-1-[(3-methylbut-2-en-1-yl)oxy] benzene

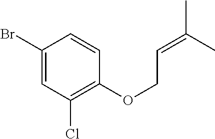

To a mixture of 4-bromo-2-chlorophenol (1.00 g, 4.82 mmol), and cesium carbonate (3.14 g, 9.64 mmol) in DMF (6.89 mL) at room temperature was added 4-bromo-2-methyl-2-butene (0.68 mL, 5.78 mmol) dropwise via a syringe. The reaction mixture was stirred at 85° C. for 1 h before it was allowed to cool to room temperature. Water (30.0 mL) was then added, and the mixture was extracted with hexane (75.0 mL, 2×). The combined organic layers were dried over sodium sulfate, filtered, and concentrated under vacuum. The crude mixture was purified by t silica gel chromatography eluting with 0-5% EtOAc/hexanes to afford the product as a colorless oil.

¹H NMR (CDCl₃, 400 MHz): δ 7.47 (d, J=2.4 Hz, 1H), 7.27 (dd, J=8.8 Hz, 2.3 Hz, 1H), 6.78 (d, J=8.8 Hz, 1H), 5.43-5.47 (m, 1H), 4.55 (d, J=6.5 Hz, 2H), 1.77 (s, 3H), 1.72 (s, 3H).

### Step B: Synthesis of 6-bromo-8-chloro-2,2-dimethyl-3,4-dihydro-2H-chromene

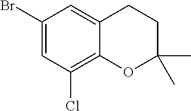

Aluminum chloride (0.71 g, 5.35 mmol) in CH₂Cl₂ (15.0 mL) was added 4-bromo-2-chloro-1-[(3-methylbut-2-en-1-yl)oxy] benzene (1.34 g, 4.86 mmol) dissolved in CH₂Cl₂ (5.00 mL) at −78° C. via a cannula. The reaction mixture was stirred at −78° C. for 10 min before addition of a 10% KOH (2.00 mL) solution at the same temperature. The mixture was then allowed to warm to room temperature and extracted with CH₂Cl₂ (50.0 mL, 2×). The combined organic layers were washed with brine, dried over sodium sulfate, filtered, and concentrated under vacuum. The crude residue was purified silica gel chromatography eluting with 0-5% EtOAc/hexanes to give the desired product as a colorless oil.

¹H NMR (CDCl₃, 400 MHz) δ 7.29 (d, J=2.2 Hz, 1H), 7.08 (s, 1H), 2.75 (t, J=6.7 Hz, 2H), 1.79 (t, J=6.8 Hz, 2H), 1.35 (s, 6H).

### Step C: Synthesis of 8-chloro-2,2-dimethyl-3,4-dihydro-2H-chromene-6-carbaldehyde

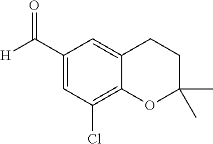

6-Bromo-8-chloro-2,2-dimethyl-3,4-dihydro-2H-chromene (483 mg, 1.75 mmol) in THF (8.76 mL) was cooled to −78° C., and n-BuLi (841 μL, 2.10 mmol) was added dropwise via a syringe. The reaction mixture was stirred at −78° C. for 10 min, and then DMF (543 μL, 7.01 mmol) was added dropwise via a syringe. The resulting mixture was allowed to warm to room temperature, and wet silica gel (5.0 g/0.5 mL of water) was added. The mixture was allowed to stir at room temperature for 10 min before it was filtered. The silica gel was rinsed with EtOAc, and the filtrate was concentrated under vacuum. The crude mixture was purified by silica gel chromatography eluting with 0-15% EtOAc/hexanes to obtain the desired product as a pale-yellow oil.

¹H NMR (CD₃OD, 500 MHz): δ 7.20 (d, J=1.8 Hz, 1H), 7.06 (d, J=0.9 Hz, 1H), 2.82 (t, J=6.9 Hz, 2H), 1.84 (t, J=6.7 Hz, 2H), 1.36 (s, 6H).

### Intermediate 56

### 8-Cyclopropyl-2,2-dimethyl-3,4-dihydro-2H-chromene-6-carbaldehyde

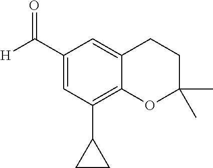 [SMILES: CC1(C)CCc2cc(C=O)cc(C3CC3)c2O1]

A vial was charged with Pd(OAc)₂ (7.99 mg, 0.04 mmol), XPhos (25.5 mg, 0.05 mmol), potassium carbonate (148 mg, 1.07 mmol), potassium cyclopropyltrifluoroborate (58.0 mg, 0.39 mmol), and 8-chloro-2,2-dimethyl-3,4-dihydro-2H-chromene-6-carbaldehyde (80 mg, 0.36 mmol, INTERMEDIATE 55). The mixture was dissolved in cyclopropylmethyl ether (1.62 mL):water (0.16 mL), and purged with Ar. The reaction mixture was then stirred at 100° C. overnight, cooled to room temperature, and filtered through a pad of celite. The filtrate was concentrated under vacuum, and the crude residue was purified by silica gel chromatography eluting with 0-20% EtOAc/hexanes to afford the desired product as a pale-yellow oil.

¹H NMR (CDCl₃, 400 MHz): δ 9.75 (s, 1H), 7.39-7.41 (m, 1H), 7.18 (d, J=1.7 Hz, 1H), 2.83 (t, J=6.8 Hz, 2H), 2.09-2.16 (m, 1H), 1.84 (t, J=6.8 Hz, 2H), 1.37 (s, 6H), 0.92 (ddd, J=10.7 Hz, 6.4 Hz, 4.5 Hz, 2H), 0.66 (ddd, J=9.6 Hz, 6.1 Hz, 4.3 Hz, 2H).

### Intermediate 57

### 2,2,8-Trimethyl-3,4-dihydro-2H-chromene-6-carbaldehyde

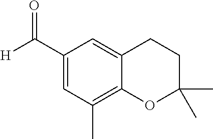

### Step A: Synthesis of 4-bromo-2-methyl-1-[(3-methylbut-2-en-1-yl)oxy] benzene

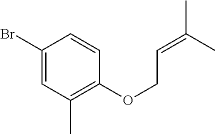

Using essentially the same procedure as INTERMEDIATE 55, Step A: 4-bromo-2-methylphenol (1.00 g, 5.35 mmol), cesium carbonate (3.48 g, 10.7 mmol) and 4-bromo-2-methyl-2-butene (0.75 mL, 6.42 mmol) afforded the desired product as a pale-yellow oil.

¹H NMR (CDCl₃, 400 MHz): δ 7.19-7.24 (m, 2H), 6.67 (d, J=8.3 Hz, 1H), 5.42-5.46 (m, 1H), 4.47 (d, J=6.4 Hz, 2H), 2.17 (s, 3H), 1.77 (s, 3H), 1.71 (s, 3H).

### Step B: Synthesis of 6-bromo-2,2,8-trimethyl-3,4-dihydro-2H-chromene

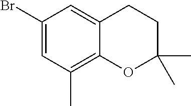

Aluminum chloride (0.76 g, 5.69 mmol) in CH₂Cl₂ (15.0 mL) at −78° C. was added 4-bromo-2-methyl-1-[(3-methylbut-2-en-1-yl)oxy] benzene (1.32 g, 5.17 mmol) dissolved in CH₂Cl₂ (4.00 mL) via a syringe. The reaction mixture was allowed to warm to room temperature and stirred for another 5 min. A cold 10% NaOH solution (20.0 mL) was then added, and the mixture was extracted with CH₂Cl₂ (50.0 mL, 2×). The combined organic layers were washed with brine, dried over sodium sulfate, filtered, and concentrated under vacuum. The crude residue was purified by silica gel chromatography eluting with 0-5% EtOAc/hexanes to give the desired product as a colorless oil.

¹H NMR (CDCl₃, 400 MHz): δ 6.97-7.09 (m, 2H), 2.71 (t, J=6.8 Hz, 2H), 2.10 (s, 3H), 1.75 (t, J=6.7 Hz, 2H), 1.29 (s, 6H).

### Step C: Synthesis of 2,2,8-trimethyl-3,4-dihydro-2H-chromene-6-carbaldehyde

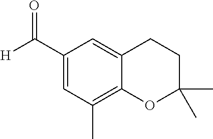

Using essentially the same procedure as INTERMEDIATE 55, Step C: 6-bromo-2,2,8-trimethyl-3,4-dihydro-2H-chromene (684 mg, 2.68 mmol), n-BuLi (1.30 mL, 3.22 mmol) and DMF (830 μL, 10.7 mmol) afforded the desired product as a pale-yellow oil.

¹H NMR (CDCl₃, 500 MHz): δ 9.82 (s, 1H), 7.53 (s, 1H), 7.50 (s, 1H), 2.86 (t, J=6.7 Hz, 2H), 2.23 (s, 3H), 1.86 (t, J=6.7 Hz, 2H), 1.39 (s, 6H).

### Intermediate 58

### 2,2-Dimethyl-3,4-dihydro-2H-chromene-8-carbaldehyde

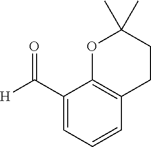

2,2-Dimethyl-3,4-dihydro-2H-chromene-8-carboxylic acid (300 mg, 1.45 mmol) dissolved in THF (3.60 mL) was cooled to 0° C., and BH3.THF (5.82 mL, 5.82 mmol) was added dropwise via a syringe. The resulting solution was stirred at 65° C. for 1 h, and then allowed to cool to room temperature before addition of 6 N HCl (2.50 mL) at 0° C. The mixture was again heated at 65° C. for 30 min. The solution was cooled to room temperature, made basic with 1N NaOH, and extracted with EtOAc (40.0 mL, 3×). The combined organic layers were dried over sodium sulfate, filtered, and concentrated under vacuum to obtain the crude alcohol as a colorless oil, which was directly used to the next step. The crude alcohol was dissolved in CH₂Cl₂ (5.00 mL), and Dess-Martin Periodinane (1.23 g, 2.91 mmol) was added in one portion at room temperature. The reaction mixture was stirred for 1 h at room temperature, and concentrated under vacuum on silica gel. The crude residue was purified by silica gel chromatography eluting with 0-15% EtOAc/hexanes to give the desired product as a colorless oil.

¹H NMR (CDCl₃, 400 MHz): δ 10.4 (s, 1H), 7.63 (d, J=7.7 Hz, 1H), 7.25 (d, J=7.7 Hz, 1H), 6.85 (t, J=7.5 Hz, 1H), 2.80 (t, J=6.7 Hz, 2H), 1.85 (t, J=6.9 Hz, 2H), 1.38 (s, 6H).

### Intermediate 59

### 6-Chloro-2,2-dimethyl-3,4-dihydro-2H-chromene-8-carbaldehyde

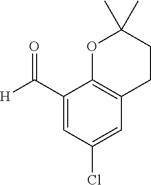

### Step A: Synthesis of methyl 2,2-dimethyl-3,4-dihydro-2H-chromene-8-carboxylate

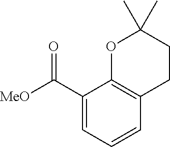 [SMILES: COC(=O)c1cccc2c1OC(C)(C)CC2]

To a solution of 2,2-dimethyl-3,4-dihydro-2H-chromene-8-carboxylic acid (300 mg, 1.45 mmol) in methanol (2.10 mL): toluene (5.20 mL) cooled to 0° C. was added TMS-diazomethane (1.82 mL, 3.64 mmol) dropwise via a syringe. The reaction mixture was warmed to room temperature, and stirred for 45 min. It was then allowed to cool to 0° C., and acetic acid (2.00 mL) was added carefully. The solvent was evaporated under vacuum, and the residue was re-dissolved in EtOAc (80.0 mL), washed with a sat NaHCO₃ solution (20.0 mL, dried over sodium sulfate, filtered, and concentrated under vacuum. The crude product was used directly to the next step without further purification.

¹H NMR (CDCl₃, 400 MHz) δ: 7.57 (d, J=7.7 Hz, 1H), 7.16 (d, J=7.4 Hz, 1H), 6.79 (t, J=7.7 Hz, 1H), 3.84 (s, 3H), 2.79 (t, J=6.7 Hz, 2H), 1.82 (t, J=6.7 Hz, 2H), 1.35 (s, 6H).

### Step B: Synthesis of methyl 6-chloro-2,2-dimethyl-3,4-dihydro-2H-chromene-8-carboxylate

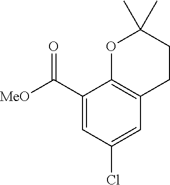 [SMILES: COC(=O)c1cc(Cl)cc2c1OC(C)(C)CC2]

To a solution of methyl 2,2-dimethyl-3,4-dihydro-2H-chromene-8-carboxylate (330 mg, 1.50 mmol) in CH₂Cl₂ (3.74 mL): methanol (3.74 mL) was added concentrated HCl (61.5 μL, 0.75 mmol) at 10° C. and then portions of NCS (206 mg, 1.54 mmol). The solution was stirred for 1 h at 8-12° C. The solution was then poured into a mixture of water (5.00 mL), sat sodium thiosulfate (5.00 mL), 1 N NaOH (5.00 mL), and CH₂Cl₂ (30.0 mL). The mixture was stirred for min at room temperature, and the two phases were separated. The organic phase was acidified to pH<2 with 1 N HCl. The mixture was then extracted with CH₂Cl₂ (10.0 mL, 2×). The combined organic layers were dried over sodium sulfate, filtered, and concentrated under vacuum. The crude residue was purified by silica gel chromatography eluting with 0-15% EtOAc/hexanes to give the desired product as a colorless oil.

¹H NMR (CDCl₃, 400 MHz): δ 7.54 (d, J=2.7 Hz, 1H), 7.14 (app d, J=2.6 Hz, 1H), 3.84 (s, 3H), 2.77 (t, J=6.8 Hz, 2H), 1.81 (t, J=6.8 Hz, 2H), 1.34 (s, 6H).

### Step C: Synthesis of 6-chloro-2,2-dimethyl-3,4-dihydro-2H-chromene-8-carbaldehyde

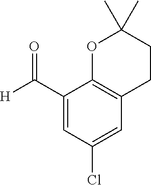

6-Chloro-2,2-dimethyl-3,4-dihydro-2H-chromene-8-carboxylate (350 mg, 1.37 mmol) was dissolved in CH₂Cl₂ (6.87 mL) and cooled to −78° C. DIBAL-H (4.12 mL, 4.12 mmol) was added dropwise. The reaction mixture was warmed to room temperature and stirred for 1 h. It was then allowed to cool to 0° C., diluted with CH₂Cl₂ (10.0 mL), and water (2.00 mL) was added slowly, followed by a 15% NaOH solution (5.00 mL). The mixture was warmed to room temperature, and stirred for 15 min. The organic layer was separated, dried over sodium sulfate, filtered, and concentrated under vacuum. The crude alcohol was used directly to the next step without further purification. The crude alcohol was dissolved in CH₂Cl₂ (7.00 mL), and Dess-Martin Periodinane (1.20 g, 2.75 mmol) was added in one portion at room temperature. The reaction mixture was stirred at room temperature for 30 min, and then concentrated under vacuum on silica gel. It was then purified by silica gel chromatography eluting with 0-15% EtOAc/hexanes to give the desired product as a white solid.

¹H NMR (DMSO-d6, 400 MHz): δ 10.2 (s, 1H), 7.44 (app d, J=2.7 Hz, 1H), 7.36 (d, J=2.6 Hz, 1H), 2.76 (t, J=6.8 Hz, 2H), 1.79 (t, J=6.7 Hz, 2H), 1.30 (s, 6H).

### Intermediate 60

### 6-Cyclopropyl-2,2-dimethyl-3,4-dihydro-2H-chromene-8-carbaldehyde

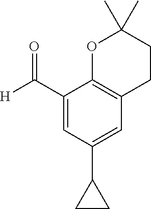 [SMILES: CC1(C)CCc2cc(C3CC3)cc(C=O)c2O1]

Using essentially the same procedure as INTERMEDIATE 54: Pd(OAc)₂ (7.04 mg, 0.03 mmol), X-Phos (29.9 mg, 0.06 mmol), potassium carbonate (434 mg, 3.14 mmol), potassium cyclopropyltrifluoroborate (170 mg, 1.15 mmol), and 6-chloro-2,2-dimethyl-3,4-dihydro-2H-chromene-8-carbaldehyde (235 mg, 1.05 mmol, INTERMEDIATE 59) afforded the desired product as a pale-yellow oil.

¹H NMR (DMSO-d6, 400 MHz): δ 10.2 (s, 1H), 7.14 (d, J=2.2 Hz, 1H), 7.08 (d, J=2.0 Hz, 1H), 2.70 (t, J=6.7 Hz, 2H), 1.76-1.85 (m, 1H), 1.76 (t, J=6.7 Hz, 2H), 1.28 (s, 6H), 0.82 (ddd, J=10.5 Hz, 6.3 Hz, 4.3 Hz, 2H), 0.53 (ddd, J=9.3 Hz, 6.2 Hz, 4.3 Hz, 2H).

### Intermediate 61

### 2,2-Dimethyl-2,3-dihydrospiro[chromene-4,1′-cyclopropane]-6-carbaldehyde

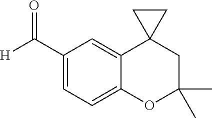 [SMILES: CC1(C)CC2(CC2)c2cc(C=O)ccc2O1]

### Step A: Synthesis of methyl 2,2-dimethyl-4-oxo-3,4-dihydro-2H-chromene-6-carboxylate

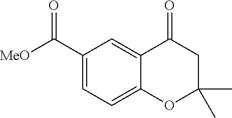 [SMILES: COC(=O)c1ccc2c(c1)C(=O)CC(C)(C)O2]

Methyl 3-acetyl-4-hydroxybenzoate (200 mg, 1.03 mmol), acetone (151 μL, 2.06 mmol), and pyrrolidine (25.6 μL, 0.31 mmol) were mixed in toluene (515 μL), and stirred at room temperature for 1 h. It was then heated to 100° C. for 5 h, allowed to cool to room temperature, diluted with EtOAc (50.0 mL), and poured into ice. The two layers were separated. The organic layer was washed with 2 N HCl (10.0 mL), 2 N NaOH (10.0 mL), water (10.0 mL), dried over sodium sulfate, filtered, and concentrated under vacuum. The crude mixture was purified by silica gel chromatography eluting with 0-30% EtOAc/hexanes to give the desired product as a pale-yellow oil.

¹H NMR (CDCl₃, 400 MHz): δ: 8.54 (d, J=2.2 Hz, 1H), 8.11 (dd, J=8.8 Hz, 2.2 Hz, 1H), 6.95 (d, J=8.8 Hz, 1H), 3.88 (s, 3H), 2.74 (s, 2H), 1.46 (s, 6H).

### Step B: Synthesis of methyl 2,2-dimethyl-4-methylidene-3,4-dihydro-2H-chromene-6-carboxylate

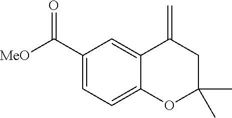 [SMILES: C=C1CC(C)(C)Oc2ccc(C(=O)OC)cc21]

Potassium tert-butoxide (96.0 mg, 0.85 mmol) was added to a suspension of triphenylmethylphosphonium bromide (305 mg, 0.85 mmol) in toluene (3.00 mL) while stirring at −30° C. under N₂ atmosphere. After stirring for 1 h at −30° C., a solution of methyl 2,2-dimethyl-4-oxo-3,4-dihydro-2H-chromene-6-carboxylate (100 mg, 0.43 mmol) in toluene (2.00 mL) was added slowly via a syringe. The suspension was stirred for a further 30 min at −30° C., and then heated to reflux overnight. The mixture was allowed to cool to room temperature, diluted with EtOAc (50.0 mL), and washed with water (10.0 mL). The organic layer was dried over sodium sulfate, filtered, and concentrated under vacuum. The crude residue was purified by silica gel chromatography eluting with 0-25% EtOAc/hexanes to give the desired product as a pale-yellow oil.

¹H NMR (CDCl₃, 500 MHz): δ 8.30 (d, J=2.2 Hz, 1H), 7.87 (dd, J=8.5 Hz, 2.0 Hz, 1H), 6.85 (d, J=8.6 Hz, 1H), 5.69 (s, 1H), 5.00 (s, 1H), 3.92 (s, 3H), 2.52 (s, 2H), 1.37 (s, 6H).

### Step C: Synthesis of methyl 2,2-dimethyl-2,3-dihydrospiro[chromene-4,1′-cyclopropane]-6-carboxylate

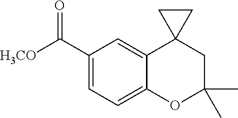 [SMILES: COC(=O)c1ccc2c(c1)C1(CC1)CC(C)(C)O2]

To a mixture of methyl 2,2-dimethyl-4-methylidene-3,4-dihydro-2H-chromene-6-carboxylate (34.0 mg, 0.15 mmol) and diethylzinc (1.17 mL, 1.17 mmol) in toluene (2.90 mL) under N₂ atmosphere was added diiodomethane (189 μL, 2.34 mmol) dropwise via a syringe at room temperature. The mixture was stirred at room temperature overnight and then partitioned between diethyl ether (10.0 mL) and 5% aqueous HCl (10.0 mL). The aqueous layer was extracted with diethyl ether (20.0 mL, 2×). The combined organic layers were dried over sodium sulfate, filtered, and concentrated under vacuum. The crude residue was purified by silica gel chromatography eluting with 0-15% EtOAc/hexanes to afford the desired product as a pale-yellow solid.

[M+H⁺]: m/z 247.

### Step D: Synthesis of 2,2-dimethyl-2,3-dihydrospiro[chromene-4,1′-cyclopropane]-6-carbaldehyde

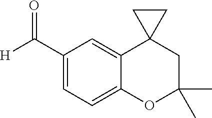 [SMILES: CC1(C)CC2(CC2)c2cc(C=O)ccc2O1]

Methyl 2,2-dimethyl-2,3-dihydrospiro[chromene-4,1′-cyclopropane]-6-carboxylate (33.0 mg, 0.13 mmol) was dissolved in dry toluene (1.34 mL), and cooled to −78° C. DIBAL-H (536 μL, 0.54 mmol) was added dropwise via a syringe. The reaction mixture was stirred for 2 h at −78° C. It was then quenched with methanol (500 μL) at the same temperature, and diluted with EtOAc (15.0 mL). The organic layer was washed with water (10.0 mL), dried over sodium sulfate, filtered, and concentrated under vacuum to afford the crude alcohol, which was used directly to the next step. The crude alcohol was dissolved in CH₂Cl₂ (4.00 mL), and Dess-Martin Periodinane (114 mg, 0.27 mmol) was added in one portion at room temperature. The resulting mixture was stirred at room temperature for 1 h, and concentrated under vacuum on silica gel. It was then purified by silica gel chromatography eluting with 0-15% EtOAc/hexanes to give the desired product as a colorless oil.

¹H NMR (CDCl₃, 500 MHz): δ 9.83 (s, 1H), 7.59 (dd, J=8.3 Hz, 1.9 Hz, 1H), 7.25 (d, J=2.0 Hz, 1H), 6.91 (d, J=8.3 Hz, 1H), 1.83 (s, 2H), 1.45 (s, 6H), 1.16 (dd, J=6.9 Hz, 5.0 Hz, 2H), 0.96 (dd, J=6.5 Hz, 4.7 Hz, 2H).

### Intermediate 62

### 8-Chloro-2,2-dimethyl-2,3-dihydrospiro[chromene-4,1′-cyclopropane]-6-carbaldehyde

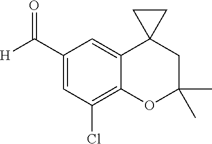 [SMILES: CC1(C)CC2(CC2)c2cc(C=O)cc(Cl)c2O1]

### Step A: Synthesis of methyl 8-chloro-2,2-dimethyl-2,3-dihydrospiro[chromene-4,1′-cyclopropane]-6-carboxylate

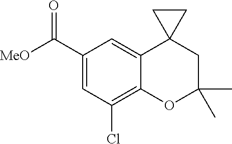 [SMILES: COC(=O)c1cc(Cl)c2c(c1)C1(CC1)CC(C)(C)O2]

To a solution of methyl 2,2-dimethyl-2,3-dihydrospiro[chromene-4,1′-cyclopropane]-6-carboxylate (384 mg, 1.56 mmol, INTERMEDIATE 61) in CH₂Cl₂ (3.90 mL): methanol (3.90 mL) was added concentrated HCl (64.0 μL, 0.78 mmol) at 10° C., and then portions of NCS (214 mg, 1.61 mmol). The reaction mixture was stirred for 1 h at 8-12° C. Since only starting material was observed by LC-MS, it was then allowed to warm to room temperature for 2 h before another LC-MS was obtained. The reaction seemed to progress slowly. Another portion of NCS (100 mg) was added, and stirred at room temperature for 2 h. The solution was then poured into a mixture of water (5.00 mL), sat sodium thiosulfate (5.00 mL), 1 N NaOH (5.00 mL), and CH₂Cl₂ (30.0 mL). The mixture was stirred for 15 min at room temperature, and the two layers were separated. The organic layer was acidified to pH<2 with 1 N HCl. The mixture was then extracted with CH₂Cl₂ (30.0 mL, 2×). The combined organic layers were dried over sodium sulfate, filtered, and concentrated under vacuum. The crude residue was purified by silica gel chromatography eluting with 0-5% EtOAc/hexanes to give the desired product as a colorless oil.

¹H NMR (CD₃OD, 500 MHz): δ 7.75 (d, J=1.9 Hz, 1H), 7.29 (d, J=1.9 Hz, 1H), 3.85 (s, 3H), 1.84 (s, 2H), 1.44 (s, 6H), 1.10 (dd, J=7.2 Hz, 5.1 Hz, 2H), 1.01 (dd, J=6.3 Hz, 4.2 Hz, 2H).

### Step B: Synthesis of 8-chloro-2,2-dimethyl-2,3-dihydrospiro[chromene-4,1′-cyclopropane]-6-carbaldehyde

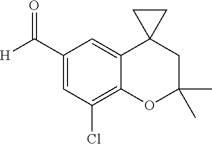 [SMILES: CC1(C)CC2(CC2)c2cc(C=O)cc(Cl)c2O1]

Methyl 8-chloro-2,2-dimethyl-2,3-dihydrospiro[chromene-4,1′-cyclopropane]-6-carboxylate (289 mg, 1.03 mmol) was dissolved in dry CH₂Cl₂ (5.15 mL), and cooled to −78° C. DIBAL-H (3.09 mL, 3.09 mmol) was added dropwise via a syringe. The reaction mixture was stirred at −78° C. for 2 h. It was quenched with methanol (1.00 mL), and diluted with CH₂Cl₂ (10.0 mL). Water (1.00 mL) was then added slowly, followed by a 15% NaOH solution (5.00 mL). The mixture was warmed to room temperature, and stirred for 15 min. The organic layer was separated, dried over sodium sulfate, filtered, and concentrated under vacuum. The crude alcohol was used directly to the next step without further purification. The crude alcohol was dissolved in CH₂Cl₂ (7.00 mL), and Dess-Martin Periodinane (873 mg, 2.06 mmol) was added in one portion at room temperature. The resulting mixture was stirred at room temperature for 30 min, and concentrated under vacuum on silica gel. Purification by the ISCO, eluted with 0-10% EtOAc/hexanes afforded the desired product as a colorless oil.

[M+H+]: m/z 251.

### Intermediate 63

### 8-Cyclopropyl-2,2-dimethyl-2,3-dihydrospiro[chromene-4,1′-cyclopropane]-6-carboxaldehyde

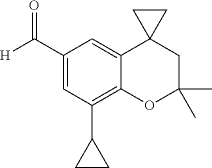 [SMILES: CC1(C)CC2(CC2)c2cc(C=O)cc(C3CC3)c2O1]

Using essentially the same procedure as INTERMEDIATE 54: Pd(OAc)₂ (17.5 mg, 0.08 mmol), X-Phos (55.6 mg, 0.12 mmol), potassium carbonate (322 mg, 2.33 mmol), potassium cyclopropyltrifluoroborate (127 mg, 0.86 mmol), and 8-chloro-2,2-dimethyl-2,3-dihydrospiro[chromene-4,1′-cyclopropane]-6-carbaldehyde (195 mg, 0.78 mmol, INTERMEDIATE 62) afforded the desired product as a yellow oil.

¹H NMR (CD₃OD, 500 MHz): δ 9.68 (s, 1H), 7.20 (d, J=2.0 Hz, 1H), 7.12 (d, J=2.0 Hz, 1H), 2.13-2.21 (m, 1H), 1.84 (s, 2H), 1.45 (s, 6H), 1.12 (dd, J=6.9 Hz, 4.8 Hz, 2H), 0.98 (dd, J=6.3 Hz, 4.3 Hz, 2H), 0.94 (ddd, J=10.7 Hz, 6.4 Hz, 4.5 Hz, 2H), 0.66 (ddd, J=9.6 Hz, 5.9 Hz, 4.2 Hz, 2H).

### Intermediate 64

### 8-Cyclopropyl-4,4-dimethyl-3,4-dihydro-2H-thiochromene-6-carbaldehyde

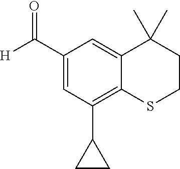 [SMILES: CC1(C)CCSc2c(C3CC3)cc(C=O)cc21]

### Step A: Synthesis of 4-bromo-2-chloro-1-[(3-methylbut-3-en-1-yl)sulfanyl]benzene

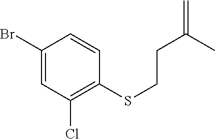

To a mixture of 4-bromo-2-chlorobenzenethiol (1.68 g, 7.54 mmol), and CsCO₃ (4.09 g, 12.6 mmol) in DMF (8.98 mL) at room temperature was added 3-methylbut-3-en-1-yl diphenyl phosphate (2.00 g, 6.28 mmol, synthesized according to a procedure in U.S., 5006550, 9 Apr. 1991) dropwise via a syringe. The reaction mixture was heated to 85° C. for 1 h. It was then allowed to cool to room temperature, and diluted with water (20.0 mL). The resulting mixture was extracted with EtOAc:hexane mixture (1:1, 75.0 mL, 2×), and the combined organic layers were dried over sodium sulfate, filtered, and concentrated under vacuum. The crude mixture was purified by silica gel chromatography eluting with 0-10% EtOAc/hexanes to give the desired product as a colorless oil.

¹H NMR (CDCl₃, 500 MHz): δ 7.55 (d, J=2.0 Hz, 1H), 7.37 (dd, J=8.4 Hz, 2.0 Hz, 1H), 7.16 (d, J=8.4 Hz, 1H), 4.86 (s, 1H), 4.81 (s, 1H), 3.05 (t, J=7.6 Hz, 2H), 2.40 (t, J=7.7 Hz, 2H), 1.80 (s, 3H).

### Step B: Synthesis of 6-bromo-8-chloro-4,4-dimethyl-3,4-dihydro-2H-thiochromene

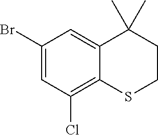

To a mixture of aluminum chloride (0.58 g, 4.34 mmol) and CH₂Cl₂ (23.2 mL) cooled to −78° C. was added 4-bromo-2-chloro-1-[(3-methylbut-3-en-1-yl)sulfanyl]benzene (1.15 g, 3.94 mmol) in CH₂Cl₂ (6.00 mL) via a cannula to give a light yellow solution. The reaction mixture was allowed to warm to room temperature, stirred for 5 min, and then poured into an Erlenmeyer flask containing a cold 10% NaOH solution (75.0 mL). The mixture was extracted with CH₂Cl₂ (40.0 mL, 2×), and the combined organic layers were dried over sodium sulfate, filtered, and concentrated under vacuum. The crude was purified by silica gel chromatography eluting with 0-20% EtOAc/hexanes to afford the desired product as a pale-yellow oil.

¹H NMR (CDCl₃, 500 MHz): δ 7.42 (d, J=2.0 Hz, 1H), 7.35 (d, J=2.1 Hz, 1H), 3.04-3.07 (m, 2H), 1.94-1.97 (m, 2H), 1.35 (s, 6H).

### Step C: Synthesis of 8-chloro-4,4-dimethyl-3,4-dihydro-2H-thiochromene-6-carbaldehyde

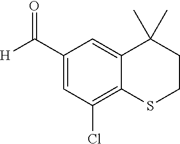

Using essentially the same procedure as INTERMEDIATE 53, Step C: 6-bromo-8-chloro-4,4-dimethyl-3,4-dihydro-2H-thiochromene (954 mg, 3.27 mmol), n-BuLi (1.57 mL, 3.93 mmol) and DMF (1.01 mL, 13.1 mmol) afforded the desired product as a pale-yellow oil.

¹H NMR (CDCl₃, 500 MHz): δ 9.88 (s, 1H), 7.82 (d, J=1.8 Hz, 1H), 7.68 (d, J=1.6 Hz, 1H), 3.11-3.15 (m, 2H), 1.98-2.02 (m, 2H), 1.41 (s, 6H).

### Step D: Synthesis of 8-cyclopropyl-4,4-dimethyl-3,4-dihydro-2H-thiochromene-6-carbaldehyde

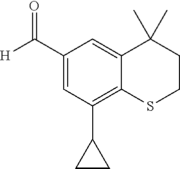 [SMILES: CC1(C)CCSc2c(C3CC3)cc(C=O)cc21]

Using essentially the same procedure as INTERMEDIATE 54: Pd(OAc)₂ (5.18 mg, 0.02 mmol), X-Phos (22.0 mg, 0.05 mmol), potassium carbonate (319 mg, 2.30 mmol), potassium cyclopropyltrifluoroborate (125 mg, 0.84 mmol), and 8-chloro-4,4-dimethyl-3,4-dihydro-2H-thiochromene-6-carbaldehyde (185 mg, 0.77 mmol) afforded the desired product as a yellow oil.

¹H NMR (CDCl₃, 500 MHz): δ 9.88 (s, 1H), 7.77 (d, J=1.7 Hz, 1H), 7.39 (d, J=1.0 Hz, 1H), 3.10-3.13 (m, 2H), 1.98-2.02 (m, 2H), 1.84-1.91 (m, 1H), 1.41 (s, 6H), 1.02 (ddd, J=10.8 Hz, 6.3 Hz, 4.6 Hz, 2H), 0.72 (ddd, J=9.9 Hz, 5.9 Hz, 4.4 Hz, 2H).

### Intermediate 65

### 8-Chloro-4,4-dimethyl-3,4-dihydro-2H-thiochromene-6-carbaldehyde 1,1-dioxide

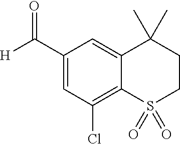 [SMILES: CC1(C)CCS(=O)(=O)c2c(Cl)cc(C=O)cc21]

A solution of 8-chloro-4,4-dimethyl-3,4-dihydro-2H-thiochromene-6-carbaldehyde (88.0 mg, 0.37 mmol) in ethanol (3.26 mL), THF (1.30 mL) and hydrogen peroxide (1.28 mL, 14.6 mmol) was treated with ammonium molybdate (14.3 mg, 0.07 mmol) at 25° C. The mixture was stirred at room temperature for 80 min before being diluted with CH₂Cl₂ (8.00 mL) and sat aqueous NH₄Cl (1.00 mL). The organic layer was separated, and the aqueous layer was extracted with CH₂Cl₂ (10.0 mL, 2×). The combined organic layers were dried over sodium sulfate, filtered, and concentrated under vacuum. The crude sulfoxide was used directly to the nest step. [M+H⁺]: m/z 257. The crude sulfoxide (100 mg, 0.39 mmol), oxone (479 mg, 0.78 mmol), methanol (6.95 mL), water (2.78 mL) mixture was stirred at room temperature for 50 min. It was then diluted with EtOAc (20.0 mL), and filtered through a pad of celite. The filtrate was concentrated under vacuum. The residue was partitioned between EtOAc (50.0 mL) and water (10.0 mL). The organic layer was separated, dried over sodium sulfate, filtered, and concentrated under vacuum. The crude residue was purified by silica gel chromatography eluting with 0-30% CH₂Cl₂/methanol to afford 8-chloro-4,4-dimethyl-3,4-dihydro-2H-thiochromene-6-carboxylic acid 1,1-dioxide as a pale-orange solid.

¹H NMR (CDCl₃, 500 MHz): δ: 8.09 (d, J=1.2 Hz, 1H), 8.07 (d, J=1.6 Hz, 1H), 3.52-3.56 (m, 2H), 2.38-2.42 (m, 2H), 1.50 (s, 6H).

[M+H⁺]: m/z 289.

8-Chloro-4,4-dimethyl-3,4-dihydro-2H-thiochromene-6-carboxylic acid 1,1-dioxide (89.0 mg, 0.31 mmol) dissolved in THF (3.85 mL) was cooled to 0° C., and BH₃.THF (462 μL, 0.46 mmol) was added dropwise via a syringe. The reaction mixture was heated to 55° C. for 2 h, cooled to room temperature, and methanol (500 μL) was added carefully. Water (500 μL) was then added, and the resulting mixture was extracted with EtOAc (30.0 mL, 3×). The combined organic layers were dried over sodium sulfate, filtered, and concentrated under vacuum to give the crude alcohol, which was used directly to the next step. The crude alcohol was dissolved in CH₂Cl₂ (4.00 mL), and Dess-Martin Periodinane (261 mg, 0.62 mmol) was added in one portion. The reaction mixture was stirred at room temperature for 1 h before it was evaporated under vacuum on silica gel. The crude mixture was purified by silica gel chromatography eluting with 0-5% CH₂Cl₂/methanol to give the desired product as a yellow solid.

[M+H⁺]: m/z 273.

### Intermediate 66

### 8-Cyclopropyl-4,4-dimethyl-3,4-dihydro-2H-thiochromene-6-carbaldehyde 1,1-dioxide

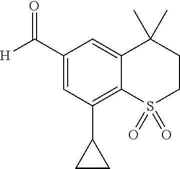 [SMILES: CC1(C)CCS(=O)(=O)c2c(C3CC3)cc(C=O)cc21]

A solution of 8-cyclopropyl-4,4-dimethyl-3,4-dihydro-2H-thiochromene-6-carbaldehyde (100 mg, 0.41 mmol, INTERMEDIATE 64) in ethanol (3.62 mL), TI-IF (1.45 mL) and hydrogen peroxide (1.42 mL, 16.2 mmol) was treated with ammonium molybdate (16.0 mg, 0.08 mmol) at 25° C. The mixture was stirred at room temperature for 90 min before being diluted with CH₂Cl₂ (10.0 mL) and sat aqueous NH₄Cl (2.00 mL). The organic layer was separated, and the aqueous phase was extracted with CH₂Cl₂ (10.0 mL, 2×). The combined organic layers were dried over sodium sulfate, filtered, and concentrated under vacuum. The crude mixture was purified by silica gel chromatography eluting with 0-50% EtOAc/hexanes to give the product as a colorless oil.

[M+H⁺]: m/z 279.

### EXAMPLES

The following EXAMPLES were made using the appropriate spiro-piperidine from INTERMEDIATES 1, 2 or 8, Step a or B, and the appropriate benzyl halide (see WO10056717) or benzaldehyde (commercial or from INTERMEDIATES 3-7, 9-66 or analogs prepared via similar procedures).

### Example 1

### 4-{8-[(2,6-Diethoxy-4′-fluorobiphenyl-4-yl)methyl]-1-oxa-2,8-diazaspiro[4.5]dec-2-en-3-yl}benzoic acid, TFA salt

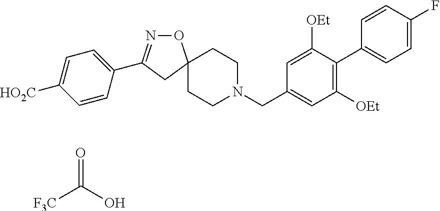 [SMILES: CCOc1cc(CN2CCC3(CC2)CC(c2ccc(C(=O)O)cc2)=NO3)cc(OCC)c1-c1ccc(F)cc1 ; O=C(O)C(F)(F)F]

### Step A: Synthesis of methyl 4-{8-[(2,6-diethoxy-4′-fluorobiphenyl-4-yl)methyl]-1-oxa-2,8-diazaspiro[4.5]dec-2-en-3-yl}benzoate

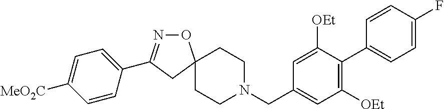 [SMILES: CCOc1cc(CN2CCC3(CC2)CC(c2ccc(C(=O)OC)cc2)=NO3)cc(OCC)c1-c1ccc(F)cc1]

To a 20 mL scintillation vial, 3-[4-(methoxycarbonyl)phenyl]-1-oxa-2,8-diazaspiro[4.5]dec-2-ene hydrochloride (30 g, 0.10 mmol) (INTERMEDIATE 1), DIPEA (0.060 mL, 0.30 mmol), 4-(chloromethyl)-2,6-diethoxy-4′-fluorobiphenyl (50 mg, 0.16 mmol) and DMF (5 mL) were added. The mixture was stirred at 60° C. for 2 hours until the LCMS showed no further conversion. After cooling to room temperature, the reaction mixture was diluted with 0.5 mL of water and acidified with TFA. It was loaded on to a reverse phase (C-18) HPLC column and purified with a Gilson HPLC eluting with a water/acetonitrile gradient solvent to afford the intermediate methyl ester as a colorless residue.

### Step B: Synthesis of 4-{8-[(2,6-diethoxy-4′-fluorobiphenyl-4-yl)methyl]-1-oxa-2,8-diazaspiro[4.5]dec-2-en-3-yl}benzoic acid, TFA salt

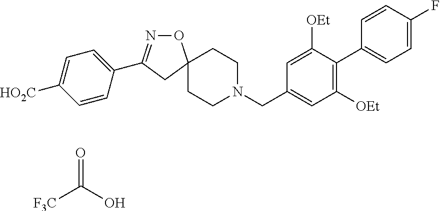 [SMILES: CCOc1cc(CN2CCC3(CC2)CC(c2ccc(C(=O)O)cc2)=NO3)cc(OCC)c1-c1ccc(F)cc1 ; O=C(O)C(F)(F)F]

To the residue in methanol (1 mL), KOH (0.145 mL, 0.290 mmol, 2N) solution and water (0.5 mL) were added and the mixture was heated at 65° C. for 2 hours. The reaction mixture was cooled to room temperature and concentrated. Water (1 mL) followed by trifluoroacetic acid (0.3 mL) were added to the residue. Acetonitrile (2 mL) was added to the solution and the mixture was purified by preparative HPLC reverse phase (C-18) HPLC (C-18) HPLC (C-18) chromatography eluting with acetonitrile/water+0.1% TFA to give the title as the TFA salt.

¹H NMR (CD₃OD, 500 MHz): δ 8.0 (d, J=8.1 Hz, 2H), 7.8 (d, J=8.3 Hz, 2H), 7.3 (m, J=8.3 Hz, 2H), 7.1 (t, J=8.8 Hz, 2H), 6.86 (s, 2H), 4.4 (s, 2H), 4.0 (q, 4H), 3.9 (s, 3H), 3.57 (b, 2H), 3.4 (b, 4H), 2.25 (b, 2H), 2.18 (b, 2H), 1.24 (t, J=6.9 Hz, 6H).

[M+H⁺]: m/z 547.

### Example 2

### 4-{8-[(4,4-Dimethyl-3,4-dihydro-2-H-chromen-6-yl)methyl]-1-oxa-2,8-diazaspiro[4.5]dec-2-en-3-yl}benzoic acid, TFA salt

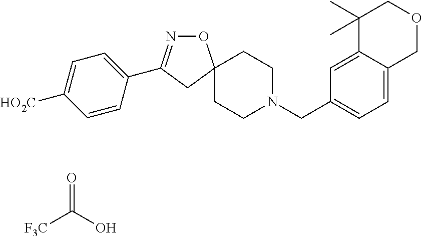 [SMILES: CC1(C)COCc2ccc(CN3CCC4(CC3)CC(c3ccc(C(=O)O)cc3)=NO4)cc21 ; O=C(O)C(F)(F)F]

To a vial, 3-[4-(methoxycarbonyl)phenyl]-1-oxa-2,8-diazaspiro[4.5]dec-2-ene hydrochloride (33 mg, 0.105 mmol) (INTERMEDIATE 1), 4,4-dimethyl-3,4-dihydro-2H-chromene-6-carboxaldehyde (20 mg, 0.105 mmol) (INTERMEDIATE 6), sodium triacetoxyborohydride (67 mg, 0.32 mmol), AcOH (0.036 mL, 0.63 mmol) and DMSO (1 mL) were added. The resulting reaction mixture was stirred at 50° C. overnight. The reaction was cooled to room temperature, diluted with DMSO and water, acidified with TFA, loaded on to a reverse phase (C-18) HPLC column and purified with a Gilson HPLC eluting with a solvent gradient of water/acetonitrile. The fractions containing the desired product were collected and concentrated to give the intermediate methyl ester.

The residue was dissolved in MeOH (5 mL) and treated with KOH (1 mL, 10% aqueous solution) at 60° C. for 2 hours. LC-Mass indicated complete hydrolysis. The volatiles were removed and the residue was diluted with 10 mL water and acidified with TFA, which caused a heavy precipitation. The precipitate was filtered and the solid was washed with water and air dried to give the title compound as a white solid.

¹H NMR (CD₃OD, 500 MHz): δ 8.07 (d, J=8.4 Hz, 2H), 7.78 (d, J=8.4 Hz, 2H), 7.48 (d, J=2 Hz, 1H), 7.2 (dd, J=8.4 Hz, 2.1 Hz, 1H), 6.85 (d, J=8.4 Hz, 2H), 4.29 (s, 2H), 4.23 (t, J=5.3 Hz, 2H), 3.4 (b, 6H), 2.2 (m, 4H) 1.87 (t, J=5.4 Hz, 2H), 1.38 (s, 6H).

[M+H⁺]: m/z 434.

### Example 3

### 4-(8-{[1-tert-Butyl-3-(2,3,4-trifluorophenyl)-1H-pyrazol-4-yl]methyl})-1-oxa-2,8-diazaspiro[4.5]dec-2-en-3-yl)benzoic acid, TFA salt

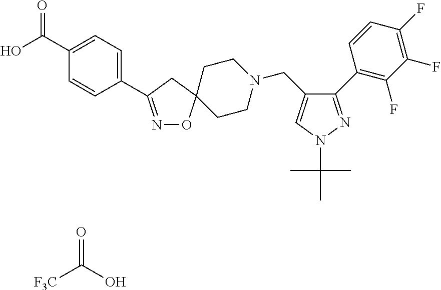 [SMILES: CC(C)(C)n1cc(CN2CCC3(CC2)CC(c2ccc(C(=O)O)cc2)=NO3)c(-c2ccc(F)c(F)c2F)n1 ; O=C(O)C(F)(F)F]

Triethylamine (0.067 mL, 0.483 mmol) was added to a stirred, cooled room temperature mixture of 3-[4-(methoxycarbonyl)phenyl]-1-oxa-2-aza-8-azoniaspiro[4.5]dec-2-ene hydrochloride (30 mg, 0.097 mmol) (INTERMEDIATE 1) in methanol (1.5 mL) and the mixture was stirred at room temperature for 5 minutes. Then 1-tert-butyl-3-(2,3,4-trifluorophenyl)-1H-pyrazole-4-carboxaldehyde (27.2 mg, 0.097 mmol) (INTERMEDIATE 7) and decaborane (3.54 mg, 0.029 mmol) were added and the solution was stirred for 16 hours at room temperature. The reaction mixture was concentrated and the residue was partitioned between ethyl acetate (20 mL) and saturated sodium bicarbonate (10 mL) and washed with brine (10 mL). The organic layer was dried over sodium sulfate, filtered and concentrated to afford the intermediate methyl ester. To the residue in methanol (1 mL), KOH (0.145 mL, 0.290 mmol, 2N) and water (0.5 mL) were added and the mixture was heated at 65° C. for 2 hours. The reaction mixture was cooled to room temperature and concentrated. Water (1 mL) followed by trifluoroacetic acid (0.3 mL) were added to the residue. Acetonitrile (2 mL) was added to the solution and the mixture was purified by preparative HPLC on a reverse phase (C-18) column eluting with acetonitrile/water+0.1% TFA to give the title compound as a colorless solid after lypholization of the acetonitrile/water.

¹H NMR (CD₃OD, 500 MHz): δ 8.14 (s, 1H), 8.06 (d, 2H, J=8.3 Hz), 7.76 (d, 2H, J=8.3 Hz), 7.38-7.36 (m, 1H), 7.28-7.26 (m, 1H), 4.40 (s, 2H), 3.42-3.41 (m, 2H), 3.31-3.16 (m, 2H), 2.14-2.09 (m, 4H), 1.67 (s, 9H).

[M+H⁺]: m/z 527.

### Example 4

### 4-{8-[(4-Ethoxy-2′,4′-difluorobiphenyl-2-yl)methyl]-1-oxa-2,8-diazaspiro[4.5]dec-2-en-3-yl}benzoic acid

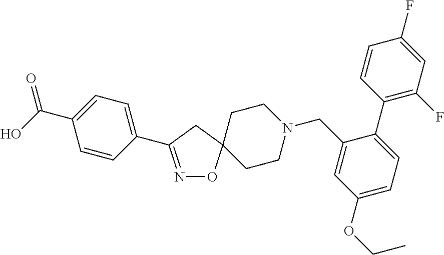 [SMILES: CCOc1ccc(-c2ccc(F)cc2F)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 507.

### Example 5

### 4-{8-[3,5-Diethoxy-4-(2,2,2-trifluoroethoxy)benzyl]-1-oxa-2,8-diazaspiro[4.5]dec-2-en-3-yl}benzoic acid

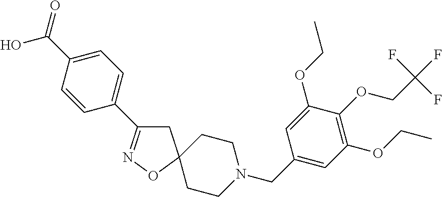 [SMILES: CCOc1cc(CN2CCC3(CC2)CC(c2ccc(C(=O)O)cc2)=NO3)cc(OCC)c1OCC(F)(F)F]

The title compound was prepared from INTERMEDIATE 1 and the appropriate aldehyde following essentially the same procedure described in EXAMPLE 1.

[M+H⁺]: m/z 537.

### Example 6

### 4-(8-{[6-Ethoxy-3-(2,3,4-trifluorophenyl)pyridin-2-yl]methyl}-1-oxa-2,8-diazaspiro[4.5]dec-2-en-3-yl)benzoic acid

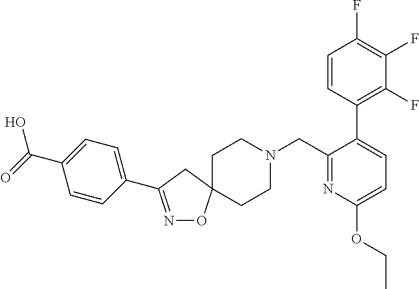 [SMILES: CCOc1ccc(-c2ccc(F)c(F)c2F)c(CN2CCC3(CC2)CC(c2ccc(C(=O)O)cc2)=NO3)n1]

The title compound was prepared from INTERMEDIATE 1 and the appropriate aldehyde following essentially the same procedure described in EXAMPLE 2.

[M+H⁺]: m/z 526.

### Example 7

### 4-(8-{[2-Ethoxy-5-(2,3,4-trifluorophenyl)pyridin-4-yl]methyl}-1-oxa-2,8-diazaspiro[4.5]dec-2-en-3-yl)benzoic acid

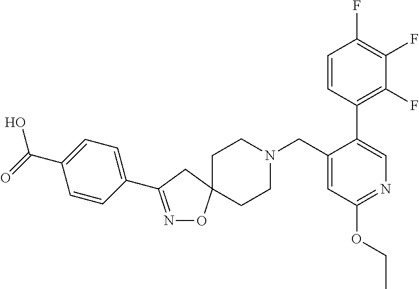 [SMILES: CCOc1cc(CN2CCC3(CC2)CC(c2ccc(C(=O)O)cc2)=NO3)c(-c2ccc(F)c(F)c2F)cn1]

The title compound was prepared from INTERMEDIATE 1 and the appropriate aldehyde following essentially the same procedure described in EXAMPLE 2.

[M+H⁺]: m/z 526.

### Example 8

### 4-{8-[(4-Ethoxy-3′,4′-difluorobiphenyl-2-yl)methyl]-1-oxa-2,8-diazaspiro[4.5]dec-2-en-3-yl}benzoic acid

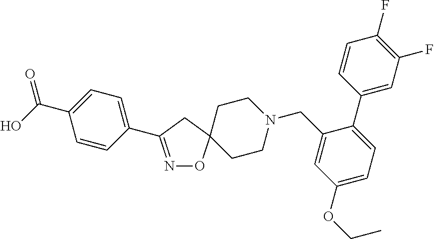 [SMILES: CCOc1ccc(-c2ccc(F)c(F)c2)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 507.

### Example 9

### 4-{8-[(4-Ethoxy-2′,3′,4′-trifluorobiphenyl-2-yl)methyl]-1-oxa-2,8-diazaspiro[4.5]dec-2-en-3-yl}benzoic acid

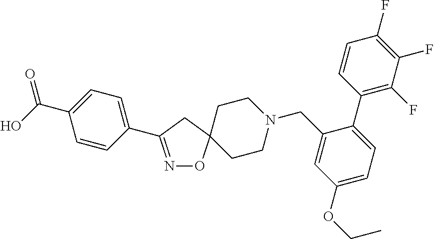 [SMILES: CCOc1ccc(-c2ccc(F)c(F)c2F)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 525.

### Example 10

### 4-{8-[(4-Ethoxy-2′,4′,5′-trifluorobiphenyl-2-yl)methyl]-1-oxa-2,8-diazaspiro[4.5]dec-2-en-3-yl}benzoic acid

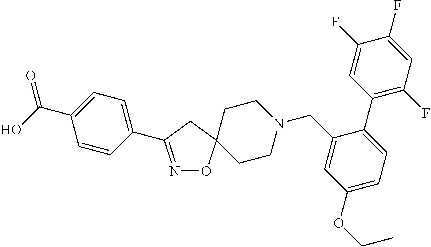 [SMILES: CCOc1ccc(-c2cc(F)c(F)cc2F)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 525.

### Example 11

### 4-[8-(3,5-Diethoxy-4-methoxybenzyl)-1-oxa-2,8-diazaspiro[4.5]dec-2-en-3-yl]benzoic acid

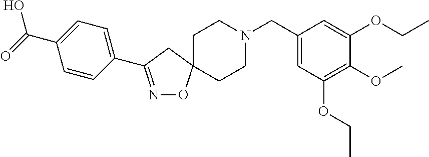 [SMILES: CCOc1cc(CN2CCC3(CC2)CC(c2ccc(C(=O)O)cc2)=NO3)cc(OCC)c1OC]

The title compound was prepared from INTERMEDIATE 1 and the appropriate aldehyde following essentially the same procedure described in EXAMPLE 1.

[M+H⁺]: m/z 469.

### Example 12

### 4-[8-(4-Chloro-3-ethoxybenzyl)-1-oxa-2,8-diazaspiro[4.5]dec-2-en-3-yl]benzoic acid

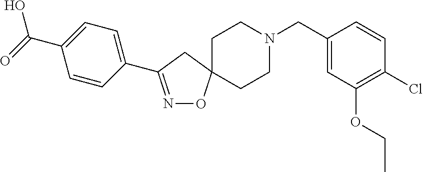 [SMILES: CCOc1cc(CN2CCC3(CC2)CC(c2ccc(C(=O)O)cc2)=NO3)ccc1Cl]

The title compound was prepared from INTERMEDIATE 1 and the appropriate aldehyde following essentially the same procedure described in EXAMPLE 2.

[M+H⁺]: m/z 429.

### Example 13

### 4-(8-{[3′,4′-Difluoro-4-(propan-2-yloxy)biphenyl-2-yl]methyl}-1-oxa-2,8-diazaspiro[4.5]dec-2-en-3-yl)benzoic acid

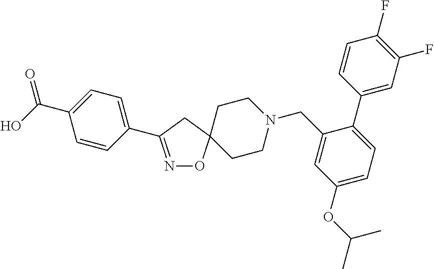 [SMILES: CC(C)Oc1ccc(-c2ccc(F)c(F)c2)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 521.

### Example 14

### 4-(8-{[2′,3′,4′-(propan-2-yloxy)biphenyl-2-yl]methyl}-1-oxa-2,8-diazaspiro[4.5]dec-2-en-3-yl)benzoic acid

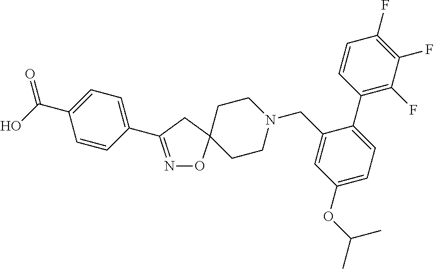 [SMILES: CC(C)Oc1ccc(-c2ccc(F)c(F)c2F)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 539.

### Example 15

### 4-(8-{[2′,4′,5′-Trifluoro-4-(propan-2-yloxy)biphenyl-2-yl]methyl}-1-oxa-2,8-diazaspiro[4.5]dec-2-en-3-yl)benzoic acid

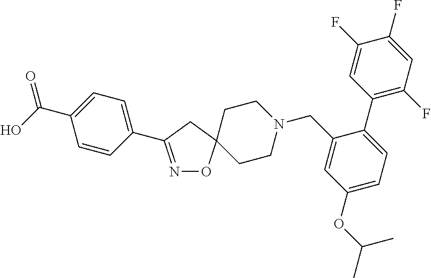 [SMILES: CC(C)Oc1ccc(-c2cc(F)c(F)cc2F)c(CN2CCC3(CC2)CC(c2ccc(C(=O)O)cc2)=NO3)c1]

The title compound was prepared from INTERMEDIATE 1 and the appropriate aldehyde following essentially the same procedure described in EXAMPLE 2.

[M+H⁺]: m/z 539.

### Example 16

### 4-{8-[3-Bromo-4-(trifluoromethoxy)benzyl]-1-oxa-2,8-diazaspiro[4.5]dec-2-en-3-yl}benzoic acid

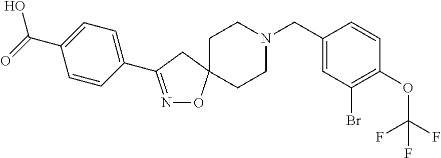 [SMILES: O=C(O)c1ccc(C2=NOC3(CCN(Cc4ccc(OC(F)(F)F)c(Br)c4)CC3)C2)cc1]

The title compound was prepared from INTERMEDIATE 1 and the appropriate aldehyde following essentially the same procedure described in EXAMPLE 2.

[M+H⁺]: m/z 513.

### Example 17

### 4-{8-[(4-Ethoxy-2′,3,4′-trifluoro-5-methylbiphenyl-2-yl)methyl]-1-oxa-2,8-diazaspiro[4.5]dec-2-en-3-yl}benzoic acid

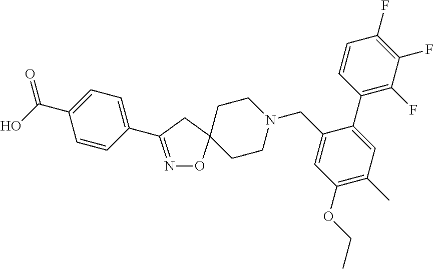 [SMILES: CCOc1cc(CN2CCC3(CC2)CC(c2ccc(C(=O)O)cc2)=NO3)c(-c2ccc(F)c(F)c2F)cc1C]

The title compound was prepared from INTERMEDIATE 1 and the appropriate aldehyde following essentially the same procedure described in EXAMPLE 2.

[M+H⁺]: m/z 539.

### Example 18

### 4-{8-[3-Ethoxy-4-(1,3-thiazol-2-yl)benzyl]-1-oxa-2,8-diazaspiro[4.5]dec-2-en-3-yl}benzoic acid

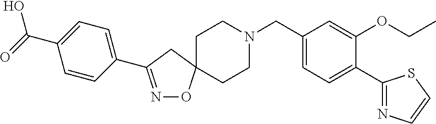 [SMILES: CCOc1cc(CN2CCC3(CC2)CC(c2ccc(C(=O)O)cc2)=NO3)ccc1-c1nccs1]

The title compound was prepared from INTERMEDIATE 1 and the appropriate aldehyde following essentially the same procedure described in EXAMPLE 2.

[M+H⁺]: m/z 478.

### Example 19

### 4-{8-[5-Ethoxy-4-methyl-2-(1,3-thiazol-2-yl)benzyl]-1-oxa-2,8-diazaspiro[4.5]dec-2-en-3-yl}benzoic acid

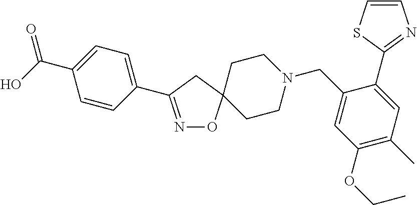 [SMILES: CCOc1cc(CN2CCC3(CC2)CC(c2ccc(C(=O)O)cc2)=NO3)c(-c2nccs2)cc1C]

The title compound was prepared from INTERMEDIATE 1 and the appropriate aldehyde following essentially the same procedure described in EXAMPLE 2.

[M+H⁺]: m/z 492.

### Example 20

### 4-[8-(3,4-Dichloro-5-ethoxybenzyl)-1-oxa-2,8-diazaspiro[4.5]dec-2-en-3-yl]benzoic acid

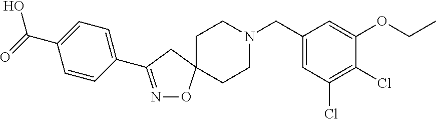 [SMILES: CCOc1cc(CN2CCC3(CC2)CC(c2ccc(C(=O)O)cc2)=NO3)cc(Cl)c1Cl]

The title compound was prepared from INTERMEDIATE 1 and the appropriate aldehyde following essentially the same procedure described in EXAMPLE 2.

[M+H⁺]: m/z 463.

### Example 21

### Methyl 6-{8-[(2,6-diethoxy-4′-fluorobiphenyl-4-yl)methyl]-1-oxa-2,8-diazaspiro[4.5]dec-2-en-3-yl}pyridine-3-carboxylate

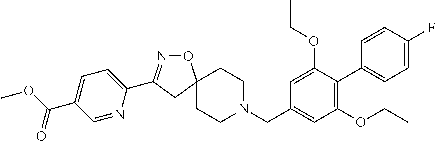 [SMILES: CCOc1cc(CN2CCC3(CC2)CC(c2ccc(C(=O)OC)cn2)=NO3)cc(OCC)c1-c1ccc(F)cc1]

The title compound was prepared from INTERMEDIATE 8, Step A, and the appropriate aldehyde following essentially the same procedure described in EXAMPLE 1.

[M+⁺]: m/z 548.

### Example 22

### Methyl 6-(8-{[1-tert-butyl-3-(2,3,4-trifluorophenyl)-1H-pyrazol-4-yl]methyl}-1-oxa-2,8-diazaspiro[4.5]dec-2-en-3-yl)pyridine-3-carboxylate

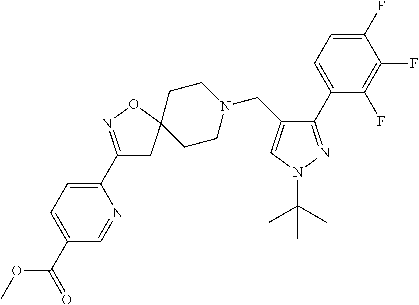 [SMILES: COC(=O)c1ccc(C2=NOC3(CCN(Cc4cn(C(C)(C)C)nc4-c4ccc(F)c(F)c4F)CC3)C2)nc1]

The title compound was prepared from INTERMEDIATE 8, Step A, and the appropriate aldehyde following essentially the same procedure described in EXAMPLE 3.

[M+H⁺]: m/z 542.

### Example 23

### 4-(8-{[1-tert-Butyl-3-(2,3,4-trifluorophenyl)-1H-pyrazol-4-yl]methyl}-1-oxa-2,8-diazaspiro[4.5]dec-2-en-3-yl)pyridine-3-carboxylic acid

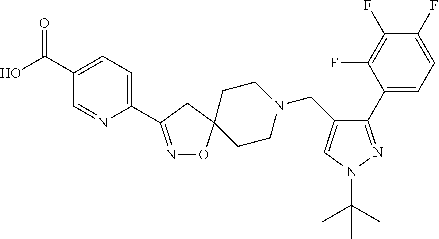 [SMILES: CC(C)(C)n1cc(CN2CCC3(CC2)CC(c2ccc(C(=O)O)cn2)=NO3)c(-c2ccc(F)c(F)c2F)n1]

The title compound was prepared from INTERMEDIATE 8 and the appropriate aldehyde following essentially the same procedure described in EXAMPLE 3.

[M+H⁺]: m/z 528.

### Example 24

### 4-(8-{[1-tert-Butyl-3-(2,3,4-trifluorophenyl)-1H-pyrazol-4-yl]methyl}-1-oxa-2,8-diazaspiro[4.5]dec-2-en-3-yl)benzoic acid

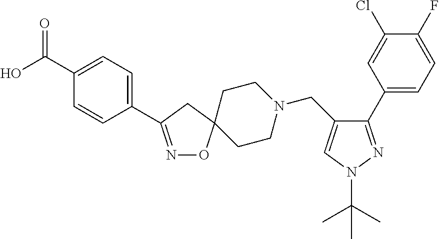 [SMILES: CC(C)(C)n1cc(CN2CCC3(CC2)CC(c2ccc(C(=O)O)cc2)=NO3)c(-c2ccc(F)c(Cl)c2)n1]

The title compound was prepared from INTERMEDIATE 1 and the appropriate aldehyde following essentially the same procedure described in EXAMPLE 3.

[M+H⁺]: m/z 525.

### Example 25

### 6-{8-[(2,6-Diethoxy-4′-fluorobiphenyl-4-yl)methyl]-1-oxa-2,8-diazaspiro[4.5]dec-2-en-3-yl}pyridine-3-carboxylic acid

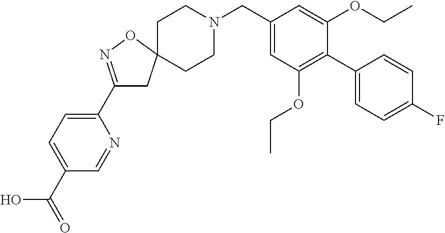 [SMILES: CCOc1cc(CN2CCC3(CC2)CC(c2ccc(C(=O)O)cn2)=NO3)cc(OCC)c1-c1ccc(F)cc1]

The title compound was prepared from INTERMEDIATE 8 and the appropriate aldehyde following essentially the same procedure described in EXAMPLE 1.

[M+H⁺]: m/z 534.

### Example 26

### 4-{8-[5-Ethoxy-2-(6-fluoropyridin-3-yl)-4-methylbenzyl]-1-oxa-2,8-diazaspiro[4.5]dec-2-en-3-yl}benzoic acid

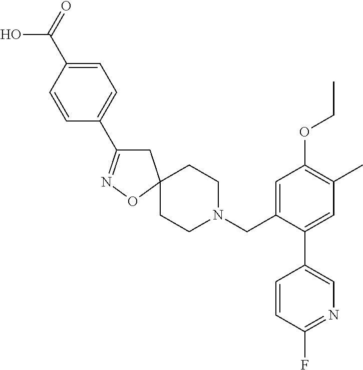 [SMILES: CCOc1cc(CN2CCC3(CC2)CC(c2ccc(C(=O)O)cc2)=NO3)c(-c2ccc(F)nc2)cc1C]

The title compound was prepared from INTERMEDIATE 1 and the appropriate aldehyde following essentially the same procedure described in EXAMPLE 2.

[M+H⁺]: m/z 504.

### Example 27

### 6-(8-{(1-tert-Butyl-3-(3-chloro-4-fluorophenyl)-1H-pyrazol-4-yl]methyl}-1-oxa-2,8-diazaspiro[4.5]dec-2-en-3-yl)pyridine-3-carboxylic acid

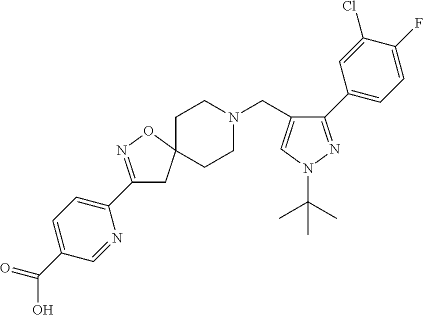 [SMILES: CC(C)(C)n1cc(CN2CCC3(CC2)CC(c2ccc(C(=O)O)cn2)=NO3)c(-c2ccc(F)c(Cl)c2)n1]

The title compound was prepared from INTERMEDIATE 8 and the appropriate aldehyde following essentially the same procedure described in EXAMPLE 3.

[M+H⁺]: m/z 526.

### Example 28

### 6-{8-[5-Ethoxy-2-(1,3-thiazol-2-yl)benzyl]-1-oxa-2,8-diazaspiro[4.5]dec-2-en-3-yl}pyridine-3-carboxylic acid

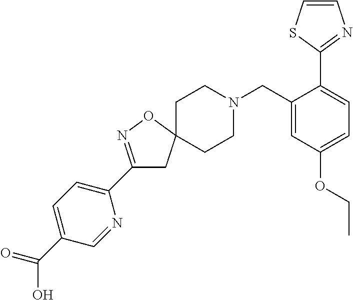 [SMILES: CCOc1ccc(-c2nccs2)c(CN2CCC3(CC2)CC(c2ccc(C(=O)O)cn2)=NO3)c1]

The title compound was prepared from INTERMEDIATE 8 and the appropriate aldehyde following essentially the same procedure described in EXAMPLE 2.

[M+H⁺]: m/z 479.

### Example 29

### 6-{8-[(4-Ethoxy-2′,3′,4′-trifluoro-5-methylbiphenyl-2-yl)methyl]-1-oxa-2,8-diazaspiro[4.5]dec-2-en-3-yl}pyridine-3-carboxylic acid

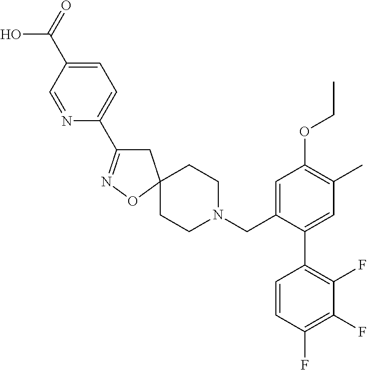 [SMILES: CCOc1cc(CN2CCC3(CC2)CC(c2ccc(C(=O)O)cn2)=NO3)c(-c2ccc(F)c(F)c2F)cc1C]

The title compound was prepared from INTERMEDIATE 8 and the appropriate aldehyde following essentially the same procedure described in EXAMPLE 2.

[M+H⁺]: m/z 540.

### Example 30

### 6-{8-[(4-Ethoxy-2′,3′,4′-trifluorobiphenyl-2-yl)methyl]-1-oxa-2,8-diazaspiro[4.5]dec-2-en-3-yl}pyridine-3-carboxylic acid

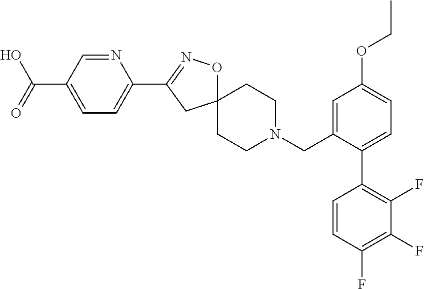 [SMILES: CCOc1ccc(-c2ccc(F)c(F)c2F)c(CN2CCC3(CC2)CC(c2ccc(C(=O)O)cn2)=NO3)c1]

The title compound was prepared from INTERMEDIATE 8 and the appropriate aldehyde following essentially the same procedure described in EXAMPLE 2.

[M+H⁺]: m/z 526.

### Example 31

### 4-(8-{[2′,3′,4′-Trifluoro-4-(trifluoromethoxy)biphenyl-2-yl]methyl}-1-oxa-2,8-diazaspiro[4.5]dec-2-en-3-yl)benzoic acid

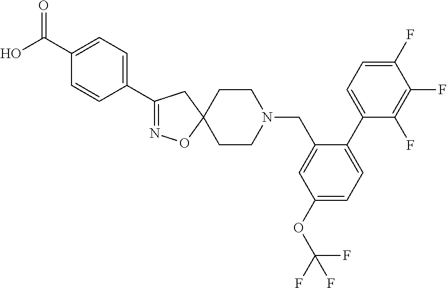 [SMILES: O=C(O)c1ccc(C2=NOC3(CCN(Cc4cc(OC(F)(F)F)ccc4-c4ccc(F)c(F)c4F)CC3)C2)cc1]

The title compound was prepared from INTERMEDIATE 1 and the appropriate aldehyde following essentially the same procedure described in EXAMPLE 2.

[M+H⁺]: m/z 565.

### Example 32

### 4-{8-[2-(6-Fluoropyridin-3-yl)-5-(trifluoromethoxy)benzyl]-1-oxa-2,8-diazaspiro[4.5]dec-2-en-3-yl}benzoic acid

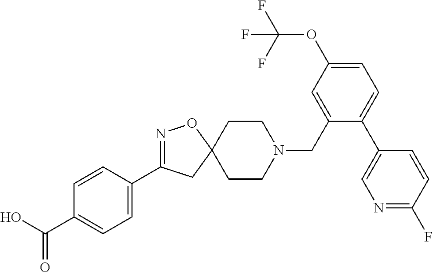 [SMILES: O=C(O)c1ccc(C2=NOC3(CCN(Cc4cc(OC(F)(F)F)ccc4-c4ccc(F)nc4)CC3)C2)cc1]

The title compound was prepared from INTERMEDIATE 1 and the appropriate aldehyde following essentially the same procedure described in EXAMPLE 2.

[M+H⁺]: m/z 530.

### Example 33

### 4-{8-[3-tert-Butyl-4-(6-fluoropyridin-3-yl)benzyl]-1-oxa-2,8-diazaspiro[4.5]dec-2-en-3-yl}benzoic acid

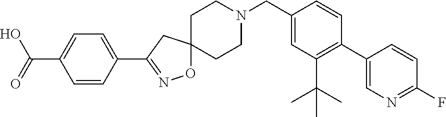 [SMILES: CC(C)(C)c1cc(CN2CCC3(CC2)CC(c2ccc(C(=O)O)cc2)=NO3)ccc1-c1ccc(F)nc1]

The title compound was prepared from INTERMEDIATE 1 and the appropriate aldehyde following essentially the same procedure described in EXAMPLE 2.

[M+H⁺]: m/z 502.

### Example 34

### 4-{8-[(2,6-Dichloro-4′-fluorobiphenyl-4-yl)methyl]-1-oxa-2,8-diazaspiro[4.5]dec-2-en-3-yl}benzoic acid, TFA salt

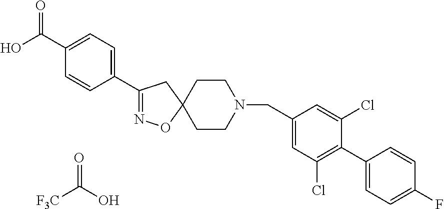 [SMILES: O=C(O)c1ccc(C2=NOC3(CCN(Cc4cc(Cl)c(-c5ccc(F)cc5)c(Cl)c4)CC3)C2)cc1 ; O=C(O)C(F)(F)F]

To a solution of 3-[4-(carboxy)phenyl]-1-oxa-2,8-diazaspiro[4.5]dec-2-ene hydrochloride (27 mg, 0.070 mmol) (INTERMEDIATE 2) and 2,6-dichloro-4′-fluorobiphenyl-4-carboxaldehyde (15 mg, 0.056 mmol) (INTERMEDIATE 12) in DMF (2 mL) in a 20 mL scintillation vial was added acetic acid (5 uL, 0.08 mmol) and MP-cyanoborohydride resin (45 mg, 0.111 mmol, 2.49 mmol/g). The mixture was shaken at room temperature for 18 hours and then filtered to remove the resin. The product was isolated directly from the filtrate by reverse phase (C-18) HPLC chromatography (10-55% acetonitrile/water gradient) to afford the title compound (22 mg) as a solid TFA salt after evaporation and trituration with ether.

[M+H⁺]: m/z 513.

### Example 35

### 4-{8-[(2,6-Dichloro-2′,4′-difluorobiphenyl-4-yl)methyl]-1-oxa-2,8-diazaspiro[4.5]dec-2-en-3-yl}benzoic acid, TFA salt

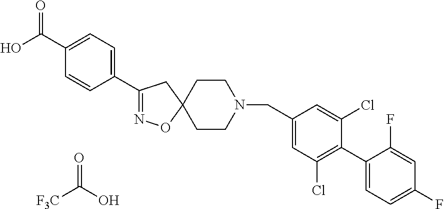 [SMILES: O=C(O)c1ccc(C2=NOC3(CCN(Cc4cc(Cl)c(-c5ccc(F)cc5F)c(Cl)c4)CC3)C2)cc1 ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (20 mg, 0.065 mmol) (INTERMEDIATE 2) and 2,6-dichloro-2′,4′-difluorobiphenyl-4-carboxaldehyde (15 mg, 0.052 mmol) (INTERMEDIATE 13) afforded the title compound (21 mg) as a solid TFA salt after trituration with ether.

[M+H⁺]: m/z 531.

### Example 36

### 4-{8-[(2,6-Dichloro-3′,4′-difluorobiphenyl-4-yl)methyl]-1-oxa-2,8-diazaspiro[4.5]dec-2-en-3-yl}benzoic acid, TFA salt

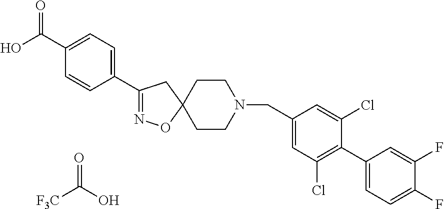 [SMILES: O=C(O)c1ccc(C2=NOC3(CCN(Cc4cc(Cl)c(-c5ccc(F)c(F)c5)c(Cl)c4)CC3)C2)cc1 ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (20 mg, 0.065 mmol) (INTERMEDIATE 2) and 2,6-dichloro-3′,4′-difluorobiphenyl-4-carboxaldehyde (15 mg, 0.052 mmol) (INTERMEDIATE 14) afforded the title compound (20 mg) as a solid TFA salt after trituration with ether.

[M+H⁺]: m/z 531.

### Example 37

### 4-{8-[(2,6-Dimethyl-4′-fluorobiphenyl-4-yl)methyl]-1-oxa-2,8-diazaspiro[4.5]dec-2-en-3-yl}benzoic acid, TFA salt

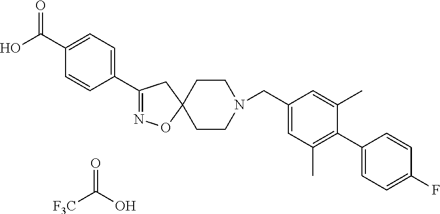 [SMILES: Cc1cc(CN2CCC3(CC2)CC(c2ccc(C(=O)O)cc2)=NO3)cc(C)c1-c1ccc(F)cc1 ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (28 mg, 0.094 mmol) (INTERMEDIATE 2) and 2,6-dimethyl-4′-fluorobiphenyl-4-carboxaldehyde (44 mg, 0.193 mmol) (INTERMEDIATE 15) afforded the title compound (33 mg) as a solid TFA salt.

[M+H⁺]: m/z 473.

### Example 38

### 4-{8-[(2,6-Dimethyl-2′,4′-difluorobiphenyl-4-yl)methyl]-1-oxa-2,8-diazaspiro[4.5]dec-2-en-3-yl}benzoic acid, TFA salt

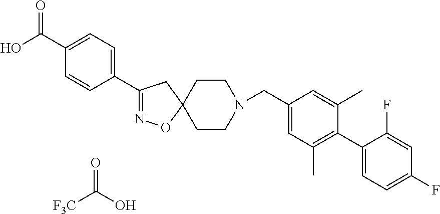 [SMILES: Cc1cc(CN2CCC3(CC2)CC(c2ccc(C(=O)O)cc2)=NO3)cc(C)c1-c1ccc(F)cc1F ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (24 mg, 0.081 mmol) (INTERMEDIATE 2) and 2,6-dichloro-3′,4′-difluorobiphenyl-4-carboxaldehyde (58 mg, 0.24 mmol) (INTERMEDIATE 16) afforded the title compound (29 mg) as a solid TFA salt.

[M+H⁺]: m/z 491.

### Example 39

### 4-(8-{[4-(4-Fluorophenyl)-1-(propan-2-yl)-1H-indol-3-yl]methyl}-1-oxa-2,8-diazaspiro[4.5]dec-2-en-3-yl)benzoic acid, TFA salt

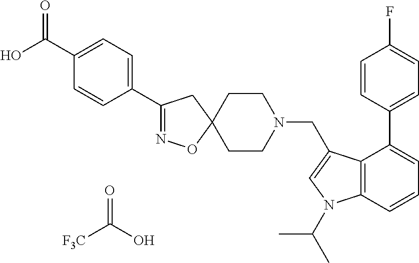 [SMILES: CC(C)n1cc(CN2CCC3(CC2)CC(c2ccc(C(=O)O)cc2)=NO3)c2c(-c3ccc(F)cc3)cccc21 ; O=C(O)C(F)(F)F]

To a solution of 3-[4-(carboxy)phenyl]-1-oxa-2,8-diazaspiro[4.5]dec-2-ene hydrochloride (20 mg, 0.067 mmol) (INTERMEDIATE 2) and 4-(4-fluorophenyl)-1-(propan-2-yl)-1H-indol-3-carboxaldehyde (19 mg, 0.067 mmol) (INTERMEDIATE 17) in DMF (2 mL) was added sodium triacetoxyborohydride (57 mg, 0.27 mmol). The reaction was stirred at room temperature for 16 hours and was then quenched with water (1 mL) and filtered. The mixture was purified by reverse phase (C-18) HPLC chromatography (5-95% acetonitrile/water with 0.1% TFA) to afford the title compound (10 mg) as a solid TFA salt.

[M+H⁺]: m/z 526.

### Example 40

### 4-(8-{[4-(6-Fluoropyridin-3-yl)-1-(propan-2-yl)-1H-indol-3-yl]methyl}-1-oxa-2,8-diazaspiro[4.5]dec-2-en-3-yl)benzoic acid, TFA salt

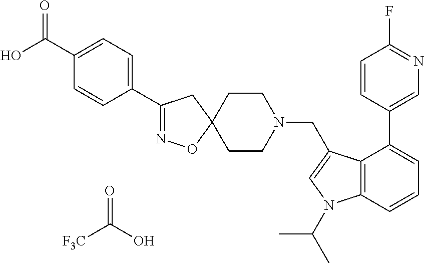 [SMILES: CC(C)n1cc(CN2CCC3(CC2)CC(c2ccc(C(=O)O)cc2)=NO3)c2c(-c3ccc(F)nc3)cccc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (20 mg, 0.067 mmol) (INTERMEDIATE 2) and 4-(6-fluoropyridin-3-yl)-1-(propan-2-yl)-1H-indole-3-carboxaldehyde (19 mg, 0.067 mmol) (INTERMEDIATE 18) afforded the title compound (10 mg) as a solid TFA salt.

[M+H⁺]: m/z 527.

### Example 41

### 4-(8-{[4-(2,4,5-Trifluorophenyl)-1-(propan-2-yl)-1H-indol-3-yl]methyl}-1-oxa-2,8-diazaspiro[4.5]dec-2-en-3-yl)benzoic acid, TFA salt

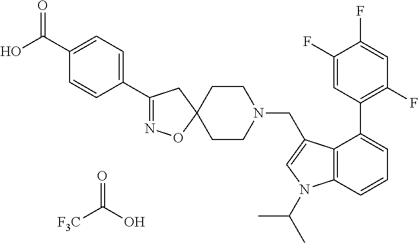 [SMILES: CC(C)n1cc(CN2CCC3(CC2)CC(c2ccc(C(=O)O)cc2)=NO3)c2c(-c3cc(F)c(F)cc3F)cccc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (20 mg, 0.067 mmol) (INTERMEDIATE 2) and 4-(2,4,5-trifluorophenyl)-1-(propan-2-yl)-1H-indole-3-carboxaldehyde (21 mg, 0.067 mmol) (INTERMEDIATE 19) afforded the title compound (10 mg) as a solid TFA salt.

[M+H⁺]: m/z 562.

### Example 42

### 4-(8-{[4-(4-Fluorophenyl)-1-(propan-2-yl)-1H-indazol-3-yl]methyl}-1-oxa-2,8-diazaspiro[4.5]dec-2-en-3-yl)benzoic acid, TFA salt

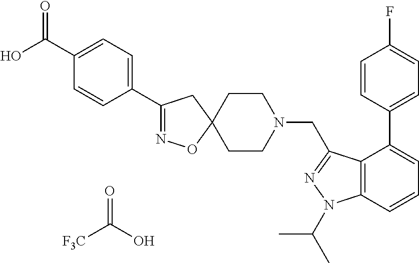 [SMILES: CC(C)n1nc(CN2CCC3(CC2)CC(c2ccc(C(=O)O)cc2)=NO3)c2c(-c3ccc(F)cc3)cccc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (15 mg, 0.051 mmol) (INTERMEDIATE 2) and 4-(4-fluorophenyl)-1-(propan-2-yl)-1H-indazole-3-carboxaldehyde (14 mg, 0.051 mmol) (INTERMEDIATE 20) afforded the title compound (10 mg) as a solid TFA salt.

¹H NMR (CD₃Cl, 500 MHz): δ 8.18 (br s, 1H), 8.12 (d, J=8.0 Hz, 2H), 7.69 (d, J=8.0 Hz, 2 H), 7.48 (d, J=8.0 Hz, 1H), 7.43 (dd, 7.5 and 8.0 Hz, 2H), 7.33 (d, J=7.5 Hz, 1H), 7.25-7.32 (m, 2H), 7.04 (d, J=7.0 Hz, 1H), 4.77 (m, 1H), 4.07 (s, 2H), 3.14 (m, 4H), 2.4-2.6 (m, 4H), 1.9 (m, 2H), 1.64 (d, J=6.6, 6H).

[M+H⁺]: m/z 527.

### Example 43

### 4-(8-{[4-(4-Fluorophenyl)-1-(propan-2-yl)-1H-pyrrolo[2,3-b]pyridin-3-yl]methyl}-1-oxa-2,8-diazaspiro[4.5]dec-2-en-3-yl)benzoic acid, TFA salt

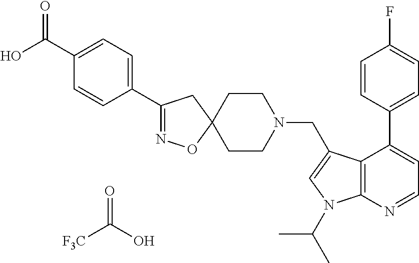 [SMILES: CC(C)n1cc(CN2CCC3(CC2)CC(c2ccc(C(=O)O)cc2)=NO3)c2c(-c3ccc(F)cc3)ccnc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (27 mg, 0.088 mmol) (INTERMEDIATE 2) and 4-(4-fluorophenyl)-1-(propan-2-yl)-1H-pyrrolo[2,3-b]pyridine-3-carboxaldehyde (35 mg, 0.088 mmol) (INTERMEDIATE 21) afforded the title compound (26 mg) as a solid TFA salt.

[M+H⁺]: m/z 527.

### Example 44

### 4-(8-{[7-Methyl-1-(propan-2-yl)-1H-indol-3-yl]methyl}-1-oxa-2,8-diazaspiro[4.5]dec-2-en-3-yl)benzoic acid, TFA salt

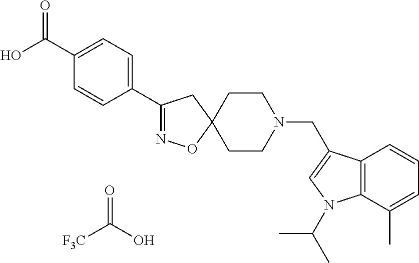 [SMILES: Cc1cccc2c(CN3CCC4(CC3)CC(c3ccc(C(=O)O)cc3)=NO4)cn(C(C)C)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (30 mg, 0.099 mmol) (INTERMEDIATE 2) and 7-methyl-1-(propan-2-yl)-1H-indole-3-carboxaldehyde (20 mg, 0.099 mmol) (INTERMEDIATE 22) afforded the title compound (25 mg) as a solid TFA salt.

¹H NMR (CD₃OD, 500 MHz): δ 8.07 (d, J=8.4 Hz, 2H), 7.77 (d, J=8.4 Hz, 2H), 7.709 (s, 1H), 7.59 (d, J=7.7 Hz, 1H), 7.087 (br t, J=7.7 Hz, 1H), 7.009 (d, J=7.1 Hz, 1H), 5.279 (hep, J=6.6 Hz, 1H), 4.569 (s, 2H), 3.59 (m, 2H), 3.40 (m, 2H), 3.338 (s, 2H), 2.766 (s, 3H), 2.21 (m, 2H), 2.11 (m, 2H), 1.569 (d, J=6.6, 6H).

[M+H⁺]: m/z 446.

### Example 45

### 4-(8-{[7-Methyl-1-(cyclopropyl)-1H-indol-3-yl]methyl}-1-oxa-2,8-diazaspiro[4.5]dec-2-en-3-yl)benzoic acid, TFA salt

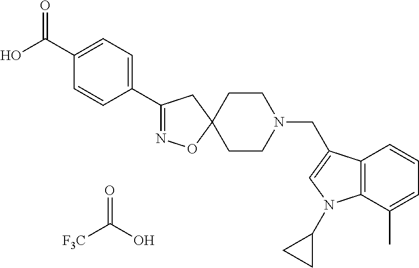 [SMILES: Cc1cccc2c(CN3CCC4(CC3)CC(c3ccc(C(=O)O)cc3)=NO4)cn(C3CC3)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (45 mg, 0.15 mmol) (INTERMEDIATE 2) and 7-methyl-1-(cyclopropyl)-1H-indole-3-carboxaldehyde (30 mg, 0.15 mmol) (INTERMEDIATE 23) afforded the title compound (40 mg) as a solid TFA salt.

¹H NMR (CD₃OD, 500 MHz): δ 8.07 (d, J=8.3 Hz, 2H), 7.77 (d, J=8.5 Hz, 2H), 7.52 (d, J=7.9 Hz, 1H), 7.50 (s, 1H), 7.08 (dd, J=7.4 and 7.7 Hz, 1H), 7.02 (d, J=7.0 Hz, 1H), 4.52 (s, 2H), 3.81 (m, 1H), 3.58 and 3.39 (2 m, 4H), 3.33 (s, 2H), 2.92 (s, 3H), 2.1-2.3 (2 m, 4H), 1.08 (m, 4H).

[M+H⁺]: m/z 444.

### Example 46

### 4-(8-{[1-(propan-2-yl)-4,5,6,7-(tetrafluoro)-1H-indol-3-yl]methyl}-1-oxa-2,8-diazaspiro[4.5]dec-2-en-3-yl)benzoic acid, TFA salt

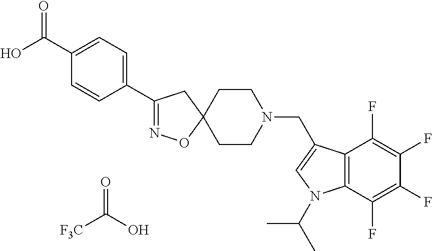 [SMILES: CC(C)n1cc(CN2CCC3(CC2)CC(c2ccc(C(=O)O)cc2)=NO3)c2c(F)c(F)c(F)c(F)c21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (34 mg, 0.12 mmol) (INTERMEDIATE 2) and 1-(propan-2-yl)-4,5,6,7-(tetrafluoro)-1H-indole-3-carboxaldehyde (30 mg, 0.12 mmol) (INTERMEDIATE 24) afforded the title compound (35 mg) as a solid TFA salt.

[M+H⁺]: m/z 504.

### Example 47

### 4-(8-{[4-(Cyclopropyl)-1-(propan-2-yl)-1H-indol-3-yl]methyl}-1-oxa-2,8-diazaspiro[4.5]dec-2-en-3-yl)benzoic acid, TFA salt

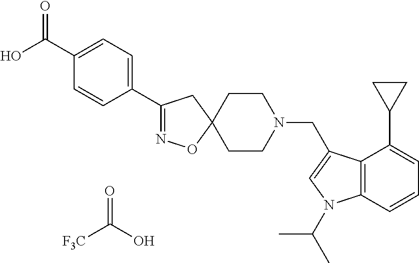 [SMILES: CC(C)n1cc(CN2CCC3(CC2)CC(c2ccc(C(=O)O)cc2)=NO3)c2c(C3CC3)cccc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (20 mg, 0.066 mmol) (INTERMEDIATE 2) and 4-(cyclopropyl)-1-(propan-2-yl)-1H-indole-3-carboxaldehyde (15 mg, 0.066 mmol) (INTERMEDIATE 25) afforded the title compound (20 mg) as a solid TFA salt.

¹H NMR (CD₃OD, 500 MHz): δ 8.07 (d, J=8.3 Hz, 2H), 7.78 (d, J=8.4 Hz, 2H), 7.72 (s, 1H), 7.40 (d, J=8.2 Hz, 1H), 7.17 (dd, J=7.6 and 8.1 Hz, 1H), 6.95 (d, J=7.3 Hz, 1H), 4.91 (s, 2H), 4.81 (hep, J=6.6 Hz, 1H), 3.65 and 3.45 (2 m, 4H), 3.35 (s, 2H), 2.37 (m, 1H), 2.1-2.3 (2 m, 4H), 1.56 (d, J=6.6 Hz, 6H), 1.09 (m, 2H), 0.87 (m, 2H).

[M+H⁺]: m/z 472.

### Example 48

### 4-(8-{[7-(Cyclopropyl)-1-(propan-2-yl)-1H-indol-3-yl]methyl}-1-oxa-2,8-diazaspiro[4.5]dec-2-en-3-yl)benzoic acid, TFA salt

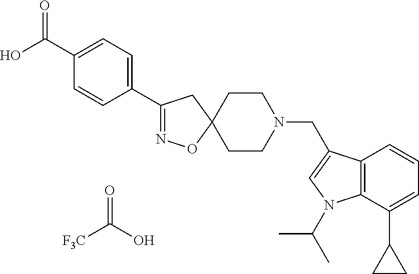 [SMILES: CC(C)n1cc(CN2CCC3(CC2)CC(c2ccc(C(=O)O)cc2)=NO3)c2cccc(C3CC3)c21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (26 mg, 0.088 mmol) (INTERMEDIATE 2) and 7-(cyclopropyl)-1-(propan-2-yl)-1H-indole-3-carboxaldehyde (20 mg, 0.088 mmol) (INTERMEDIATE 26) afforded the title compound (23 mg) as a solid TFA salt.

[M+H⁺]: m/z 472.

### Example 49

### 4-(8-{[1,5-(Dicyclopropyl)-1H-indol-3-yl]methyl}-1-oxa-2,8-diazaspiro[4.5]dec-2-en-3-yl)benzoic acid, TFA salt

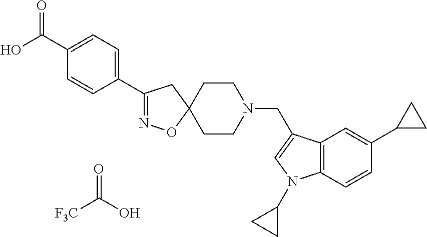 [SMILES: O=C(O)c1ccc(C2=NOC3(CCN(Cc4cn(C5CC5)c5ccc(C6CC6)cc45)CC3)C2)cc1 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (26.3 mg, 0.089 mmol) (INTERMEDIATE 2) and 1,5-(dicyclopropyl)-1H-indole-3-carboxaldehyde (20 mg, 0.089 mmol) (INTERMEDIATE 27) afforded the title compound (25 mg) as a solid TFA salt.

[M+H⁺]: m/z 470.

### Example 50

### 4-(8-{[1,7-(Dicyclopropyl)-1H-indol-3-yl]methyl}-1-oxa-2,8-diazaspiro[4.5]dec-2-en-3-yl)benzoic acid, TFA salt

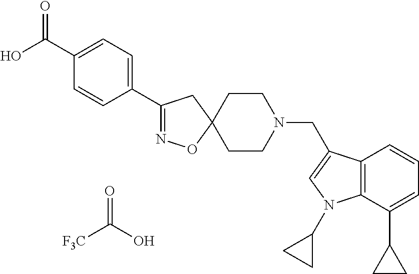 [SMILES: O=C(O)c1ccc(C2=NOC3(CCN(Cc4cn(C5CC5)c5c(C6CC6)cccc45)CC3)C2)cc1 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (26.3 mg, 0.089 mmol) (INTERMEDIATE 2) and 1,7-(dicyclopropyl)-1H-indole-3-carboxaldehyde (20 mg, 0.089 mmol) (INTERMEDIATE 28) afforded the title compound (24 mg) as a solid TFA salt.

[M+H⁺]: m/z 470.

### Example 51

### 4-(8-{[5-Fluoro-1-(propan-2-yl)-1H-indol-3-yl]methyl}-1-oxa-2,8-diazaspiro[4.5]dec-2-en-3-yl)benzoic acid, TFA salt

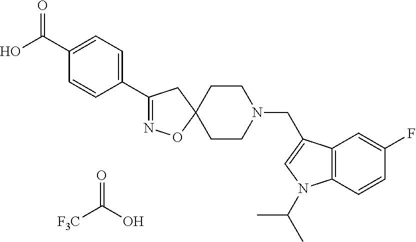 [SMILES: CC(C)n1cc(CN2CCC3(CC2)CC(c2ccc(C(=O)O)cc2)=NO3)c2cc(F)ccc21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (29 mg, 0.097 mmol) (INTERMEDIATE 2) and 5-fluoro-1-(propan-2-yl)-1H-indole-3-carboxaldehyde (20 mg, 0.097 mmol) (INTERMEDIATE 35) afforded the title compound (25 mg) as a solid TFA salt.

[M+H⁺]: m/z 450.

### Example 52

### 4-(8-{[7-Chloro-1-(propan-2-yl)-1H-indol-3-yl]methyl}-1-oxa-2,8-diazaspiro[4.5]dec-2-en-3-yl)benzoic acid, TFA salt

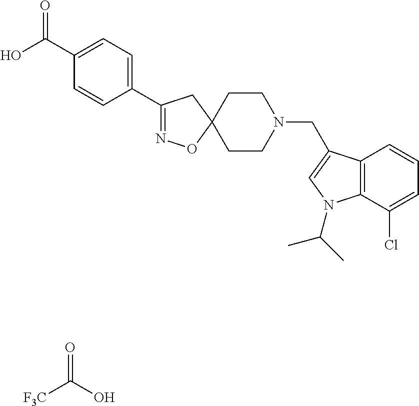 [SMILES: CC(C)n1cc(CN2CCC3(CC2)CC(c2ccc(C(=O)O)cc2)=NO3)c2cccc(Cl)c21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (27 mg, 0.090 mmol) (INTERMEDIATE 2) and 7-chloro-1-(propan-2-yl)-1H-indole-3-carboxaldehyde (20 mg, 0.090 mmol) (INTERMEDIATE 36) afforded the title compound (25 mg) as a solid TFA salt.

[M+H⁺]: m/z 466/468.

### Example 53

### 4-(8-{[5-Cyclopropyl-7-methyl-1-(propan-2-yl)-1-indol-3-yl]methyl}-1-oxa-2,8-diazaspiro[4.5]dec-2-en-3-yl)benzoic acid, TFA salt

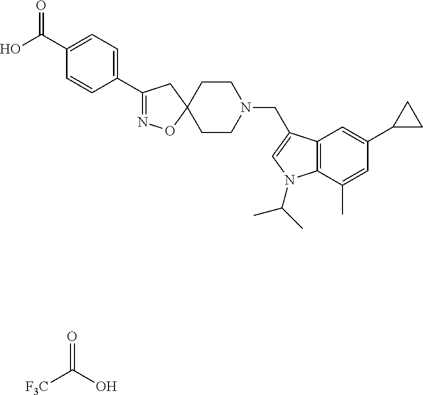 [SMILES: Cc1cc(C2CC2)cc2c(CN3CCC4(CC3)CC(c3ccc(C(=O)O)cc3)=NO4)cn(C(C)C)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.083 mmol) (INTERMEDIATE 2) and 5-cyclopropyl-7-methyl-1-(propan-2-yl)-1H-indole-3-carboxaldehyde (20 mg, 0.083 mmol) (INTERMEDIATE 37) afforded the title compound (24 mg) as a solid TFA salt.

[M+H⁺]: m/z 486.

### Example 54

### 4-(8-{[5-Fluoro-7-methyl-1-(propan-2-yl)-1H-indol-3-yl]methyl}-1-oxa-2,8-diazaspiro[4.5]dec-2-en-3-yl)benzoic acid, TFA salt

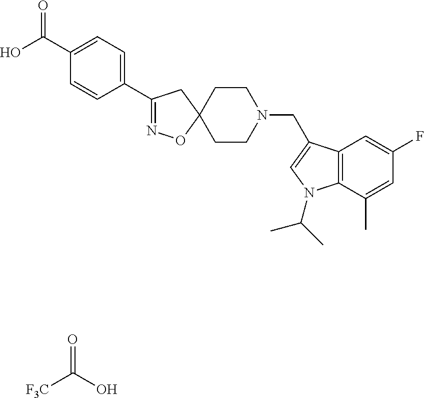 [SMILES: Cc1cc(F)cc2c(CN3CCC4(CC3)CC(c3ccc(C(=O)O)cc3)=NO4)cn(C(C)C)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (27 mg, 0.091 mmol) (INTERMEDIATE 2) and 5-fluoro-7-methyl-1-(propan-2-yl)-1H-indole-3-carboxaldehyde (20 mg, 0.091 mmol) (INTERMEDIATE 38) afforded the title compound (24 mg) as a solid TFA salt.

¹H NMR (CD₃Cl, 500 MHz): δ 8.13 (d, J=8.4 Hz, 2H), 7.71 (d, J=8.4 Hz, 2H), 7.64 (s, 1H), 7.04 (dd, J=2.5 and 8.4 Hz, 1H), 6.79 (dd, J=2.0 and 10 Hz, 1H), 5.11 (hep, J=6.6 Hz, 1H), 4.38 (s, 2H), 3.61 (m, 2H), 3.23 (m, 4H), 2.74 (s, 3H), 2.46 (m, 2H), 2.15 (m, 2H), 1.54 (d, J=6.6, 6H).

[M+H⁺]: m/z 464.

### Example 55

### 4-(8-{[5-Fluoro-7-methyl-1-(cyclopropyl)-1H-indol-3-yl]methyl})-1-oxa-2,8-diazaspiro[4.5]dec-2-en-3-yl)benzoic acid, TFA salt

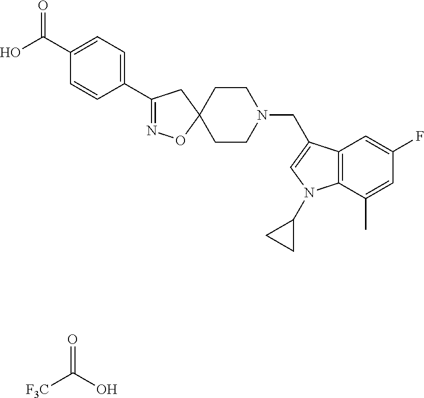 [SMILES: Cc1cc(F)cc2c(CN3CCC4(CC3)CC(c3ccc(C(=O)O)cc3)=NO4)cn(C3CC3)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (27 mg, 0.092 mmol) (INTERMEDIATE 2) and 5-fluoro-7-methyl-1-(cyclopropyl)-1H-indole-3-carboxaldehyde (20 mg, 0.092 mmol) (INTERMEDIATE 39) afforded the title compound (25 mg) as a solid TFA salt.

[M+H⁺]: m/z 462.

### Example 56

### 4-(8-{[1,5-Dicyclopropyl-7-methyl-1-1H-indol-3-yl]methyl}-1-oxa-2,8-diazaspiro[4.5]dec-2-en-3-yl)benzoic acid, TFA salt

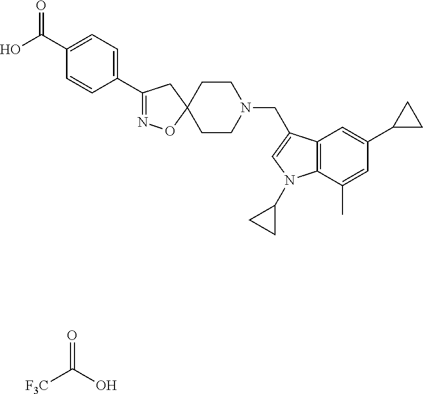 [SMILES: Cc1cc(C2CC2)cc2c(CN3CCC4(CC3)CC(c3ccc(C(=O)O)cc3)=NO4)cn(C3CC3)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.084 mmol) (INTERMEDIATE 2) and 1,5-dicyclopropyl-7-methyl-1H-indole-3-carboxaldehyde (20 mg, 0.084 mmol) (INTERMEDIATE 40) afforded the title compound (10 mg) as a solid TFA salt.

[M+H⁺]: m/z 484.

### Example 57

### 4-(8-{[7-Chloro-1-(cyclopropyl)-1-1H-indol-3-yl]methyl}-1-oxa-2,8-diazaspiro[4.5]dec-2-en-3-yl)benzoic acid, TFA salt

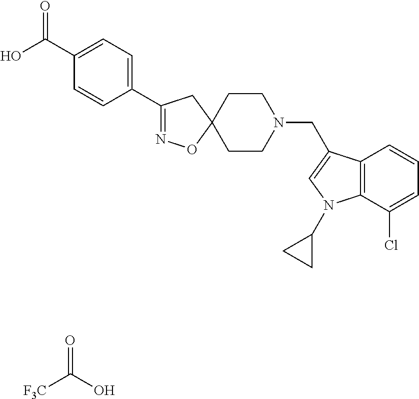 [SMILES: O=C(O)c1ccc(C2=NOC3(CCN(Cc4cn(C5CC5)c5c(Cl)cccc45)CC3)C2)cc1 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (27 mg, 0.091 mmol) (INTERMEDIATE 2) and 7-chloro-1-cyclopropyl-1H-indole-3-carboxaldehyde (20 mg, 0.091 mmol) (INTERMEDIATE 41) afforded the title compound (20 mg) as a solid TFA salt.

[M+H⁺]: m/z 464/466.

### Example 58

### 4-(8-{[7-Chloro-4-methyl-1-(propan-2-yl)-1H-indol-3-yl]methyl}-1-oxa-2,8-diazaspiro[4.5]dec-2-en-3-yl)benzoic acid, TFA salt

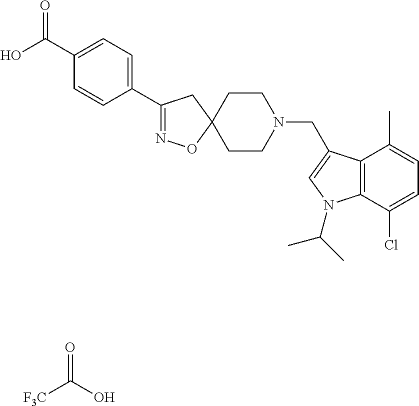 [SMILES: Cc1ccc(Cl)c2c1c(CN1CCC3(CC1)CC(c1ccc(C(=O)O)cc1)=NO3)cn2C(C)C ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.085 mmol) (INTERMEDIATE 2) and 7-chloro-4-methyl-1-(propan-2-yl)-1H-indole-3-carboxaldehyde (20 mg, 0.085 mmol) (INTERMEDIATE 42) afforded the title compound (25 mg) as a solid TFA salt.

[M+H⁺]: m/z 480/482.

### Example 59

### 4-(8-{[5-Chloro-7-methyl-1-(propan-2-yl)-1H-indol-3-yl]methyl}-1-oxa-2,8-diazaspiro[4.5]dec-2-en-3-yl)benzoic acid, TFA salt

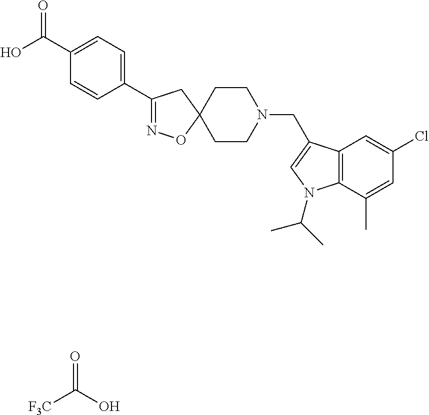 [SMILES: Cc1cc(Cl)cc2c(CN3CCC4(CC3)CC(c3ccc(C(=O)O)cc3)=NO4)cn(C(C)C)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.085 mmol) (INTERMEDIATE 2) and 5-chloro-7-methyl-1-(propan-2-yl)-1H-indole-3-carboxaldehyde (20 mg, 0.085 mmol) (INTERMEDIATE 43) afforded the title compound (20 mg) as a solid TFA salt.

[M+H⁺]: m/z 480/482.

### Example 60

### 4-(8-{[4,7-Dichloro-1-(propan-2-yl)-1H-indol-3-yl]methyl}-1-oxa-2,8-diazaspiro[4.5]dec-2-en-3-yl)benzoic acid, TFA salt

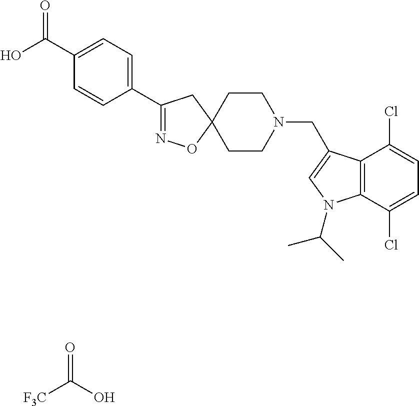 [SMILES: CC(C)n1cc(CN2CCC3(CC2)CC(c2ccc(C(=O)O)cc2)=NO3)c2c(Cl)ccc(Cl)c21 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (23 mg, 0.078 mmol) (INTERMEDIATE 2) and 4,7-dichloro-1-(propan-2-yl)-1H-indole-3-carboxaldehyde (21 mg, 0.078 mmol) (INTERMEDIATE 44) afforded the title compound (20 mg) as a solid TFA salt.

¹H NMR (CD₃OD, 500 MHz): b 8.07 (d, J=8.4 Hz, 2H), 7.959 (s, 1H), 7.78 (d, J=8.3 Hz, 2 H), 7.25 and 7.18 (ABq, J=8.3 Hz, 2H), 5.796 (hep, J=6.6 Hz, 1H), 4.832 (s, 2H), 3.66 and 3.48 (2 m, 4H), 3.353 (s, 2H), 2.1-2.3 (m, 4H), 1.59 (d, J=6.6 Hz, 6H).

[M+H⁺]: m/z 500/502.

### Example 61

### 4-(8-{[5-Chloro-1-(cyclopropyl)-7-methyl-1H-indol-3-yl]methyl}-1-oxa-2,8-diazaspiro[4.5]dec-2-en-3-yl)benzoic acid, TFA salt

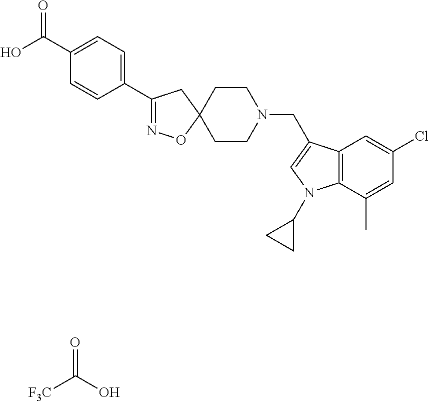 [SMILES: Cc1cc(Cl)cc2c(CN3CCC4(CC3)CC(c3ccc(C(=O)O)cc3)=NO4)cn(C3CC3)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.086 mmol) (INTERMEDIATE 2) and 5-chloro-1-(cyclopropyl)-7-methyl-1H-indole-3-carboxaldehyde (20 mg, 0.086 mmol) (INTERMEDIATE 45) afforded the title compound (18 mg) as a solid TFA salt.

[M+H⁺]: m/z 478/480.

### Example 62

### 4-(8-{[7-Chloro-1-(cyclopropyl)-4-methyl-1H-indol-3-yl]methyl}-1-oxa-2,8-diazaspiro[4.5]dec-2-en-3-yl)benzoic acid, TFA salt

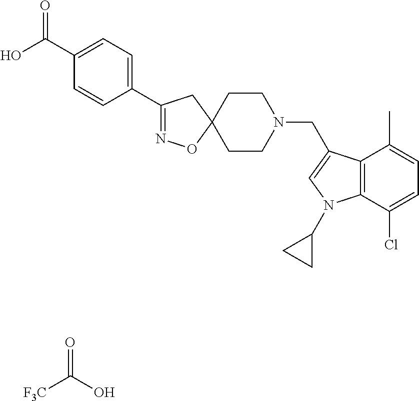 [SMILES: Cc1ccc(Cl)c2c1c(CN1CCC3(CC1)CC(c1ccc(C(=O)O)cc1)=NO3)cn2C1CC1 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.086 mmol) (INTERMEDIATE 2) and 7-chloro-1-(cyclopropyl)-4-methyl-1H-indole-3-carboxaldehyde (20 mg, 0.086 mmol) (INTERMEDIATE 46) afforded the title compound (20 mg) as a solid TFA salt.

[M+H⁺]: m/z 478/480.

### Example 63

### 4-(8-{[1-(Cyclopropyl)-4,7-dichloro-1H-indol-3-yl]methyl}-1-oxa-2,8-diazaspiro[4.5]dec-2-en-3-yl)benzoic acid, TFA salt

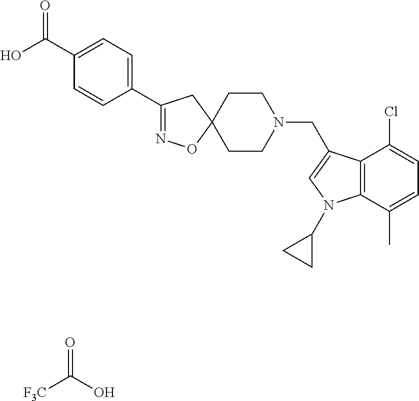 [SMILES: Cc1ccc(Cl)c2c(CN3CCC4(CC3)CC(c3ccc(C(=O)O)cc3)=NO4)cn(C3CC3)c12 ; O=C(O)C(F)(F)F]

Using essentially the same procedure as for EXAMPLE 39, 3-[4-(carboxy)phenyl]-1-oxa-2,8-diazaspiro[4.5]dec-2-ene hydrochloride (25 mg, 0.086 mmol) (INTERMEDIATE 2) and 1-(cyclopropyl)-4,7-dichloro-1H-indole-3-carboxaldehyde (20 mg, 0.086 mmol) (INTERMEDIATE 47) afforded the title compound (20 mg) as a solid TFA salt.

[M+H⁺]: m/z 498/500.

### Example 64

### 4-(8-{[1-(Propan-2-yl)-5,7-dichloro-1H-indol-3-yl]methyl}-1-oxa-2,8-diazaspiro[4.5]dec-2-en-3-yl)benzoic acid, TFA salt

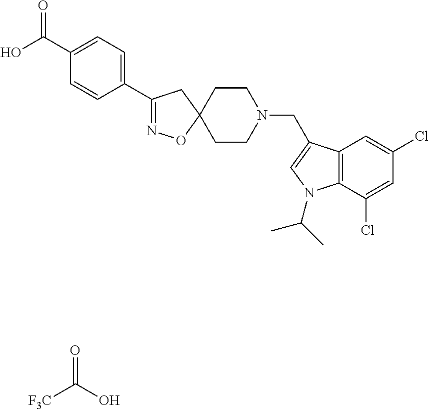 [SMILES: CC(C)n1cc(CN2CCC3(CC2)CC(c2ccc(C(=O)O)cc2)=NO3)c2cc(Cl)cc(Cl)c21 ; O=C(O)C(F)(F)F]

To a solution of 3-[4-(carboxy)phenyl]-1-oxa-2,8-diazaspiro[4.5]dec-2-ene hydrochloride (23 mg, 0.078 mmol) (INTERMEDIATE 2) and 1-(propan-2-yl)-5,7-dichloro-1H-indole-3-carboxaldehyde (20 mg, 0.078 mmol) (INTERMEDIATE 48) in DMF (3 mL) in a 20 mL scintillation vial was stirred for 30 minutes and then acetic acid (28 uL, 0.464 mmol) and MP-cyanoborohydride resin (160 mg, 0.372 mmol, 2.49 mmol/g) were added. The mixture was shaken at 55° C. for 18 hours. The reaction was quenched with 3 drops of water and was then filtered to remove the resin. The product was isolated directly from the filtrate by reverse phase (C-18) HPLC chromatography (5-95% acetonitrile/water gradient) to afford the title compound (15 mg) as a solid TFA salt after evaporation.

[M+H⁺]: m/z 500/502.

### Example 65

### 4-(8-{[4-(4-Fluorophenyl)-1H-indol-3-yl]methyl)}-1-oxa-2,8-diazaspiro[4.5]dec-2-en-3-yl)benzoic acid, TFA salt

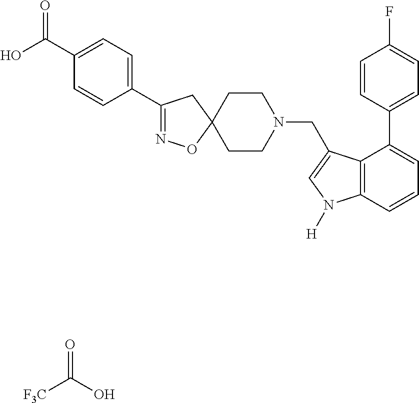 [SMILES: O=C(O)c1ccc(C2=NOC3(CCN(Cc4c[nH]c5cccc(-c6ccc(F)cc6)c45)CC3)C2)cc1 ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (25 mg, 0.084 mmol) (INTERMEDIATE 2) and 4-(4-fluorophenyl)-1H-indole-3-carboxaldehyde (20 mg, 0.084 mmol) (INTERMEDIATE 49) afforded the title compound (15 mg) as a solid TFA salt.

[M+H⁺]: m/z 484.

### Example 66

### 4-(8-{[1-(Propan-2-yl)-7-chloro-1H-indol-5-yl]methyl}-1-oxa-2,8-diazaspiro[4.5]dec-2-en-3-yl)benzoic acid, TFA salt

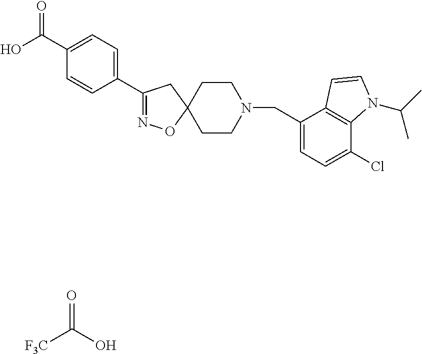 [SMILES: CC(C)n1ccc2c(CN3CCC4(CC3)CC(c3ccc(C(=O)O)cc3)=NO4)ccc(Cl)c21 ; O=C(O)C(F)(F)F]

Using essentially the same procedure as for EXAMPLE 34, 3-[4-(carboxy)phenyl]-1-oxa-2,8-diazaspiro[4.5]dec-2-ene hydrochloride (27 mg, 0.090 mmol) (INTERMEDIATE 2) and 1-(propan-2-yl)-7-chloro-1H-indole-5-carboxaldehyde (20 mg, 0.090 mmol) (INTERMEDIATE 50) afforded the title compound (20 mg) as a solid TFA salt.

¹H NMR (CD₃OD, 500 MHz): δ 8.07 (d, J=8.1 Hz, 2H), 7.78 (d, J=8.2 Hz, 2H), 7.69 (d, J=3.5 Hz, 1H), 7.28 and 7.22 (ABq, J=7.9 Hz, 2H), 6.89 (d, J=3.4 Hz, 1H), 5.79 (hep, J=6.6 Hz, 1H), 4.65 (s, 2H), 3.49 (m, 4H), 3.33 (s, 2H), 2.19 (m, 4H), 1.56 (d, J=6.7 Hz, 6H).

[M+H⁺]: m/z 466/468.

### Example 67

### 4-(8-{[2,3-Dimethyl-1-(propan-2-yl)-1H-indol-6-yl]methyl}-1-oxa-2,8-diazaspiro[4.5]dec-2-en-3-yl)benzoic acid, TFA salt

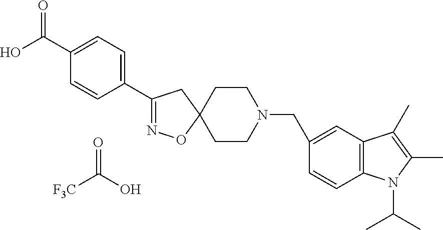

Using essentially the same procedure as for EXAMPLE 34, 3-[4-(carboxy)phenyl]-1-oxa-2,8-diazaspiro[4.5]dec-2-ene hydrochloride (28 mg, 0.093 mmol) (INTERMEDIATE 2) and 2,3-dimethyl 1-(propan-2-yl)-1H-indole-5-carboxaldehyde (20 mg, 0.093 mmol) (INTERMEDIATE 51) afforded the title compound (15 mg) as a solid TFA salt.

¹H NMR (CD₃OD, 500 MHz): δ 8.07 (d, J=8.4 Hz, 2H), 7.77 (d, J=8.4 Hz, 2H), 7.61 (s, 1H), 7.59 (d, J=7.5 Hz, 1H), 7.18 (d, J=7.5 Hz, 1H), 4.79 (hep, J=6.6 Hz, 1H), 4.45 (s, 2H), 3.2-3.6 (m, 6H), 2.41 (s, 3H), 2.29 (s, 3H), 2.1-2.3 (m, 4H), 1.58 (d, J=6.6, 6H).

[M+H⁺]: m/z 460.

### Example 68

### 8-{[4-Methyl-2-(pyridin-3-yl)-5-(trifluoromethyl)phenyl]methyl}-1-oxa-2,8-diazaspiro[4.5]dec-2-en-3-yl)benzoic acid

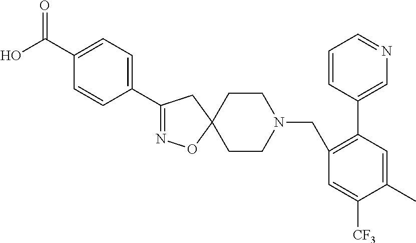 [SMILES: Cc1cc(-c2cccnc2)c(CN2CCC3(CC2)CC(c2ccc(C(=O)O)cc2)=NO3)cc1C(F)(F)F]

### Step A: Synthesis of methyl 8-{[2-bromo-4-methyl-5-(trifluoromethyl)phenyl]methyl}-1-oxa-2,8-diazaspiro[4.5]dec-2-en-3-yl)benzoate

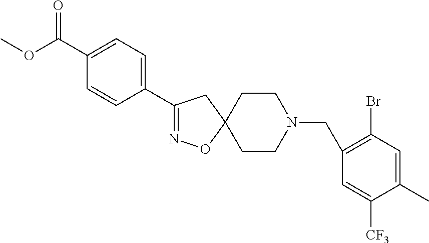

To a solution of 2-bromo-4-methyl-5-(trifluoromethyl)benzaldehyde (86 mg, 0.32 mmol) (INTERMEDIATE 52) in DMF (2 mL) at room temperature were added methyl 1-oxa-2,8-diazaspiro[4.5]dec-2-en-3-yl)benzoate (100 mg, 0.32 mmol) in DMF (3 mL) and DIPEA (0.056 mL, 0.32 mmol). The reaction was stirred at room temperature for 5 minutes and then acetic acid (0.018 mL, 0.32 mmol) and sodium triacetoxyborohydride (68 mg, 0.32 mmol) were added and the mixture was stirred for 20 hours. The reaction was quenched with water (10 mL) and the mixture was extracted twice with ethyl acetate. The organic layers were washed with brine, dried over sodium sulfate and concentrated in vacuo. The residue was purified on a silica gel column (12 g) eluting with 10-30% ethyl acetate/hexanes to give the title compound (45 mg) as a light yellow solid.

### Step B: Synthesis of 8-{[4-methyl-2-(pyridin-3-yl)-5-(trifluoromethyl)phenyl]methyl}-1-oxa-2,8-diazaspiro[4.5]dec-2-en-3-yl)benzoic acid

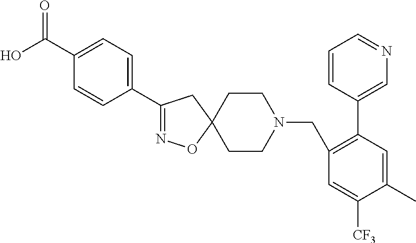 [SMILES: Cc1cc(-c2cccnc2)c(CN2CCC3(CC2)CC(c2ccc(C(=O)O)cc2)=NO3)cc1C(F)(F)F]

To a solution of methyl 8-{[2-bromo-4-methyl-5-(trifluoromethyl)phenyl]methyl}-1-oxa-2,8-diazaspiro[4.5]dec-2-en-3-yl)benzoate (22.5 mg, 0.043 mmol) in dioxane (1.5 mL) and water (0.5 mL) in a 5 mL microwave reaction vial was added pyridin-3-ylboronic acid (5.3 mg, 0.043 mmol), dichloro 1,1′-bis(diphenylphosphino)ferrocene palladium (II) (6.9 mg, 0.00857 mmol) and lithium hydroxide monohydrate (3.59 mg, 0043 mmol). The mixture was placed under nitrogen and heated in a microwave at 120° C. for 15 minutes. The reaction was diluted with acetonitrile (2 mL) and water (1 mL), filtered, and directly purified by reverse phase chromatography (eluting with an acetonitrile/water with 0.1% ammonium hydroxide gradient). The combined product fractions were combined and freeze dried to afford the title compound (1.8 mg) as a white solid.

[M+H⁺]: m/z 510.

### Example 69

### 8-{[2-(Pyridin-3-yl)-5-(trifluoromethyl)phenyl]methyl}-1-oxa-2,8-diazaspiro[4.5]dec-2-en-3-yl)benzoic acid

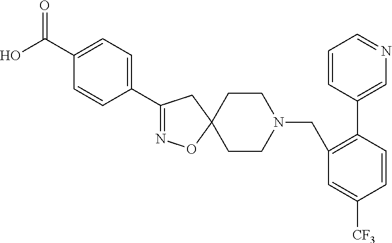 [SMILES: O=C(O)c1ccc(C2=NOC3(CCN(Cc4cc(C(F)(F)F)ccc4-c4cccnc4)CC3)C2)cc1]

### Step A: Synthesis of methyl 8-{[2-bromo-5-(trifluoromethyl)phenyl]methyl}-1-oxa-2,8-diazaspiro[4.5]dec-2-en-3-yl)benzoate

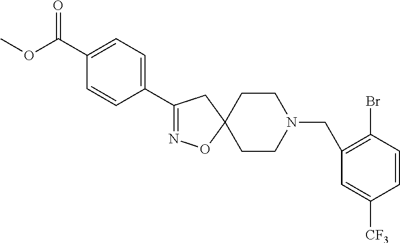 [SMILES: COC(=O)c1ccc(C2=NOC3(CCN(Cc4cc(C(F)(F)F)ccc4Br)CC3)C2)cc1]

Using essentially the same procedure as for EXAMPLE 68, Step A, but using 2-bromo-5-(trifluoromethyl)benzaldehyde (86 mg, 0.32 mmol) and methyl 1-oxa-2,8-diazaspiro[4.5]dec-2-en-3-yl)benzoate (100 mg, 0.32 mmol) pyridin-3-ylboronic acid (5.3 mg, 0.043 mmol), the title compound (45 mg) was prepared.

### Step B: Synthesis of 8-{[2-(pyridine-3-yl)-5-(trifluoromethyl)phenyl]methyl}-1-oxa-2,8-diazaspiro[4.5]dec-2-en-3-yl)benzoic acid

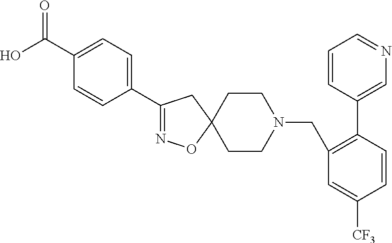 [SMILES: O=C(O)c1ccc(C2=NOC3(CCN(Cc4cc(C(F)(F)F)ccc4-c4cccnc4)CC3)C2)cc1]

Using essentially the same procedure as for EXAMPLE 68, Step B, but using methyl 8-{[2-bromo-5-(trifluoromethyl)phenyl]methyl}-1-oxa-2,8-diazaspiro[4.5]dec-2-en-3-yl)benzoate (22.5 mg, 0.043 mmol) and pyridin-3-ylboronic acid (5.3 mg, 0.043 mmol), the title compound (18 mg) was prepared as a white solid.

[M+H⁺]: m/z 496.

### Example 70

### 4-[8-(3-Bromo-4-chlorobenzyl)-1-oxa-2,8-diazaspiro[4.5]dec-2-en-3-yl]benzoic acid

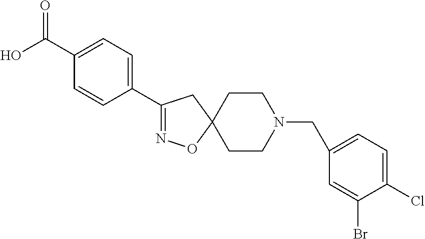 [SMILES: O=C(O)c1ccc(C2=NOC3(CCN(Cc4ccc(Cl)c(Br)c4)CC3)C2)cc1]

To INTERMEDIATE 2 (15 mg, 0.051 mmol), MP-cyanoborohydride (65.6 mg, 0.152 mmol), 3-bromo-4-chlorobenzaldehyde (12.2 mg, 0.056 mmol) and AcOH (4.34 μl, 0.076 mmol) was added DMF (1.5 mL). The mixture was shaken overnight at room temperature. The reaction mixture was filtered, the resin was washed with DMF and the solvent was evaporated. The residue was purified by reverse phase (C-18) HPLC eluting with acetonitrile/water+0.1% NH₃. The solvent was evaporated to provide the title compound as a white solid (6 mg).

¹H NMR (DMSO-d₆, 600 MHz): δ 7.94 (d, J=8.4 Hz, 2H), 7.71 (d, J=8.4 Hz, 2H), 7.67 (d, J=1.2 Hz, 1H), 7.55 (d, J=7.8 Hz, 1H), 7.33 (dd, J=1.2 Hz, 7.8 Hz, 1H), 3.48 (s, 2H), 3.18 (s, 2H), 2.47 (d, J=2.4 Hz, 2H), 2.36-2.34 (m, 2H), 1.76-1.73 (m, 4H).

[M+H⁺]: m/z 463.

### Example 71

### 4-[8-(4-Chloro-3-ethylbenzyl)-1-oxa-2,8-diazaspiro[4.5]dec-2-en-3-yl]benzoic acid

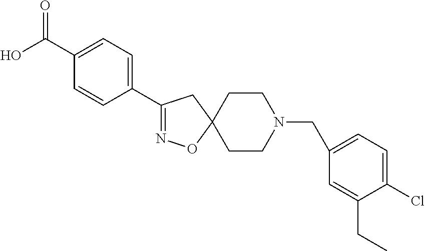 [SMILES: CCc1cc(CN2CCC3(CC2)CC(c2ccc(C(=O)O)cc2)=NO3)ccc1Cl]

The title compound was prepared from INTERMEDIATE 2 (15 mg, 0.051 mmol) and 4-chloro-3-ethylbenzaldehyde (9.38 mg, 0.056 mmol), following essentially the same procedure described in EXAMPLE 70. The title compound was obtained as a white solid (6 mg).

¹H NMR (DMSO-d₆, 600 MHz): δ 7.94 (d, J=8.4 Hz, 2H), 7.71 (d, J=8.4 Hz, 2H), 7.31 (d, J=8.4 Hz, 1H), 7.24 (s, 1H), 7.14 (d, J=8.4 Hz, 1H), 3.18 (s, 2H), 2.65 (q, J=8.4 Hz, 2H), 2.47-2.38 (m, 6H), 1.78-1.70 (m, 4H), 1.13 (t, J=7.2 Hz, 3H).

[M+H⁺]: m/z 413.

### Example 72

### 4-{8-[4-Chloro-3-(trifluoromethoxy)benzyl]-1-oxa-2,8-diazaspiro[4.5]dec-2-en-3-yl}benzoic acid

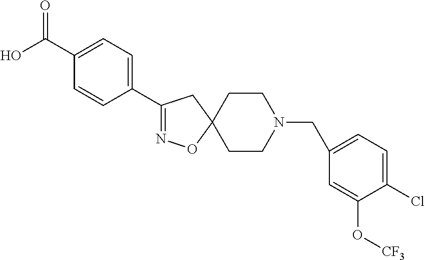 [SMILES: O=C(O)c1ccc(C2=NOC3(CCN(Cc4ccc(Cl)c(OC(F)(F)F)c4)CC3)C2)cc1]

The title compound was prepared from INTERMEDIATE 2 (15 mg, 0.051 mmol) and 4-chloro-3-trifluoromethoxybenzaldehyde (12.49 mg, 0.056 mmol) following essentially the same procedure described in EXAMPLE 70. The title compound was obtained as a white solid (6 mg).

¹H NMR (DMSO-d₆, 600 MHz): δ 7.94 (d, J=8.4 Hz, 2H), 7.71 (d, J=8.4 Hz, 2H), 7.61 (d, J=8.4 Hz, 1H), 7.46 (s, 1H), 7.37 (d, J=8.4 Hz, 1H), 3.54 (s, 2H), 3.18 (s, 2H), 2.46-2.45 (m, 2H), 2.39-2.34 (m, 2H), 1.79-1.72 (m, 4H).

[M+H⁺]: m/z 469.

### Example 73

### 4-{8-[4-Chloro-3-(trifluoromethyl)benzyl]-1-oxa-2,8-diazaspiro[4.5]dec-2-en-3-yl}benzoic acid

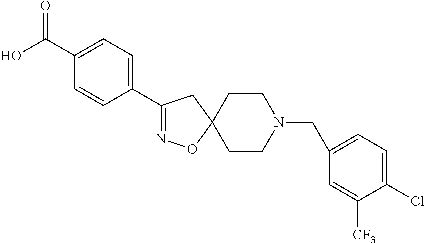 [SMILES: O=C(O)c1ccc(C2=NOC3(CCN(Cc4ccc(Cl)c(C(F)(F)F)c4)CC3)C2)cc1]

The title compound was prepared from INTERMEDIATE 2 (15 mg, 0.051 mmol) and 4-chloro-3-trifluoromethylbenzaldehyde (11.6 mg, 0.056 mmol) following essentially the same procedure described in EXAMPLE 70. The title compound was obtained as a white solid (6 mg,).

¹H NMR (DMSO-d₆, 600 MHz): δ 7.94 (d, J=8.4 Hz, 2H), 7.74 (s, 1H), 7.71 (d, J=8.4 Hz, 2H), 7.65 (d, J=8.4 Hz, 1H), 7.62-7.60 (m, 1H), 3.56 (s, 2H), 3.18 (s, 2H), 2.46-2.45 (m, 2H), 2.39-2.35 (m, 2H), 1.79-1.71 (m, 41H).

[M+H⁺]: m/z 453.

### Example 74

### 4-[8-(4-Chloro-3-cyclopropylbenzyl)-1-oxa-2,8-diazaspiro[4.5]dec-2-en-3-yl]benzoic acid, TFA salt

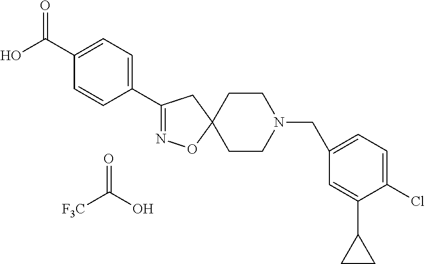 [SMILES: O=C(O)c1ccc(C2=NOC3(CCN(Cc4ccc(Cl)c(C5CC5)c4)CC3)C2)cc1 ; O=C(O)C(F)(F)F]

### Step A: Synthesis of methyl 4-[8-(3-bromo-4-chlorobenzyl)-1-oxa-2,8-diazaspiro[4.5]dec-2-en-3-yl]benzoate

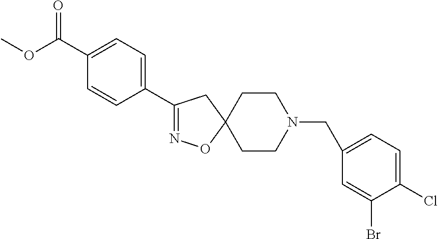 [SMILES: COC(=O)c1ccc(C2=NOC3(CCN(Cc4ccc(Cl)c(Br)c4)CC3)C2)cc1]

To INTERMEDIATE 1 (50 mg, 0.161 mmol), MP-cyanoborohydride (209 mg, 0.483 mmol), 3-bromo-4-chlorobenzaldehyde (39 mg, 0.177 mmol) and AcOH (13.82 μl, 0.241 mmol) was added 3 mL of DMF. The reaction mixture was filtered, the resin was washed with DMF and the solvent was evaporated. The residue was dissolved in saturated aqueous NaHCO₃ and extracted with DCM. The combined organic layers were evaporated to yield the title compound as a yellow oil (71 mg) that was used without any further purification.

[M+H⁺]: m/z 477.

### Step B: Synthesis of 4-[8-(4-chloro-3-cyclopropylbenzyl)-1-oxa-2,8-diazaspiro[4.5]dec-2-en-3-yl]benzoic, TFA salt

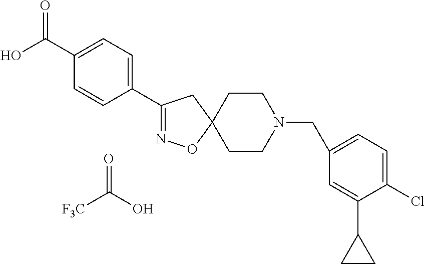 [SMILES: O=C(O)c1ccc(C2=NOC3(CCN(Cc4ccc(Cl)c(C5CC5)c4)CC3)C2)cc1 ; O=C(O)C(F)(F)F]

To methyl 4-[8-(3-bromo-4-chlorobenzyl)-1-oxa-2,8-diazaspiro[4.5]dec-2-en-3-yl]benzoate (60 mg, 0.126 mmol), K₂CO₃ (52.1 mg, 0.377 mmol), Pd(OAc)₂ (0.846 mg, 3.77 μmol), potassium cyclopropyltrifluoroborate (22.30 mg, 0.151 mmol) and 2-dicyclohexylphosphino-2′,4′,6′-trisisopropylbiphenyl (3.59 mg, 7.53 μmol) was added toluene (471 uL) and water (157 uL). The reaction mixture was heated to 100° C. overnight. The reaction mixture was diluted with water, extracted with DCM and the organic layer was evaporated in vacuo. The residue was then dissolved in water (300 uL) and THF (900 uL) and LiOH (7.52 mg, 0.314 mmol) was added. The reaction mixture was stirred at 70° C. for 3 hours. The mixture was evaporated in vacuo and the residue was purified by reverse phase (C-18) HPLC eluting with acetonitrile/water+0.1% TFA. The solvent was evaporated to provide the title compound as a white solid (22 mg).

¹H NMR (CD₃OD, 400 MHz): δ 8.03 (d, J=8.4 Hz, 2H), 7.74 (d, J=8.4 Hz, 2H), 7.46 (d, J=8.0 Hz, 1H), 7.26 (dd, J=2.0, 8.0 Hz, 1H), 7.16 (d, J=2.0 Hz, 1H), 4.30 (s, 2H), 3.28-3.26 (m, 6H), 2.23-2.14 (m, 5H), 1.07-1.02 (m, 2H), 0.75-0.71 (m, 2H).

[M+H⁺]: m/z 425.

### Example 75

### 4-{8-[3-Cyclopropyl-4-(trifluoromethoxy)benzyl]-1-oxa-2,8-diazaspiro[4.5]dec-2-en-3-yl}benzoic acid, TFA salt

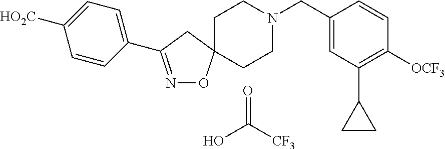

### Step A: Synthesis of methyl 4-{8-[3-bromo-4-(trifluoromethoxy)benzyl]-1-oxa-2,8-diazaspiro[4.5]dec-2-en-3-yl}benzoate

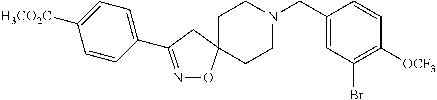 [SMILES: COC(=O)c1ccc(C2=NOC3(CCN(Cc4ccc(OC(F)(F)F)c(Br)c4)CC3)C2)cc1]

The title compound was prepared from INTERMEDIATE 1 (50 mg, 0.161 mmol) and 3-bromo-4-trifluoromethoxybenzaldehyde (47.6 mg, 0.177 mmol) following essentially the same procedure described in Step A of EXAMPLE 74. The title compound was obtained as a yellow oil (75 mg) that was used without any further purification.

[M+H⁺]: m/z 527.

### Step B: Synthesis of 4-{8-[3-cyclopropyl-4-(trifluoromethoxy)benzyl]-1-oxa-2,8-diazaspiro[4.5]dec-2-en-3-yl}benzoic acid, TFA salt

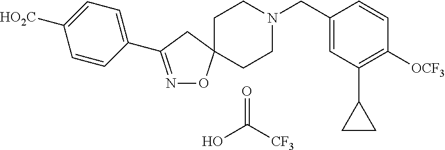

The title compound was prepared from methyl 4-{8-[3-bromo-4-(trifluoromethoxy)benzyl]-1-oxa-2,8-diazaspiro[4.5]dec-2-en-3-yl}benzoate (60 mg, 0.114 mmol) following essentially the same procedure described in Step B of EXAMPLE 74. The title compound was obtained as a white solid (14 mg):

¹H NMR (CD₃OD, 400 MHz): δ 8.03 (d, J=8.4 Hz, 2H), 7.74 (d, J=8.4 Hz, 2H), 7.38-7.32 (m, 2H), 7.17 (d, J=2.0 Hz, 1H), 4.30 (s, 2H), 3.28-3.25 (m, 6H), 2.17-2.05 (m, 5H), 1.07-1.03 (m, 2H), 0.78-0.76 (m, 2H).

[M+H⁺]: m/z 475.

### Example 76

### 4-[8-(3-Bromo-4,5-dichlorobenzyl)-1-oxa-2,8-diazaspiro[4.5]dec-2-en-3-yl]benzoic acid, formic acid salt

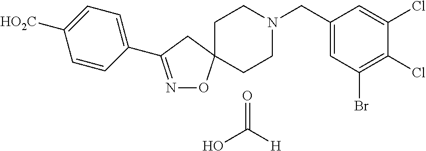

### Step A: Synthesis of methyl 4-[8-(3-bromo-4,5-dichlorobenzyl)-1-oxa-2,8-diazaspiro[4.5]dec-2-en-3-yl]benzoate

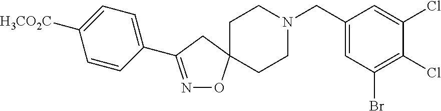 [SMILES: COC(=O)c1ccc(C2=NOC3(CCN(Cc4cc(Cl)c(Cl)c(Br)c4)CC3)C2)cc1]

To INTERMEDIATE 1 (50 mg, 0.161 mmol) and 1-bromo-5-(bromomethyl)-2,3-dichlorobenzene (56.4 mg, 0.177 mmol; from Patent US2007/070805) was added DMF (1.5 mL) and DIPEA (112 μL, 0.644 mmol). The mixture was heated to 55° C. for 2 hours. The mixture was evaporated and the residue was dissolved in saturated aqueous NaHCO₃ and then extracted with DCM. The organic layer was evaporated to provide the title compound as a yellow oil (73 mg) that was used without further purification.

[M+H⁺]: m/z 511.

### Step B: Synthesis of 4-[8-(3-bromo-4,5-dichlorobenzyl)-1-oxa-2,8-diazaspiro[4.5]dec-2-en-3-yl]benzoic acid, formic acid salt

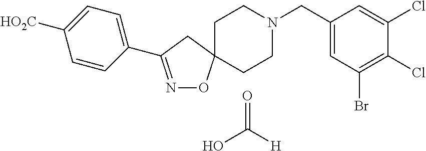

To methyl 4-[8-(3-bromo-4,5-dichlorobenzyl)-1-oxa-2,8-diazaspiro[4.5]dec-2-en-3-yl]benzoate (15 mg, 0.029 mmol) and LiOH (2.05 mg, 0.086 mmol) was added THF (900 μL) and water (300 μL). The reaction mixture stirred at 70° C. for 3 hours. The mixture was evaporated in vacuo and the residue was purified by reverse phase (C-18) HPLC eluting with acetonitrile/water+0.1% formic acid. The solvent was evaporated to provide the title compound as a white solid (7 mg).

¹H NMR (DMSO-d₆, 600 MHz): δ 7.94 (d, J=8.4 Hz, 2H), 7.71 (d, J=8.4 Hz, 2H), 7.69 (d, J=1.8 Hz, 1H), 7.59 (d, J=1.8 Hz, 1H), 3.18 (s, 2H), 2.50-2.34 (m, 6H), 1.79-1.72 (m, 4H).

[M+H⁺]: m/z 497.

### Example 77

### 4-[8-(3,4-Dichloro-5-cyclopropylbenzyl)-1-oxa-2,8-diazaspiro[4.5]dec-2-en-3-yl]benzoic acid, TFA salt

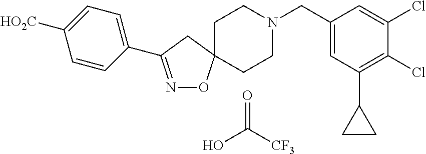

The title compound was prepared from methyl 4-[8-(3-bromo-4,5-dichlorobenzyl)-1-oxa-2,8-diazaspiro[4.5]dec-2-en-3-yl]benzoate (60 mg, 0.117 mmol) following essentially the same procedure described in Step B of EXAMPLE 74. The title compound was obtained as a white solid (14 mg).

¹H NMR (CD₃OD, 400 MHz): δ 8.03 (d, J=8.4 Hz, 2H), 7.74 (d, J=8.4 Hz, 2H), 7.53 (d, J=1.8 Hz, 1H), 7.13 (d, J=1.8 Hz, 1H), 4.27 (s, 2H), 3.31-3.26 (m, 6H), 2.27-2.05 (m, 5H), 1.10-1.05 (m, 2H), 0.77-0.73 (m, 2H).

[M+H⁺]: m/z 459.

### Example 78

### 4-[8-(4-Chloro-3,5-dicyclopropylbenzyl)-1-oxa-2,8-diazaspiro[4.5]dec-2-en-3-yl]benzoic acid, TFA salt

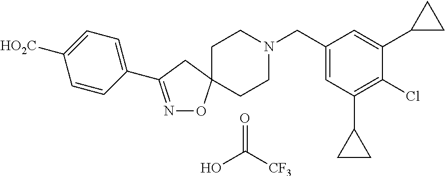

### Step A: Synthesis of methyl 4-[8-(3,5-dibromo-4-chlorobenzyl)-1-oxa-2,8-diazaspiro[4.]dec-2-en-3-yl]benzoate

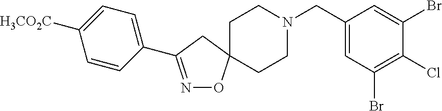 [SMILES: COC(=O)c1ccc(C2=NOC3(CCN(Cc4cc(Br)c(Cl)c(Br)c4)CC3)C2)cc1]

The title compound was prepared from INTERMEDIATE 1 (50 mg, 0.155 mmol) and Intermediate 30 (56.4 mg, 0.177 mmol) following essentially the same procedure described in Step A of EXAMPLE 76. The title compound was obtained as a white solid (86 mg) and was used without further purification.

[M+H⁺]: m/z 555.

### Step B: Synthesis of 4-[8-(4-chloro-3,5-dicyclopropylbenzyl)-1-oxa-2,8-diazaspiro[4.5]dec-2-en-3-yl]benzoic acid, TFA salt

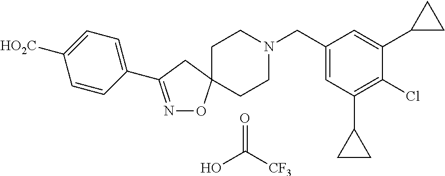

To methyl 4-[8-(3,5-dibromo-4-chlorobenzyl)-1-oxa-2,8-diazaspiro[4.5]dec-2-en-3-yl]benzoate (88 mg, 0.158 mmol), Cs₂CO₃ (309 mg, 0.948 mmol), Pd(OAc)₂ (1.42 mg, 6.32 μmol), potassium cyclopropyltrifluoroborate (56.1 mg, 0.379 mmol) and di(1-adamantyl)-n-butylphosphine (3.40 mg, 9.48 μmol) was added toluene (1437 uL) and water (144 uL). The reaction mixture was heated to 100° C. overnight. The reaction mixture was diluted with water, extracted with DCM and evaporated in vacuo. The residue was then dissolved in water (300 uL) and THF (900 uL) and LiOH (9.46 mg, 0.395 mmol) was added. The reaction mixture stirred at 70° C. for 3 hours. The mixture was evaporated in vacuo and the residue was purified by reverse phase (C-18) HPLC eluting with acetonitrile/water+0.1% TFA. The solvent was evaporated to provide the title compound as a white solid (33 mg).

¹H NMR (CD₃OD, 400 MHz): δ 8.03 (d, J=8.4 Hz, 2H), 7.74 (d, J=8.4 Hz, 2H), 6.98 (s, 2H), 4.23 (s, 2H), 3.31-3.26 (m, 6H), 2.25-2.05 (m, 6H), 1.03-1.00 (m, 4H), 0.71-0.68 (m, 4H). [M+H⁺]: m/z 479.

### Example 79

### 4-{8-[3,5-Dicyclopropyl-4-(trifluoromethoxy)benzyl]-1-oxa-2,8-diazaspiro[4.5]dec-2-en-3-yl}benzoic acid, TFA salt

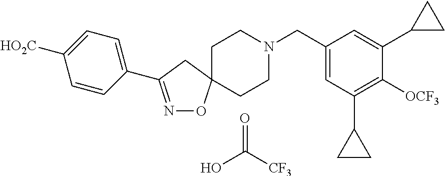

The title compound was prepared from INTERMEDIATE 2 (20 mg, 0.065 mmol) and Intermediate 31 (20.7 mg, 0.077 mmol) following essentially the same procedure described in EXAMPLE 70. The title compound was obtained as a white solid (20 mg).

¹H NMR (CD₃OD, 400 MHz): δ 8.03 (d, J=8.4 Hz, 2H), 7.74 (d, J=8.4 Hz, 2H), 6.90 (s, 2H), 4.22 (s, 2H), 3.31-3.26 (m, 6H), 2.17-2.07 (m, 6H), 1.05-1.02 (m, 4H), 0.76-0.72 (m, 4H).

[M+H⁺]: m/z 515.

### Example 80

### 4-{8-[3,5-Dichloro-4-(trifluoromethoxy)benzyl]-1-oxa-2,8-diazaspiro[4.5]dec-2-en-3-yl}benzoic acid, TFA salt

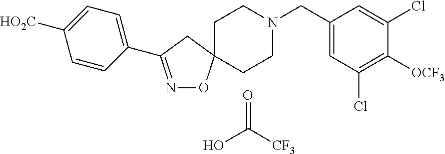

To INTERMEDIATE 1 (29 mg, 0.092 mmol) and INTERMEDIATE 32 (40.7 mg, 0.101 mmol) were added DMF (1 mL) and DIPEA (64.1 μL, 0.367 mmol). The mixture was heated to 55° C. for 2 hours. The mixture was evaporated and the residue was dissolved in saturated aqueous NaHCO₃ and then extracted with DCM. The organic layer was evaporated to provide the crude intermediate compound as a yellow oil that was used without further purification. The residue was dissolved in AcOH (1 mL) and zinc powder (24 mg, 0.367 mmol) was added. The mixture was heated to 35° C. for 10 min. The reaction mixture was filtered and evaporated in vacuo. The residue was dissolved in saturated aqueous NaHCO₃ and then extracted with DCM. The organic layer was evaporated and the residue was used without further purification. The residue was then dissolved in water (300 uL) and THF (900 uL) and LiOH (5.5 mg, 0.230 mmol) was added. The reaction mixture stirred at 70° C. for 3 hours. The mixture was evaporated in vacuo and the residue was purified by reverse phase (C-18) HPLC eluting with acetonitrile/water+0.1% TFA. The solvent was evaporated to provide the title compound as a white solid (11 mg).

¹H NMR (CD₃OD, 400 MHz) δ 8.04 (d, J=8.4 Hz, 2H), 7.76-7.73 (m, 4H), 4.35 (s, 2H), 3.40-3.26 (m, 6H), 2.19-2.07 (m, 4H).

[M+H⁺]: m/z 503.

### Example 81

### 4-[8-(2,4-Dichloro-5-cyclopropylbenzyl)-1-oxa-2,8-diazaspiro[4.5]dec-2-en-3-yl]benzoic acid, TFA salt

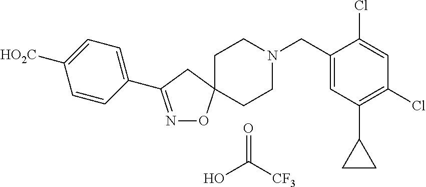

### Step A: Synthesis of methyl 4-[8-(5-bromo-2,4-dichlorobenzyl)-1-oxa-2,8-diazaspiro[4.5]dec-2-en-3-yl]benzoate

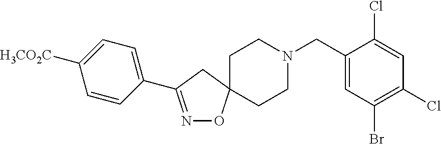 [SMILES: COC(=O)c1ccc(C2=NOC3(CCN(Cc4cc(Br)c(Cl)cc4Cl)CC3)C2)cc1]

The title compound was prepared from INTERMEDIATE 1 (80 mg, 0.245 mmol) and Intermediate 33 (86 mg, 0.269 mmol) following essentially the same procedure described in Step A of EXAMPLE 76. The title compound was obtained as a white solid (125 mg) and was used without further purification.

[M+H⁺]: m/z 511.

### Step B: Synthesis of 4-[8-(2,4-dichloro-5-cyclopropylbenzyl)-1-oxa-2,8-diazaspiro[4.5]dec-2-en-3-yl]benzoic acid, TFA salt

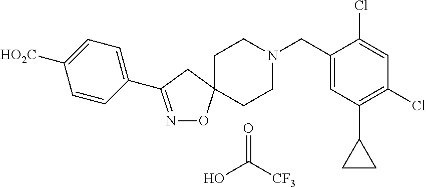

To methyl 4-[8-(5-bromo-2,4-dichlorobenzyl)-1-oxa-2,8-diazaspiro[4.5]dec-2-en-3-yl]benzoate (65 mg, 0.127 mmol), potassium phosphate tribasic (89 mg, 0.419 mmol), potassium cyclopropyltrifluoroborate (24.41 mg, 0.165 mmol) and palladium tetrakis (7.33 mg, 6.34 μmol) was added toluene (476 μL) and water (159 μL). The reaction mixture was heated to 100° C. overnight. The reaction mixture was diluted with water, extracted with DCM and evaporated in vacuo. The residue was then dissolved in water (300 uL) and THF (900 uL) and LiOH (7.6 mg, 0.317 mmol) was added. The reaction mixture stirred at 70° C. for 3 hours. The mixture was evaporated in vacuo and the residue was purified by reverse phase (C-18) HPLC eluting with acetonitrile/water+0.1% TFA. The solvent was evaporated to provide the title compound as a white solid (39 mg).

¹H NMR (CD₃OD, 400 MHz): δ 8.04 (d, J=8.4 Hz, 2H), 7.75 (d, J=8.4 Hz, 2H), 7.60 (s, 1H), 7.29 (s, 1H), 4.22 (s, 2H), 3.41-3.26 (m, 6H), 2.20-2.11 (m, 5H), 1.09-1.04 (m, 2H), 0.77-0.73 (m, 2H).

[M+H⁺]: m/z 459.

### Example 82

### 4-{8-[3-Chloro-5-cyclopropyl-4-(trifluoromethoxy)benzyl]-1-oxa-2,8-diazaspiro[4.5]dec-2-en-3-yl}benzoic acid, TFA salt

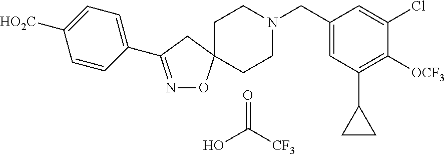

### Step A: Synthesis of methyl 4-{8-[3,5-dichloro-4-(trifluoromethoxy)benzyl]-1-oxa-2,8-diazaspiro[4.5]dec-2-en-3-yl}benzoate

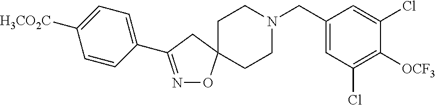 [SMILES: COC(=O)c1ccc(C2=NOC3(CCN(Cc4cc(Cl)c(OC(F)(F)F)c(Cl)c4)CC3)C2)cc1]

To INTERMEDIATE 1 (29 mg, 0.092 mmol) and INTERMEDIATE 32 (40.7 mg, 0.101 mmol) were added DMF (1 mL) and DIPEA (64.1 μL, 0.367 mmol). The mixture was heated to 55° C. for 2 hours. The mixture was evaporated and the residue was dissolved in saturated aqueous NaHCO₃ and then extracted with DCM. The organic layer was evaporated to provide the crude intermediate as a yellow oil that was used without further purification. The residue was dissolved in AcOH (1 mL) and zinc powder (24 mg, 0.367 mmol) was added. The mixture was heated to 35° C. for 10 min. The reaction mixture was filtered and evaporated in vacuo. The residue was dissolved in saturated aqueous NaHCO₃ and then extracted with DCM. The organic layer was evaporated and the residue was used without further purification.

[M+H⁺]: m/z 517.

### Step B: Synthesis of 4-{8-[3-chloro-5-cyclopropyl-4-(trifluoromethoxy)benzyl]-1-oxa-2,8-diazaspiro[4.5]dec-2-en-3-yl}benzoic acid, TFA salt

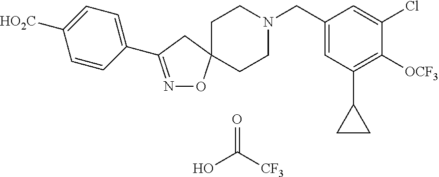 [SMILES: O=C(O)c1ccc(C2=NOC3(CCN(Cc4cc(Cl)c(OC(F)(F)F)c(C5CC5)c4)CC3)C2)cc1 ; O=C(O)C(F)(F)F]

The title compound was prepared from methyl 4-{8-[3,5-dichloro-4-(trifluoromethoxy)benzyl]-1-oxa-2,8-diazaspiro[4.5]dec-2-en-3-yl}benzoate (65 mg, 0.126 mmol) following essentially the same procedure described in Step B of EXAMPLE 74. The title compound was obtained as a white solid (19 mg).

¹H NMR (CD₃OD, 400 MHz): δ 8.04 (d, J=8.4 Hz, 2H), 7.75 (d, J=8.4 Hz, 2H), 7.51 (d, J=1.8 Hz, 1H), 7.06 (d, J=1.8 Hz, 1H), 4.18 (s, 2H), 3.31-3.26 (m, 6H), 2.19-2.03 (m, 5H), 1.13-1.08 (m, 2H), 0.82-0.78 (m, 2H).

[M+H⁺]: m/z 509.

### Example 83

### 4-[8-(2,4,5-Tricyclopropylbenzyl)-1-oxa-2,8-diazaspiro[4.5]dec-2-en-3-yl]benzoic acid, TFA salt

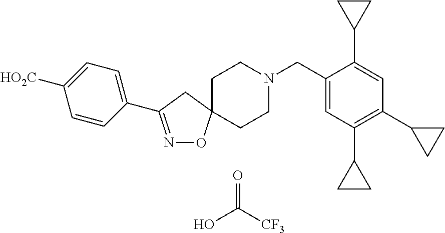 [SMILES: O=C(O)c1ccc(C2=NOC3(CCN(Cc4cc(C5CC5)c(C5CC5)cc4C4CC4)CC3)C2)cc1 ; O=C(O)C(F)(F)F]

To methyl 4-[8-(5-bromo-2,4-dichlorobenzyl)-1-oxa-2,8-diazaspiro[4.5]dec-2-en-3-yl]benzoate (65 mg, 0.127 mmol), Cs₂CO₃ (496 mg, 1.52 mmol), Pd(OAc)₂ (2.28 mg, 10.2 μmol), potassium cyclopropyltrifluoroborate (90 mg, 0.609 mmol) and di(1-adamantyl)-n-butylphosphine (5.46 mg, 0.015 mmol) was added toluene (2307 uL) and water (231 uL). The reaction mixture was heated to 100° C. overnight. The reaction mixture was diluted with water, extracted with DCM and evaporated in vacuo. The residue was then dissolved in water (300 uL) and THF (900 uL) and LiOH (7.6 mg, 0.317 mmol) was added. The reaction mixture stirred at 70° C. for 3 hours. The mixture was evaporated in vacuo and the residue was purified by reverse phase (C-18) HPLC eluting with acetonitrile/water+0.1% TFA. The solvent was evaporated to provide the title compound as a white solid (24 mg).

¹H NMR (CD₃OD, 400 MHz) δ 8.04 (d, J=8.4 Hz, 2H), 7.75 (d, J=8.4 Hz, 2H), 7.06 (s, 1H), 6.62 (s, 1H), 4.50 (s, 2H), 3.41-3.26 (m, 6H), 2.21-1.97 (m, 7H), 1.04-0.94 (m, 6H), 0.68-0.61 (m, 6H).

[M+H⁺]: m/z 471.

### Example 84

### 4-[8-(2-Chloro-5-cyclopropylbenzyl)-1-oxa-2,8-diazaspiro[4.5]dec-2-en-3-yl]benzoic acid, TFA salt

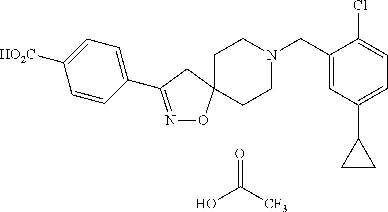 [SMILES: O=C(O)c1ccc(C2=NOC3(CCN(Cc4cc(C5CC5)ccc4Cl)CC3)C2)cc1 ; O=C(O)C(F)(F)F]

### Step A: Synthesis of methyl 4-[8-(5-bromo-2-chlorobenzyl)-1-oxa-2,8-diazaspiro[4.5]dec-2-en-3-yl]benzoate

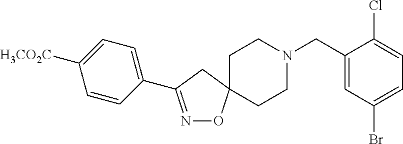 [SMILES: COC(=O)c1ccc(C2=NOC3(CCN(Cc4cc(Br)ccc4Cl)CC3)C2)cc1]

The title compound was prepared from INTERMEDIATE 1 (50 mg, 0.161 mmol) and 5-bromo-2-chlorobenzaldehyde (45.9 mg, 0.209 mmol) following essentially the same procedure described in Step A of EXAMPLE 74. The title compound was obtained as a brown oil (77 mg) that was used without any further purification.

[M+H⁺]: m/z 477.

### Step B: Synthesis of 4-[8-(2-chloro-5-cyclopropylbenzyl)-1-oxa-2,8-diazaspiro[4.5]dec-2-en-3-yl]benzoic acid, TFA salt

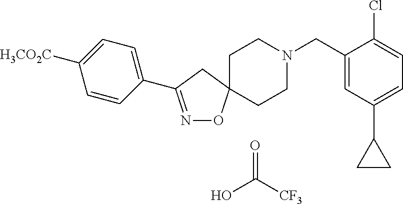

The title compound was prepared from methyl 4-[8-(5-bromo-2-chlorobenzyl)-1-oxa-2,8-diazaspiro[4.5]dec-2-en-3-yl]benzoate (77 mg, 0.161 mmol) following essentially the same procedure described in Step B of EXAMPLE 81. The title compound was obtained as a white solid (4.3 mg).

¹H NMR (DMSO-d₆, 600 MHz): δ 7.94 (d, J=8.4 Hz, 2H), 7.72 (d, J=8.4 Hz, 2H), 7.24 (d, J=8.4 Hz, 1H), 7.18 (d, J=2.0 Hz, 1H), 6.92 (dd, J=2.0, 8.0 Hz, 1H), 3.19 (s, 2H), 2.62-2.34 (m, 6H), 1.91-1.87 (m, 1H), 1.79-1.71 (m, 4H), 0.94-0.91 (m, 2H), 0.62-0.59 (m, 2H).

[M+H⁺]: m/z 425.

### Example 85

### 4-[8-(3-Chloro-5-cyclopropylbenzyl)-1-oxa-2,8-diazaspiro[4.5]dec-2-en-3-yl]benzoic acid, TFA salt

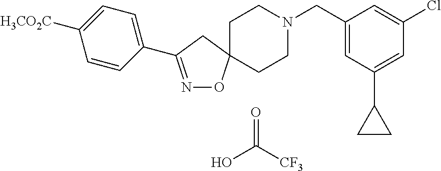

### Step A: Synthesis of methyl 4-[8-(5-bromo-3-chlorobenzyl)-1-oxa-2,8-diazaspiro[4.5]dec-2-en-3-yl]benzoate

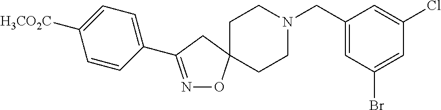 [SMILES: COC(=O)c1ccc(C2=NOC3(CCN(Cc4cc(Cl)cc(Br)c4)CC3)C2)cc1]

The title compound was prepared from INTERMEDIATE 1 (50 mg, 0.161 mmol) and 5-bromo-3-chlorobenzaldehyde (45.9 mg, 0.209 mmol) following essentially the same procedure described in Step A of EXAMPLE 74. The title compound was obtained as a brown oil (77 mg) that was used without any further purification.

[M+H⁺]: m/z 477.

### Step B: Synthesis of 4-[8-(3-chloro-5-cyclopropylbenzyl)-1-oxa-2,8-diazaspiro[4.5]dec-2-en-3-yl]benzoic acid, TFA salt

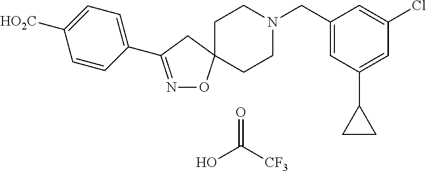

The title compound was prepared from methyl 4-[8-(5-bromo-3-chlorobenzyl)-1-oxa-2,8-diazaspiro[4.5]dec-2-en-3-yl]benzoate (77 mg, 0.161 mmol) following essentially the same procedure described in Step B of EXAMPLE 81. The title compound was obtained as a white solid (11.1 mg).

¹H NMR (DMSO-d₆, 600 MHz): δ 7.94 (d, J=8.4 Hz, 2H), 7.72 (d, J=8.4 Hz, 2H), 7.09 (s, 1H), 6.96-6.95 (m, 2H), 2.95 (s, 2H), 2.52-2.34 (m, 6H), 1.92-1.87 (m, 1H), 1.79-1.71 (m, 4H), 0.94-0.91 (m, 2H), 0.66-0.64 (m, 2H).

[M+H⁺]: m/z 425.

### Example 86

### 4-{8-[3-Cyclopropyl-4-(trifluoromethyl)benzyl]-1-oxa-2,8-diazaspiro[4.5]dec-2-en-3-yl}benzoic acid, TFA salt

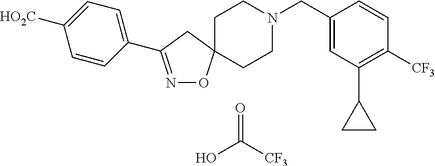

### Step A: Synthesis of methyl 4-{8-[3-bromo-4-(trifluoromethyl)benzyl]-1-oxa-2,8-diazaspiro[4.5]dec-2-en-3-yl}benzoate

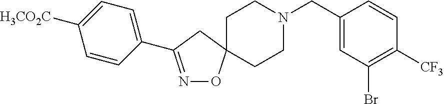 [SMILES: COC(=O)c1ccc(C2=NOC3(CCN(Cc4ccc(C(F)(F)F)c(Br)c4)CC3)C2)cc1]

The title compound was prepared from INTERMEDIATE 1 (47.6 mg, 0.153 mmol) and INTERMEDIATE 34 (53.5 mg, 0.168 mmol) following essentially the same procedure described in Step A of EXAMPLE 76. The title compound was obtained as a yellowish brown oil (78 mg) that was used without any further purification.

[M+H⁺]: m/z 511.

### Step B: Synthesis of 4-{8-[3-cyclopropyl-4-(trifluoromethyl)benzyl]-1-oxa-2,8-diazaspiro[4.5]dec-2-en-3-yl}benzoic acid, TFA salt

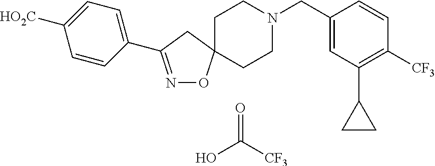

To methyl 4-{8-[3-bromo-4-(trifluoromethyl)benzyl]-1-oxa-2,8-diazaspiro[4.5]dec-2-en-3-yl}benzoate (75 mg, 0.147 mmol), cyclopropylboronic acid pinacol ester (61.6 mg, 0.367 mmol), 1,1′-bis(diphenylphosphino)ferrocene-palladium(II)

dichloride dichloromethane complex (35.9 mg, 0.044 mmol) and LiOH (14.1 mg, 0.587 mmol) in a microwave vial was added 1,4-dioxane (367 μL) and water (122 μL). The mixture was irradiated at 120° C. for 30 min. The mixture was evaporated in vacuo and the residue was purified by reverse phase (C-18) HPLC eluting with acetonitrile/water+0.1% TFA. The solvent was evaporated to provide the title compound as a white solid (15 mg).

¹H NMR (CD₃OD, 400 MHz): δ 8.04 (d, J=8.4 Hz, 2H), 7.75-7.70 (m, 3H), 7.44 (d, J=8.0 Hz, 1H), 7.29 (s, 1H), 4.38 (s, 2H), 3.38-3.26 (m, 6H), 2.21-2.08 (m, 5H), 1.09-1.03 (m, 2H), 0.89-0.82 (m, 2H).

[M+H⁺]: m/z 459.

### Example 87

### 4-{8-[(8-Chloro-4,4-dimethyl-3,4-dihydro-2H-chromen-6-yl)methyl]-1-oxa-2,8-diazaspiro[4.5]dec-2-en-3-yl}benzoic acid trifluoroacetate

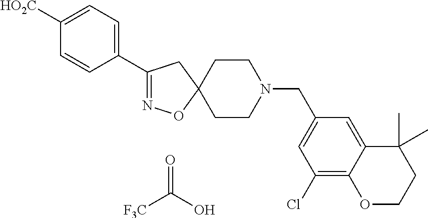

4-(1-Oxa-2,8-diazaspiro[4,5]dec-2-en-3-yl)benzoic acid hydrochloride (10.0 mg, 0.03 mmol, Intermediate 2) and 8-chloro-4,4-dimethyl-3,4-dihydro-2H-chromene-6-carbaldehyde (9.09 mg, 0.04 mmol, INTERMEDIATE 53) were dissolved in DMF (337 μL), and MP-cyanoborohydride (58.4 mg, 0.13 mmol) was added followed by acetic acid (5.79 μL, 0.10 mmol). The reaction mixture was placed in a sand-bath shaker at 55° C. overnight, allowed to cool to room temperature, filtered, and concentrated under vacuum. The crude mixture was purified by reverse-phase Shimadzu HPLC (Sunfire prep C18 30 mm×100 mm), eluting with Acetonitrile/Water+0.1% TFA to give the title compound as a white solid after lypholization.

¹H NMR (DMSO-d₆, 500 MHz): δ 8.00 (d, J=7.9 Hz, 2H), 7.75 (d, J=8.1 Hz, 2H), 7.44 (b, 2H), 4.28 (t, J=5.2 Hz, 4H), 3.36 (b, 4H), 3.12 (b, 2H), 2.06-2.17 (m, 2H), 1.94-205 (m, 2H), 1.83 (t, J=5.3 Hz, 2H), 1.31 (s, 6H).

[M+H⁺]: [M+H⁺]: m/z 469.

### Example 88

### 4-{8-[(8-Cyclopropyl-4,4-dimethyl-3,4-dihydro-2H-chromen-6-yl)methyl]-1-oxa-2,8-diazaspiro[4.5]dec-2-en-3-yl}benzoic acid, TFA salt

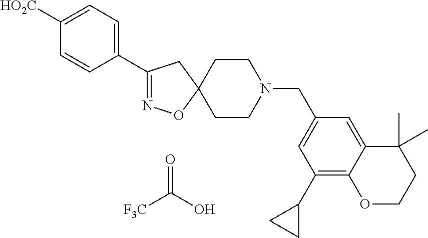

4-(1-Oxa-2,8-diazaspiro[4,5]dec-2-en-3-yl)benzoic acid hydrochloride (20.0 mg, 0.07 mmol, INTERMEDIATE 2) and 8-cyclopropyl-4,4-dimethyl-3,4-dihydro-2H-chromene-6-carbaldehyde (18.6 mg, 0.08 mmol, INTERMEDIATE 54) were dissolved in DMF (674 μL), and MP-cyanoborohydride (117 mg, 0.27 mmol) was added followed by acetic acid (11.6 μL, 0.20 mmol). The reaction mixture was placed in a sand-bath shaker at 55° C. overnight, allowed to cool to room temperature, filtered, and concentrated under vacuum. The crude mixture was purified by reverse-phase Gilson HPLC (Sunfire prep C18 30 mm×100 mm), eluting with Acetonitrile/Water+0.1% TFA to give the title compound as a white solid after lypholization.

¹H NMR (CD₃OD, 400 MHz): δ 8.02 (d, J=8.2 Hz, 2H), 7.73 (d, J=8.1 Hz, 2H), 7.24 (s, 1H), 6.76 (d, J=1.6 Hz, 1H), 4.23 (t, J=5.3 Hz, 2H), 4.19 (s, 2H), 3.41 (app d, J=10.2 Hz, 2H), 3.22-3.31 (m, 4H), 2.17-2.04 (m, 5H), 1.82 (t, J=5.3 Hz, 2H), 1.32 (s, 6H), 0.86 (ddd, J=10.4 Hz, 6.1 Hz, 4.5 Hz, 2H), 0.59 (ddd, J=10.1 Hz, 6.0 Hz, 4.8 Hz, 2H).

[M+H⁺]: m/z 475.

### Example 89

### 4-{8-[(8-Chloro-2,2-dimethyl-3,4-dihydro-2H-chromen-6-yl)methyl]-1-oxa-2,8-diazaspiro[4.5]dec-2-en-3-yl}benzoic acid, TFA salt

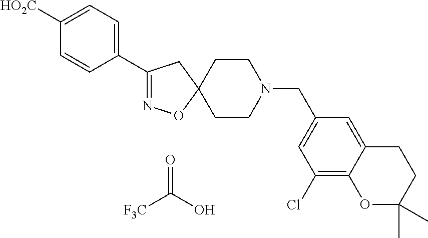

4-(1-Oxa-2,8-diazaspiro[4,5]dec-2-en-3-yl)benzoic acid hydrochloride (14.3 mg, 0.05 mmol, Intermediate 2) and 8-chloro-2,2-dimethyl-3,4-dihydro-2H-chromene-6-carbaldehyde (13.0 mg, 0.06 mmol, INTERMEDIATE 55) were dissolved in DMF (483 μL), and MP-cyanoborohydride (84.0 mg, 0.19 mmol) was added followed by acetic acid (8.29 μL, 0.14 mmol). The reaction mixture was placed in a sand-bath shaker at 55° C. overnight, allowed to cool to room temperature, filtered, and concentrated under vacuum. The crude residue was purified by reverse-phase Shimadzu HPLC (Sunfire prep C18 30 mm×100 mm), eluting with Acetonitrile/Water+0.1% TFA to give the title compound as a white solid after lypholization.

¹H NMR (CD₃OD, 400 MHz): δ 8.03 (d, J=8.4 Hz, 2H), 7.74 (d, J=8.4 Hz, 2H), 7.34 (d, J=1.8 Hz, 1H), 7.15 (s, 1H), 4.21 (s, 2H), 3.36 (b, 4H), 3.26-3.27 (m, 2H), 2.84 (t, J=6.6 Hz, 2H), 2.17-2.04 (m, 4H), 1.83 (t, J=6.6 Hz, 2H), 1.34 (s, 6H).

[M+H⁺]: m/z 469.

### Example 90

### 4-{8-[(8-Cyclopropyl-2,2-dimethyl-3,4-dihydro-2H-chromen-6-yl)methyl]-1-oxa-2,8-diazaspiro[4.5]dec-2-en-3-yl}benzoic acid, TFA salt

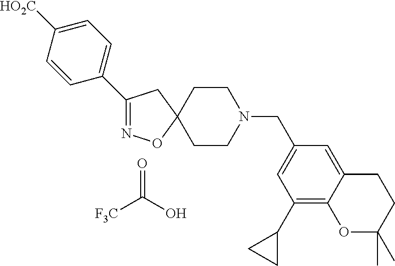

4-(1-Oxa-2,8-diazaspiro[4,5]dec-2-en-3-yl)benzoic acid hydrochloride (14.3 mg, 0.05 mmol, Intermediate 2) and 8-cyclopropyl-2,2-dimethyl-3,4-dihydro-2H-chromene-6-carbaldehyde (12.2 mg, 0.05 mmol, INTERMEDIATE 56) were dissolved in DMF (483 μL), and MP-cyanoborohydride (84.0 mg, 0.19 mmol) was added followed by acetic acid (8.29 μL, 0.14 mmol). The reaction mixture was placed in a sand-bath shaker at 55° C. overnight, allowed to cool to room temperature, filtered, and concentrated under vacuum. The crude mixture was purified by reverse-phase Shimadzu HPLC (Sunfire prep C18 30 mm×100 mm), eluting with Acetonitrile/Water+0.1% TFA to give the title compound as a white solid after lypholization.

¹H NMR (CD₃OD, 400 MHz): δ 8.02 (d, J=8.2 Hz, 2H), 7.73 (d, J=8.1 Hz, 2H), 6.98 (s, 1H), 6.76 (s, 1H), 4.17 (b, 2H), 3.42 (app d J=9.7 Hz, 2H), 3.23-3.31 (m, 4H), 2.80 (t, J=6.8 Hz, 2H), 1.99-2.18 (m, 5H), 1.80 (t, J=6.7 Hz, 2H), 1.32 (s, 6H), 0.87 (ddd, J=10.4 Hz, 6.3 Hz, 4.4 Hz, 2H), 0.61 (ddd, J=10.0 Hz, 6.2 Hz, 5.6 Hz, 2H).

[M+H⁺]: m/z 475.

### Example 91

### 4-{8-[(2,2,8-Trimethyl-3,4-dihydro-2H-chromen-6-yl)methyl]-1-oxa-2,8-diazaspiro[4.5]dec-2-en-3-yl}benzoic acid, TFA salt

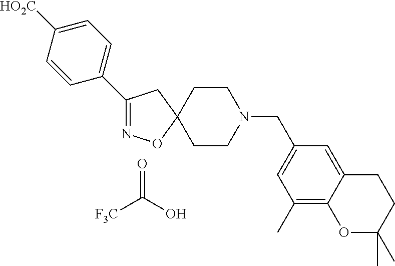

Using essentially the same procedure as EXAMPLE 89: 4-(1-Oxa-2,8-diazaspiro[4,5]dec-2-en-3-yl)benzoic acid hydrochloride (15.0 mg, 0.05 mmol, INTERMEDIATE 2), 2,2,8-trimethyl-3,4-dihydro-2H-chromene-6-carbaldehyde (12.4 mg, 0.06 mmol, INTERMEDIATE 57), MP-cyanoborohydride (88.0 mg, 0.20 mmol), and acetic acid (8.68 μL, 0.15 mmol) afforded the title compound as a white solid.

¹H NMR (CD₃OD, 400 MHz): δ 8.02 (d, J=8.3 Hz, 2H), 7.73 (d, J=8.2 Hz, 2H), 7.05 (s, 1H), 7.03 (s, 1H), 4.19 (s, 2H), 3.45 (app d, J=10.1 Hz, 2H), 3.26-3.30 (m, 4H), 2.79 (t, J=6.7 Hz, 2H), 2.03-2.18 (m, 7H), 1.79 (t, J=6.7 Hz, 2H), 1.30 (s, 6H), 0.85-0.90 (m, 2H), 0.60-0.64 (m, 2H).

[M+H⁺]: m/z 449.

### Example 92

### 4-{8-[(2,2-Dimethyl-3,4-dihydro-2H-chromen-8-yl)methyl]-1-oxa-2,8-diazaspiro[4.5]dec-2-en-3-yl}benzoic acid, TFA salt

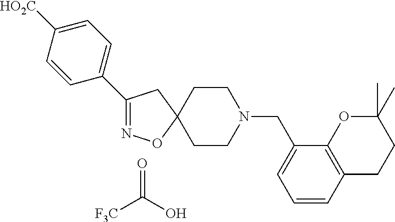

Using essentially the same procedure as EXAMPLE 87: 4-(1-Oxa-2,8-diazaspiro[4,5]dec-2-en-3-yl)benzoic acid hydrochloride (14.3 mg, 0.05 mmol, INTERMEDIATE 2), 2,2-dimethyl-3,4-dihydro-2H-chromene-8-carbaldehyde (11.0 mg, 0.06 mmol, INTERMEDIATE 58), MP-cyanoborohydride (84.0 mg, 0.19 mmol), and acetic acid (8.29 μL, 0.14 mmol) afforded the title compound as a colorless oil.

¹H NMR (CD₃OD, 400 MHz): δ 8.02 (d, J=8.2 Hz, 2H), 7.73 (d, J=8.4 Hz, 2H), 7.22 (app t, J=8.6 Hz, 2H), 6.89 (t, J=7.5 Hz, 2H), 4.31 (s, 1H), 3.94 (s, 1H), 3.51 (app d, J=12.6 Hz, 2H), 3.30-3.37 (m, 4H), 2.82 (t, J=6.7 Hz, 2H), 2.05-2.19 (m, 4H), 1.85 (t, J=6.8 Hz, 2H), 1.37 (s, 6H).

[M+H⁺]: m/z 435.

### Example 93

### 4-{8-[(6-Chloro-2,2-dimethyl-3,4-dihydro-2H-chromen-8-yl)methyl]-1-oxa-2,8-diazaspiro[4.5]dec-2-en-3-yl}benzoic acid, TFA salt

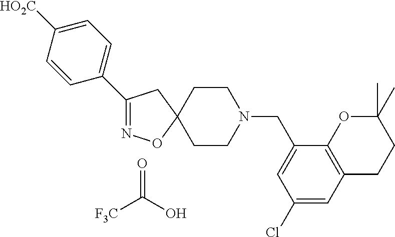

Using essentially the same procedure as EXAMPLE 87: 4-(1-Oxa-2,8-diazaspiro[4,5]dec-2-en-3-yl)benzoic acid hydrochloride (14.3 mg, 0.05 mmol, INTERMEDIATE 2), 6-chloro-2,2-dimethyl-3,4-dihydro-2H-chromene-8-carbaldehyde (13.0 mg, 0.06 mmol, INTERMEDIATE 59), MP-cyanoborohydride (84.0 mg, 0.19 mmol), and acetic acid (8.29 μL, 0.14 mmol) afforded the title compound as a colorless oil.

¹H NMR (CD₃OD, 400 MHz): δ 8.03 (d, J=8.3 Hz, 2H), 7.74 (d, J=8.1 Hz, 2H), 7.30 (d, J=2.3 Hz, 1H), 7.24 (d, J=2.3 Hz, 1H), 4.30 (b, 2H), 3.26-3.52 (m, 6H), 2.82 (t, J=6.8 Hz, 2H), 2.06-2.20 (m, 4H), 1.85 (t, J=6.8 Hz, 2H), 1.37 (s, 6H).

[M+H⁺]: m/z 469.

### Example 94

### 4-({8-[(6-Cyclopropyl-2,2-dimethyl-3,4-dihydro-2H-chromen-8-yl)methyl]-1-oxa-2,8-diazaspiro[4.5]dec-2-en-3-yl}benzoic acid, TFA salt

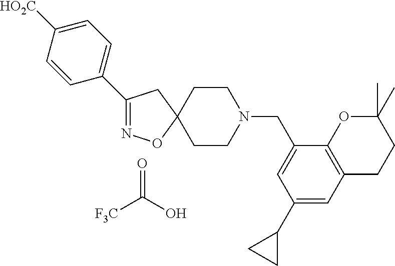

Using essentially the same procedure as EXAMPLE 87: 4-(1-Oxa-2,8-diazaspiro[4,5]dec-2-en-3-yl)benzoic acid hydrochloride (14.3 mg, 0.05 mmol, INTERMEDIATE 2), 6-cyclopropyl-2,2-dimethyl-3,4-dihydro-2H-chromene-8-carbaldehyde (13.3 mg, 0.06 mmol, INTERMEDIATE 60), MP-cyanoborohydride (84.0 mg, 0.19 mmol), and acetic acid (8.29 μL, 0.14 mmol) afforded the title compound as a white solid.

¹H NMR (CD₃OD, 400 MHz): δ 8.02 (d, J=8.4 Hz, 2H), 7.73 (d, J=8.4 Hz, 2H), 6.98 (d, J=1.7 Hz, 1H), 6.93 (d, J=1.7 Hz, 1H), 4.26 (s, 2H), 3.49 (app d, J=12.5 Hz, 2H), 3.26-3.35 (m, 4H), 2.77 (t, J=6.7 Hz, 2H), 2.04-2.26 (m, 4H), 1.79-1.85 (m, 3H), 1.34 (s, 6H), 0.88 (ddd, J=10.7 Hz, 6.4 Hz, 4.5 Hz, 2H), 0.59 (ddd, J=9.5 Hz, 6.2 Hz, 4.6 Hz, 2H).

[M+H⁺]: m/z 475.

### Example 95

### 4-{8-[(2,2-Dimethyl-2,3-dihydrospiro[chromene-4,1′-cyclopropan]-6-yl)methyl]-1-oxa-2,8-diazaspiro[4.5]dec-2-en-3-yl}benzoic acid, TFA salt

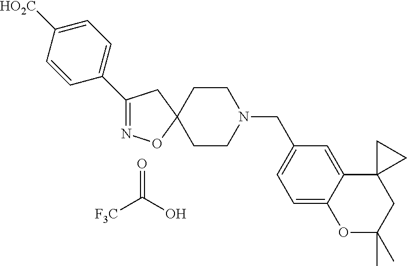

Using essentially the same procedure as EXAMPLE 87: 4-(1-Oxa-2,8-diazaspiro[4,5]dec-2-en-3-yl)benzoic acid hydrochloride (23.0 mg, 0.08 mmol, INTERMEDIATE 2), 2,2-dimethyl-2,3-dihydrospiro[chromene-4,1′-cyclopropane]-6-carbaldehyde (20.0 mg, 0.09 mmol, Intermediate 61), MP-cyanoborohydride (134 mg, 0.31 mmol), and acetic acid (13.3 μL, 0.23 mmol) afforded the title compound as a white solid.

¹H NMR (CD₃OD, 400 MHz): δ 8.03 (d, J=8.4 Hz, 2H), 7.74 (d, J=8.4 Hz, 2H), 7.11 (dd, J=8.4 Hz, 2.0 Hz, 1H), 6.83 (d, J=2.0 Hz, 1H), 6.77 (d, J=8.4 Hz, 1H), 4.21 (s, 2H), 3.26-3.46 (m, 6H), 2.00-2.20 (m, 4H), 1.76 (s, 2H), 1.34 (s, 6H), 1.06 (dd, J=6.4 Hz, 4.8 Hz, 2H), 0.93 (dd, J=6.0 Hz, 4.0 Hz, 2H).

[M+H⁺]: m/z 461.

### Example 96

### 4-{8-[(8-Chloro-2,2-dimethyl-2,3-dihydrospiro[chromene-4,1′-cyclopropan]-6-yl)methyl]-1-oxa-2,8-diazaspiro[4.5]dec-2-en-3-yl}benzoic acid, TFA salt

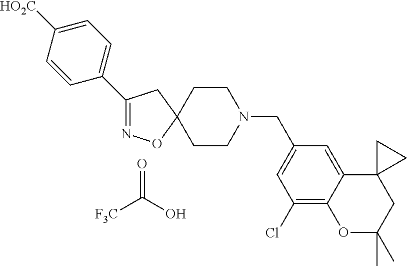

Using essentially the same procedure as EXAMPLE 87: 4-(1-Oxa-2,8-diazaspiro[4,5]dec-2-en-3-yl)benzoic acid hydrochloride (14.3 mg, 0.05 mmol, INTERMEDIATE 2), 8-chloro-2,2-dimethyl-2,3-dihydrospiro[chromene-4,1′-cyclopropane]-6-carbaldehyde (14.5 mg, 0.06 mmol, INTERMEDIATE 62), MP-cyanoborohydride (89.0 mg, 0.19 mmol), and acetic acid (8.29 μL, 0.14 mmol) afforded the title compound as a white solid.

¹H NMR (CD₃OD, 400 MHz): δ 8.03 (d, J=8.4 Hz, 2H), 7.73 (d, J=8.3 Hz, 2H), 7.30 (d, J=2.0 Hz, 1H), 6.79 (d, J=2.0 Hz, 1H), 4.20 (s, 2H), 3.26-3.50 (m, 6H), 1.97-2.20 (m, 4H), 1.79 (s, 2H), 1.38 (s, 6H), 1.09 (dd, J=7.0 Hz, 4.1 Hz, 2H), 0.98 (dd, J=6.0 Hz, 4.3 Hz, 2H).

[M+H⁺]: m/z 495.

### Example 97

### 4-{8-[(8-Cyclopropyl-2,2-dimethyl-2,3-dihydrospiro[chromene-4,1′-cyclopropan]-6-yl)methyl]-1-oxa-2,8-diazaspiro[4.5]dec-2-en-3-yl}benzoic acid, TFA salt

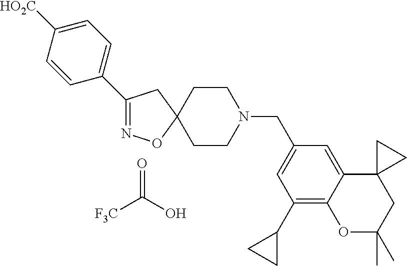

Using essentially the same procedure as EXAMPLE 87: 4-(1-Oxa-2,8-diazaspiro[4,5]dec-2-en-3-yl)benzoic acid hydrochloride (14.3 mg, 0.05 mmol, INTERMEDIATE 2), 8-cyclopropyl-2,2-dimethyl-2,3-dihydrospiro[chromene-4,1′-cyclopropane]-6-carbaldehyde (15.0 mg, 0.06 mmol, Intermediate 63), MP-cyanoborohydride (89.0 mg, 0.19 mmol), and acetic acid (8.29 μL, 0.14 mmol) afforded the title compound as a white solid.

¹H NMR (CD₃OD, 400 MHz): δ 8.02 (d, J=8.3 Hz, 2H), 7.73 (d, J=8.2 Hz, 2H), 6.72 (app d, J=2.4 Hz, 1H), 6.61 (app d, J=2.4 Hz, 1H), 4.12-4.20 (m, 2H), 3.39 (app d, J=12.4 Hz, 2H), 3.20-3.33 (m, 4H), 2.00-2.21 (m, 5H), 1.76 (s, 2H), 1.37 (s, 6H), 1.03 (dd, J=6.8 Hz, 4.1 Hz, 2H), 0.85-0.94 (m, 4H), 0.62 (ddd, J=9.9 Hz, 6.0 Hz, 4.6 Hz, 2H).

[M+H⁺]: m/z 501.

### Example 98

### 4-{8-[(8-Cyclopropyl-4,4-dimethyl-3,4-dihydro-2H-thiochromen-6-yl)methyl]-1-oxa-2,8-diazaspiro[4.5]dec-2-en-3-yl}benzoic acid, TFA salt

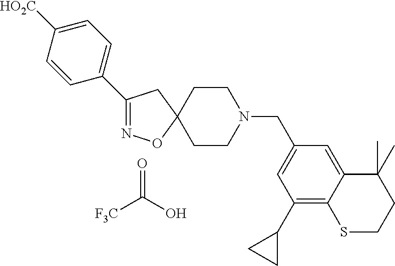

Using essentially the same procedure as EXAMPLE 87: 4-(1-Oxa-2,8-diazaspiro[4,5]dec-2-en-3-yl)benzoic acid hydrochloride (15.0 mg, 0.05 mmol, INTERMEDIATE 2), 8-cyclopropyl-4,4-dimethyl-3,4-dihydro-2H-thiochromene-6-carbaldehyde (15.0 mg, 0.06 mmol, INTERMEDIATE 64), MP-cyanoborohydride (88.0 mg, 0.20 mmol), and acetic acid (8.68 μL, 0.15 mmol) afforded the title compound as a colorless oil.

¹H NMR (CD₃OD, 400 MHz): δ 8.02 (d, J=8.2 Hz, 2H), 7.73 (d, J=8.3 Hz, 2H), 7.39 (s, 1H), 6.98 (s, 1H), 4.20-4.30 (m, 2H), 3.42 (app d, J=8.4 Hz, 2H), 3.24-3.35 (m, 4H), 3.00-3.07 (m, 2H), 2.17 (app d, J=13.7 Hz, 2H), 2.02-2.12 (m, 2H), 1.90-1.96 (m, 2H), 1.76-1.85 (m, 1H), 1.32 (s, 6H), 0.92 (ddd, J=10.7 Hz, 6.3 Hz, 4.5 Hz, 2H), 0.60 (ddd, J=10.1 Hz, 6.0 Hz, 4.7 Hz, 2H).

[M+H⁺]: m/z 491.

### Example 99

### 4-{8-[(8-Chloro-4,4-dimethyl-1,1-dioxido-3,4-dihydro-2H-thiochromen-6-yl)methyl]-1-oxa-2,8-diazaspiro[4.5]dec-2-en-3-yl}benzoic acid, TFA salt

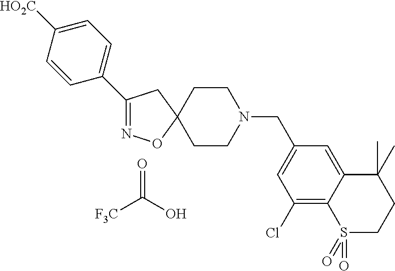

Using essentially the same procedure as EXAMPLE 87: 4-(1-Oxa-2,8-diazaspiro[4,5]dec-2-en-3-yl)benzoic acid hydrochloride (10.0 mg, 0.03 mmol, INTERMEDIATE 2), 8-chloro-4,4-dimethyl-3,4-dihydro-2H-thiochromene-6-carbaldehyde 1,1-dioxide (11.0 mg, 0.04 mol, INTERMEDIATE 65), MP-cyanoborohydride (58.0 mg, 0.13 mmol), and acetic acid (5.79 μL, 0.10 mmol) afforded the title compound as a white solid.

¹H NMR (DMSO-d6, 500 MHz): δ 8.00 (d, J=8.0 Hz, 2H), 7.80 (s, 1H), 7.75 (d, J=8.2 Hz, 2H), 7.67 (s, 1H), 4.42 (s, 2H), 3.10-3.70 (m, 8H), 2.20-2.25 (m, 2H), 1.95-2.16 (m, 4H), 1.38 (s, 6H).

[M+H⁺]: m/z 517.

### Example 100

### 4-{8-[(8-Cyclopropyl-4,4-dimethyl-1,1-dioxido-3,4-dihydro-2H-thiochromen-6-yl)methyl]-1-oxa-2,8-diazaspiro[4.5]dec-2-en-3-yl}benzoic acid, TFA salt

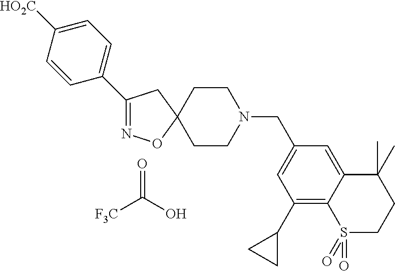

Using essentially the same procedure as EXAMPLE 87: 4-(1-Oxa-2,8-diazaspiro[4,5]dec-2-en-3-yl)benzoic acid hydrochloride (14.0 mg, 0.05 mmol, INTERMEDIATE 2), 8-cyclopropyl-4,4-dimethyl-3,4-dihydro-2H-thiochromene-6-carbaldehyde 1,1-dioxide (16.1 mg, 0.06 mmol, INTERMEDIATE 66), MP-cyanoborohydride (84.0 mg, 0.19 mmol), and acetic acid (8.29 μL, 0.14 mmol) afforded the title compound as a white solid.

¹H NMR (CD₃OD, 400 MHz): δ 8.03 (d, J=8.4 Hz, 2H), 7.74 (d, J=8.4 Hz, 2H), 7.54 (s, 1H), 7.14 (s, 1H), 4.34 (s, 2H), 3.45-3.51 (m, 2H), 3.26-3.44 (m, 6H), 2.71-2.80 (m, 1H), 2.28-2.35 (m, 2H), 2.05-2.23 (m, 4H), 1.42 (s, 6H), 1.08 (ddd, J=11.5 Hz, 6.8 Hz, 4.9 Hz, 2H), 0.85 (ddd, J=10.4 Hz, 6.5 Hz, 5.0 Hz, 2H).

[M+H⁺]: m/z 523.

### Example 101

### 8-{[2-(3,5-Dimethylisooxazol-4-yl)-5-(trifluoromethyl)phenyl]methyl}-1-oxa-2,8-diazaspiro[4.5]dec-2-en-3-yl)benzoic acid

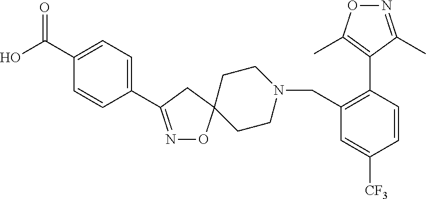 [SMILES: Cc1noc(C)c1-c1ccc(C(F)(F)F)cc1CN1CCC2(CC1)CC(c1ccc(C(=O)O)cc1)=NO2]

Using essentially the same procedure as for EXAMPLE 69, Step B, but using methyl 8-{[2-bromo-5-(trifluoromethyl)phenyl]methyl}-1-oxa-2,8-diazaspiro[4.5]dec-2-en-3-yl)benzoate (22.5 mg, 0.043 mmol) (EXAMPLE 69, Step A) and 3,5-dimethylisooxazol-4-yl)boronic acid pinacol ester (22 mg, 0.096 mmol), the title compound (7 mg) was prepared as a white solid.

[M+H⁺]: m/z 514.

### Example 102

### 8-{[2′,4′-Dichloro-3-methyl-4-(trifluoromethyl)biphen-2-yl]methyl}-1-oxa-2,8-diazaspiro[4.5]dec-2-en-3-yl)benzoic acid

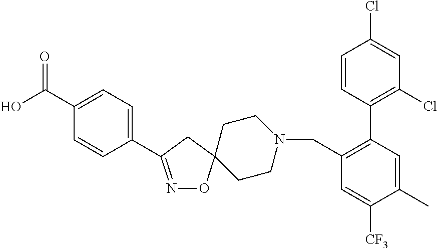 [SMILES: Cc1cc(-c2ccc(Cl)cc2Cl)c(CN2CCC3(CC2)CC(c2ccc(C(=O)O)cc2)=NO3)cc1C(F)(F)F]

Using essentially the same procedure as for EXAMPLE 68, Step B, but using methyl 8-{[2-bromo-4-methyl-5-(trifluoromethyl)phenyl]methyl}-1-oxa-2,8-diazaspiro[4.5]dec-2-en-3-yl)benzoate (22.5 mg, 0.043 mmol) (EXAMPLE 68, Step A) and 2,4-dichloroboronic acid (8.2 mg, 0.043 mmol), the title compound (5 mg) was prepared as a white solid.

[M+H⁺]: m/z 577/579.

### Example 103

### 8-{[2-Cyclopropyl-5-(trifluoromethyl)phenyl]methyl}-1-oxa-2,8-diazaspiro[4.5]dec-2-en-3-yl)benzoic acid

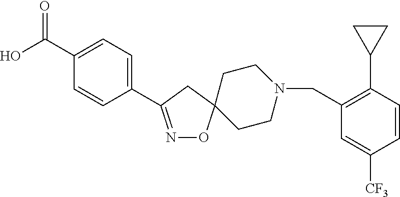 [SMILES: O=C(O)c1ccc(C2=NOC3(CCN(Cc4cc(C(F)(F)F)ccc4C4CC4)CC3)C2)cc1]

Using essentially the same procedure as for EXAMPLE 69, Step B, but using methyl 8-{[2-bromo-5-(trifluoromethyl)phenyl]methyl}-1-oxa-2,8-diazaspiro[4.5]dec-2-en-3-yl)benzoate (35 mg, 0.069 mmol) (EXAMPLE 69, Step A) and cyclopropylboronic acid pinacol ester (23 mg, 0.137 mmol), the title compound (22 mg) was prepared as a white solid.

[M+H⁺]: m/z 459.

### Biological Assays

SSTR5 antagonists can be identified using SSTR5 and nucleic acid encoding for SSTR5. Suitable assays include detecting compounds competing with a SSTR5 agonist for binding to SSTR5 and determining the functional effect of compounds on a SSTR5 cellular or physiologically relevant activity. SSTR5 cellular activities include cAMP inhibition, phospholipase C increase, tyrosine phsophatases increase, endothelial nitric oxide synthase (eNOS) decrease, K+ channel increase, Na+/H+ exchange decrease, and ERK decrease. (Lahlou et al., Ann. N.Y. Acad. Sci. 1014:121-131, 2004.) Functional activity can be determined using cell lines expressing SSTR5 and determining the effect of a compound on one or more SSTR5 activities (e.g., Poitout et al., J. Med. Chem. 44:29900-3000, (2001); Hocart et al., J. Med. Chem. 41:1146-1154, (1998); J. Med. Chem. 50, 6292-6295 (2007) and J. Med. Chem. 50, 6295-6298 (2007)).

SSTR5 binding assays can be performed by labeling somatostatin and determining the ability of a compound to inhibit somatostatin binding. (Poitout et al., J. Med. Chem. 44:29900-3000, (2001); Hocart et al., J. Med. Chem. 41:1146-1154, (1998); J. Med. Chem. 50, 6292-6295 (2007) and J. Med. Chem. 50, 6295-6298 (2007)). Additional formats for measuring binding of a compound to a receptor are well-known in the art.

A physiologically relevant activity for SSTR5 inhibition is stimulating insulin secretion. Stimulation of insulin secretion can be evaluated in vitro or in vivo.

Antagonists can be characterized based on their ability to bind to SSTR5 (Ki) and effect SSTR5 activity (IC50), and to selectively bind to SSTR5 and selectively affect SSTR5 activity. Preferred antagonists strongly and selectively bind to SSTR5 and inhibit SSTR5 activity. Ki can be measured as described by Poitout et al., J. Med. Chem. 44:29900-3000, (2001) and described herein.

A selective SSTR5 antagonist binds SSTR5 at least 10 times stronger than it binds SSTR1, SSTR2, SSTR3, and SSTR4. In different embodiments concerning selective SSTR5 binding, the antagonist binds to each of SSTR1, SSTR2, SSTR3, and SSTR4 with a Ki greater than 1000 nM, or preferably greater than 2000 nM and/or binds SSTR5 at least 40 times, more preferably at least 100 times, or more preferably at least 500 times, greater than it binds to SSTR1, SSTR2, SSTR3, and SSTR4.

IC50 can be determined by measuring inhibition of somatostatin-14 or somatostatin-28 induced reduction of cAMP accumulation due to forskolin (1 μM) in CHO-K1 cells expressing SSTR5, as described by Poitout et al., J. Med. Chem. 44:29900-3000, (2001).

### SSTR Binding Assays:

The receptor-ligand binding assays of all 5 subtype of SSTRs were performed with membranes isolated from Chinese hamster ovary (CHO)-K1 cells stably expressing the cloned human somatostatin receptors in 96-well format as previous reported. (Yang et al. PNAS 95:10836-10841, (1998), Birzin et al. Anal. Biochem. 307:159-166, (2002)).

The stable cell lines for SSTR1-SSTR5 were developed by stably transfecting with DNA for all five SSTRs using Lipofectamine. Neomycin-resistant clones were selected and maintained in medium containing 400 μg/mL G418 (Rohrer et al. Science 282:737-740, (1998)). Binding assays were performed using (3-125I-Tyr11)-SRIF-14 or (3-125I-Tyrl 1)-SRIF-28 as the radioligand (used at 0.1 nM) and The Packard Unifilter assay plate. The assay buffer consisted of 50 mM TrisHCl (pH 7.8) with 1 mM EGTA, 5 mM MgCl2, leupeptin (10 μg/mL), pepstatin (10 μg/mL), bacitracin (200 μg/mL), and aprotinin (0.5 μg/mL). CHO-K1 cell membranes, radiolabeled somatostatin, and unlabeled test compounds were resuspended or diluted in this assay buffer. Unlabeled test compounds were examined over a range of concentrations from 0.01 nM to 10,000 nM. The Ki values for compounds were determined as described by Cheng and Prusoff Biochem Pharmacol. 22:3099-3108 (1973).

The compounds of the present invention, particularly the compounds of Examples 1-17, were tested in the SSTR5 binding assay and found to have Ki values in the range of 0.1 nM to 1 μM against SSTR5 and were found to have Ki values greater than 100 nM against SSTR1, SSTR2, SSTR3, and SSTR4 receptors. Preferred compounds of the present invention were found to have Ki values in the range of 0.1 nM to 100 nM against SSTR5, and Ki values greater than 100 nM against SSTR1, SSTR2, SSTR3, and SSTR4 receptors. More preferred compounds of the present invention were found to have Ki values in the range of 0.1 nM to 10 nM against SSTR5, and Ki values greater than 100 nM against SSTR1, SSTR2, SSTR3, and SSTR4 receptors.

### Functional Assay to Assess the Inhibition of SSTR5 Mediated Cyclic AMP Production:

The effects of compounds that bind to human and murine SSTR5 with various affinities on the functional activity of the receptor were assessed by measuring cAMP production in the presence of Forskolin (FSK) alone or FSK plus SS-28 in SSTR5 expressing CHO cells. FSK acts to induce cAMP production in these cells by activating adenylate cyclases, whereas SS-28 suppresses cAMP production in the SSTR5 stable cells by binding to SSTR5 and the subsequent inhibition of adenylate cyclases via an alpha subunit of GTP-binding protein.

To measure the agonism activity of the compounds, human or mouse SSTR5 stable CHO cells were pre-incubated with the compounds for 15 min, followed by a one-hour incubation of the cells with 5 μM FSK (in the continuous presence of the compounds). The amount of cAMP produced during the incubation was quantified with the Lance cAMP assay kit (PerkinElmer, CA) according to the manufacturer's instruction, as well as, an IC50 value was obtained by an eight-point titration.

The compounds of the present invention, particularly the compounds shown in Table 4, were tested in the SSTR5 binding assay and found to have cAMP IC50 values in the range of 0.1 nM to 1 μM against SSTR5, as shown in Table 4, and were found to have cAMP IC50 values greater than 100 nM against SSTR1, SSTR2, SSTR3, and SSTR4 receptors. Preferred compounds of the present invention were found to have cAMP IC50 values in the range of 0.1 nM to 100 nM against SSTR5, and IC50 values greater than 100 nM against SSTR1, SSTR2, SSTR3, and SSTR4 receptors. More preferred compounds of the present invention were found to have cAMP IC50 values in the range of 0.1 nM to 10 nM against SSTR5, and IC50 values greater than 100 nM against SSTR1, SSTR2, SSTR3, and SSTR4 receptors.

TABLE 4

| Example | Bnd IC50 (nM) | cAMP IC50 (nM) |
|---|---|---|
| 1 | 1.2 | 0.72 |
| 2 | 4.3 | 5.2 |
| 3 | 2.8 | 8.2 |
| 4 | 2.9 | 3 |
| 5 | 6 | 2.4 |
| 10 | 1.8 | 5.7 |
| 12 | 7.5 | 1.3 |
| 13 | 2.9 | 2.9 |
| 17 | 0.69 | 1.7 |
| 18 | 12 | 11 |
| 19 | 1.5 | 0.94 |
| 24 | 2.3 | 1.8 |
| 25 | 1.2 | 2.6 |
| 26 | 0.33 | 0.32 |
| 27 | 10 | 8.8 |
| 29 | 0.99 | 5.6 |
| 30 | 0.41 | 0.76 |
| 39 | | 0.18 |
| 40 | | 0.40 |
| 41 | | 0.3 |
| 43 | | 2.5 |
| 44 | | 14.4 |
| 45 | | 9.15 |
| 46 | | 12.1 |
| 47 | | 0.28 |
| 48 | | 3.36 |
| 49 | | 2.5 |
| 50 | | 13.5 |
| 51 | | 8.32 |
| 68 | | 13.2 |
| 70 | | 26.5 |
| 85 | | 2.25 |
| 87 | | 1.5 |
| 91 | | 72.7 |
| 95 | | 18.7 |
| 96 | | 9.4 |
| 97 | | 4.05 |
| 98 | | 12.6 |
| 99 | | 472 |

### Enhancement of Glucose Dependent Insulin Secretion (GDIS) by SSTR3 Antagonists in Isolated Mouse Islet Cells:

Pancreatic islets of Langerhans were isolated from the pancreas of normal C57BL/6J mice (Jackson Laboratory, Maine) by collagenase digestion and discontinuous Ficoll gradient separation, a modification of the original method of Lacy and Kostianovsky (Lacy et al., Diabetes 16:35-39, 1967). The islets were cultured overnight in RPMI 1640 medium (11 mM glucose) before GDIS assay.

To measure GDIS, islets were first preincubated for 30 minutes in the Krebs-Ringer bicarbonate (KRB) buffer with 2 mM glucose (in petri dishes). The KRB medium contains 143.5 mM Na+, 5.8 mM K+, 2.5 mM Ca2+, 1.2 mM Mg2+, 124.1 mM Cl−, 1.2 mM PO43−, 1.2 mM SO42+, 25 mM CO32−, 2 mg/mL bovine serum albumin (pH 7.4). The islets were then transferred to a 96-well plate (one islet/well) and incubated at 37° C. for 60 minutes in 200 μl of KRB buffer with 2 or 16 mM glucose, and other agents to be tested such as octreotide and a SST3 antagonist. (Zhou et al., J. Biol. Chem. 278:51316-51323, 2003.) Insulin was measured in aliquots of the incubation buffer by ELISA with a commercial kit (ALPCO Diagnostics, Windham, N.H.).

### Glucose Tolerance Test in Mice:

Male C57BL/6N mice (7-12 weeks of age) are housed 10 per cage and given access to normal diet rodent chow and water ad libitum. Mice are randomly assigned to treatment groups and fasted 4 to 6 h. Baseline blood glucose concentrations are determined by glucometer from tail nick blood. Animals are then treated orally with vehicle (0.25% methylcellulose) or test compound. Blood glucose concentration is measured at a set time point after treatment (t=0 min) and mice are then challenged with dextrose intraperitoneally- (2-3 g/kg) or orally (3-5 g/kg). One group of vehicle-treated mice is challenged with saline as a negative control. Blood glucose levels are determined from tail bleeds taken at 20, 40, 60 minutes after dextrose challenge. The blood glucose excursion profile from t=0 to t=60 min is used to integrate an area under the curve (AUC) for each treatment. Percent inhibition values for each treatment are generated from the AUC data normalized to the saline-challenged controls. A similar assay may be performed in rats. Compounds of the present invention are active after an oral dose in the range of 0.1 to 100 mg/kg.

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are unsubstituted or film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

What is claimed is:

1. A compound of structural formula I: 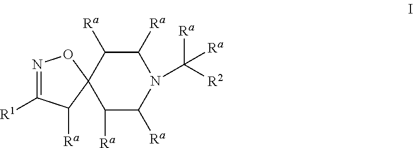
or a pharmaceutically acceptable salt thereof, wherein each occurrence of R^{a} is independently selected from the group consisting of hydrogen, halogen, C₁-C₁₀alkyl, halogen-substitutedC₁-C₁₀alkyl;
R¹ is selected from the group consisting of phenyl and heterocycle, wherein the phenyl and heterocycle are substituted with at least one substituent selected from α;
R² is selected from the group consisting of phenyl and heterocycle, wherein the phenyl and heterocycle is substituted with 1-3 substituents independently selected from α;
α is selected from the group consisting of:
halogen,
C₁-C₁₀alkyl,
halogen-substitutedC₁-C₁₀alkyl,
C₃-C₁₀cycloalkyl,
halogen-substitutedC₃-C₁₀cycloalkyl,
—OH,
—O—C₁-C₁₀alkyl,
—O-halogen-substitutedC₁-C₁₀alkyl,
—O—C₃-C₁₀cycloalkyl,
—O-halogen-substitutedC₃-C₁₀cycloalkyl,
—O-aryl,
—O-heterocycle,
—O-halogen-substituted heterocycle,
—O-halogen-substituted aryl,
—NR^{b}S(O)₂R^{d},
—NR^{b}R^{c},
—CN,
—NR^{b}C(O)R^{c},
aryl,
heterocycle,
halogen-substituted heterocycle,
C₁-C₁₀alkyl-substituted heterocycle,
halogen-substituted aryl,
—S(O)₂R^{d},
—S(O)₂NR^{b}R^{c},
—C(O)NR^{b}R^{c},
—NR^{b}C(O)OR^{c},
—NR^{b}C(O)NR^{c}R^{d},
—NR^{b}C(O)NH₂,
—NR^{b}S(O)₂R^{d},
—NO₂,
—C(O)R^{d},
—COOH,
—CO₂R^{d}, and
—OC(O)R^{d},
wherein, R^{b} and R^{c} are independently selected from the group consisting of hydrogen, C₁-C₁₀alkyl, C₃-C₁₀cycloalkyl, aryl, and heterocycle; and R^{d} is selected from the group consisting of C₁-C₁₀alkyl, C₃-C₁₀cycloalkyl, aryl, and heterocycle.

2. A compound of claim 1 or a pharmaceutically acceptable salt thereof, wherein at each occurrence of R^{a}, R^{a} is hydrogen.

3. A compound of claim 1 or a pharmaceutically acceptable salt thereof, wherein R¹ is phenyl.

4. A compound of claim 1 or a pharmaceutically acceptable salt thereof, wherein R¹ is pyridine.

5. A compound of claim 1 or a pharmaceutically acceptable salt thereof, wherein R¹ is substituted with —COOH or —O—C₁-C₁₀alkyl.

6. A compound of claim 1 or a pharmaceutically acceptable salt thereof, wherein R² is phenyl, prydine or pyrazole.

7. A compound of claim 1 or a pharmaceutically acceptable salt thereof, wherein R² is phenyl.

8. A compound of claim 1 or a pharmaceutically acceptable salt thereof, wherein R² is prydine.

9. A compound of claim 1 or a pharmaceutically acceptable salt thereof, wherein R² is pyrazole.

10. A compound of claim 1 or a pharmaceutically acceptable salt thereof, wherein R² is substituted with two substituents independently selected from α.

11. A compound of claim 1 or a pharmaceutically acceptable salt thereof, wherein R² is substituted with three substituents independently selected from α.

12. A compound of claim 1 or a pharmaceutically acceptable salt thereof, wherein R² is substituted with 1-3 substituents selected from the group consisting of halogen, C₁-C₁₀alkyl, halogen-substitutedC₁-C₁₀alkyl, —O—C₁-C₁₀alkyl, —O-halogen-substitutedC₁-C₁₀alkyl, aryl, heterocycle, halogen-substituted heterocycle, C₁-C₁₀alkyl-substituted heterocycle, halogen-substituted aryl and —COOH.

13. A compound of claim 1 or a pharmaceutically acceptable salt thereof, wherein R² is substituted with 1-3 substituents selected from the group consisting of halogen, C₁-C₁₀alkyl, —O—C₁-C₁₀alkyl, —O-halogen-substitutedC₁-C₁₀alkyl, heterocycle, halogen-substituted heterocycle or halogen-substituted aryl.

14. A compound of claim 1 or a pharmaceutically acceptable salt thereof, wherein R² is substituted with 1-3 substituents selected from the group consisting of —O—C₁-C₁₀alkyl, —O-halogen-substitutedC₁-C₁₀alkyl and halogen-substituted aryl.

15. A compound of claim 1 or pharmaceutically acceptable salt thereof selected from the group consisting of:

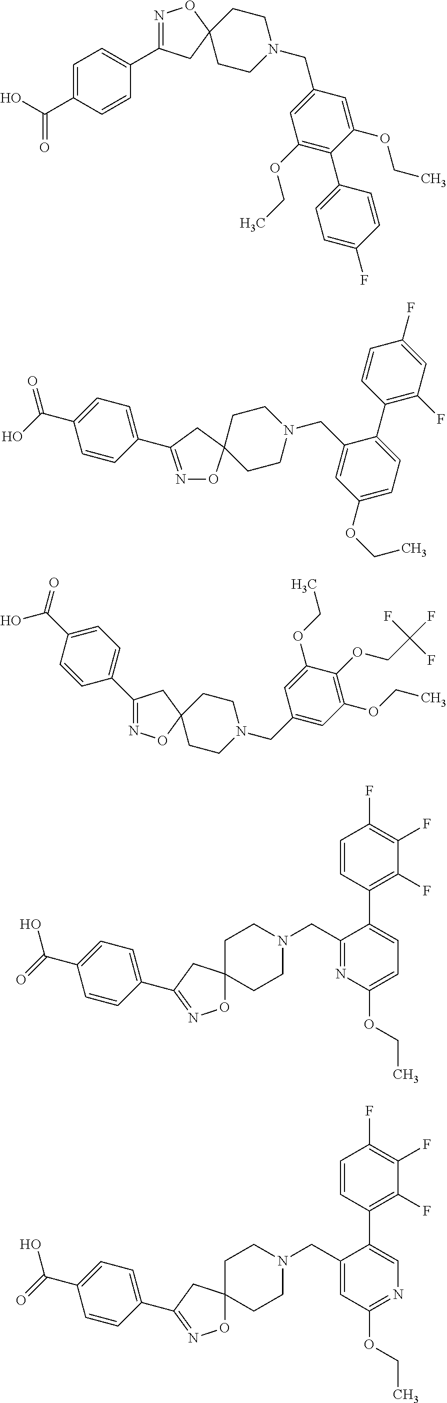 [SMILES: CCOc1cc(CN2CCC3(CC2)CC(c2ccc(C(=O)O)cc2)=NO3)cc(OCC)c1-c1ccc(F)cc1 ; CCOc1ccc(-c2ccc(F)cc2F)c(CN2CCC3(CC2)CC(c2ccc(C(=O)O)cc2)=NO3)c1 ; CCOc1cc(CN2CCC3(CC2)CC(c2ccc(C(=O)O)cc2)=NO3)cc(OCC)c1OCC(F)(F)F ; CCOc1ccc(-c2ccc(F)c(F)c2F)c(CN2CCC3(CC2)CC(c2ccc(C(=O)O)cc2)=NO3)n1 ; CCOc1cc(CN2CCC3(CC2)CC(c2ccc(C(=O)O)cc2)=NO3)c(-c2ccc(F)c(F)c2F)cn1]

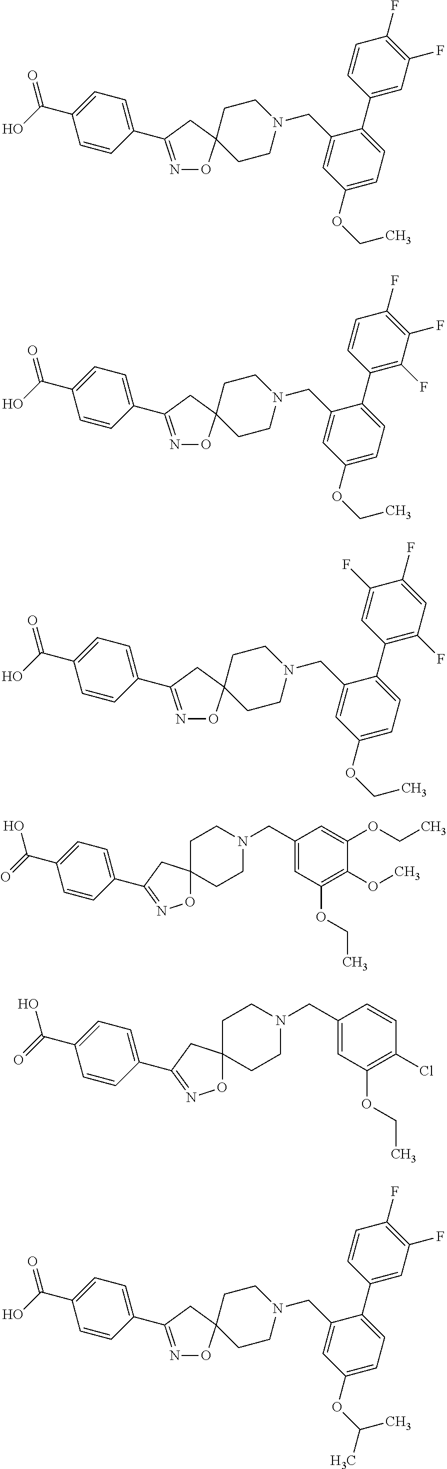

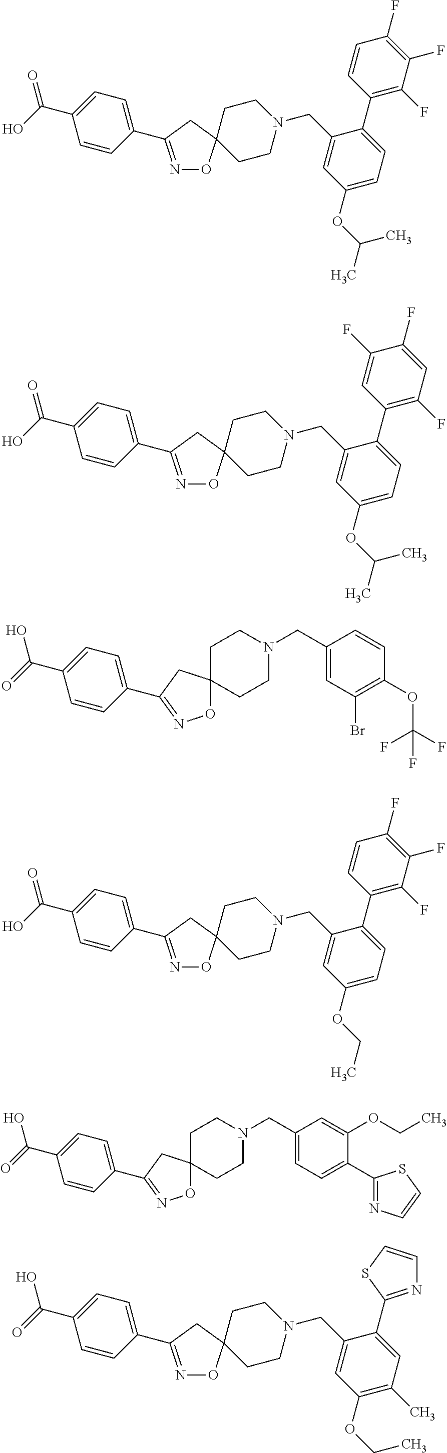

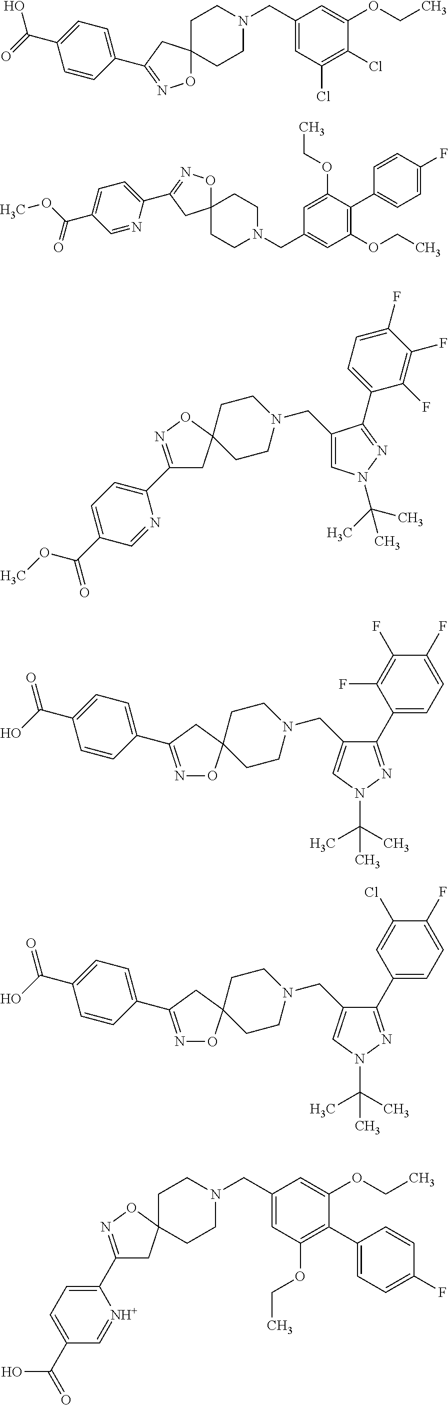

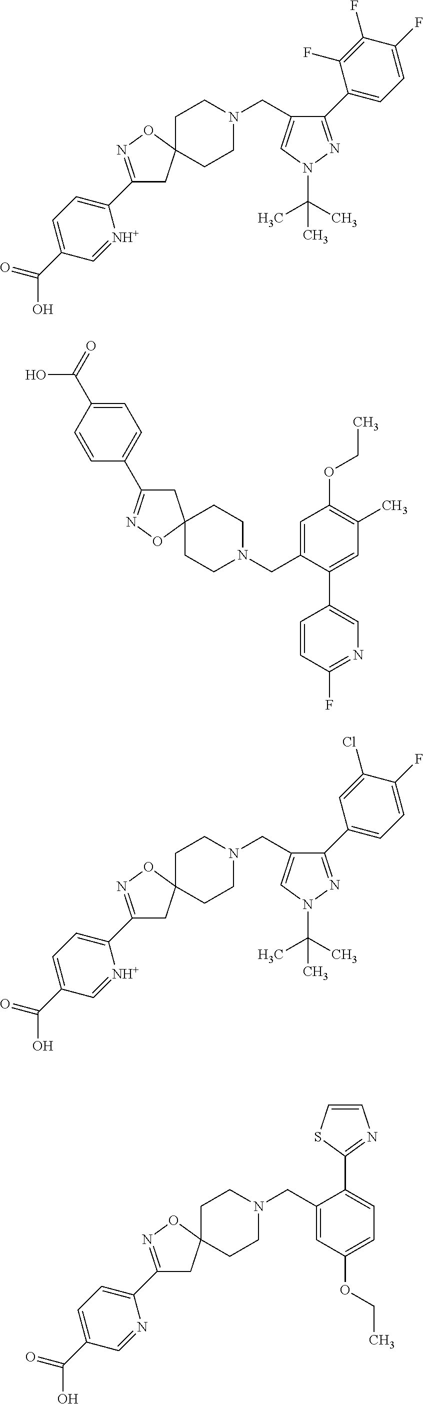 [SMILES: CC(C)(C)n1cc(CN2CCC3(CC2)CC(c2ccc(C(=O)O)c[nH+]2)=NO3)c(-c2ccc(F)c(F)c2F)n1 ; CCOc1cc(CN2CCC3(CC2)CC(c2ccc(C(=O)O)cc2)=NO3)c(-c2ccc(F)nc2)cc1C ; CC(C)(C)n1cc(CN2CCC3(CC2)CC(c2ccc(C(=O)O)c[nH+]2)=NO3)c(-c2ccc(F)c(Cl)c2)n1 ; CCOc1ccc(-c2nccs2)c(CN2CCC3(CC2)CC(c2ccc(C(=O)O)cn2)=NO3)c1]

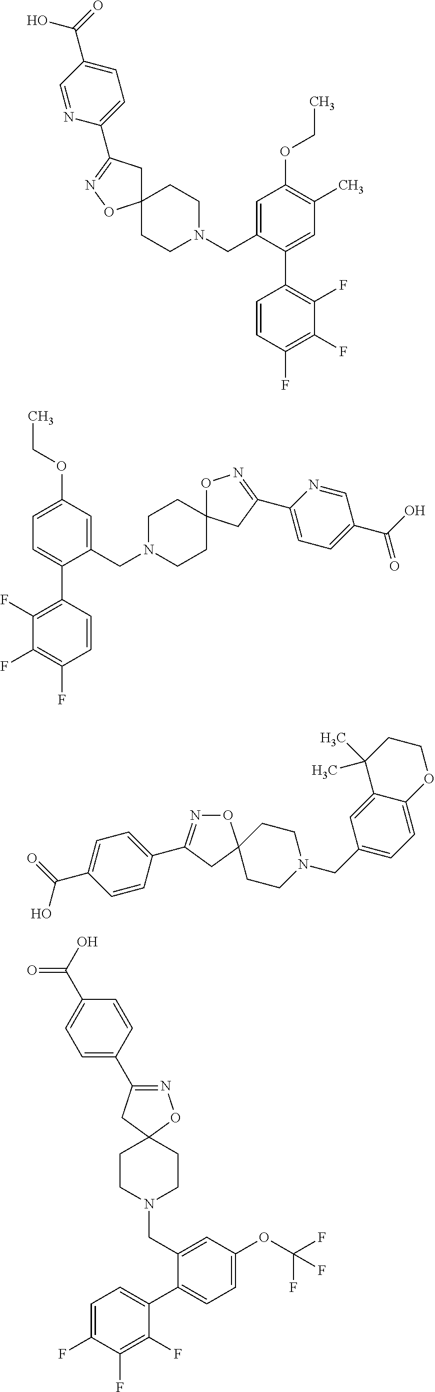

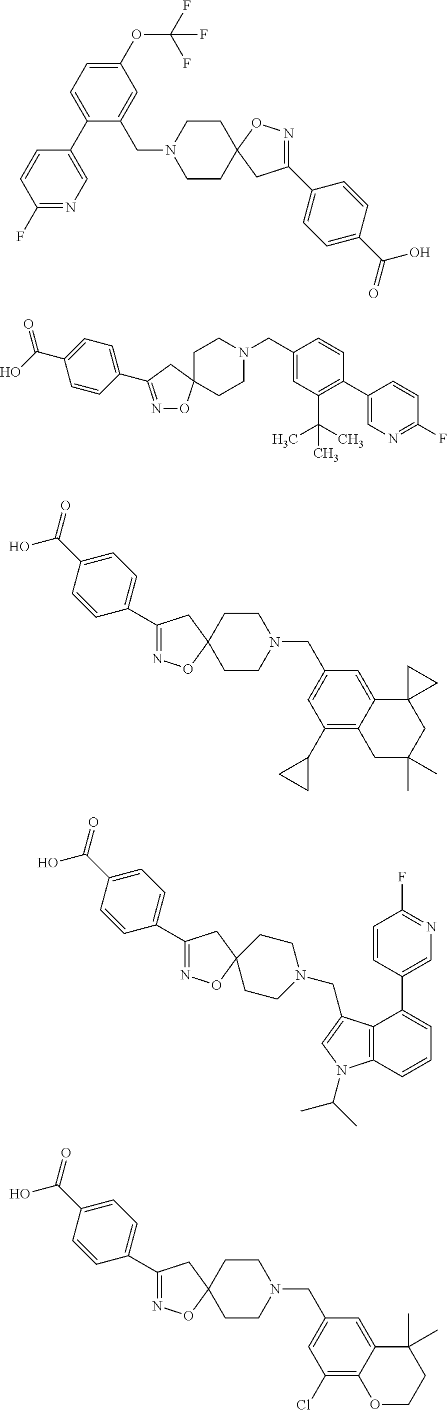 [SMILES: O=C(O)c1ccc(C2=NOC3(CCN(Cc4cc(OC(F)(F)F)ccc4-c4ccc(F)nc4)CC3)C2)cc1 ; CC(C)(C)c1cc(CN2CCC3(CC2)CC(c2ccc(C(=O)O)cc2)=NO3)ccc1-c1ccc(F)nc1 ; CC1(C)Cc2c(C3CC3)cc(CN3CCC4(CC3)CC(c3ccc(C(=O)O)cc3)=NO4)cc2C2(CC2)C1 ; CC(C)n1cc(CN2CCC3(CC2)CC(c2ccc(C(=O)O)cc2)=NO3)c2c(-c3ccc(F)nc3)cccc21 ; CC1(C)CCOc2c(Cl)cc(CN3CCC4(CC3)CC(c3ccc(C(=O)O)cc3)=NO4)cc21]

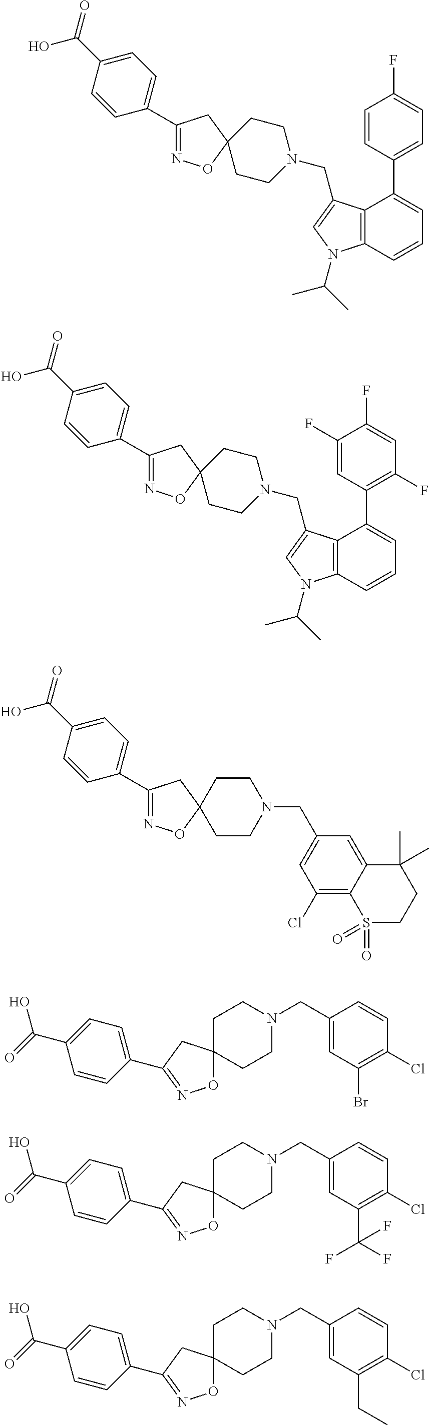

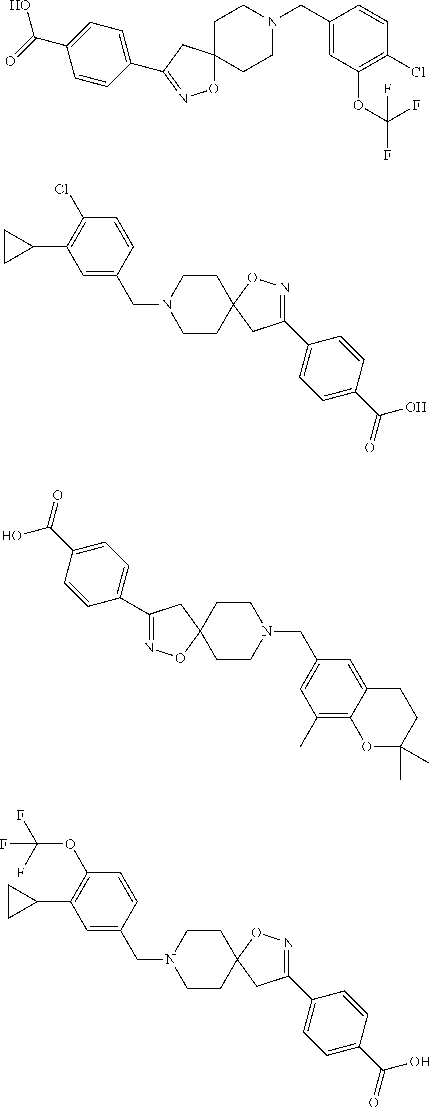 [SMILES: O=C(O)c1ccc(C2=NOC3(CCN(Cc4ccc(Cl)c(OC(F)(F)F)c4)CC3)C2)cc1 ; O=C(O)c1ccc(C2=NOC3(CCN(Cc4ccc(Cl)c(C5CC5)c4)CC3)C2)cc1 ; Cc1cc(CN2CCC3(CC2)CC(c2ccc(C(=O)O)cc2)=NO3)cc2c1OC(C)(C)CC2 ; O=C(O)c1ccc(C2=NOC3(CCN(Cc4ccc(OC(F)(F)F)c(C5CC5)c4)CC3)C2)cc1]

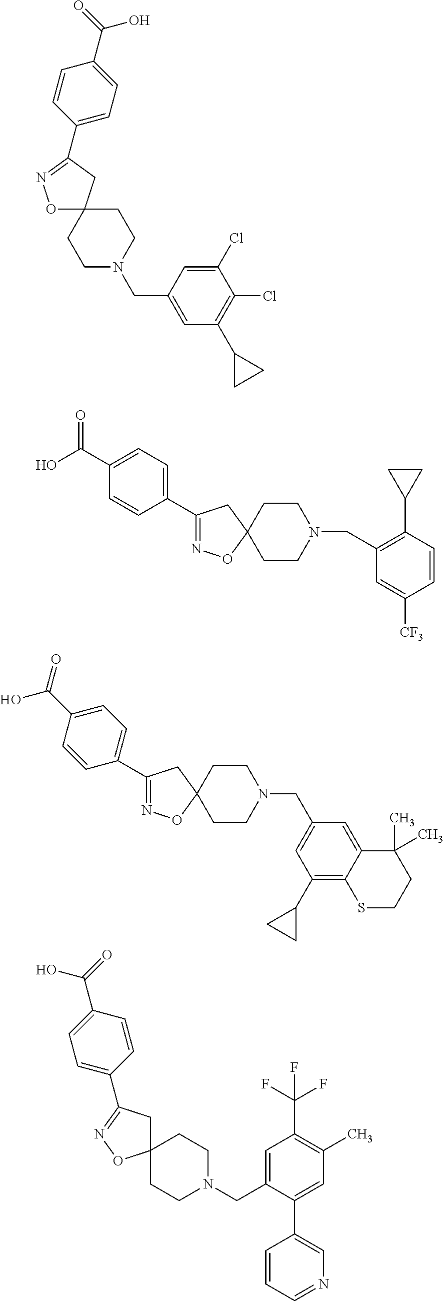 [SMILES: O=C(O)c1ccc(C2=NOC3(CCN(Cc4cc(Cl)c(Cl)c(C5CC5)c4)CC3)C2)cc1 ; O=C(O)c1ccc(C2=NOC3(CCN(Cc4cc(C(F)(F)F)ccc4C4CC4)CC3)C2)cc1 ; CC1(C)CCSc2c(C3CC3)cc(CN3CCC4(CC3)CC(c3ccc(C(=O)O)cc3)=NO4)cc21 ; Cc1cc(-c2cccnc2)c(CN2CCC3(CC2)CC(c2ccc(C(=O)O)cc2)=NO3)cc1C(F)(F)F]

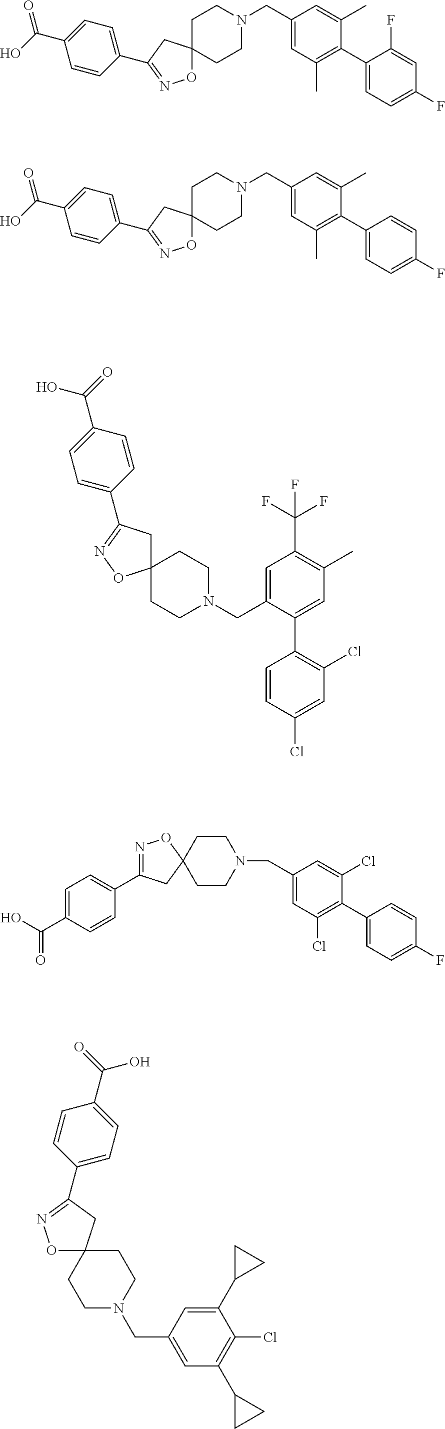 [SMILES: Cc1cc(CN2CCC3(CC2)CC(c2ccc(C(=O)O)cc2)=NO3)cc(C)c1-c1ccc(F)cc1F ; Cc1cc(CN2CCC3(CC2)CC(c2ccc(C(=O)O)cc2)=NO3)cc(C)c1-c1ccc(F)cc1 ; Cc1cc(-c2ccc(Cl)cc2Cl)c(CN2CCC3(CC2)CC(c2ccc(C(=O)O)cc2)=NO3)cc1C(F)(F)F ; O=C(O)c1ccc(C2=NOC3(CCN(Cc4cc(Cl)c(-c5ccc(F)cc5)c(Cl)c4)CC3)C2)cc1 ; O=C(O)c1ccc(C2=NOC3(CCN(Cc4cc(C5CC5)c(Cl)c(C5CC5)c4)CC3)C2)cc1]

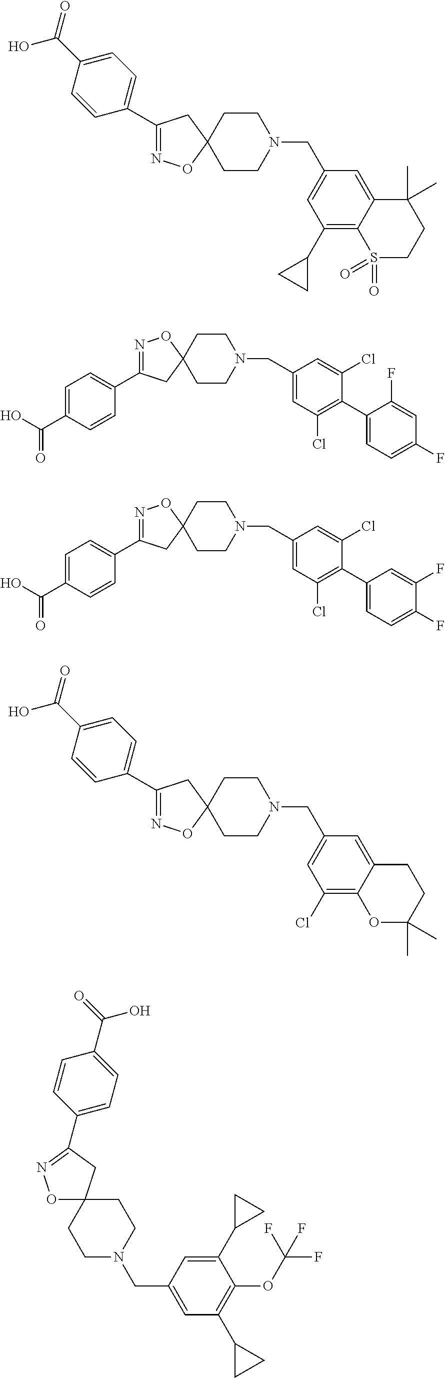 [SMILES: CC1(C)CCS(=O)(=O)c2c(C3CC3)cc(CN3CCC4(CC3)CC(c3ccc(C(=O)O)cc3)=NO4)cc21 ; O=C(O)c1ccc(C2=NOC3(CCN(Cc4cc(Cl)c(-c5ccc(F)cc5F)c(Cl)c4)CC3)C2)cc1 ; O=C(O)c1ccc(C2=NOC3(CCN(Cc4cc(Cl)c(-c5ccc(F)c(F)c5)c(Cl)c4)CC3)C2)cc1 ; CC1(C)CCc2cc(CN3CCC4(CC3)CC(c3ccc(C(=O)O)cc3)=NO4)cc(Cl)c2O1 ; O=C(O)c1ccc(C2=NOC3(CCN(Cc4cc(C5CC5)c(OC(F)(F)F)c(C5CC5)c4)CC3)C2)cc1]

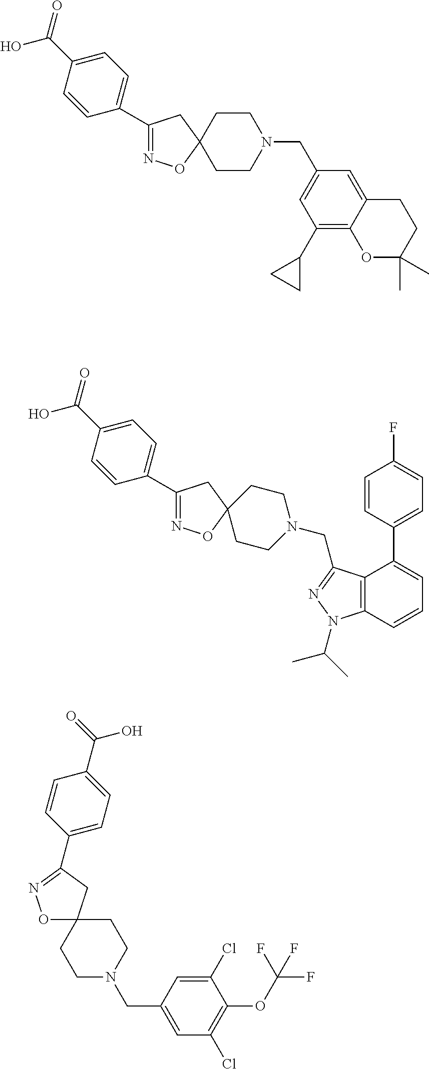 [SMILES: CC1(C)CCc2cc(CN3CCC4(CC3)CC(c3ccc(C(=O)O)cc3)=NO4)cc(C3CC3)c2O1 ; CC(C)n1nc(CN2CCC3(CC2)CC(c2ccc(C(=O)O)cc2)=NO3)c2c(-c3ccc(F)cc3)cccc21 ; O=C(O)c1ccc(C2=NOC3(CCN(Cc4cc(Cl)c(OC(F)(F)F)c(Cl)c4)CC3)C2)cc1]

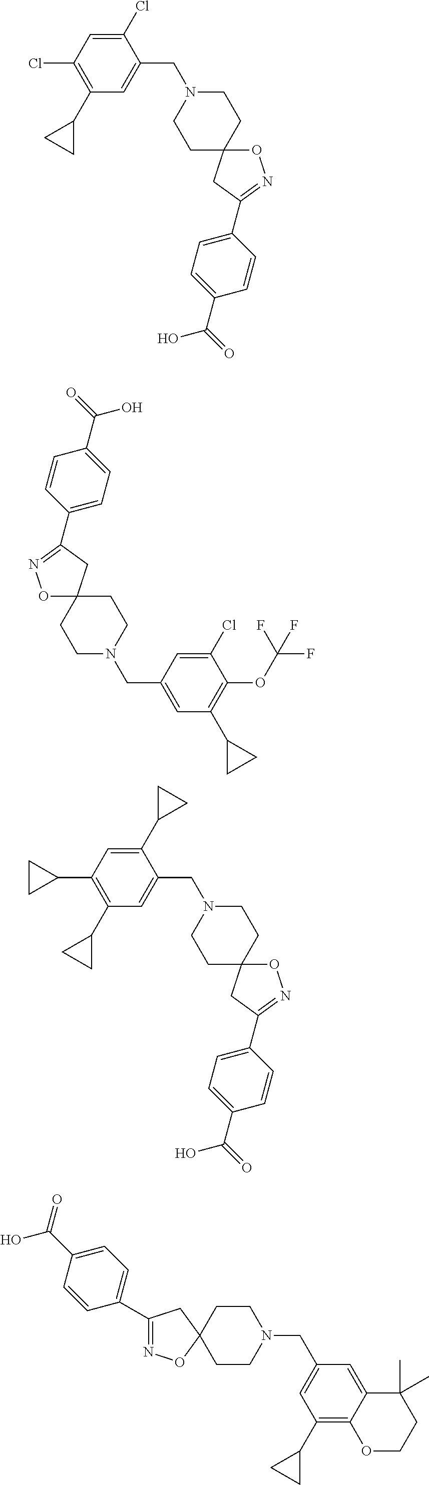 [SMILES: O=C(O)c1ccc(C2=NOC3(CCN(Cc4cc(C5CC5)c(Cl)cc4Cl)CC3)C2)cc1 ; O=C(O)c1ccc(C2=NOC3(CCN(Cc4cc(Cl)c(OC(F)(F)F)c(C5CC5)c4)CC3)C2)cc1 ; O=C(O)c1ccc(C2=NOC3(CCN(Cc4cc(C5CC5)c(C5CC5)cc4C4CC4)CC3)C2)cc1 ; CC1(C)CCOc2c(C3CC3)cc(CN3CCC4(CC3)CC(c3ccc(C(=O)O)cc3)=NO4)cc21]

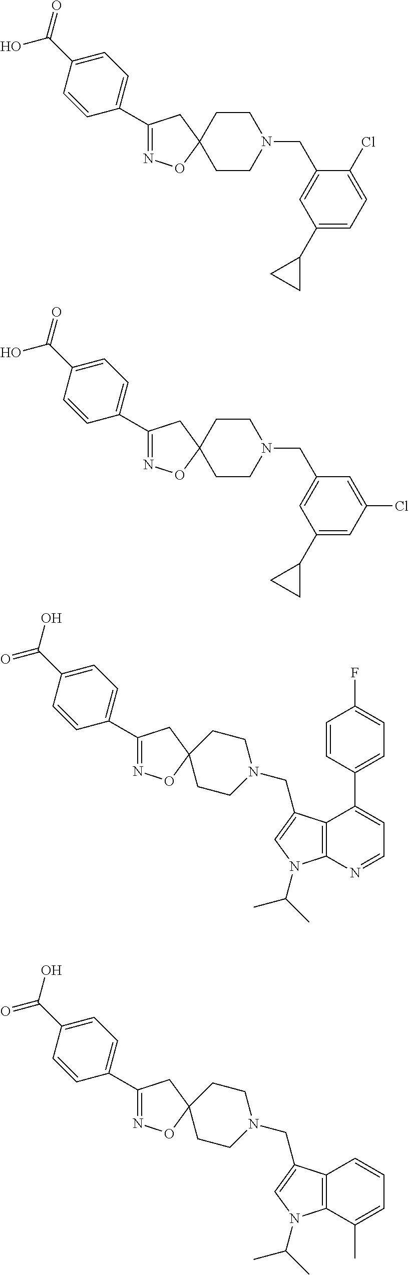 [SMILES: O=C(O)c1ccc(C2=NOC3(CCN(Cc4cc(C5CC5)ccc4Cl)CC3)C2)cc1 ; O=C(O)c1ccc(C2=NOC3(CCN(Cc4cc(Cl)cc(C5CC5)c4)CC3)C2)cc1 ; CC(C)n1cc(CN2CCC3(CC2)CC(c2ccc(C(=O)O)cc2)=NO3)c2c(-c3ccc(F)cc3)ccnc21 ; Cc1cccc2c(CN3CCC4(CC3)CC(c3ccc(C(=O)O)cc3)=NO4)cn(C(C)C)c12]

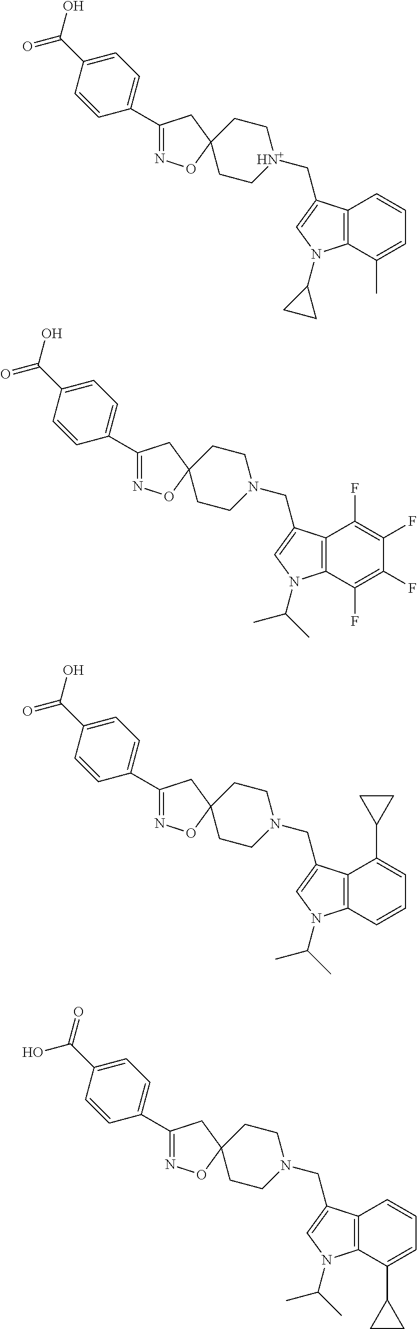 [SMILES: Cc1cccc2c(C[NH+]3CCC4(CC3)CC(c3ccc(C(=O)O)cc3)=NO4)cn(C3CC3)c12 ; CC(C)n1cc(CN2CCC3(CC2)CC(c2ccc(C(=O)O)cc2)=NO3)c2c(F)c(F)c(F)c(F)c21 ; CC(C)n1cc(CN2CCC3(CC2)CC(c2ccc(C(=O)O)cc2)=NO3)c2c(C3CC3)cccc21 ; CC(C)n1cc(CN2CCC3(CC2)CC(c2ccc(C(=O)O)cc2)=NO3)c2cccc(C3CC3)c21]

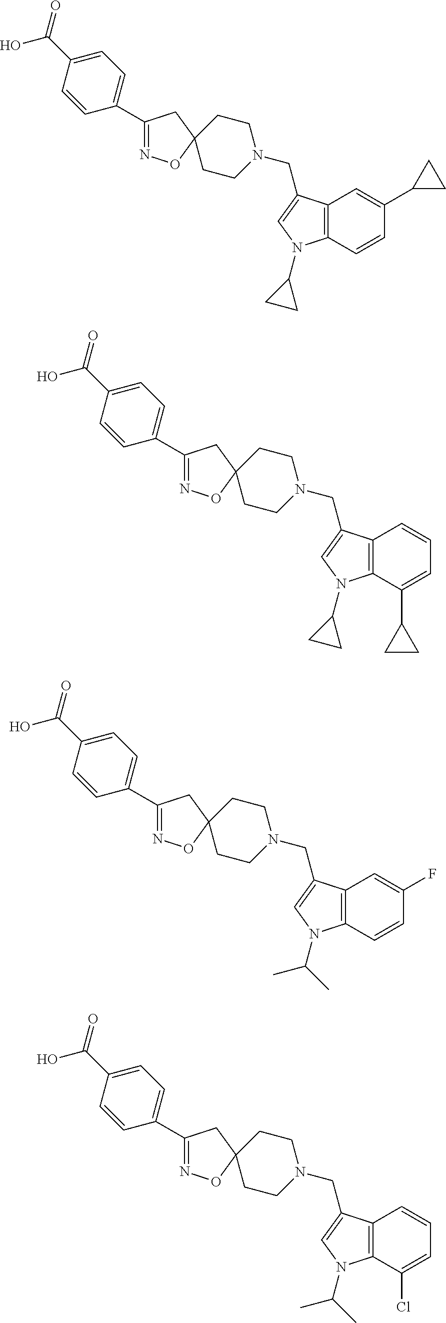 [SMILES: O=C(O)c1ccc(C2=NOC3(CCN(Cc4cn(C5CC5)c5ccc(C6CC6)cc45)CC3)C2)cc1 ; O=C(O)c1ccc(C2=NOC3(CCN(Cc4cn(C5CC5)c5c(C6CC6)cccc45)CC3)C2)cc1 ; CC(C)n1cc(CN2CCC3(CC2)CC(c2ccc(C(=O)O)cc2)=NO3)c2cc(F)ccc21 ; CC(C)n1cc(CN2CCC3(CC2)CC(c2ccc(C(=O)O)cc2)=NO3)c2cccc(Cl)c21]

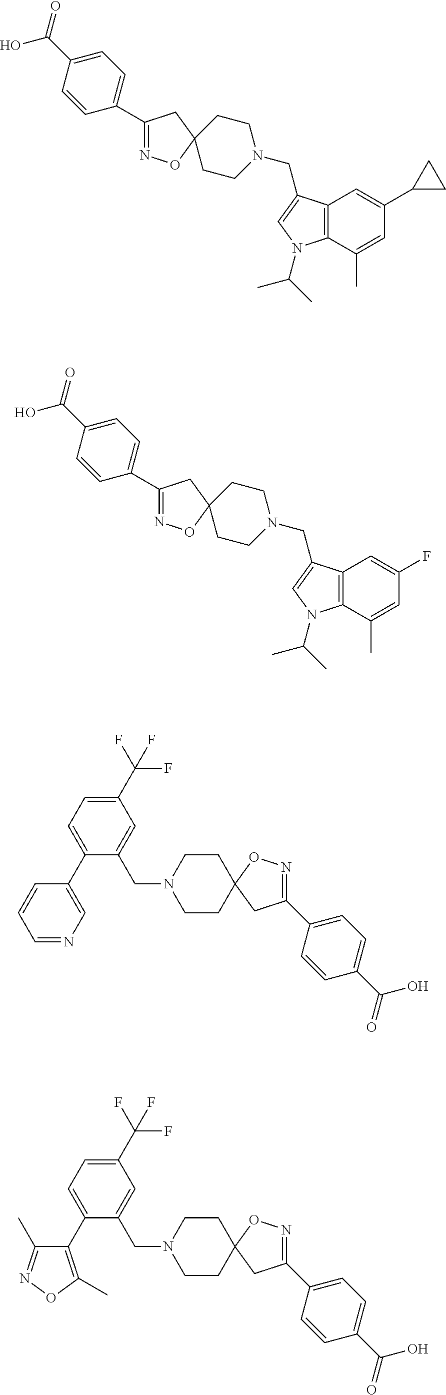 [SMILES: Cc1cc(C2CC2)cc2c(CN3CCC4(CC3)CC(c3ccc(C(=O)O)cc3)=NO4)cn(C(C)C)c12 ; Cc1cc(F)cc2c(CN3CCC4(CC3)CC(c3ccc(C(=O)O)cc3)=NO4)cn(C(C)C)c12 ; O=C(O)c1ccc(C2=NOC3(CCN(Cc4cc(C(F)(F)F)ccc4-c4cccnc4)CC3)C2)cc1 ; Cc1noc(C)c1-c1ccc(C(F)(F)F)cc1CN1CCC2(CC1)CC(c1ccc(C(=O)O)cc1)=NO2]

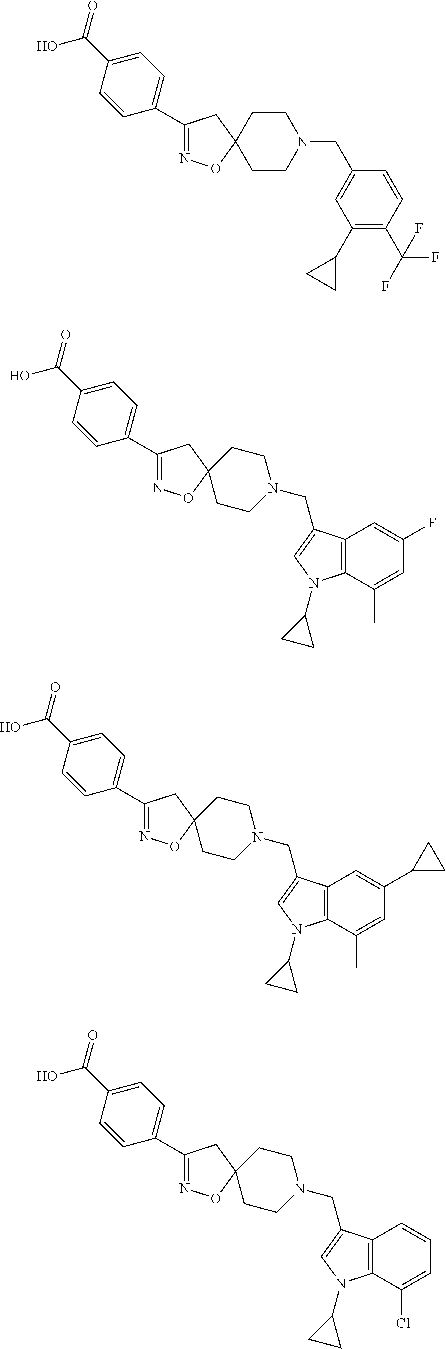 [SMILES: O=C(O)c1ccc(C2=NOC3(CCN(Cc4ccc(C(F)(F)F)c(C5CC5)c4)CC3)C2)cc1 ; Cc1cc(F)cc2c(CN3CCC4(CC3)CC(c3ccc(C(=O)O)cc3)=NO4)cn(C3CC3)c12 ; Cc1cc(C2CC2)cc2c(CN3CCC4(CC3)CC(c3ccc(C(=O)O)cc3)=NO4)cn(C3CC3)c12 ; O=C(O)c1ccc(C2=NOC3(CCN(Cc4cn(C5CC5)c5c(Cl)cccc45)CC3)C2)cc1]

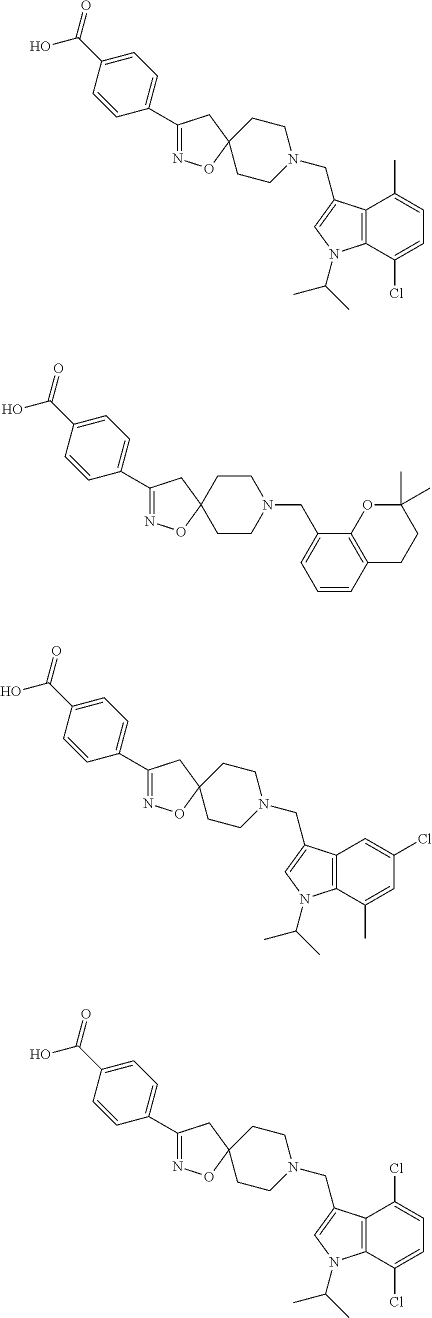 [SMILES: Cc1ccc(Cl)c2c1c(CN1CCC3(CC1)CC(c1ccc(C(=O)O)cc1)=NO3)cn2C(C)C ; CC1(C)CCc2cccc(CN3CCC4(CC3)CC(c3ccc(C(=O)O)cc3)=NO4)c2O1 ; Cc1cc(Cl)cc2c(CN3CCC4(CC3)CC(c3ccc(C(=O)O)cc3)=NO4)cn(C(C)C)c12 ; CC(C)n1cc(CN2CCC3(CC2)CC(c2ccc(C(=O)O)cc2)=NO3)c2c(Cl)ccc(Cl)c21]

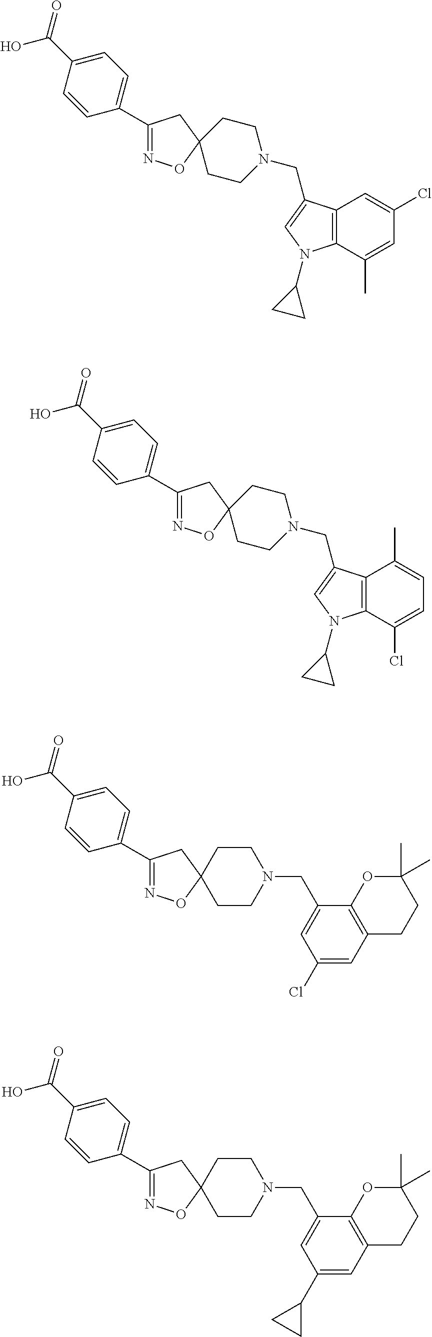 [SMILES: Cc1cc(Cl)cc2c(CN3CCC4(CC3)CC(c3ccc(C(=O)O)cc3)=NO4)cn(C3CC3)c12 ; Cc1ccc(Cl)c2c1c(CN1CCC3(CC1)CC(c1ccc(C(=O)O)cc1)=NO3)cn2C1CC1 ; CC1(C)CCc2cc(Cl)cc(CN3CCC4(CC3)CC(c3ccc(C(=O)O)cc3)=NO4)c2O1 ; CC1(C)CCc2cc(C3CC3)cc(CN3CCC4(CC3)CC(c3ccc(C(=O)O)cc3)=NO4)c2O1]

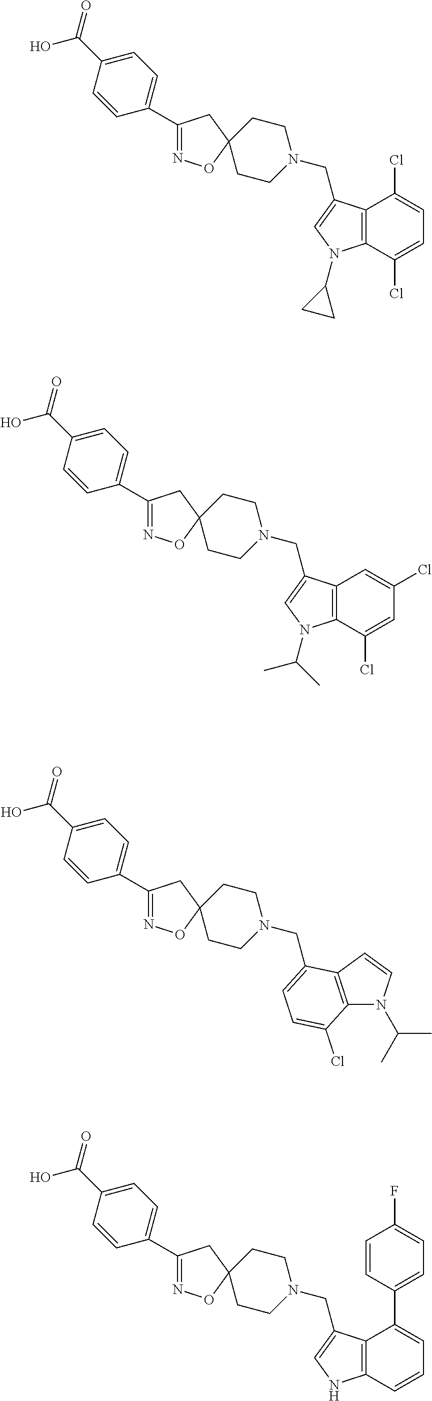 [SMILES: O=C(O)c1ccc(C2=NOC3(CCN(Cc4cn(C5CC5)c5c(Cl)ccc(Cl)c45)CC3)C2)cc1 ; CC(C)n1cc(CN2CCC3(CC2)CC(c2ccc(C(=O)O)cc2)=NO3)c2cc(Cl)cc(Cl)c21 ; CC(C)n1ccc2c(CN3CCC4(CC3)CC(c3ccc(C(=O)O)cc3)=NO4)ccc(Cl)c21 ; O=C(O)c1ccc(C2=NOC3(CCN(Cc4c[nH]c5cccc(-c6ccc(F)cc6)c45)CC3)C2)cc1]

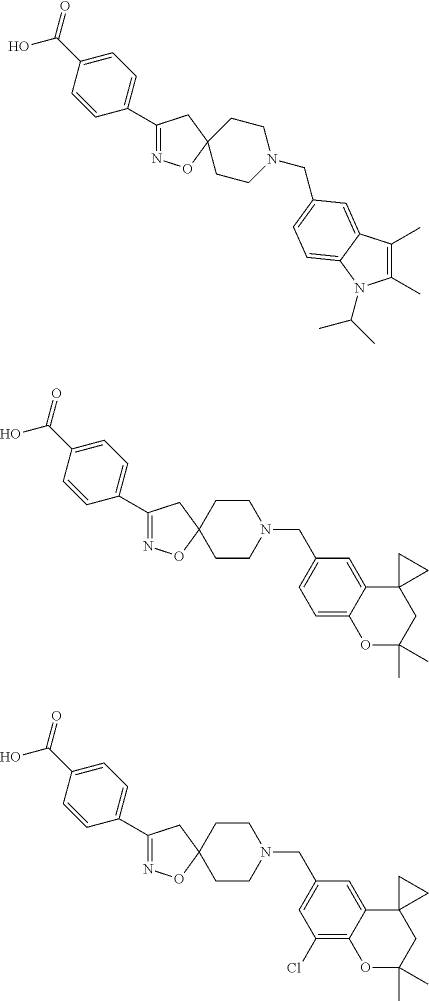 [SMILES: Cc1c(C)n(C(C)C)c2ccc(CN3CCC4(CC3)CC(c3ccc(C(=O)O)cc3)=NO4)cc12 ; CC1(C)CC2(CC2)c2cc(CN3CCC4(CC3)CC(c3ccc(C(=O)O)cc3)=NO4)ccc2O1 ; CC1(C)CC2(CC2)c2cc(CN3CCC4(CC3)CC(c3ccc(C(=O)O)cc3)=NO4)cc(Cl)c2O1]

16. A compound or pharmaceutically acceptable salt thereof selected from the group consisting of:

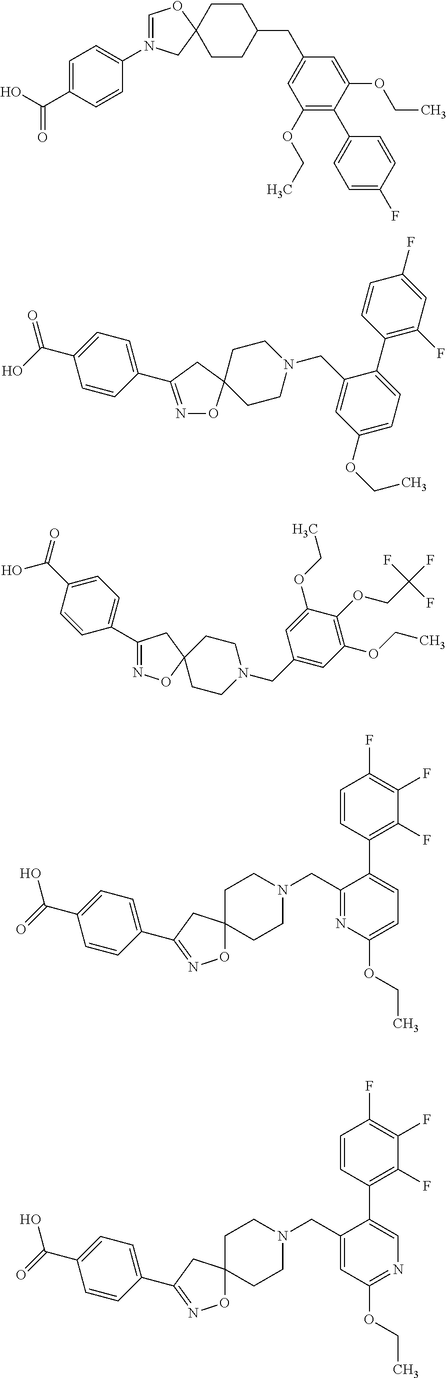

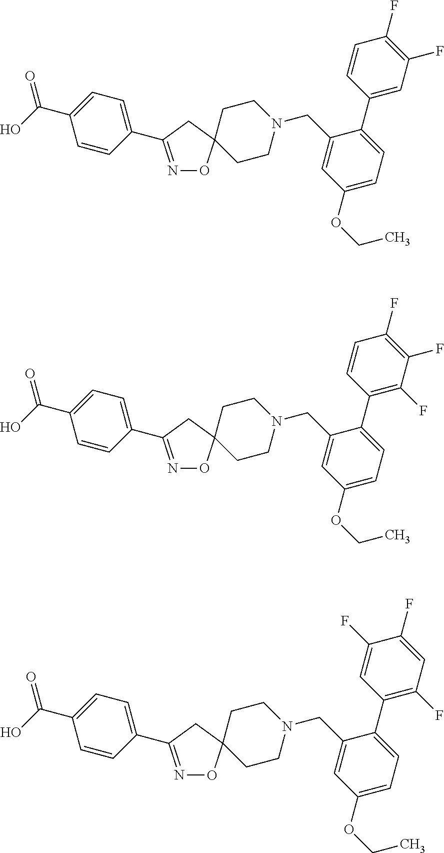 [SMILES: CCOc1ccc(-c2ccc(F)c(F)c2)c(CN2CCC3(CC2)CC(c2ccc(C(=O)O)cc2)=NO3)c1 ; CCOc1ccc(-c2ccc(F)c(F)c2F)c(CN2CCC3(CC2)CC(c2ccc(C(=O)O)cc2)=NO3)c1 ; CCOc1ccc(-c2cc(F)c(F)cc2F)c(CN2CCC3(CC2)CC(c2ccc(C(=O)O)cc2)=NO3)c1]

17. A method of treating a disorder, condition, or disease selected from the group consisting of Type 2 diabetes, insulin resistance, a lipid disorder, obesity, Metabolic Syndrome, depression and anxiety comprising administering a compound of claim 1 to a subject in need thereof.

18. (canceled)
